(19) United States
(12) Patent Application Publication
(10) Pub. No.: US 2023/0157955 A1
(43) Pub. Date: May 25, 2023

(54) VESICLE COMPOSITIONS FOR ORAL DELIVERY

(71) Applicant: PURETECH LYT, INC., Boston, MA (US)

(72) Inventors: Joseph BOLEN, Boston, MA (US); Rishab SHYAM, Arlington, MA (US); Roman BOGORAD, Newton, MA (US); Katerina KRUMOVA, Swampscott, MA (US); Bhushan PATTNI, Canton, MA (US); Nicholas PILLA, Cambridge, MA (US); Amit SINGH, Quincy, MA (US)

(21) Appl. No.: 17/791,411

(22) PCT Filed: Jan. 8, 2021
(86) PCT No.: PCT/US2021/012796

§ 371 Date: Jul. 7, 2022

Related U.S. Application Data
(60) Provisional application No. 62/958,681, filed on Jan. 8, 2020.
(60) Provisional application No. 62/959,107, filed on Jan. 9, 2020.
(60) Provisional application No. 63/007,314, filed on Apr. 8, 2020.
(60) Provisional application No. 63/113,737, filed on Nov. 13, 2020.
(60) Provisional application No. 63/113,786, filed on Nov. 13, 2020.

(51) Int. Cl. A61K 9/127; A61K 9/48; A61K 9/00

(52) U.S. Cl. CPC A61K 9/1276; A61K 9/0053; A61K 9/1271; A61K 9/4825

(57) ABSTRACT

Cargo-loaded vesicles and compositions comprising such vesicles for oral delivery are provided, wherein the vesicles comprise one or more components from milk purified vesicles. Methods for producing such cargo loaded vesicles are also provided.

### CROSS-REFERENCE TO RELATED APPLICATIONS

This application claims the benefit of priority to U.S. Provisional Pat. Application No. 62/958,681, filed Jan. 8, 2020, U.S. Provisional Pat. Application No. 62/959,107, filed Jan. 9, 2020, U.S. Provisional Pat. Application No. 63/007,314, filed Apr. 8, 2020, U.S. Provisional Pat. Application No. 63/113,737, filed Nov. 13, 2020, and U.S. Provisional Pat. Application No. 63/113,786, filed Nov. 13, 2020. The entire contents of each of the prior application are hereby incorporated by reference in their entireties.

### BACKGROUND OF THE INVENTION

Recent years have seen tremendous development of biologics and related therapeutic agents to treat, diagnose, and monitor disease. However, the challenge of generating suitable vehicles to package, stabilize and deliver payloads to sites of interest remains unaddressed. Many therapeutics suffer from degradation due to their inherent instability and active clearance mechanisms in vivo. Poor in vivo stability is particularly problematic when delivering these payloads orally. The harsh conditions of the digestive tract, including acidic conditions in the stomach, peristaltic motions coupled with the action of proteases, lipases, amylases, and nucleases that break down ingested components in the gastrointestinal tract, make it particularly challenging to deliver many biologics orally. The scale of this challenge is evidenced by the number of biologics limited to delivery via non-oral means, including IV, transdermal, and sub-cutaneous administration. A general oral delivery vehicle for biologics and related therapeutic agents would profoundly impact healthcare.

Recent efforts have focused on the packaging of biologics into polymer-based, liposomal, or biodegradable and erodible matrices that result in biologic-encapsulated nanoparticles. Despite their advantageous encapsulation properties, such nanoparticles have not achieved widespread use due to toxicity or poor release properties. Additionally, current nanoparticle synthesis techniques are limited in their ability to scale for manufacturing purposes, and are not capable of oral delivery. The development of an effective, non-toxic, and scalable delivery platform thus remains an unmet need.

Milk, which is orally ingested and known to contain a variety of miRNAs important for immune development, protects and delivers these miRNAs in exosomes. Milk vesicles therefore represent a gastrointestinally-privileged (GI-privileged), evolutionarily conserved means of communicating important messages from mother to baby while maintaining the integrity of these complex biomolecules. As one example, milk exosomes have been observed to have a favorable stability profile at acidic pH and other high-stress or degradative conditions (See, e.g., Int J Biol Sci. 2012; 8(1): 118-23. Epub 2011 Nov 29). Additionally, bovine miRNA levels in circulation have been observed to increase in a dose-dependent manner after consuming varying quantities of milk (See, e.g., PLoS One 2015; 10(3): e0121123).

### SUMMARY OF THE INVENTION

The present disclosure is based, at least in part, on the development of cargo-loaded vesicles for oral delivery of a cargo, e.g., a therapeutic cargo (e.g., nucleic acid-based or protein-based) to sites of interest. In particular, the methods and compositions disclosed herein address the challenges associated with packaging, stabilizing and oral delivery of therapeutics, which suffer from degradation due to their inherent instability and active in vivo clearance mechanisms. Such vesicles may comprise one or more components from milk purified vesicles (MPVs), which may be modified as compared with the counterpart vesicles found in milk. The vesicles disclosed herein may be loaded with various types of cargos (e.g., hydrophobic, hydrophylic, and/or anionic cargos) and/or cargos of various sizes and structures. In some embodiments, the cargo loaded into the vesicle can be a peptide, a protein, a nucleic acid, a polysaccharide, or a small molecule.

Accordingly, one aspect of the present disclosure features a cargo-loaded vesicle, and compositions of such cargo-loaded vesicles. The cargo-loaded vesicle comprises: (i) one or more component(s) of a lipid nanoparticle (LNP); and (ii) one or more component(s) of a milk purified vesicle (MPV). Such vesicles are referred to herein as “LNP-MPVs”. In some embodiments, a vesicle of the disclosure comprises one or more components of an MPV, which is a whey purified vesicle (WPV). In some embodiments, the MPVs for making the LNP-MPVs disclosed herein are modified as compared with the natural counterparts.

In some embodiments, the vesicle comprises one or more components of an LNP, which is a liposome, a multilamellar vesicle, or a solid lipid nanoparticle. In some embodiments, the LNP comprises one or more cationic lipids. In some embodiments, the one or more cationic lipids are non-ionizable cationic lipids. Non-limiting examples of such non-ionizable cationic lipids include DOTAP, DODAC, DOTMA, DDAB, DOSPA, DMRIE, DORIE, DOMPAQ, DOAAQ, DC-6-14, DOGS, and DODMA-AN. In other embodiments, the one or more cationic lipids are ionizable cationic lipids. Non-limiting examples of such ionizable cationic lipids include KL10, KL22, DLin-DMA, DLin-K-DMA, DLin-MC3-DMA, DLin-KC2-DMA, DODAP, DODMA, and DSDMA.

In some embodiments, the vesicle of the disclosure comprises an LNP comprising one or more phospholipids. Non-limiting examples of such phospholipids include 1,2-Dioleoyl-sn-glycero-3-phosphocholine (DOPC), 1,2-distearoyl-sn-glycero-3-phosphocholine (DSPC), 1,2-Dioleoyl-sn-glycero-3-phosphoserine (DOPS), PEG-1,2-Distearoyl-sn-glycero-3-phosphoethanolamine (PEG-DSPE), 1,2-Dioleoyl-sn-glycero-3-phosphoethanolamine (DOPE), 1,2-Dipalmitoyl-sn-glycero-3-phosphoethanolamine-PEG, 1,2-Bis(diphenylphosphino)ethane (DPPE)-PEG, GL67A-DOPE-DMPE-PEG, and any combination thereof.

In some embodiments, the vesicle of the disclosure comprises an LNP comprising cholesterol, or DC-cholesterol. In some embodiments, the LNP comprises:
(a) about 50 mol % to about 70 mol % of DOPC,
(b) about 10 mol % to about 50 mol % of cholesterol,
(c) about 5 mol % to about 50 mol % of DOTAP and/or DODMA,
(d) about 5 mol % to about 30 mol % of DOPE, DSPC, and/or DOPC,
(e) about 0.5-10 mol % of DPPC-PEG and/or DSPE-PEG; or
(f) a combination thereof.

In some embodiments, the LNP comprises about 50 mol % to about 70 mol % of DOPC, about 10 mol % to about 30 mol % of cholesterol, about 5 mol % to about 15 mol % of DOTAP, from about 5 mol % to about 15 mol % of DOPE, and about 0.5 mol % to about 3.0 mol % of DPPE-PEG2000.

In some embodiments, the LNP comprises about 10-50 mol% of a cationic lipid, about 20-40 mol% cholesterol, and about 0.5-3.0 mol% lipid-mPEG2000. In some embodiments, the cationic lipid is DOTAP or DODMA. In some embodiments, the lipid in the lipid-mPEG2000 is DSPE, DMPE, DMPG, or a combination thereof.

In some embodiments, the LNP further comprises a dye-conjugated helper lipid at about 0.2-1 mol%. In some embodiments, the helper lipid is DPPE. In some embodiments, the lipid content in the LNP is substantially similar to the lipid content in the MPV.

Any of the LNP components disclosed here can be included in the cargo-loaded vesicles disclosed herein.

In some embodiments, the cargo-loaded vesicles disclosed herein may further comprise one or more binding moieties on the surface of the vesicle. In some embodiments, the binding moiety is a lectin. Non-limiting examples of such lectins include Con A, RCA, WGA, DSL, Jacalin, and any combination thereof. In some embodiments, the lectin is covalently attached to the vesicle surface. In some embodiments, the lectin is attached to the surface of the cargo-loaded vesicle through a biotin-streptavidin linkage.

In some embodiments, the vesicle of the disclosure comprises components from MPVs (e.g., WPVs). The size of the MPVs may be about 20-1,000 nm. In some examples, the size of the MPV is about 80-200 nm. In some examples, the size of the MPV is about 100-160 nm.

In some embodiments, the MPV comprises a lipid membrane to which one or more proteins are associated. Non-limiting examples of the one or more proteins associated with the lipid membrane of the MPV include Butyrophilin Subfamily 1 Member A1 (BTN1A1) or a transmembrane fragment thereof, Butyrophilin Subfamily 1 Member A2 (BTN1A2) or a transmembrane fragment thereof, fatty acid binding protein, lactadherin, platelet glycoprotein 4, xanthine dehydrogenase, ATP-binding cassette subfamily G, perilipin, RAB1A, peptidyl-prolyl cis-transisomerase A, Ras-related protein Rab-18, EpCAM, CD63, CD81, TSG101, HSP70, lactoferrin or a transmembrane fragment thereof, ALG-2-interacting protein X, alpha-lactalbumin, serum albumin, polymeric immunoglobulin, lactoperoxidase, or a combination thereof. In some examples, the MPV comprises BTN1A1, CD81, and/or XOR. In some embodiments, the one or more proteins associated with the lipid membrane of the MPVs comprise glycans attached to glycoproteins and/or glycolipids. Any of such lipid membrane structure of MPVs and/or one or more of the proteins disclosed herein may present in the cargo-loaded vesicles disclosed herein.

In some embodiments, the MPV is obtained from cow milk, goat milk, camel milk, buffalo milk, yak milk, or human milk. In some embodiments, the MPV can be lactosome, milk fat globule (MFG), exosome, extracellular vesicles, whey-particle, aggregates thereof, or any combination thereof.

In some embodiments, the MPVs comprise one or more of the following features:
(i) stability under freeze-thaw cycles and/or temperature treatment;
(ii) colloidal stability when the milk vesicles are loaded with the biological molecule;
(iii) a loading capacity of at least 5000 cholesterol modified oligonucleotides per milk vesicle;
(iv) stability under acidic pH;
(v) stability upon sonication;
(vi) resistance to enzyme digestion; and
(vii) resistance to nuclease treatment upon loading of the milk vesicles with oligonucleotides.

In some examples, the MPVs are stable under an acidic pH ≤ 4.5. In some examples, the MPVs are stable under an acidic pH ≤ 2.5. Alternatively or in addition, the MPVs are resisitant to digestion by one or more digestive enzymes.

In some embodiments, the cargo is a peptide, a protein, a nucleic acid, a polysaccharide, or a small molecule.

In some embodiments, the LNP-MPV disclosed herein comprisises one or more of the properties associated with MPVs, e.g., those disclosed herein. For example, the vesicle of the present disclosure is stable at pH ≤ 4.5, e.g., ≤ pH 4.5, ≤ pH 4.0, ≤ pH 3.5, ≤ pH 3.0, or stable at pH ≤ 2.5, e.g., ≤ pH 2.5, ≤ pH 2.0 and lower. In some embodiments, the vesicle of the present disclosure is resistant to digestive enzymes. In some embodiments, the vesicle is suitable for oral administration of a cargo loaded therein. In some examples, the vesicle comprises BTN1A1. In some examples, the vesicle comprises CD81. In some examples, the vesicle comprises XOR. In some examples, the vesicle comprises any combination of BTN1A1, CD18, and XOR.

In some embodiments, the vesicle is formulated in a composition comprising a pharmaceutically acceptable carrier. In some embodiments, the composition is formulated for oral administration.

In other aspects, the present disclosure also features methods of producing the cargo-loaded vesicles disclosed herein, which may comprise one or more components from MPVs and one or more components from LNPs such as those disclosed herein and any of the cargos also disclosed herein, e.g., a cargo-loaded LNP-MPV.

In some embodiments, the method disclosed herein comprise:
(i) contacting a LNP comprising a cargo with a MPV, thereby causing fusion of the LNP and the MPV to produce LNP-MPV loaded with the cargo;
(ii) collecting the LNP-MPV loaded with the cargo; and optionally
(iii) attaching a targeting moiety to the LNP-MPV loaded with the cargo.

In some embodiments, step (i) is performed in a solution comprising about 5 to about 40% (w/v) polyethylene glycol (PEG). In some embodiments, the solution comprises about 10% to about 35% (w/v) PEG. In some examples, the solution comprises about 20% to about 30% (w/v) PEG. Alternatively or in addition, the PEG in the solution has an average molecular weight of about 6 kD to about 12 kD. In some examples, the PEG in the solution has an average molecular weight of about 8 kD to about 10 kD.

In some embodiments, step (i) comprises extruding a suspension comprising the lipid nanoparticle and the MPVs through a filter under pressure. In some embodiments, the filter is a polycarbonate membrane filter having a pore size of about 50 nm to about 200 nm.

In some embodiments, the step (i) of the method comprises sonication. In some embodiments, step (i) is performed using a microfluidic device. In some examples, the microfluidic device comprises one or more channels having a diameter of about 0.02-2 mm. In some examples, the microfluidic device comprises glass and/or polymer materials.

In any of the methods disclosed herein, step (ii) of the method may comprise collecting the LNP-MPVs by positive selection. Alternatively, step (ii) of the method may comprise collecting the LNP-MPVs by negative selection. In some embodiments, step (ii) of the method is performed using a lectin to collect the LNP-MPVs. Nonlimiting examples of suitable lectins include Con A, RCA, WGA, DSL, Jacalin, and any combination thereof. In other embodiments, step (ii) of the method comprises one or more chromatography approaches, for example, ion-exchange chromatography, affinity chromatography, or a combination thereof.

In some embodiments, a method disclosed herein comprise step (iii) for modifying the cargo-loaded LNP-MPV collected in step (ii). The modifying step may comprise attaching a target moiety that binds gut cells, for example, small intestinal cells.

In some embodiments, the LNP comprising the cargo is produced by a process comprising: mixing an alcohol solution comprising one or more lipids and an aqueous solution comprising the cargo to form the cargo-loaded lipid nanoparticle. In some examples, the mixing step may comprise contacting the alcohol solution comprising one or more lipids with the aqueous solution comprising the cargo at a T junction or a Y junction in one or more tubes, which are connected to one or more pumps. In some examples, the one or more tubes have a diameter of about 0.2-2 mm. In some embodiments, the mixing step can be performed using a microfluidic device. For example, the microfluidic device may comprise one or more channels having a diameter of about 0.02-2 mm. In some examples, the microfluidic device comprises glass and/or polymer materials.

In some embodiments, the LNP comprising the cargo is produced by a process comprising: rehydrating a lipid film with a solution comprising the cargo followed by vortexing, sonication, extrusion, or a combination thereof.

In some embodiments, the method disclosed herein comprises:
(i) loading a cargo into an LNP;
(ii) contacting an LNP comprising a cargo with a MPV, thereby causing fusion of the LNP and the MPV to produce LNP-MPV loaded with the cargo;
(iii) collecting the LNP-MPV loaded with the cargo; and optionally
(iv) attaching a targeting moiety to the LNP-MPV loaded with the cargo.

Also within the scope of the present disclosure are cargo-loaded vesicles prepared by any of the methods disclosed herein and pharmaceutical compositions comprising such, which may be formulated for oral administration. Further, provided herein are methods for oral delivery of a cargo (e.g., a therapeutic cargo as disclosed herein) comprising administering any of the cargo-loaded vesicle or a composition comprising such orally to a subject in need thereof.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1B include schematic illustrations of exemplary fusion processes and cargo-carrying lipid nanoparticle formation processes. FIG. 1A: a schematic illustration showing the fusion of an exemplary cargo-loaded liposome with a whey purified vesicle (WPV), producing a fused liposome-WPV, which can further be programmed with surface ligands. FIG. 1B: a schematic illustration showing the oral administration of surface programmed LNP-MPVs. Vescles produced using Orasome technology, such as LNP-MPVs or liposome-WPVs, transit through the GI tract. In the intestinal lumen, the surface programmed vesicles bind to the intestinal mucosa of a targeted intestinal cell type. Inside the cell, the vector is translated and the resulting proteins are basolaterally secreted into the intestinal submucosa and are taken up via the lympatic vessel system and brought into the systemic circulation.

FIGS. 2A and 2B include charts showing fluorometric analysis for evaluating liposome-exosome fusion facilitated by temperature. FIG. 2A: a chart showing mixing of lipids from liposome and exosome: elevation of fluorescence signal (750-800 nm) – DiI:DiR FRET signal, indicates liposome -exosome fusion. FIG. 2B: a chart showing interaction between liposome and siRNA-conjugated exosome: elevation of fluorescence signal (700-750 nm) – DiI:DY677 FRET signal, indicates liposome -exosome fusion.

FIG. 3 is a diagram showing particle number changes associated with liposome-exosome fusion facilitated by polyethylene glycol (PEG) at various PEG concentrations. Bars from left to right for each PEG molecular weight: 30% PEG, 25% PEG, 20% PEG, 15% PEG, 10% PEG, ad 5% PEG.

FIGS. 4A- 4C include diagram showing particle sizes in association with liposome-exosome fusion facilitated by polyethylene glycol (PEG) of different molecular masses (6-12 kD). FIG. 4A: 10% PEG. FIG. 4B: 20% PEG. FIG. 4C: 30% PEG.

FIGS. 5A-5C include diagrams showing Nanoparticle Tracking Analysis (NTA) of 5-CF loaded liposome fractions purified purified by Size Exclusion Chromatography using a 1.5 X 15 cm column packed with Sephacryl S-500. Fraction 7-12 showed presence of liposomes. FIG. 5A: a diagram showing particle size distribution of 5-CF loaded liposomes in various fractions resulting from SEC. FIG. 5B: a diagram showing particle concentration in various SEC fractions. FIG. 5C: a diagram showing the mean particle size in various SEC fractions.

FIGS. 6A-6F include diagrams showing cargo transfer to fused vesicles via liposome-exosome fusion facilitated by extrusion. FIGS. 6A and 6B: fluorescence intensity released from cargo observed in trial 1. FIGS. 6C and 6D: fluorescence intensity released from cargo observed in trial 2. FIG. 6E: a diagram showing percentage in WGA captured exosomes in trial 1 and trial 2. FIG. 6F: a diagram showing particle size distribution observed in trial 1 and trial 2. 6A and 6C: Upper curve: “extruded” and lower curve: “Liposome”.

FIGS. 7A-7E include diagrams showing cargo transfer to exosome using PEG-facilitated fusion between exosomes and cationic liposomes. FIG. 7A is a schematic illustration of an exemplary process for fusion between cationic liposome and milk exosome vesicles facilitated by PEG. FIG. 7B: a photo showing presence of labelled oligonucleotide cargo in fused vesicles as detected by PAGE (lanes 9-12). Lanes 1-8 are standards and controls as indicated. FIG. 7C: a diagram showing fluorescence spectra from pellet after PEG-facilitated exosome-cationic liposome fusion in presence of various concentration of PEG. 30%: highest curve; 20% second highest curve: 10% and 0%: overlapping lowest curves. FIG. 7D: a diagram showing total fluorescence from pellet after PEG-facilitated exosome-cationic liposome fusion. FIG. 7E: a diagram showing particle size distribution in reaction mixtures in presence of various concentrations of PEG.

FIG. 8 is a photo showing cargo (fluorescently labelled oligonucleotide) transfer to exosome using PEG-facilitated fusion between exosome and neutral liposome as detected by PAGE. Lane 1-7: fluorescently labelled oligonucleotide standards 5 µM, blank, 2.5 µM, 1 µM, 0.5 µM, 0.25 µM, 0.125 µM; Lane 8: milk exosomes, Lane 9: LNP loaded with oligonucleotide, Lane 10: 30% PEG -MEV+Liposome.

FIGS. 9A and 9B include photos showing that oligonucleotides loaded into milk vesicles are protected from S1 nuclease digestion. FIG. 9A: a photo showing protection of oligonucleotides from S1 nuclease digestion by LNPs (variable lipids comprising LNP as indicated) in the absence of 1% Triton X-100 but no protection in the presene of 1% Triton X-100. FIG. 9B: a photo showing protection of oligonucleotides from S1 nuclease digestion by fused vesicles (“fused EVs”) in the presence and absence of 1% Triton X-100.

FIG. 10 is a diagram showing particle size distribution of milk exosomes (EVs) after lyophilization and rehydration to initial volume.

FIG. 11 is a diagram showing particle size distribution of fused vesicles (LipoMEVs) after lyophilisation and rehydration to initial volume.

FIGS. 12A-12D include diagrams showing characteristics of DOTAP liposomes and fused MEV-liposome vesicles prepared by incubating MEVs with LNPs for 2 hours at 40 C at pH 5.5. No significant differences were observed in MEV size after fusion of liposomes at ratios of up to 10:1 Liposome: MEV. FIG. 12A: a diagram showing particle sizes of DOTAP liposomes at pH 5.5. FIG. 12B: a diagram showing particle size of MEV and of MEV-LNP after fusion of EV with DOTAP LNP at pH 5. FIG. 12C: a diagram showing particle size of MEV and of MEV-LNP after fusion of EV with DOTAP LNP at pH 5.5 and 100:1 Liposome: MEV ratio. FIG. 12D: a diagram showing particle size of MEV and of MEV-LNP after fusion of EV with DOTAP LNP at pH 5.5 and 100:1 and 500:1 Liposome: MEV ratio. DOTAP2k are liposomes made from DOTAP and DSPE-mPEG2k. DOTAP5k are liposomes made from DOTAP and DSPE-mPEG5k.

FIG. 13 is a diagram showing fluorescence of labeled DOTAP or DODMA liposomes fused with MEVs at pH 5.5 or pH 8. Stock = prior to ultracentrifugation.

FIGS. 14A and 14B include diagrams showing loading of oligonucleotide (ON) cargo into milk vesicles via fusion. FIG. 14A: a diagram showing particle sizes after fusion at pH 5.5 at the indicated LNP/EV ratios. FIG. 14B: a diagram showing particle size after fusion of EV with LNP loaded with ON at pH 8 and 1:1 ratio.

FIGS. 15A and 15B include diagrams showing loading of siRNA cargo into milk vesicles via fusion. FIG. 15A: a diagram showing particle sizes after fusion at pH 5.5 and pH8.5 at the indicated LNP/EV ratios. FIG. 15B: a diagram showing particle size after fusion of EV with LNP loaded with chol-siRNA0Cy5.5 at pH 5.5 and ½ ratio.

FIGS. 16A and 16B include diagrams showing loading of oligonucleotide (ON) cargo into milk vesicles via fusion comparing LNPs comprising DOPC or DSPC as helper lipids. FIG. 16A: a diagram showing particle sizes after fusion at pH 5.5 of LNPs with the indicated helper lipids with MEVs. FIG. 16B: a diagram showing particle sizes after fusion at pH 7.4 of LNPs with the indicated helper lipids with MEVs.

FIGS. 17A-17C include diagrams showing siRNA post RCA precipitation. FIG. 17A: is a photo showing presence of siRNA in pellets and supernatant after RCA precipitation. FIG. 17B: is a diagram showing particle sizes of siRNA LNP/EV fusion before RCA pull-down. FIG. 17C is a diagram showing sizes of particles in supernatant after RCA pull-down.

FIG. 18 is a diagram showing particle size and concentration after TFF concentration of a siRNA loaded LNP/EV.

FIGS. 19A-19G include diagrams showing loading of antisense oligonucleotide (ASO) cargo into milk vesicles via fusion. FIG. 19A is a photo showing presence of ASO in the pellet and supernatant after RCA precipitation of EV fused with DOTAP LNP. FIG. 19B is a photo showing presence of ASO in the pellet and supernatant after RCA precipitation of EV fused with DODMA LNP. FIG. 19C is a photo showing presence of ASO in the pellet and supernatant after precipitation by RCA-Dyna beads. FIG. 19D is a diagram showing sizes of particles in the supernatant after precipitation by RCA-Dyna beads. No LNP particles were found in the supernatant when fusion was carried out at pH 5.5. FIGS. 19E and 19F are diagrams showing levels of MV²⁺ quenching in the absence (19E) or presence of Triton X (19F). Inaccessibility to MV²⁺ was >95% and ~ 75%, respectively. FIG. 19G is a photo showing presenceof ASO in the pellet and supernatant after lectin pull down.

FIGS. 20A-20E include diagrams showing loading of mRNA cargo into milk vesicles via fusion. FIG. 20A: a diagram showing particle sizes after fusion of mRNA-carrying LNP with EV. FIG. 20B is a photo showing mRNA degradation in the presence or absence of RNAase inhibitors. FIG. 20C is a photo showing mRNA degradation in the presence or absence of RNAase inhibitors when fusioned EVs are treated by Proteinase K. FIGS. 20D and 20E are photos showing cell uptake of mRNA, mRNA-LNP, and mRNA/LNP/EV with lipofectamine and without lipofectamine, respectively.

FIGS. 21A and 21B include diagrams showing particle size distribution measured by nanoparticle tracking analysis (NTA). FIG. 21A: AAV-Lipid particles. FIG. 21B: Exsome/AAV-Lipid fusion particles.

FIGS. 22A-C are diagrams showing PEG-mediated fusion between liposome and MEV by FRET Assay. FIG. 22A: FRET assay measuring fusion between MEVs and non-pegylated liposomes (50% DOTAP, 47% DOPE, 1.5% NBD-PS, 1.5% Rho-PE; Size = 158 nm, PDI = 0.18, ZP = 12.9 mV). FIG. 22B: FRET assay measuring fusion between MEVs and pegylated liposomes (50% DOTAP, 42% DOPE, 1.5% NBD-PS, 1.5% Rho-PE, 5% PEG 2000-PE; Size = 102 nm, PDI = 0.08, ZP = 0.31 mV). FIG. 22C: Comparison of non-pegylated and pegulated liposomes at 120 minutes.

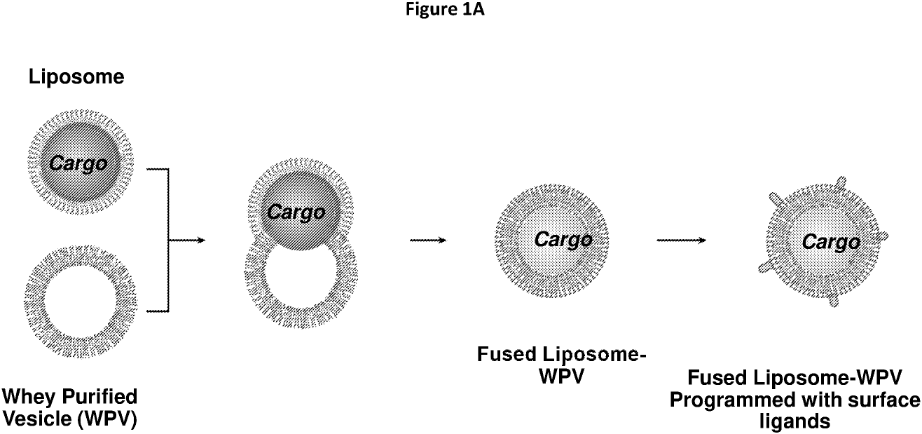

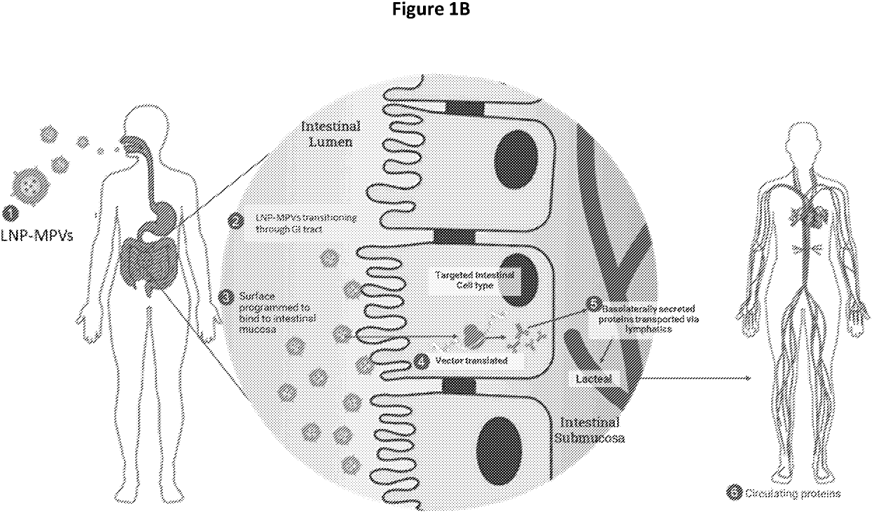

### DETAILED DESCRIPTION OF THE INVENTION

Exosomes are a type of extracellular vesicle approximately 100 nm in diameter that are produced in the endosomal compartment and secreted from most types of eukaryotic cells. Human cell-derived exosomes have attractive promise as vehicles for systemic drug delivery due to their tolerability over synthetic polymer-based delivery technologies. However, the fragile nature of exosomes derived from human cells limits the type of post-isolation manipulations that can be applied in order to optimise such vesicles for exogenous drug cargo loading, administration and storage. This contrasts with vesicles isolated from milk, such as exosomes, which have evolved in all mammals to remain stable following oral consumption and transit through the upper GI tract. Moreover, bovine milk is a rich, readily available and inexpensive source of exosomes harbouring approximately 10¹¹ to 10¹² purifiable exosomes per millilitre. By comparison, serum or plasma contains approximately 1,000-fold fewer exosomes (10⁸ to 10⁹ exosomes) per millilitre.

One problem associated with development of milk vesicle-based drug delivery system is the lack of suitable methods for efficient loading of cargos into the milk vesicles. Direct incubation of cargos with particle-based carriers is known; however, the loading efficiency is very low and therefore not scaleable. Electroporation has been explored for cargo loading, which makes loading of large molecules possible. However, loading efficiency with this approach is also low, particularly when the cargo is hydrophobic. Electroporation may disrupt integrity of the milk vesicles and/or cause cargo aggregation. Similarly, sonication and extrusion may increase loading efficiency; however, these approaches bear the risk of deforming the membranes of the milk vesicles. Sonication is also not suitable for loading hydrophobic drugs. Freeze/thaw methods could result in medium loading efficiency and make membrane fusion possible; however, such methods could cause milk exosome aggregation and moreover, the loading efficiency is still not satisfactory. Finally, saponin-assisted loading could lead to high drug loading efficiency as compared with other approaches; however, saponin could generate pores in exosomes and would raise toxicity concerns.

The present disclosure is based, at least in part, on the development of methods for loading various types of cargos into vesicles derived from milk, such as exosomes (e.g., milk purified exosomes or MPVs such as whey purified vesicles or WPVs) and the cargo-loaded vesicles thus produced. The instant disclosure relates to vesicles comprising one or more components from vesicles such as MPVs or WPVs, which can be loaded with a cargo, such as a therapeutic cargo, and methods of producing such. The MPVs may comprise one or more modifications relative to the natural counterparts. The therapeutic vesicles described herein can be harnessed to provide new treatments for diseases, such as rheumatoid arthritis, diabetes and cancer for which the standard of care requires intravenous infusion or subcutaneous injection of monoclonal antibodies (e.g. anti-PD1, anti-TNF) or protein/ peptides (e.g., GLP-1, P-glucocerebrosidase, Factor IX, Erythropoietin). Moreover, the novel vesicles described herein hold promise for expanding a variety of modalities, such as messenger RNA and antisense, to new disease areas and treatment regimens. Having passed through the stomach protected, the therapeutic cargo can act either directly in the GI tract, transit through the mucosa to the underlying lymphatic vascular network or, in the case of cargos that yield mRNAs, produce complex biologics such as antibodies within mucosal cells that are secreted into the mucosal lymphatic vascular network for subsequent systemic distribution.

Within the context of infectious disease, the vesicles described herein can support oral administration of neutralizing monoclonal antibodies or antibody combinations to supply passive immune therapies for infected individuals and passive immune protection for healthcare and first responder professionals. Often, the time required to produce sufficient supplies of such monoclonal antibodies by standard manufacturing processes, accompanied by the significant manufacturing cost as well as the need for intravenous monoclonal antibody infusion, render the conventional passive immunotherapy approach difficult. Moreover, in some instances, more than one anti-virus antibody may need to be combined in order to achieve virus control. Using vesicles described herein, comprising one or more components from vesicles purified from milk or whey, as a delivery strategy may allow for rapid transfer of the DNA sequences or other nucleic acid expression systems coding for the monoclonal antibodies into the milk exosomes, thereby enabling the body to make its own “drug” (e.g., through oral administration of mRNA or other gene delivery system) and permitting oral administration at significantly lower cost than traditional approaches. Importantly, this approach will permit the generation of multiple antibody combinations where needed for more optimal therapeutic efficacy.

Oral administration of vesicles described herein, comprising one or more components from vesicles purified from milk or whey, e.g., such as those made according to the methods described herein, to a subject in need of treatment in certain instances will permit the subject’s own GI tract cells to make therapeutic protein. This approach also has the potential to provide a more convenient and significantly less expensive means to deliver biological medicines.

Provided herein are vesicles comprising one or more components from vesicles purified from milk or whey, further comprising a cargo, e.g., a therapeutic cargo. A vesicle purified from milk, referred to herein as a “vesicle isolated from milk”, “milk-derived vesicle”, “vesicle derived from milk”, “vesicle purified from milk,” “milk purified vesicles” or “MPV,” described herein can be any type(s) of particles found in milk. Examples include, but are not limited to, lactosome, milk fat globules (MFG), milk exosomes, and whey particles. A vesicle purified from whey (also referred to as “WPV”) is a type of MPV. The term “milk extracellular vesicle” or “milk exosome vesicle” or “MEV” refers to a vesicle that is a type of MPV. An MPV or WPV comprises one or more components of an MPV or WPV. Also provided herein are methods for producing said vesicles comprising one or more milk vesicle components described herein, comprising a cargo. In some emodiments, the vesicles of the disclosure further comprise one or more components of a lipid nanoparticle. Methods described herein involves fusion between lipid nanoparticles, such as liposomes carrying a suitable cargo with vesicles purified from milk to provide a fused vesicle, i.e., an LNP-MPV, loaded with a cargo.

In some aspects the present disclosure provides novel vesicles, comprising one or more components from a milk purified vesicle, referred to herein as an “MPV” and one or more components from a lipid nanoparticle (LNP), and having the cargo encapsulated therein. Such vesicles of the disclosure are referred to herein as “fused vesicle” or “fused vesicles”, as “LNP-MPV” or “LNP-MPVs”, “fused LNP-MPV” or “fused LNP-MPVs”, or as “duosome” or “duosomes.” One non-limiting example of such an LNP-MPV is a liposome-WPV, which comprises one or more components from a liposome and one or more components from a WPV, having the cargo encapsulated therein. A “fused EV” (fused extracellular vesicle) is a type of LNP-MTV. Cargos include for example peptides, proteins, nucleic acids, polysaccharides, or small molecules. Exemplary cargos are described elsewhere herein.

The method disclosed herein results in luminal loading of cargos into the vesicles resulting from the fusion, i.e., the LNP-MPVs, and confers various advantageous properties, including high loading efficiency, an approach universally applicable to various types of cargo (e.g., hydrophobic or anionic cargos), and/or luminal loading of cargo into the LNP-MPVs, leading to better protection of the cargo, particularly macromolecule-based cargos, e.g., as required for oral administration and/or delivery. As used herein, the term “luminal loading” includes cargo that is fully (e.g., entirely or wholly) encapsulated as well as cargo that is partially encapsulated. The use of vesicles purified from milk or whey in the fusion methods disclosed herein confers certain components of vesicles purified from milk or whey to the resultant the LNP-MPVs, resulting in the transfer of beneficial characteristics to the resultant fused LNP-MPVs not found in other vesicles used to transport cargo.

In some embodiments, the surface of the vesicles comprising one or more components from a vesicle purified from milk or whey, is programmed or functionalized with ligands or targeting moieties to improve intestinal uptake for improved oral delivery, as described herein. The fusion-based method disclosed herein may use vesicles purified from whey, i.e., whey-purified extracellular vesicles or “WPVs”, as a starting material, yielding LNP-WPVs, such as liposome-WPVs, resulting from fusion of the WPVs vesicles and cargo-carrying lipid nanoparticles.

Additionally, LNP-MPVs, e.g., liposome-WPVs, may be subject to surface modification, i.e., surface programming. For example, a moiety (e.g., PEG-lectin) having binding activity to specific gut cells (e.g., small intestine cells) may be attached to the LNP-MPV to produce the final product for oral administration. Such vesicles are referred to as surface programmed LNP-MPVS. Such surface programmed LNP-MPVs are an example a type of vesicle which can be produced using Orasome technology.

Orasome technology is designed to enable the oral administration of biotherapeutics, including nucleic acid-based and protein-based biotherapeutics, e.g., those disclosed herein. Examples include, but are not limited to, antisense oligonucleotides, short interfering RNA, mRNA, modular expression systems for therapeutic proteins, peptides and nanoparticles. Orasome technology involves the use of vesicles isolated from milk, such as exosomes, which may be modified or engineered for transport through the gastro-intestinal tract. In some instances, Orasome technology may utilize multiple components from vesicles isolated from milk. Such vesicles may be engineered to remain stable following oral consumption and transit through the upper GI tract. Orasome vesicles are readily amenable to manufacturing at scale and relatively low cost based on the easily accessible and engineerable components.

### I. Vesicles Purified From Milk

Milk vesicles, for example milk exosomes, microvesicles, and other vesicles found in milk of a suitable mammalian source, are small assemblies of lipids about 20-1000 nm in size, which can encapsulate or otherwise carry miRNA species, can enable oral delivery of a variety of therapeutic agents. The present disclosure harnesses certain properties of vesicles isolated from milk or whey, such as exosomes, to meet the urgent need for suitable delivery vehicles for therapeutics that were previously not orally administrable or suffered from other delivery challenges such as poor bioavailability, storage instability, metabolism, off-target toxicity, or decomposition in vivo.

Provided herein are compositions comprising MPVs, e.g., WPVs, as disclosed herein, wherein the MPV compositions have a relative abundance of proteins with a molecular weight of about 25-30 kDa (e.g., casein) no greater than about 40% and/or a relative abundance of proteins with a molecular weight of about 10-20 kDa (e.g., lactoglobulin) no greater than 25%. “Relative abundance of a protein” refers to the percentage of that protein relative to the total proteins in a vesicle or composition.

Any of the MPVs, e.g., WPVs, described herein are suitable for use in any of fusion, cargo-loading, purification, and enrichment methods described herein. Such methods can comprise contacting a lipid nanoparticle (LNP), e.g., a liposome, carrying a cargo with a composition comprising milk vesicles under suitable conditions that allow for fusion of the lipid nanoparticle with the MPVs, thereby producing an LNP-MPV, such as a Liposome-WPVhaving the cargo encapsulated therein. In some embodiments, the cargo-loaded LNP-MPV, e.g., fused liposome-WPV, may be collected, for example, by negative selection or positive selection.

### A. Size of Vesicles Purified From Milk

In some descriptions, e.g., where diameter is a relevant measurement, such as in spherical and other shaped vesicles having a measurable diameter, the terms “size” and “diameter” are used interchangeably. The MPV, e.g., WPV, can be about 20 nm - 1000 nm in diameter or size. In some embodiments, MPV, e.g., an WPV, is about 20 nm to about 200 nm in size. In some embodiments, the MPV is about 20 nm to about 190 nm or about 25 nm to about 190 nm in size. In some embodiments, the MPV, e.g., WPV, is about 30 nm to about 180 nm in size. In some embodiments, the MPV, e.g., WPV, is about 35 nm to about 170 nm in size. In some embodiments, the MPV, e.g., WPV, is about 40 nm to about 160 nm in size. In some embodiments, the MPV, e.g., WPV, is about 50 nm to about 150 nm, about 60 nm to about 140 nm, about 70 nm to about 130 nm, about 80 nm to about 120 nm, or about 90 nm to about 110 nm in size. In some embodiments, the MPV, e.g., WPV, is about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in size or diameter. In some embodiments, an average MPV size in a vesicle composition or plurality of MPVs, e.g., WPVs, is about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in average size. In some embodiments, an average MPV size in a vesicle composition or plurality of MPVs, e.g., WPVs, is about 20 nm to about 200 nm, about 20 nm to about 190 nm, about 25 nm to about 190 nm, about 30 nm to about 180 nm, about 35 nm to about 170 nm, about 40 nm to about 160 nm, about 50 nm to about 150, about 60 to about 140 nm, about 70 to about 130, about 80 to about 120, or about 90 to about 110 nm in average size.

In some embodiments, the MPV, e.g., WPV, is about 20 nm to about 100 nm in size. In some embodiments, the MPV, e.g., WPV, is about 25 nm to about 95 nm in size. In some embodiments, the MPV, e.g., WPV, is about 20 nm to about 90 nm in size. In some embodiments, the MPV is about 20 nm to about 85 nm in size. In some embodiments, the MPV, e.g., WPV, is about 20 nm to about 80 nm in size. In some embodiments, the MPV, e.g., WPV, is about 20 nm to about 75 nm in size. In some embodiments, the MPV, e.g., WPV, is about 20 nm to about 70 nm in size. In some embodiments, the MPV, e.g., WPV, is about 25 nm to about 80 nm in size. In some embodiments, the MPV, e.g., WPV, is about 30 nm to about 70 nm in size. In some embodiments, the MPV is about 30 nm to about 60 nm in size. In some embodiments, the MPV, e.g., WPV, is about 40 nm to about 70 nm in size. In some embodiments, the MPV, e.g., WPV, is about 40 nm to about 60 nm in size. In some embodiments, an average MPV, e.g., WPV, size in a vesicle composition or plurality of vesicles isolated or purified from milk is about 20 nm to about 100 nm, about 20 nm to about 95 nm, about 20 nm to about 90 nm, about 20 nm to about 85 nm, about 20 nm to about 80 nm, about 20 to about 75 nm, about 25 nm to about 85 nm, about 25 nm to about 80, about 25 to about 75 nm, about 30 to about 80 nm, about 30 to about 85 nm, about 30 to about 75 nm, about 40 to about 80, about 40 to about 85 nm, about 40 to about 75 nm, about 45 to about 80 nm, about 45 to about 85, about 45 to about 75 nm, about 50 to about 75 nm, about 50 to about 80 nm, about 50 to about 85 nm, about 55 to about 75 nm, about 55 to about 80 nm, about 55 to about 85 nm, about 60 to about 75 nm, about 60 to about 80 nm, about 60 to about 85 nm, about 25 to about 70 nm, about 30 to about 70 nm, about 40 to about 70 nm, about 50 to about 70 nm, about 30 to about 60 nm, about 30 to about 50 nm in average size.

In some embodiments, the MPV, e.g., WPV, is about 80 nm to about 200 nm in size. In some embodiments, the MPV, e.g., WPV, is about 85 nm to about 195 nm in size. In some embodiments, the MPV, e.g., WPV, is about 90 nm to about 190 nm in size. In some embodiments, the MPV is about 95 nm to about 185 nm in size. In some embodiments, the MPV, e.g., WPV, is about 100 nm to about 180 nm in size. In some embodiments, the MPV, e.g., WPV, is about 105 nm to about 175 nm in size. In some embodiments, the MPV, e.g., WPV, is about 110 nm to about 170 nm in size. In some embodiments, the MPV is about 115 nm to about 165 nm in size. In some embodiments, the MPV, e.g., WPV, is about 120 nm to about 160 nm in size. In some embodiments, the MPV, e.g., WPV, is about 125 nm to about 155 nm in size. In some embodiments, the MPV is about 130 nm to about 150 nm in size. In some embodiments, the MPV, e.g., WPV, is about 135 nm to about 145 nm in size. In some embodiments, the MPV is about 110 nm to about 150 nm in size. In some embodiments, an average vesicle size in a MPV composition or plurality of MPVs, e.g., WPVs, is about 80 nm to about 200 nm, about 80 nm to about 190 nm, about 80 nm to about 180 nm, about 80 nm to about 170 nm, about 80 nm to about 160 nm, about 80 to about 150 nm, about 80 nm to about 140 nm, about 80 nm to about 130, about 80 to about 120 nm, about 80 to about 110 nm, about 80 to about 100 nm, about 30 to about 75 nm, about 40 to about 80, about 40 to about 85 nm, about 40 to about 75 nm, about 45 to about 80 nm, about 45 to about 85, about 45 to about 75 nm, about 50 to about 75 nm, about 50 to about 80 nm, about 50 to about 85 nm, about 55 to about 75 nm, about 55 to about 80 nm, about 55 to about 85 nm, about 60 to about 75 nm, about 60 to about 80 nm, about 60 to about 85 nm, about 25 to about 70, about 30 to about 70, about 40 to about 70 nm, about 50 to about 70 nm, about 30 to about 60 nm, about 30 to about 50 nm in average size.

In some embodiments, the MPV, e.g., WPV, is greater than 200 nm in size. In some embodiments, the MPV, e.g., WPV, is about 200 to about 1000 nm in size. In some embodiments, the MPV, e.g., WPV, is about 200 to about 400 nm in size, e.g., about 200 nm to about 250 nm, about 250 nm to about 300 nm, about 300 to about 350 nm, about 350 nm to about 400 nm in size. In some embodiments, the MPV, e.g., WPV, is about 400 to about 600 nm in size, e.g., about 400 nm to about 450 nm, about 450 nm to about 500 nm, about 500 to about 550 nm, about 550 nm to about 600 nm in size. In some embodiments, the MPV, e.g., WPV, is about 600 to about 800 nm in size, e.g., about 600 nm to about 650 nm, about 650 nm to about 700 nm, about 700 to about 750 nm, about 750 nm to about 800 nm in size. In some embodiments, the MPV, e.g., WPV, is about 800 to about 1000 nm in size, e.g., about 800 nm to about 850 nm, about 850 nm to about 900 nm, about 900 to about 950 nm, about 950 nm to about 1000 nm in size. In some embodiments, an average MPV, e.g., WPV, size in a vesicle composition or plurality of MPVs, e.g., WPVs, is about 200 nm to about 1000 nm, about 200 nm to about 900 nm, about 200 nm to about 800 nm, about 200 nm to about 700 nm, about 200 nm to about 600 nm, about 200 to about 500 nm, about 200 nm to about 400 nm, about 200 nm to about 300, about 300 to about 1000 nm, about 300 to about 900 nm, about 300 to about 800 nm, about 300 to about 700 nm, about 300 to about 600, about 300 to about 500 nm, about 300 to about 400 nm, about 400 to about 1000 nm, about 400 to about 900, about 400 to about 800 nm, about 400 to about 700 nm, about 400 to about 600 nm, about 400 to about 500 nm, about 500 to about 1000 nm, about 500 to about 900 nm, about 500 to about 800 nm, about 500 about 700 nm, about 500 to about 600 nm, about 600 to about 1000 nm, about 600 to about 900 nm, about 600 to about 800 nm, about 600 to about 700 nm, about 700 to about 1000 nm, about 700 to about 900 nm, about 700 to about 800 nm, about 800 to about 1000 nm, about 800 to about 900 nm, about 900 to about 1000 nm in average size.

The size of the MPVs disclosed herein is determined by Dynamic Light Scattering (DLS) or nanoparticle tracking analysis (NTA).

### B. Source of Milk Vesicles

The milk purified vesicles described herein can be purified from any form of milk or milk component of any suitable mammal. The term “milk” as used herein refers to the opaque liquid containing proteins, fats, lactose, and vitamins and minerals that is produced by the mammary glands of mature female mammals including, but not limited to, after the mammals have given birth to provide nourishment for their young. In some embodiments, the term “milk” is further inclusive of colostrum, which is the liquid secreted by the mammary glands of mammals shortly after parturition that is rich in antibodies and minerals. In some embodiments, the term “milk” is further inclusive of whey. The milk purified vesicles (MPVs) can be from any mammalian species, including but not limited to, primates (e.g., human, ape, monkey, lemur), rodentia (e.g., mouse, rat, etc), carnivora (e.g., cat, dog, etc.), lagomorpha (e.g., rabbit, etc), cetartiodactyla (e.g., pig, cow, deer, sheep, camel, goat, bufflo, yak, etc.), perissodactyla (e.g., horse, donkey, etc.). In certain embodiments, the milk or colostrum, or vesicles purified therefrom, is from human, cow, buffalo, pig, goat, rat, mouse, sheep, camel, donkey, horse, llama, alpaca, vicuña, reindeer, moose, or yak milk or colostrum. In some embodiments, the milk is cow milk or whey from cow milk.

Milk as used herein encompass milk of any form, including raw milk (whole milk), colostrum, skim milk, pasteurized milk, homogenized milk, acidified milk (milk with casein removed), or milk component, such as whey.

In some embodiments, the vesicles are purified from colostrum, which is the first form of milk produced by the mammary glands of mammals immediately following delivery of the newborn. In some embodiments, the milk is whole milk or raw milk, which is obtained directly from a female mammal with no further processing. In some embodiments, the milk is fat-free milk or skim milk, which typically has milk fat removed substantially. In some embodiments, the milk is reduced fat milk, e.g., milk having 1 % or 2% milk fat. In some embodiments, the milk is pasteurized milk, which is typically prepared by heating milk up and then quickly cooling it down to eliminate certain bacteria. In some embodiments, the milk is HTST (High Temperature Short Time) or flash pasteurized. In some embodiments, the milk is UHT or UP (Ultra High Temperature) pasteurized. In some embodiments, the milk is sterilized milk, for example, irradiated milk. In some embodiments, the milk is homogenized milk, which can be prepared by a process in which the fat molecules in milk (e.g., pasteurized milk) have been broken down so that they stay integrated rather than separating as cream. It is a usually a physical process with no additives. In some embodiments, the milk is processed using a combination of one or more of homogenization, pasteurization, sterilization and/or irradiation.

In some embodiments, the vesicles are purified from whey, i.e., WPVs In some embodiments, the WPVs can be made from skimmed and casein depleted milk via macrofiltration, tangential flow filtration, size exclusion chromatography, or a combination thereof. In certain embodiments, the whey can produced from milk from human, cow, buffalo, pig, goat, rat, mouse, sheep, camel, donkey, horse, llama, alpaca, vicuña, reindeer, moose, or yak.

Methods for homogenization, pasteurization, sterilization, and irradiation of milk are known in the art. For example, methods and machinery or mechanisms for homogenizing milk are known. Homogenization is a mechanical process by which fat globules in the milk are broken down such that they are reduced in size and remain suspended uniformly throughout the milk. Homogenization is accomplished by forcing milk at high pressure through small holes. Other methods of homogenization employ the use of extruders, hammermills, or colloid mills to mill (grind) solids. HTST pasteurization requires heating the milk or colostrum to 165° F. for 15 seconds. UHT or UP pasteurization requires heating the milk or colostrum to 280 - 284° F. for 2-4 seconds. Milk or colostrum can be irradiated using various methods, including gamma radiation, in which gamma rays emitted from radioactive forms of the element cobalt (Cobalt 60) or of the element cesium (Cesium 137) are used; X-ray radiation, in which x-rays are produced by reflecting a high-energy stream of electrons off a target substance (usually one of the heavy metals) into food; and electron beam or e-beam radiation, in which a stream of high-energy electrons are propelled from an electron accelerator into food.

In some embodiments, the milk or whey can be lyophilized. Lyophilized milk or whey can be reconstituted using standard procedures as recommended by manufacturer’s instruction and/or as known in the art, for example, by mixing distilled water with lyophilized milk at room temperature such that the milk is present at a final concentration of 5% by weight relative to water.

The vesicles purified from milk (MPVs) described herein can be any types of particles found in milk. Examples include, but are not limited to, lactosome, milk fat globules (MFG), milk exosomes, and whey particles. Lactosome are nanometer-sized lipid-protein particles (~ 25 nm) that do not contain triacylglycerol. Argov-Argaman et al., J. Agric Food Chem, 2010, 58(21):11234. MFGs are milk particles having a lipid-protein membrane surrounding milk fat; secreted by milk producing cells; a source of multiple bioactive compounds, such as phospholipids, glycolipids, glycoproteins, and carbohydrates. The milk fat globule is surrounded by a phospholipid trilayer containing associated proteins, carbohydrates, and lipids derived primarily from the membrane of the secreting mammary epithelial cell (lactocyte). This trilayer is collectively known as MFGM. While the MFGM only makes up an estimated 2% to 6% of the total milk fat globule, it is an especially rich phospholipid source, accounting for the majority of total milk phospholipids. In contrast, the inner core of the milk fat globule is composed predominantly of triacylglycerols. Lopez et al., (2011), Colloids and Surfaces. B, Biointerfaces. 83 (1): 29-41. Gallier et al., (2010), Journal of Agricultural and Food Chemistry. 58 (7): 4250-4257. Keenan, T. W. (2001), Journal of Mammary Gland Biology and Neoplasia. 6 (3): 365-371. Milk exosomes refer to extracellular vesicles found in milk, which are secreted by multiple cell types into the extracellular space. Typically, milk exosomes may have a size of about 80-160 nm. Samuel et al., 2017, Sci. Rep. 7:5933. Whey particles are found in milk that contain whey protein.

### C. Biological Components of Vesicles Purified From Milk

The MPVs, e.g., WPVs described herein not only differ from cellular vesicles, e.g., cellular exosomes, in the source from which they are purified, but also differ in their chemical and biological characteristics. For example, vesicles purified from milk comprise proteins not found in cellular exosomes and also comprise a glycocalyx structure which differes from cellular exosomes and imparts certain biochemical properties to MPVs. In some embodiments, the MPVs used in the methods describes herein may comprise one or more of the following molecules: lipid, protein, glycoprotein, glycolipid, lipoprotein, phospholipid, phosphoprotein, peptide, glycan, fatty acid, sterol, steroid, and combinations thereof. Typically, the MPVs described herein comprise a lipid-based membrane to which one or more proteins are associated. The proteins may be attached to the surface of the lipid membrane or embedded in the lipid membrane. Alternatively or in addition, the proteins may be encapsulated by the lipid membrane. In some instances, the milk vesicles may contain endogenous RNA, such as miRNA.

### Lipid Membrane of Vesicles Purified From Milk

The MPVs, e.g., WPVs, may comprise one or more lipids selected from fatty acid, sterol, steroid, cholesterol, and phospholipid. In some embodiments, the lipid membrane of the MPVs described herein may comprise ceramides or derivatives thereof, gangliosides, phosphatidylinositols (PI) such as alpha-lysophosphatidylinositol (LPI), phosphatidylserine (PS), cholesterol (CHOL), phosphatidic acids (PA), glycerol or derivatives thereof, such as diacylglycerol (DAG) or phosphatidylglycerol (PG), sphingolipids, or combinations thereof. Ceramides are a family of lipid molecules composed of sphingosine and a fatty acid. Examples include, but are not limited to, ceramide (Cer), lactosylceramide (LacCer), hexosylceramide (HexCer), and globotriaosylceramide (Gb3). Gangliosides are a family of molecules composed of a glycosphigolipid with one or more sialic acids, for example, n-acetylneuraminic acid (NANA). Examples include, but are not limited to, GM1, GM2, GM3, GD1a, GD1b, GD2, GT1b, GT3, and GQ1. Sphingolipids are a class of lipids containing a backbone of sphingoid bases and a set of aliphatic amino alcohols that includes sphingosine. Examples include sphingomyelin (SM).

Alternatively or in addition, the MPVs, e.g., WPVs, may contain lipids such as phosphatidylcholines (PC), cholesteryl ester (CE), phosphatidylethanolamine (PE), and/or lysophosphatidylethanolamine (LPE).

### Proteins, Polypeptides, and Peptides of Vesicles Purified From Milk

The vesicles purified from milk described herein may comprise one or more components, such as proteins, which may be associated with the lipid membranes also described herein. A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. In some instances, a peptide may contain ten or more amino acids but less than 50. In some instances, a polypeptide or a protein may contain 50 or more amino acids. In other instances, a peptide, polypeptide, or protein may have a mass from about 10 kDa to about 30 kDa, or about 30 kDa to about 150 or to about 300 kDa.

Exemplary MPV components, e.g., MPV proteins may contain only natural amino acids, although non natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

In some embodiments, the MPVcomprises one or more components, such as polypeptides selected from the following polypeptides: butyrophilin subfamily 1, butyrophilin subfamily 1 member A1, butyrophilin subfamily 1 member A1 isoform X2, butyrophilin subfamily 1 member A1 isoform X3, serum albumin, fatty-acid binding protein, fatty acid binding protein (heart), lactadherin, lactadherin isoform X1, beta-lactoglobin, beta-lactoglobin precursor, lactotransferrin precursor, alpha-S1-casein isoform X4, alpha-S2-casein precursor, casein, kappa-casein precursor, alfa-lactalbumin precursor, platelet glycoprotein 4, xanthine dehydrogenase oxidase, ATP-binding cassette sub-family G, perilipin, perilipin-2 isoform X1, RAB1A (member RAS oncogene family), peptidyl-prolyl cis-trans isomerase A, ras-related protein RAB-18, EpCam, CD81, TSG101, HSP70, polymeric immunoglobulin receptor, lactoferrin, CD63, Tsg101, Alix, CD81, and lactoperoxidase isoform X1. In some embodiments, MPV, e.g., an WPV, comprises butyrophilin. In some embodiments, the MPV, e.g., WPV, comprises butyrophilin subfamily 1. In some embodiments, the MPV, e.g., WPV, comprises butyrophilin subfamily 1 member A1 (BTN1A1). In some embodiments, the MPV, e.g., WPV, comprises lactadherin. In some embodiments, the MPV, e.g., WPV, comprises one or more of the following polypeptides: CD81, CD63, Tsg101, CD9, Alix, EpCAM, and XOR. In some embodiments, the MPV, e.g., WPV, comprises CD81. In some embodiments, the MPV, e.g., WPV, comprises XOR. In some embodiments, the MPV, e.g., WPV, comprises BTN1A1 and CD81. In some embodiments, the MPV, e.g., WPV, comprises BTN1A1 and XOR. In some embodiments, the MPV, e.g., WPV, comprises XOR and CD81. In some embodiments, the MPV, e.g., WPV, comprises BTN1A1, CD81, and XOR. In some embodiments, the MPV, e.g., WPV, may comprise a fragment of any of the proteins disclosed herein, for example, the transmembrane fragment. In particular examples, the MPV, e.g., WPV, may comprise BTN1A1, BTN1A2, or a combination thereof. One or more of these polypeptides may enhance the stability, loading of cargo, transport, uptake into cells or tissues, and/or bioavailability of the MPV.

In some embodiments, the MPVcomprises one or more components, such as polypeptides or proteins comprising moieties which may be glycosylated, i.e., linked to one or more glycans at one or more glycosylation sites. A glycan is a compound consisting of one or more monosaccharides linked glycosidically, including for example, the carbohydrate portion of a glycoconjugate, such as a glycoprotein, glycolipid, or a proteoglycan. Glycans can be homo- or heteropolymers of monosaccharide residues and can be linear or branched. Glycans can have O-glycosidic linkages (linked to oxygen in a serine or threonine residue of a peptide chain) or N-Linked linkages (linked to nitrogen in the side chain of asparagine in the sequence Asn-X-Ser or Asn-X-Thr, where X is any amino acid except proline). Glycans bind lectins and have many specific biological roles in cell-cell recognition and cell-matrix interactions.

The glycosylated proteins that can be present in the biological membrane of a MPV, e.g., WPV, as described herein can include any appropriate glycan. Examples of glycans include, without limitation, N-glycans (e.g., N-acetyl-glucosamines and N-glycan chains), O-glycans, C- glycans, sialic acid, galactose or mannose residues, and combinations thereof. In some embodiments, the glycan is selected from an alpha-linked mannose, Gal β 1-3 GalNAc 1 Ser/Thr, GalNAc, or sialic acid. In some embodiments, the MPV, e.g., WPV, comprises one or more glycoproteins or glycopolypeptides having a glycan selected from: galactose, mannose, O-glycans, N-acetyl- glucosamines, and/or N-glycan chains or any combination thereof. In some embodiments, the MPV, e.g., WPV, comprises one or more glycoproteins or glycopolypeptides having a glycan selected from: D- or L- glucose, erythrose, fucose, galactose, mannose, lyxose, gulose, xylose, arabinose, ribose, 2′-deoxyribose, glucosamine, lactosamine, polylactosamine, glucuronic acid, sialic acid, sialyl-Lewis X (SLex), N-acetyl-glucosamine, N- acetyl-galactosamine, neuraminic acid, N-glycolylneuraminic acid (Neu5Gc), N- acetylneuraminic acid (Neu5Ac), an N-glycan chain, an O-glycan chain, a Core 1, Core 2, Core 3, or Core 4 structure, or a phosphate- or acetate-modified analog thereof or a combination thereof. In some embodiments, the MPV, e.g., WPV, comprises a glycoprotein having one or more of the following glycans: terminal b-galactose, terminal a-galactose, N-acetyl-D-galactosamine, N-acetyl-D-galactosamine, and N-acetyl-D-glucosamine.

In some instances, any of the glycans described herein may exist in free form in the MPV which are also within the scope of the present disclosure.

In some embodiments, the MPVs, e.g., WPVs, or a composition comprising such contain proteins having a molecular weight of about 25-30 kDa at a relative abundance of no greater than 40% (e.g., less than about 40%, about 35%, about 30%, about 25%, about 20%, about 15%, about 10%, about 5% or less). In some instances, the proteins having a molecular weight of about 25-30 kDa are caseins. In some examples, the MPVs or the composition comprising such may be substantially free of casein, e.g., cannot be detected by a conventional method or only a trace amount can be detected by the conventional method. Alternatively or in addition, the MPVs, e.g., WPVs, or a composition comprising such contain proteins having a molecular weight of about 10-20 kDa at a relative abundance of no greater than 25% (e.g., less than about 25%, about 20%, about 15%, about 10%, about 5% or less). In some instances, the proteins having a molecular weight of about 10-20 kDa are lactoglobulins. In some examples, the MPVs, e.g., WPVs, or the composition comprising such may be substantially free of lactoglobulins.

As used herein, the term “casein” refers to a family of related phosphoprotein commonly found in mammalian milk having a molecular weight of about 25-30 kDa. Exemplary species include alpha-S1-casein (αS1), alpha-S2-casein (αS2), β-casein, κ-casein. A casein protein may refer to a specific species as known in the art, for example, those noted above. Alternatively, it may refer to a mixture of at least two different species. In some instances, casein can be the population of all casein proteins found in the milk of a mammal, for example, any of those described herein (e.g., cow, goat, sheep, yak, buffalo, camel, or human).

Lactoglobulin, including α-lactoglobulin and β-lactoglobulin, is a family of whey proteins found in mammalian milk having a molecular weight of about 10-20 kDa. β-lactoglobulin typically has a molecular weight of about 18 kDa and α-lactoglobulin typically has a molecular weight of about 15 kDa. The term “lactoglobulin” may refer to one particular species, e.g., α-lactoglobulin or β-lactoglobulin. Alternatively, it may refer to a mixture of different species, for example, a mixture of α-lactoglobulin and β-lactoglobulin.

Besides the other features disclosed herein (e.g., stability), casein and/or lactoglobulin-depleted MPVs, e.g., WPVs, or compositions comprising MPVs, e.g., WPVs, have a higher cargo loading capacity, e.g., oligonucleotide loading capacity, as compared with MPVs, e.g., WPVs, prepared by the conventional ultracentrifugation method.

### D. Stability of Vesicles Purified From Milk

The vesicles purified from milk (MPVs) described herein are stable under, for example, harsh conditions, e.g., low or high pH, sonication, enzyme digestion, freeze-thaw cycles, temperature treatment, etc. Stable or stability means that the MPVs maintain substantially the same intact physical structures and substantially the same functionality as relative to the MPVs under normal conditions. For example, a substantial portion of the MPVs, e.g., WPVs, (e.g., at least 60%, at least 70%, at least 80%, at least 90%, or above) would have no substantial structural changes when they are placed under an acidic condition (e.g., pH ≤ 6.5) for a period of time. Alternatively or in addition, the MPVs, e.g., WPVs, may be resistant to enzymatic digestion such that a substantial portion of the MPVs (e.g., at least 60%, at least 70%, at least 80%, at least 90%, or above) would have no substantial structural changes in the presence of enzymes such as digestive enzymes. Further, the MPVs, e.g., WPVs, that are stable after multiple rounds of freeze-thaw cycles (e.g., up to 6 cycles) would have a substantial portion (e.g., at least 60%, at least 70%, at least 80%, at least 90%, or above) that has no substantial structural changes and/or functionality changes after the multiple freeze-thaw cycles. Because of, at least in part, the stability of the MPVs, e.g., WPVs, described herein, are able to deliver their cargo while withstanding stressed conditions or conditions under which the therapeutic agent would become deactivated, metabolized, or decomposed, e.g., saliva, digestive enzymes, acidic conditions in the stomach, peristaltic motions, and/or exposure to the various digestive enzymes, for example, proteases, peptidases, lipases, amylases, and nucleases that break down ingested components in the gastrointestinal tract.

In some embodiments, the MPV, e.g., WPV, is stable in the gut or gastrointestinal tract of a mammalian species. In some embodiments, the MPV, e.g., WPV, is stable in the esophagus of a mammalian species. In some embodiments, the MPV, e.g., WPV, is stable in the stomach of a mammalian species. In some embodiments, the MPV, e.g., WPV, is stable in the small intestine of a mammalian species. In some embodiments, the MPV, e.g., WPV, is stable in the large intestine of a mammalian species. In some embodiments, the MPV, e.g., WPV, is stable at a pH range of about pH 1.5 to about pH 7.5. In some embodiments, the MPV, e.g., WPV, is stable at a pH range of about pH 2.5 to about pH 7.5. In some embodiments, the MPV, e.g., WPV, is stable at a pH range of about pH 4.0 to about pH 7.5. In some embodiments, the MPV, e.g., WPV, is stable at a pH range of about pH 4.5 to about pH 7.0. In some embodiments, the MPV, e.g., WPV, is stable at a pH range of about pH 1.5 to about pH 3.5. In some embodiments, the MPV, e.g., WPV, is stable at a pH range of about pH 2.5 to about pH 3.5. In some embodiments, the MPV, e.g., WPV, is stable at a pH range of about pH 2.5 to about pH 6.0. In some embodiments, the MPV, e.g., WPV, is stable at a pH range of about pH 4.5 to about pH 6.0. In some embodiments, the MPV, e.g., WPV, is stable at a pH range of about pH 6.0 to about pH 7.5. In some embodiments, the MPV, e.g., WPV, is stable at a pH range of 1.5 - 7.5. In some embodiments, the MPV, e.g., WPV, is stable at a pH range of 2.5 - 7.5. In some embodiments, the MPV, e.g., WPV, is stable at a pH range of 4.0 -7.5. In some embodiments, the MPV, e.g., WPV, is stable at a pH range of 4.5 - 7.0. In some embodiments, the MPV, e.g., WPV, is stable at a pH range of 1.5 - 3.5. In some embodiments, the MPV, e.g., WPV, is stable at a pH range of 2.5 - 3.5. In some embodiments, the MPV, e.g., WPV, is stable at a pH range of 2.5 - pH 6.0. In some embodiments, the MPV, e.g., WPV, is stable at a pH range of 4.5 - 6.0. In some embodiments, the MPV, e.g., WPV, is stable at a pH range of 6.0 - 7.5. In some embodiments, the MPV, e.g., WPV, is stable at about pH 1.5, pH 2.0, pH 2.5, pH 3.0, pH 3.5, pH 4.0, pH 4.5, pH 5.0, pH 5.5, pH 6.0, pH 6.5, pH 7.0, or pH 7.5, and increments between about pH of 1.5 and about pH 7.5.

In some embodiments, the MPV, e.g., WPV, is stable in the presence of digestive enzymes, such as, for example, proteases, peptidases, nucleases, pepsin, pepsinogen, lipase, trypsin, chymotrypsin, amylase, bile and pancreatin (digestive enzymes in pancreas). In some embodiments, the MPV, e.g., WPV, is stable in the presence of pepsin or pancreatin. In particular embodiments, the MPVs, e.g., WPVs, disclosed herein can protect cargo loaded therein (e.g., oligonucleotides) from enzyme digestion (e.g., nuclease digestion).

In some embodiments, the MPVs, e.g., WPVs, disclosed herein are stable after multiple rounds of freeze-thaw cycles. For example, the MPVs, e.g., WPVs, are stable after at least two freeze-thaw cycles, e.g., at least 3 cycles, at least 4 cycles, at least 5 cycles, or at least 6 cycles. In some instances, the MPVs, e.g., WPVs, are stable up to 10 freeze-thaw cycles, e.g., up to 9 cycles, upto to 8 cycles, up to 7 cycles, or up to 6 cycles.

In some embodiments, the MPVs, e.g., WPVs, disclosed herein are stable after temperature treatment, e.g., incubated at a low temperature (e.g., at 4° C.) for a period (e.g., 1-3 days) or at a high temperature for period, e.g., at 60-80° C. for 30 minutes to 2 hours or at 100-120° C. for 5-20 minutes.

Further, the MPVs, e.g., WPVs, disclosed herein have colloidal stability. Colloidal stability refers to the long-term integrity of dispersion and its ability to resist phenomena such as sedimentation or particle aggregation. This is typically defined by the time that dispersed phase particles can remain suspended without producing precipitates.

Alternatively or in addition, the MPVs, e.g., WPVs, may be stable under physical processes, for example, sonication, centrifugation, and filtration.

### E. Preparation of Vesicles Purified From Milk

In one aspect, a MPV, e.g., WPV, may be harvested from primary sources of a milk-producing animal. In some embodiments, the MPV, e.g., WPV, is purified (e.g., isolated or manipulated) from milk or colostrum or milk component from any of a suitable mammal source. Examples include a cow, human, buffalo, goat, sheep, camel, donkey, horse, reindeer, moose, or yak. In some embodiments, the milk is from a cow. In some embodiments, the milk or colostrum is in powder form. In some embodiments, the MPVs, e.g., WPVs, are produced and subsequently isolated from mammary epithelial cells lines adapted to recapitulate the MPV, e.g., WPV, architecture of that naturally occurring in milk or whey. In another aspect, suitable MPVs, e.g., WPVs, are isolated from milk produced by a transgenic cow or other milk-producing mammal whose characteristics are optimized for producing MPVs, e.g., WPVs, with desirable properties for drug delivery, e.g., oral drug delivery.

In one aspect, the MPVs are provided using a cell line one in a batch-like process, wherein the MPVs may be harvested periodically from the cell line media. The challenge with cell line-based production methods is the potential for contamination from exosomes present in fetal bovine serum (media used to grow cells). In another aspect, this challenge can be overcome with the use of suitable serum free media conditions so that MPVs purified from the cell line of interest are harvested from the culture medium.

In one aspect, the MPVs, e.g., WPVs, are purified from a milk solution. In one aspect, the vesicles are purified from a colostrum solution. Separation of MPVs, e.g., WPVs, from the bulk solution must be performed with care. In some embodiments, a filter such as a 0.2 micron filter is used to remove larger debris from solution. In some embodiments, the method for separation of milk MPV, e.g., WPV, (for example, in the 80-120 nanometer range) includes separation based on specific MPV, e.g., WPV, properties such as size, charge, density, morphology, protein content, lipid content, or epitopes recognized by antibodies on an immobilized surface (immuno-isolation). In some embodiments antibodies are directed against epitopes located on a polypeptide selected from one or more of BTN1A1, CD81 and XOR or any of the others described herein to be associated with MPVs, e.g., WPVs.

In some embodiments, the separation method comprises a centrifugation step. In some embodiments, the separation method comprises PEG based volume excluding polymers.

In some embodiments, the separation method comprises ultra-centrifugation to separate the desired MPVs, e.g., WPV, from bulk solution. In some embodiments, sequential steps involving initial spins at 20,000 × g for up to 30 minutes followed by multiple spins at ranges of about 100,000 × g to about 120,000 × g for about 1 to about 2 hours provides a pellet or isolate rich in milk-purified vesicles.

In some embodiments, ultracentrifugation provides MPVs that can be resuspended, for example, in phosphate buffered saline or a solution of choice. In some embodiments, the vesicles are further assessed for desired properties by assessing their attributes when exposed to a sucrose density gradient and picking the fraction in 1.13-1.19 g/mL range.

In other embodiments, isolation of vesicles of the present disclosure includes using combinations of filters that exclude different sizes of particles, for example 0.45 µM or 0.22 µM filters can be used to eliminate vesicles or particles bigger than those of interest. MPVs, e.g., WPVs, may be purified by several means, including antibodies, lectins, or other molecules that specifically bind vesicles of interest, eventually in combination with beads (e.g. agarose/sepharose beads, magnetic beads, or other beads that facilitate purification) to enrich for the desired vesicles. A marker derived from the vesicle type of interest may also be used for purifying vesicles. For example, vesicles expressing a given biomarker such as a surface-bound protein may be purified from cell-free fluids to distinguish the desired vesicle from other types. Other techniques to purify vesicles include density gradient centrifugation (e.g. sucrose or optiprep gradients), and electric charge separation. All these enrichment and purification techniques may be combined with other methods or used by themselves. A further way to purify vesicles is by selective precipitation using commercially available reagents such as ExoQuick® (System Biosciences, Inc.) or Total Exosome Isolation kit (Invitrogen® Life Technologies Corporation).

In some embodiments, isolation of the MPV, e.g., WPV, is achieved by centrifuging raw (i.e., unpasteurized and/or unhomogenized milk or colostrum) at high speeds to isolate the vesicle. In some embodiments, a milk-purified vesicle is isolated in a manner that provides amounts greater than about 50 mg (e.g., greater than about 300 mg) of vesicles per 100 mL of milk. In some embodiments, the present invention provides a method of isolating an MPV, comprising the steps of: providing a quantity of milk (e.g., raw milk or colostrum); and performing a centrifugation, e.g., sequential centrifugations, on the milk to yield greater than about 50 mg of MPV per 100 mL of milk. In some embodiments, the sequential centrifugations yield greater than 300 mg of MPVs per 100 mL of milk. In some embodiments, the series of sequential centrifugations comprises a first centrifugation at 20,000 × g at 4° C. for 30 min, a second centrifugation at 100,000 × g at 4° C. for 60 min, and a third centrifugation at 120,000 × g at 4° C. for 90 min. In some embodiments, the isolated MPVs can then be stored at a concentration of about 5 mg/mL to about 10 mg/mL to prevent coagulation and allow the isolated vesicles to effectively be used for the encapsulation or loading of one or more therapeutic agents. In some embodiments, the isolated vesicles are passed through a 0.22 µm filter to remove any coagulated particles as well as microorganisms, such as bacteria.

In some embodiments, provided here are methods for isolating or purifying an MPV, (e.g., those disclosed herein), wherein the methods involve one or more steps to reduce or eliminate caseins and/or lactoglobulins from the input milk materials. Caseins are the majority of proteins in milk that have a molecular weight or about 25-30 kDa. Lactoglobulins are the majority of proteins in milk that have a molecular weight of about 10-20 kDa. Briefly, such a method may involve one or more defatting steps to remove abundant milk proteins and/or fats to produce defatted milk samples following conventional methods or those disclosed herein. The defatted milk samples can then be subject to one or more steps to disrupt casein micelles, coagulate casein and remove casein from the milk sample. The casein-depleted milk sample can thus be subject to steps to enrich MPVs, e.g., WPVs, s, for example, those approached known in the art or disclosed herein, e.g., chromatography-based methods (e.g., for scalable preparation) and ultracentrifugation-based methods.

Any approaches known in the art for removing caseins can be used in the methods disclosed herein. In some embodiments, casein removal may be achieved chemically, e.g., by acidification. For example, a suitable acid solution (e.g., acetic acid, hydrochloric acid, citric acid, etc.) or powder of a suitable acid (e.g., citric acid powder) can be added into a milk sample such as a defatted milk sample to cause coagulation of casein or casein micelles, which can be removed by a conventional method, e.g., low-speed centrifugation (e.g., ≤ 20,000 g) or filtration. Alternatively, acidification of milk may be achieved by saturation of the milk with CO₂ gas.

In other embodiments, casein removal may be achieved using enzymes capable of coagulating or digesting casein, for example, using rennet. As used herein, “rennet” refers to a mixture of enzymes capable of curdling caseins in milk. In some examples, the rennet used in the methods disclosed herein is derived from an animal, e.g., a complex set of enzymes produced in the stomachs of a ruminant mammal such as calf. Such a rennet may comprise chymosin, which is a protease enzyme that curdles casein in milk, and optionally other enzymes such as pepsin and lipase. In other examples, the rennet used in the methods disclosed herein is derived from a plant, e.g., a vegetable rennet. Vegetable rennet can be an enzyme or a mixture of enzymes that coagulates milk and separates the curds and whey from milk. In some instances, the vegetable rennet used herein can be a commercially available vegetable rennet extracted from a mold such as mucor miehei. Alternatively, one or more recombinant casein coagulation enzymes may be used for casein removal. Such recombinant enzymes may be produced using a suitable host (e.g., bacterium, yeast, insect cell, or mammalian cell) by the conventional recombinant technology.

In yet other embodiments, the method disclosed herein may involve the use of a Ca²⁺ chelating agent such as EDTA or EGTA to disrupt casein micelles, which can be then removed.

After removal of caseins (partially or completely), the milk sample can be subject to one or more steps to enrich the MPVs, e.g., WPVs, contained therein, e.g., ultracentrifugation, size exclusion chromatography, affinity purification, tangential flow filtration, or a combination thereof. In some examples, the method disclosed herein may comprise a tangential flow filtration (TFF) step for MPV, e.g., WPV, enrichment. In some instances, the method may further comprise a size exclusion chromatography following the TFF step. Alternatively, the enrichment may be achieved by a conventional approach such as ultracentrifugation.

In some embodiments, a MPV (e.g., WPV) composition described herein further includes one or more microRNAs (miRNAs) loaded into the vesicle, either by virtue of being present in the vesicles upon their isolation or by virtue of loading a miRNA for use as a therapeutic agent into the vesicles subsequent to their initial isolation. In some embodiments, the miRNA loaded into the vesicle is naturally occurring in the source of the vesicles. In some embodiments, the miRNA loaded into the vesicle is not naturally occurring in the source of the vesicles. For example, mammalian MPVs, e.g., WPVs, sometimes include loaded miRNAs in their natural state, and such miRNAs remain loaded in the vesicles upon their isolation. Such naturally-occurring miRNAs are distinguished from any miRNA therapeutic agent (or other iRNA, oligonucleotide, or other biologic) that is artificially loaded into the vesicles.

Suitable MPVs, e.g., WPVs, may also be derived by artificial production means, such as from exosome-secreting cells and/or engineered as is known in the art.

In some embodiments, MPVs, e.g., WPVs, can be further characterized by one or more of nanoparticle tracking analysis to assess particle size, transmission electron microscopy to assess size and architecture, immunogold labeling of vesicles or their contents prior to electron microscopy to track species of interest associated with exosomes, immunoblotting, or protein content assessment using the Bradford Assay.

### F. Modification of Vesicles Purified From Milk

In some embodiments, the MPV, e.g., WPV, is a natural (unmodified) MPV, e.g., a natural (unmodified) WPV. In some embodiments one or more components of the MPV are modified, e.g., modified from their natural form. In some embodiments, the MPV, e.g., WPV, is modified to alter one or more lipids, proteins, glycoproteins, glycolipids, lipoproteins, phospholipids, phosphoproteins, peptides, glycans, fatty acids, and/or sterols present in the natural MPVs, e.g., WPVs. In some embodiments, the MPV, e.g., a WPV, modified by altering the quantity, concentration, or amount of a biomolecule naturally present, e.g., the addition or complete or partial removal of a biomolecule naturally present (e.g., carbohydrate, such as a glycan; fatty acid, lipid). In some embodiments, the MPV, e.g., WPV, is modified by the addition of a biomolecule not naturally present (e.g., carbohydrate, such as a glycan; fatty acid; lipid, or protein, e.g., a glycoprotein).

In some embodiments, the MPV comprises one or more lipid components which are modified. In some embodiments, the MPV, e.g., WPV, is modified to alter one or more lipids in the MPV. In some embodiments, the lipid component of the MPV, e.g., WPV, is modified or altered, e.g., via the addition of one or more lipids not naturally present in the MPV, or by altering the amount (increasing or decreasing) of one or more lipids naturally present in the MPV. In some embodiments, the MPV, e.g., WPV, is modified to increase one or more lipids selected from one or more of the following lipids: LPE, PEO/PEP, Cer, DAG, GM2, PA, Gb3, LacCer, GM1, GM3, HexCer, GD1, PS, Chol, LPI, and SM. The lipid component of the MPV, e.g., WPV, can be altered or modified by known methods, including, for example, fusion with another vesicle having a lipid bilayer, e.g., liposome and/or lipid nanoparticle.

In some embodiments, the MPV comprises one or more lipid components, levels or amounts of which are modified. In some embodiments, the altering the amount or content of the lipids on the MPV, e.g., WPV, affects the ability of the MPVto interact, bind and/or fuse with another vesicle, e.g., a nanoparticle, e.g., a lipid nanoparticle, such as the nanoparticles described herein. In some embodiments, altering the amount or content of lipids in the MPV, e.g., WPV, alters the overall charge of the MPV. In some embodiments, altering the amount or content of the lipids in the MPVs, e.g., WPVs, results in a MPV, e.g., WPV, with greater positive charge as compared to the unaltered vesicle. In some embodiments, altering the amount or content of lipids in the MPVs, e.g., WPVs, results in a MPV, e.g., WPV, with greater negative charge as compared to the unaltered vesicle. In some embodiments, altering the charge of the vesicle makes the vesicle more attractive for interactions, binding and/or fusion with another vesicle, e.g., a nanoparticle, e.g., a lipid nanoparticle. For example, in some embodiments, lipid nanoparticles and MPVs, e.g., WPVs, having lipid contents with opposite electrostatic charges are used to promote or improve interactions, binding and/or fusion between the two types of particles. In some embodiments, interactions, binding and/or fusion is achieved between cargo-carrying lipid nanoparticles comprising negatively charged lipids and MPVs, e.g., WPVs, comprising positively charged lipids. In other embodiments, fusion is carried out between cargo-carrying lipid nanoparticles comprising positively charged lipids and MPVs, e.g., WPVs, comprising negatively charged lipids.

In some embodiments, the MPV comprises one or more glyocprotein components which are modified. In some embodiments, the MPV, e.g., WPV, comprises one or more glycoproteins. In some embodiments, the MPV, e.g., WPV, comprises a biological membrane, wherein the biological membrane comprises one or more glycoprotein(s). In some embodiments, the biological membrane is modified as compared with the natural biological membrane of the MPV, e.g., WPVIn some embodiments, the biological membrane is modified such that it has an increased number of one or more of its native glycoprotein(s). In some embodiments, the biological membrane is modified such that it has a decreased number of one or more of its native glycoprotein(s). In some embodiments, the MPV, e.g., WPV, is modified such that it includes one or more glycoprotein(s) that is not naturally present in the natural biological membrane.

In some embodiments, a MPV, e.g., WPV, having a decreased number of one or more of its native glycoprotein(s) is produced using an enzyme selected from a serine protease, cysteine protease or metalloprotease. In some embodiments, the enzyme is selected from trypsin, AspN, GluC, ArgC, chymotrypsin, proteinase K, and Lys-C. In some embodiments, the biological membrane is modified such that one or more of its native glycoprotein(s) is eliminated or not present. In some embodiments, the biological membrane is modified such that one or more of its native glycoprotein(s) is reduced.

In some embodiments, the MPV comprises one or more glyocprotein components which are modified with respect to their carbohydrate moieties. In some embodiments, the MPV, e.g., WPV, is modified to alter the amount or content of carbohydrate moieties present on a glycopolypeptide present in or associated with the MPV, e.g., WPV. In some embodiments, the MPV, e.g., WPV, is modified to increase, decrease, or otherwise alter the glycan content of the MPV, e.g., WPV, e.g., via the addition of one or more glycans not naturally present in the MPV, e.g., WPV, or by altering the amount (increasing or decreasing) of one or more glycans naturally present in the MPV, e.g., WPV.

In some embodiments, one or more components of the biological membrane of the MPV are modified, e.g., a modification in the glycoproteins. In some embodiments, the biological membrane of the MPV, e.g., WPV, is modified such that one or more of its native glycoprotein(s) is altered. In some embodiments, the one or more native glycoprotein(s) is altered such that the number of glycan residues present on the glycoprotein(s) is increased. In some embodiments, the MPV, e.g., WPV, is produced using glycosylation that adds one or more glycans to the glycoprotein. In some embodiments, the MPV, e.g., WPV, is modified to increase one or more glycoprotein(s) having one or more of the following glycans: terminal b-galactose, terminal a-galactose, N-acetyl-D-galactosamine, N-acetyl-D-galactosamine, and N-acetyl-D-glucosamine.

In some embodiments, the one or more native glycoprotein(s) is altered such that the number of glycan residues present on the glycoprotein(s) is decreased. In some embodiments, the number of glycan residues is decreased by cleavage of one or more glycan residues present on the glycoprotein(s). In some embodiments, the MPV, e.g., WPV, is produced using an enzyme selected from a glycosidase, exoglycosidase, endoglycosidase, glycoamidase, neuraminidase, galactosidase, peptide:N- glycosidase (PNGase), glycohydrolase, and any combination thereof wherein the milk exosome is contacted with the enzyme to remove one or more glycans. In some embodiments, the enzyme is selected from a β-N-acetylglucosaminidase, PNGase F, β (1-4) Galactosidase, O-Glycosidase, N-Glycosidase, N-glycohydrolase, Endo H, Endo D, Endo F₂, EndoF₃, and any combination thereof.

In some embodiments, the number of glycan residues is decreased by cleavage of one or more glycan residues present on the glycoprotein(s). In some embodiments, the MPV, e.g., WPV, is produced using an enzyme selected from a glycosidase, exoglycosidase, endoglycosidase, glycoamidase, neuraminidase, galactosidase, peptide:N- glycosidase (PNGase), glycohydrolase, and any combination thereof wherein the milk exosome is contacted with the enzyme to remove one or more glycans. In some embodiments, the enzyme is selected from a β-N-acetylglucosaminidase, PNGase F, β (1-4) Galactosidase, O-Glycosidase, N-Glycosidase, N-glycohydrolase, Endo H, Endo D, Endo F₂, EndoF₃, and any combination thereof.

In some embodiments, two or more native glycoprotein(s) are altered such that at least one glycoprotein has an increased number of glycan residues and at least one other glycoprotein has a decreased number of glycan residues or is missing its glycan residue(s), wherein the glycoprotein(s) having an increased number of glycan residues is different from the glycoprotein(s) having a decreased number of glycan residues or missing glycan residues. In some embodiments, the one or more native glycoprotein(s) is altered such that it comprises a modified glycan. In some embodiments, the modified glycan comprises at least one carbohydrate moiety that differs from that of the glycan in the native glycoprotein(s). In some embodiments, the modified glycan comprises one or more galactose, mannose, O-glycans, N-acetyl- glucosamines, and/or N-glycan chains or any combination thereof. In some embodiments, the glycan is selected from comprises one or more D- or L- glucose, erythrose, fucose, galactose, mannose, lyxose, gulose, xylose, arabinose, ribose, 2′-deoxyribose, glucosamine, lactosamine, polylactosamine, glucuronic acid, sialic acid, sialyl-Lewis X (SLex), N-acetyl-glucosamine, N- acetyl-galactosamine, neuraminic acid, N-glycolylneuraminic acid (Neu5Gc), N- acetylneuraminic acid (Neu5Ac), an N-glycan chain, an O-glycan chain, a Core 1, Core 2, Core 3, or Core 4 structure, or a phosphate- or acetate-modified analog thereof or a combination thereof. In some embodiments, the modified glycan lacks a portion of one or more of its carbohydrate chain(s). In some embodiments, the modified glycan is missing one or more of its carbohydrate chain(s). In some embodiments, the modified glycan comprises one or more altered carbohydrate chain(s). In some embodiments, the one or more native glycoprotein(s) is altered such that at least one glycan present on the glycoprotein(s) is substituted with a glycan that is not naturally present in the native glycoprotein(s). See also WO2018170332, the relevant disclosures of which are incorporated by reference for the purpose and subject matter referenced herein.

In some embodiments, the MPV comprises one or more components, the levels or amounts of which are modified. In some examples, the MPV comprises one or more glycoproteins components, the glycan levels or amounts of which are modified. In some of these embodiments, the modifications may change the properties of the MPV. In some embodiments, altering the number or content of the glycan residues on the MPV, e.g., WPV, affects the colloidal stability of the MPV. In some embodiments, altering the number or content of the glycan residues on the MPV, e.g., WPV, modulates the interaction between MPVs and GI cells, e.g., enhances the uptake of MPVs in GI cells.

In some embodiments, the altering the number or content of the glycan residues on the MPV, e.g., WPV, affects the ability of the MPVto interact, bind and/or fuse with another vesicle, e.g., a nanoparticle, e.g., a lipid nanoparticle, such as the nanoparticles described herein. In some embodiments, altering the number or content of the glycan residues alters the overall charge of the MPV, e.g., WPV. In some embodiments, altering the number or content of the glycan residues in the MPVs, e.g., WPVs, results in a vesicle with greater positive charge as compared to the unaltered MPV. In some embodiments, altering the number or content of the glycan residues in the MPVs, e.g., WPVs, results in an MPV with greater negative charge as compared to the unaltered vesicle. In some embodiments, altering the charge of the vesicle makes the vesicle more attractive for interactions, binding and/or fusion with another vesicle, e.g., a nanoparticle, e.g., a lipid nanoparticle, e.g., a liposome. For example, in some embodiments, lipid nanoparticles, such as liposomes, having lipid contents and MPVs, e.g., WPVs, having lipid and/or glycan or glycoprotein contents with opposite electrostatic charges are used to promote or improve interactions, binding and/or fusion between the two types of particles. In some embodiments, interactions, binding and/or fusion is achieved between cargo-carrying lipid nanoparticles comprising negatively charged lipids and MPVs, e.g., WPV, comprising positively charged lipids and/or glycoprotein or glycan contents. In other embodiments, fusion is carried out between cargo-carrying lipid nanoparticles comprising positively charged lipids and MPVs, e.g., WPVs, comprising negatively charged lipids and/or glycoprotein or glycan contents.

In some embodiments, altering the number or content of the glycan residues on the MPV, e.g., WPV, improves the ability of the MPV and/or the LNP-MPV as described herein to be enriched and/or purified. In some embodiments, altering the number or content of the glycan residues on the MPV, e.g., WPV, improves the ability of the MPV and/or the LNP-MPV, such as a fused liposome-MPV or fused liposome-WPV, as described herein to be detected in vitro or in vivo. In some embodiments, anti-glycan antibodies or lectins are used to enrich and/or purify MPVs, e.g., WPVs, and/or LNP-MPVs, such as a fused Liposome-MPVs or fused liposome-WPVs, as described herein. In some embodiments, anti-glycan antibodies or lectins are used to detect and/or purify MPVs, e.g., WPVs, and/or LNP-MPVs as described herein. Accordingly, methods to enrich and/or purify these MPVs, e.g., WPVs, or LNP-MPVs are contemplated which comprise contacting anti-glycan antibodies or lectins with MPVs, e.g., WPVs, and/or LNP-MPVs. In some embodiments, methods to detect MPVs, e.g., WPVs, or LNP-MPVs using anti-glycan antibodies or lectins are contemplated.

In some embodiments, the MPVs, e.g., WPVs, are modified to alter one or more proteins in the MPV. In some embodiments, levels of existing MPV, e.g., WPV, proteins are reduced. In some embodiments, proteins which do not naturally occur in the MPV are added. In some embodiments, the MPVs, e.g., WPVs, are modified to display a lectin, which is capable of binding to glycoproteins, e.g., a glycoprotein present on a nanoparticle. Fused liposome-MPVs modified with one or more lectins are also referred to as fused LNP-MPV programmed with surface ligands or surface programmed LNP-MPVs. Fused liposome-WPVs modified with one or more lectins are also referred to as fused liposome-WPV programmed with surface ligands or surface programmed liposome-WPVs.

Accordingly, in some embodiments, the MPVs, e.g., WPVs, display lectins on their surface. In some embodiments, the MPVs, e.g., WPVs, display one or more lectins selected from Con A, RCA, WGA, DSL, Jacalin, or any combination thereof. Alternatively, the MPVs, e.g., WPVs, may be modified to display a binding moiety capable of binding to another binding moiety that is conjugated to the surface of the lipid nanoparticle. Such binding moiety pairs may be any ligand-receptor pairs such as biotin-streptavidin.

Modifications to the MPVs, e.g., WPVs, as described herein can be made via conventional methods. For example, MPVs isolated from a natural source may be subject to extrusion (e.g., once or multiple times) through a filter having a suitable size, e.g., 50 nM, 75 nM, or 100 nM, to change size distribution. In another example, MPVs, e.g., WPVs, isolated from one or more natural sources may be subject to homogenization (e.g., under high pressure in some instances) to cause fusion of particles. Alternatively, extrusion or homogenization may be performed to MPVs, e.g., WPVs, isolated from a natural source in the presence of other natural or artificial lipid membrane vesicles or protein micelles or aggregates to produce fused particles. Such fusion may lead to change of protein and/or lipid content of the resultant particles, for example, incorporating non-naturally occurring lipids, which may present in the artificial lipid membrane particles. In another example, additional lipids may be incorporated into MPVs, e.g., WPVs, isolated from a natural source via saturation of the MPVs with specific lipids of interest or incubating the MPV with lipid films, which may contain lipids of interest (e.g., cholesterol, phospholipids, ceramides and/or sphingomyelins).

In some embodiments, a MPV, e.g., WPV, may be modified to add a binding moiety on the surface to facilitate fusion with a liponanoparticle as disclosed herein for cargo loading. Alternatively or in addition, MPVs, e.g., WPVs, isolated from a natural source may be modified by removing certain lipid contents. For example, methyl-beta-cyclodextrin can be used to extract cholesterol from MPVs. Alternatively or in addition, MPVs, e.g., WPVs, may be modified by conjugating suitable moieties, such as proteins, polypeptides, peptides, glycans, etc. onto surface proteins of the MPVs, via conventional methods.

Any of the modified MPVs, e.g., WPVs, described above are suitable for any of the fusion, cargo loading, purification and enrichment methods described herein. Accordingly, in some embodiments, the modifications and resulting properties for the MPVs, e.g., WPVs, are conferred to the LNP-MPV, e.g., the fused liposome-WPV or fused liposome-WPV. In some embodiments, any of the modifications to lipids, polypeptides, glycans and others described herein may be present in an LNP-MPV, e.g., a liposome-WPV. Accordingly, in any of the above embodiments relating to modified vesicles, the MPVs, e.g., WPVs, and/or LNP-MPVs or compositions of MPVs and/or LNP-MPVs can comprise a relative abundance of casein less than about 40%, about 35%, about 30%, about 25%, about 20%, about 15%, about 10%, about 5% or less. In some of these above embodiments, the MPVs, e.g., WPVs, and/or LNP-MPVs or compositions of MPVs, e.g., WPVs, and/or LNP-MPVs produced by the fusion methods described herein are substantially free of casein. In some of these above embodiments, the MPVs, e.g., WPV, and/or LNP-MPVs or compositions of MPVs, e.g., WPVs, and/or LNP-MPVs comprise lactoglobulin at a relative abundance of no greater than 25% (e.g., less than about 25%, about 20%, about 15%, about 10%, about 5% or less). In some embodiments, the MPVs, e.g., WPVs, and/or LNP-MPVs or the composition comprising such may be substantially free of lactoglobulins.

In some of these embodiments, the size of the MPVs, e.g., WPVs, and/or LNP-MPVs is about 20-1,000 nm. In some embodiments, the size of the MPVs, e.g., WPVs, and/or LNP-MPVs is about 100-160 nm. In some of these above embodiments, the MPVs, e.g., WPVs, and/or LNP-MPVs comprise a lipid membrane to which one or more proteins described herein are associated. In some embodiments, the MPVs, e.g., WPVs, and/or LNP-MPVs comprise one or more selected from BTN1A1, CD81 and XOR. In some embodiments, one or more proteins associated with the lipid membrane of the MPVs, e.g., WPVs, and/or LNP-MPVs are glycosylated. In some embodiments, the MPVs, e.g., WPVs, and/or LNP-MPVs demonstrate stability under freeze-thaw cycles and/or temperature treatment. In some embodiments, the MPVs, e.g., WPVs, and/or LNP-MPVs demonstrate colloidal stability when loaded with the biological molecule. In some embodiments, the MPVs, e.g., WPVs, and/or LNP-MPVs demonstrate stability under acidic pH, e.g., pH of ≤ 4.5 or pH of ≤2.5. In some embodiments, the MPVs, e.g., WPVs, and/or LNP-MPVs demonstrate stability upon sonication. In some embodiments, the MPVs, e.g., WPVs, and/or LNP-MPVs demonstrate resistance to enzyme digestion, e.g., resistance to one or more digestive enzymes described herein and/or resistance to nuclease treatment. In any of these embodiments, the MPVs, e.g., WPVs, and/or LNP-MPV can be used for oral delivery of a cargo, e.g., a cargo encapsulated in the MPV, e.g., WPV, and/or LNP-MPV. In some embodiments, the MPVs, e.g., WPVs, and/or LNP-MPVs are formulated to form a suitable composition for use in oral delivery of the cargo encapsulated therein to a subject, for example, a human patient. In some embodiments, the cargo can be a peptide, a protein, a nucleic acid, a polysaccharide, or a small molecule.

### II. Cargos

Any of the LPN-MPVs disclosed herein, such as liposome-WPVs comprise s, e.g., one or more cargos.

As used herein, the term “cargo” is meant to include any biomolecule or agent that can be loaded into or by a MPV, e.g., WPV, including, for example, a biologic, small molecule, therapeutic agent, and/or diagnostic agent. The cargo (e.g., biological molecule) in the cargo-loaded MPVs, e.g., WPVs, described herein can be of any type. Examples include, but are not limited to, proteins, nucleic acids, lipids, carbohydrates, and small molecules. The cargo may be a biological molecule that is not naturally-occurring in a MPV, e.g., WPV, has been modified as described herein.

In some embodiments, the biological molecule is a biologic agent. As used herein, the term “biologic” is used interchangeably with the term “biologic therapeutic agent”. One of ordinary skill in the art will recognize that such biologic agents include those described herein. In some embodiments, the biologic agent is a peptide, a polypeptide, or protein. In other embodiments, the biologic agent is a nucleic acid. In some examples, the nucleic acid may be a therapeutic agent per se, i.e., comprises a nucleic acid based biologic agent (e.g., an interfering RNA, an antisense oligonucleotide, or an aptamer). In other examples, the nucleic acid may encode a therapeutic agent (e.g., a protein-based therapeutic agent).

Any of the cargo-loaded LNP-MPVs, disclosed herein are useful to transport the cargos (e.g., biologic agents such as macromolecular medicines) to the intestinal tract, for example, to selected mucosal cell types of the intestinal tract, e.g., the small intestine. Without wishing to be bound by any theory, the cargos can act either directly in the GI tract or transit through the mucosa to the underlying lymphatic vascular network. Alternatively, in the case of nucleic acid-based cargos encoding biologic agent(s), for example, nucleic acid-based cargos that either comprise or yield mRNAs, may be employed in some instances to produce complex biologics such as antibodies within mucosal cells, which, once produced, are secreted into the mucosal lymphatic vascular network for subsequent systemic distribution. Consequently, in some embodiments of the disclosure, an LNP-MPV made according to the methods provided herein, comprises one or more biologic agents, wherein the biologic agent acts directly in the GI tract. In some embodiments, the biologic agent is taken up by selected mucosal cell types. In some embodiments, the biologic agent is released into the lumen of the gut. In some embodiments, the biologic agents transit through the mucosa to the underlying lymphatic vascular network. In some examples, an LNP-MPV , e.g., made according to the methods provided herein, comprises one or more biologic agents comprising a nucleic acid, which comprises an mRNA or may be transcribed to mRNA, e.g., after it is taken up into a target cell type, such as a mucosal cell type. In some examples, after being taken up into the target cell, e.g., mucosal cell, the nucleic acid is expressed, resulting in the production of a therapeutic protein, e.g., as described herein. In some examples, the nucleic acid is expressed within mucosal cells, e.g., to produce a biologic agent, e.g., one or more antibodies, within mucosal cells, wherein the biologic agent is secreted into the mucosal lymphatic vascular network for subsequent systemic distribution.

### A. Nucleic Acids

In some embodiments, the biological molecule is a nucleic acid, for example, an oligonucleotide therapeutic agent, such as a single-stranded or double-stranded oligonucleotide therapeutic agent. In some examples, the oligonucleotide therapeutic agent can be a single-stranded or double-stranded DNA, iRNA, shRNA, siRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof.

In some embodiments, the nucleic acid is a ncRNA of about 30 to about 200 nucleotides (nt) in length or a long non-coding RNA (lncRNA) of about 200 to about 800 nt in length. In some embodiments, the lncRNA is a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In some embodiments, the ncRNA is selected from a piwi-interacting RNA (piRNA), primary miRNA (pri-miRNA), or premature miRNA (pre-miRNA).

In some examples, the present disclosure provides the following lipid-modified double-stranded RNA that may be loaded in and delivered by the MPVs, e.g., WPVs, described herein. In some embodiments, the RNA is one of those described in CA 2581651 or US 8,138,161, each of which is hereby incorporated by reference in its entirety.

The nucleic acid-based cargo loaded in the MPV, e.g., WPV, may not be naturally-occurring in the milk source, from which the MPV is purified.

### (a) ncRNA and lncRNA

The broad application of next-generation sequencing technologies in conjunction with improved bioinformatics has helped to illuminate the complexity of the transcriptome, both in terms of quantity and variety. In humans, 70-90% of the genome is transcribed, but only ~2% actually codes for proteins. Hence, the body produces a huge class of non-translated transcripts, called long non-coding RNAs (lncRNAs), which have received much attention in the past decade. Recent studies have illuminated the fact that lncRNAs are involved in a plethora of cellular signaling pathways and actively regulate gene expression via a broad selection of molecular mechanisms.

Human and other mammalian genomes pervasively transcribe tens of thousands of long non-coding RNAs (lncRNAs). The latest edition of data produced by the public research consortium GenCode (version #27) catalogs just under 16,000 lncRNAs in the human genome, producing nearly 28,000 transcripts; when other databases are included, more than 40,000 lncRNAs are known.

These mRNA-like transcripts have been found to play a controlling role at nearly all levels of gene regulation, and in biological processes like embryonic development. A growing body of evidence also suggests that aberrantly expressed lncRNAs play important roles in normal physiological processes as well as multiple disease states, including cancer. lncRNAs are a group that is commonly defined as transcripts of more than 200 nucleotides (e.g., about 200 to about 1200 nt, about 2500 nt, or more) that lack an extended open reading frame (ORF). The term “non-coding RNA” (ncRNA) includes lncRNA as well as shorter transcripts of, e.g., less than about 200 nt, such as about 30 to 200 nt. Several lncRNAs, e.g., gadd74 and lncRNA-RoR5, modulate cell cycle regulators such as cyclins, cyclin-dependent kinases (CDKs), CDK inhibitors and p53 and thus provide an additional layer of flexibility and robustness to cell cycle progression. In addition, some lncRNAs are linked to mitotic processes such as centromeric satellite RNA, which is essential for kinetochore formation and thus crucial for chromosome segregation during mitosis in humans and flies. Another nuclear lncRNA, MA-linc1, regulates M phase exit by functioning in cis to repress the expression of its neighboring gene Purα, a regulator of cell proliferation. Since deregulation of the cell cycle is closely associated with cancer development and growth, cell cycle regulatory lncRNAs may have oncogenic properties.

Thus, in some embodiments, delivery of a ncRNA, such as to a specific tissue or organ of interest, corrects aberrant RNA expression levels or modulates levels of disease-causing lncRNA. Accordingly, in some embodiments, the nucleic acid-based cargo loaded into MPV, e.g., WPV, can be a non-coding RNA (ncRNA). In some examples, the ncRNA is a long non-coding RNA (lncRNA) of about 200 nucleotides (nt) in length or greater. In some examples, the lncRNA can be about 200 nt to about 1,200 nt in length. In some examples, the lncRNA is about 200 nt to about 1,100, about 1,000, about 900, about 800, about 700, about 600, about500, about 400, or about 300 nt in length. In other examples, the ncRNA can be of about 25 nt or about 30 nt to about 200 nt in length.

### (b) Micro RNA (miRNA)

In some embodiments, the nucleic acid-based cargo is a miRNA. As would be recognized by those skilled in the art, miRNAs are small non-coding RNAs that are about 17 to about 25 nucleotide bases (nt) in length in their biologically active form. In some embodiments, the miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nt in length. miRNAs regulate gene expression post- transcriptionally by decreasing target mRNA translation. In some instances, miRNAs function as negative regulators.

There are generally three forms of miRNAs: primary miRNAs (pri- miRNAs), premature miRNAs (pre-miRNAs), and mature miRNAs, all of which are within the scope of the present disclosure. Primary miRNAs are expressed as stem-loop structured transcripts of about a few hundred bases to over 1 kb. The pri- miRNA transcripts are cleaved in the nucleus by Drosha, an RNase II endonuclease that cleaves both strands of the stem near the base of the stem loop. Drosha cleaves the RNA duplex with staggered cuts, leaving a 5′ phosphate and 2 nt overhang at the 3′ end. The cleaved product, the premature miRNA (pre-miRNA) is about 60 to about 110 nt long with a hairpin structure formed in a fold-back manner. Pre-miRNA is transported from the nucleus to the cytoplasm by Ran- GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length. In some embodiments, the miRNAs encapsulated by the microvesicles of the presently-disclosed subject matter are selected from miR-155, which is known to act as regulator of T- and B-cell maturation and the innate immune response, or miR-223, which is known as a regulator of neutrophil proliferation and activation. Other non-natural miRNAs such as iRNAs (e.g. siRNA) or natural or non-natural oligonucleotides may be present in the milk-purified vesicles and represent an encapsulated therapeutic agent, as the term is used herein.

### (c) Short Interfering RNA (siRNA)

In some embodiments, the nucleic acid-based cargo disclosed herein is a siRNA. Small interfering RNA (siRNA), sometimes known as short interfering RNA or silencing RNA, is a class of double-stranded RNA molecules, 20-25 base pairs in length (of similar length to miRNA). siRNAs generally exert their biological effects through the RNA interference (RNAi) pathway. siRNAs generally have 2 nucleotide overhangs that are produced through the enzymatic cleavage of longer precursor RNAs by the ribonuclease Dicer. siRNAs can limit the expression of specific genes by targeting their RNA for destruction through the RNA interference (RNAi) pathway. It interferes with the expression of specific genes with complementary nucleotide sequences by degrading mRNA after transcription, preventing translation. siRNA can also act in RNAi-related pathways as an antiviral mechanism or play a role in the shaping of the chromatin structure of a genome.

In some examples, the RNA is an siRNA molecule comprising a modified ribonucleotide, wherein said siRNA (a) comprises a two base deoxynucleotide “TT” sequence at its 3′ end, (b) is resistant to RNase, and (c) is capable of inhibiting viral replication. In some examples, the siRNA molecule is 2′ modified. In some embodiments, the 2′ modification is selected from the group consisting of fluoro-, methyl-, methoxyethyl- and propyl-modification. In some embodiments, the fluoro-modification is a 2′-fluoro-modification or a 2′, 2′-fluoro-modification.

In some embodiments, at least one pyrimidine of the siRNA is modified, and said pyrimidine is cytosine, a derivative of cytosine, uracil, or a derivative of uracil. In some embodiments, all of the pyrimidines in the siRNA are modified. In some embodiments, both strands of the siRNA contain at least one modified nucleotide. In some embodiments, the siRNA consists of about 10 to about 30 ribonucleotides. In some embodiments, the siRNA molecule consists of about 19 to about 23 ribonucleotides.

In some embodiments, the siRNA molecule comprises a nucleotide sequence at least 80% identical to the nucleotide sequence of siRNA5, siRNAC1, siRNAC2, siRNA5B1, siRNA5B2 or siRNA5B4. In some embodiments, the siRNA molecule is linked to at least one receptor-binding ligand. In some embodiments, the receptor-binding ligand is attached to a 5′-end or 3′-end of the siRNA molecule. In some embodiments, the receptor binding ligand is attached to multiple ends of said siRNA molecule. In some embodiments, the receptor-binding ligand is selected from the group consisting of a cholesterol, an HBV surface antigen, and low-density lipoprotein. In some embodiments, the receptor-binding ligand is cholesterol.

In some embodiments, the siRNA molecule comprises a modification at the 2′ position of at least one ribonucleotide, which modification at the 2′ position of at least one ribonucleotide renders said siRNA resistant to degradation. In some embodiments, the modification at the 2′ position of at least one ribonucleotide is a 2′-fluoro-modification or a 2′,2′- fluoro-modification.

In an embodiment, the present disclosure provides a double-stranded (dsRNA) molecule that mediates RNA interference in target cells wherein backbone sugars of one or more of the pyrimidines in the dsRNA are modified to include a 2′-fluorine, a 2′-O-methyl, a 2′-MOE, a phosphorothioate bond (e.g., including stereoisomers of those and other modifications of phosphodiether bonds, bridged nucleotides, e.g., locked nucleotides), or a combination thereof. In some instances, the modification may include inverted bases and/or abasic nucleotides. Alternatively or in addition, the modifications may include peptide nucleic acids (PNAs), such as gamma-PNAs and/or PNA-oligopeptide hybrids. Any of the modifications described herein may apply to other types of nucleic acid moelcules as also disclosed herein where applicable.

Any of the nucleic acid-based cargo molecules disclosed herein may comprise one or more modifications at any position applicable. For example, non-limiting examples of modifications can comprise one or more nucleotides modified at the 2′-position of the sugar, e.g., 2′-Oalkyl, 2′-O-alkyl-O-alkyl, or 2′-fluoro-modified nucleotide. In some embodiments, modifications to an RNA molecule may include 2′-fluoro, 2′-amino or 2′-O-methyl modifications on he ribose of one or more pyrimidines, abasic residues, desoxy nucleotides, or an inverted base at the 3′ end of the RNA molecule. Alternatively or in addition, the nucleic acid-based cargo molecule may include one or more modifications in the bockbones such that the modified nucleic acid molecule may be more resistant to nuclease digestion relative to the non-modified counterpart. Such backbone modifications include, but are not limited to, phosphorothioates, phosphorothyos, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH,~N(CH₃)—O—CH₂ (known as a methylene(methylimino) or MMI backbone), CH₂—O—N (CH₃)—CH₂, CH₂—N (CH₃)—N (CH₃)—CH₂ and O—N (CH₃)—CH₂—CH₂ backbones (wherein the native phosphodiester backbone is represented as O—P—O—CH); amide backbones (De Mesmaeker et al., Ace. Chem. Res. 28:366-374; 1995); morpholino backbone structures (U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone; see, e.g., Nielsen et al., Science 254:1497; 1991). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′-alkylene phosphonates and chiral phosphonates, phosphinates, phosphoramidates comprising 3′-amino phosphoramidate and aminoaklylphosphoramidates, thionophosphoramidates, thionoalkylphosphonates thionoalkylphosphotriesters, and boranophosphates having normal 3′-5′ linkages, 2′-5′ linaged analogs of these, and those having inverted polarity wherein the adjacent pairs of nucleotide units are linked 3′-5′ to 5′-3′ or 2′-5′ to 5′-2′. See, e.g., WO2017/077386, the relevant disclosures of which are incorporated by reference for the purpose and/or subject matter references herein.

In an embodiment, the nucleic acid molecule in any of the cargo-loaded MPVs, e.g., WPVs, described herein is a small interfering RNA (siRNA) that mediates RNA interference in target cells wherein backbone sugars of one or more of the pyrimidines in the siRNA are modified to include a 2′-Fluorine. In an embodiment, all of the backbone sugars of pyrimidines in the dsRNA or siRNA molecules of the first and second embodiments are modified to include a 2′-Fluorine. In an embodiment, the 2′-Fluorine dsRNA or siRNA of the third embodiment is further modified to include a two base deoxynucleotide “TT” sequence at the 3′ end of the dsRNA or siRNA.

Other types of nucleic acid-based cargos disclosed herein may also comprise any of the modifications disclosed above where applicable.

In some embodiments, the siRNA molecule is about 10 to about 30 nucleotides long, and mediates RNA interference in target cells. In some embodiments, the siRNA molecules are chemically modified to confer increased stability against nuclease degradation, but retain the ability to bind to target nucleic acids.

### (d) Messenger RNAs (mRNAs)

In some embodiments, the nucleic acid-based cargo disclosed herein is an mRNA molecule, which may be a naturally-occurring mRNA or a modified mRNA molecule. In some examples, the mRNA may be modified by introduction of non-naturally occurring nucleosides and/or nucleotides. Any modified nucleosides and/or nucleotides may be used for making the modified mRNA as disclosed herein. Examples include those described in US20160256573, the relevant disclosures are incorporated by reference for the purpose and subject matter referenced herein. In other examples, the mRNA molecule may be modified to have reduced uracil content. See, e.g., US20160237134, the relevant disclosures are incorporated by reference for the purpose and subject matter referenced herein.

mRNA is a non-infectious and non-integrating platform with no potential risk of infection or insertional mutagenesis. Moreover, mRNA molecules can be degraded by normal cellular processes. mRNA stability and immunogenicity can be manipulated by utilizing various RNA modifications which can make mRNA more stable and more highly translatable. Two major types of RNA are currently studied as gene delivery vehicles, conventional mRNA and virally derived, self-amplifying RNA. Conventional mRNA-based therapeutics encode the antigen of interest and contain 5′ and 3′ untranslated regions (UTRs), whereas self-amplifying RNAs encode not only the therapeutic protein but also the viral replication machinery that enables intracellular RNA amplification and abundant protein expression. Self-amplifying mRNA (SAM) therapeutics are based on an alphavirus genome, which comprises genes encoding the RNA replication machinery but lacks the genes encoding the structural proteins. The structural genes are substituted with the sequence encoding the antigen.

In some embodiments, the mRNA cargo, when expressed, produces one or more therapeutic agents, for example, a therapeutic polypeptide of interest or a therapeutic nucleic acid of interest as described herein. See, e.g., section titled “Polypeptides” below and Tables 3 and 4. In some examples, the mRNA cargo may collectively encode a therapeutic antibody, such as those listed in Table 3. In specific examples, the mRNA cargos may collectively encode a neutralizing antibody targeting a coronavirus, for example, SARS (e.g., SARS-CoV-2). Exemplary anti-SARS-CoV-2 antibodies include anti-S1 antibodies (e.g., IgG antibodies), for example, 311mab-31B5, 311mab-32D4, and 311mab-31B9 (Chen et al., Cellular & Molecular Immunology, 17:647-649 (2020); 47D11 (binding to S protein ectodomain, part of the RBD conserved core; Wang, C., et al., Nature Communications, 2020. 11(1): p. 2251); CR3033 (binding to a conserved epitope distinct from the RBM; Tian, X., et al., 2020. 9(1): p.382-385); VHH-72 (binding to RBD; Wrapp, D., et al., Cell, 2020. 181(5): p. 1004-1015.e15); S309 (Pinto, D., et al., BioRxiv, 2020: p. 2020.04.07.023903); B38 and H4 (Wu, Y., et al., Science, 2020. 368(6496): p. 1274); CB6 (Shi, R., et al. Nature, 2020); and 4A8 (Chi, X., et al. Science, 2020. eabc6952). In some embodiments, the mRNA may encode a hormone, growth factor, cytokine or an enzyme.

In some embodiments, the mRNA comprises one or more modifications from its natural form, i.e., the mRNA is a modified mRNA (mmRNA). In some embodiments, the therapeutic mRNA includes a structural modification that improves one or more of the stability and/or clearance in tissues, receptor uptake and/or kinetics, cellular access by the compositions, engagement with translational machinery, mRNA half-life, translation efficiency, immune evasion, protein production capacity, secretion efficiency (when applicable), accessibility to circulation, protein half-life and/or modulation of a cell’s status, function and/or activity. Typically, the basic components of an mRNA molecule include at least a coding region, a 5′UTR, a 3′UTR, a 5′ cap and a poly-A tail. Building on this wild type modular structure, the present invention provides exosomes loaded with a mRNA or a non-natural mRNA. Suitable non-natural mRNA molecules maintain a modular organization, but which comprise one or more structural and/or chemical modifications or alterations which impart useful properties to the polynucleotide including, in some embodiments, the lack of a substantial induction of the innate immune response of a cell into which the polynucleotide is introduced. It is contemplated as a part of the disclosure that such a therapeutic mRNA can encode and express in a target cell any of the polypeptide therapies described herein and known in the art.

### (e) DNA-Based Bargos

In some embodiments, the nucleic acid-based cargo is a DNA molecule. In some instances, the DNA molecule may comprise a gene delivery vehicle, e.g., an expression system. The expression system can comprise one or more genes encoding one or more therapeutic biologic agents, for example, a therapeutic peptide, polypeptide, or protein as disclosed herein.

Upon administration, the genes are expressed and therapeutic biologic agents are produced in a target cell, for example, a therapeutic polypeptide of interest or a therapeutic nucleic acid of interest as described herein. See, e.g., Section titled “Polypeptides” below and Tables 3 and 4. In some examples, the DNA cargos may collectively encode a therapeutic antibody, such as those listed in Table 3. In specific examples, the DNA cargos may collectively encode a neutralizing antibody targeting a coronavirus, for example, SARS (e.g., SARS-CoV-2). See examples provided in Table 3. In some embodiments, the DNA cargos may encode a hormone, growth factor, cytokine or an enzyme.

The gene delivery vehicle or expression system can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers known in the art. Expression of the coding sequence can be either constitutive or regulated. Viral-based vectors, which are generally more efficient in gene transduction than non-viral based vectors, for delivery of a desired polynucleotide and expression in a desired cell are well known in the art.

Recombinant viral vectors use attenuated viruses (or bacterial strains) as vectors. A gene encoding a major antigen of a pathogen can be introduced into an attenuated virus or bacterium. The attenuated organism acts as a vector that replicates and expresses the gene product of the pathogen in the host. The utility of viral vectors is based on the ability of viruses to infect cells. In general, the advantages of viral vectors are as follows: (a) high efficiency gene transduction; (b) highly specific delivery of genes to target cells; and (c) induction of robust immune responses, and increased cellular immunity. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), adenovirus, and adeno-associated virus (AAV) vectors (see, e.g., PCT Publication Nos. WO 94/12649, WO 93/03769; WO 93/19191; WO 94/28938; WO 95/11984 and WO 95/00655), herpes virus, lentivirus, pox virus, Ebstein-Barr virus, and adenovirus.

Non-viral expression systems, which are generally less immunogenic than viral expression systems, include plasmids, naked DNA, and oligonucleotides (reviewed in Hardee et al., Advances in Non-Viral DNA Vectors for Gene Therapy; Genes (Basel). 2017 Feb; 8(2): 65). Non-viral delivery vehicles include polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581, the relevant disclosures of each of which is herein incorporated by reference for the purpose and subject matter referenced herein.

Closed-end DNA (ceDNA) is another example of a non-viral expression system, which has garnered interest due to its potential for delivery and expression of large cargo. ceDNA is stably maintained in the cells but less likely to integrate into the host genome than for example viral vectors. Production and characterization of closed end DNA is described in Li et al., Production and characterization of novel recombinant adeno-associated virus replicative-form genomes: a eukaryotic source of DNA for gene transfer; PLoS One. 2013 Aug 1;8(8):e69879 and in International Patent Publication WO2017152149, the relevant disclosures of each of which is herein incorporated by reference for the purpose and subject matter referenced herein.

In some embodiments, the biologic agent comprises a nucleic acid, comprising a ceDNA. In some embodiments, the ceDNA comprises one or more genes encoding one or more neutralizing, e.g., broadly neutralizing anti-pathogenic antibodies, e.g., anti-viral antibodies, e.g., anti-COVID antibodies. In some embodiments, the biologic agent comprising a nucleic acid, e.g., mRNA, ceDNA or other expression system, is administered via inhalation. In some embodiments, the biologic agent comprising a nucleic acid, e.g., mRNA, ceDNA, or other expression system, is administered via injection (IV or SQ). In some embodiments, the biologic agent comprising a nucleic acid, e.g., RNA, e.g., siRNA, mRNA, or DNA, e.g., viral or non-viral or ceDNA or other expression system, is administered orally. In some embodiments, a ceDNA may comprise a nucleotide sequence coding for an emzyme, e.g., a lysosomal enzyme, an antibody, or a coagulation factor.

### (f) Additional Nucleic Acid-Based Cargos

Additional examples of nucleic acid-based cargos include antisense RNA, competing endogenous RNA (ceRNA), small nuclear RNA (snRNA), small nucleolar RNA (snoRNA), pseudo-gene, rRNA, tRNA or other nucleic acids and analogs thereof described herein.

In some embodiments, the nucleic acid molecules described herein target RNAs encoding the following polypeptides: vascular endothelial growth factor (VEGF); Apolipoprotein B (ApoB); luciferase (luc); Androgen Receptor (AR); coagulation factor VII (FVII); factor VIII (FVIII, also known as anti-hemophilic factor (AHF)); factor IX (FIX, also known as Christmas factor); Factor XI (FXI, also known as plasma thromboplastin antecedent); factor I (FI, also known as fibrinogen); factor II (FII, also known as protheombin); factor V (FV, also known as proaccelerin); factor X (FX, also known as Stuart-Power factor); factor XII (FXII, also known as Hageman Factor); factor XIII (FXIII, also known as fibrin stabilizing factor); hypoxia-inducible factor 1, alpha subunit (Hif-1α); placenta growth factor (PLGF); Lamin A/C; and green fluorescent protein (GFP). Exemplary single stranded oligonucleotide agents are shown in Table 1 below. Additional suitable miRNA targets are described, e.g., in John et al., PLoS Biology 2:1862-1879, 2004 (correction in PLoS Biology 3:1328, 2005), and The microRNA Registry (Griffiths- Jones S., NAR 32:D109-D111, 2004).

TABLE 1

Exemplary Oligonucleotide Agents

| AL-SQ-NO : | Sequence ( 5′ - 3′ unless otherwise indicated) | Target |
|---|---|---|
| 3186 | GCACUAGGAGAGAUGAGCUU_{G}-Chol | VEGF |
| 3191 | Naproxen-gGUCAUCACACUGAAUCCAAUg-Chol | ApoB |
| 3209 | CAUCACACUGAAUACCAUdTdTs-Chol | Luc |
| 3230 | oUsoCsoAoCoGoCoGoAoGoCoCoGoAoAoCoGoAoAoCsoAsoAsoAs-Chol | M1r-375 |
| 3234 | oCoUGGGAAAGoUoCAAGoCoCoCAoUdTedT-Chol | AR |
| 3235 | oCoUGoCAAGoUGoCoCoCAAGAoUdTedT-Chol | AR |
| 3253 | GGAfUfCAfUfCfufCAAGfUfCfUfUAfCdTadT-Chol | FVII |
| 3256 | ACUGCAGGGUGAAGAAUUAdTADTs-Chol | Hif-1a |
| 3257 | GCACAUGGAGAGAUGAGCUsUs-Chol | VEGF |
| 3258 | GAACUGUGUGUGAGAGGUCCsUs-Chol | Luc |
| 3264 | CCAGGUUUUUUUCUUACUUTsTs-Chol | VEGP |
| 3265 | UUCCUCAAAUCAAUUACCATsTs-Chol | VEGF |
| 3266 | GGAAGGCUCCCUUGAUGGAdTsdTs-Chol | VEGF |
| 3268 | GACACAGUGUGUUUGAUUUdTadTs-Chol | Hif-1a |
| 3269 | UGCCAAGCCAGAUUCUCUUdTsdTs-Chol | PLGF |
| 3271 | CUCAGGAAUUCAGUGCCUUdTsdTs-Chol | PLGF |
| 3275 | CUGGACUUCCAGAAGAACAdTdT-Chol | Lamin A/C |
| 3150 | Chol-sGUCAUCACACUGAAUACCAAsU | ApoB |
| 5225 | GUCAUCACACUGAAUACCAAUa-Chol | ApoB |
| 4967 | GcACcAUCUUCUUcAAGGACGs-Chol | GFP |
| 5225 | GUCAUCACACUGAAUACCAAUs-Chol | ApoB |
| 5221 | AGGUGUAUGGCUUCAACCCUGs-Chol | ApoB |

Additional exemplary nucleic acid-based cargos are provided in Table 2.

TABLE 2

Additional Exemplary Nucleic Acid-Based Cargos

| Molecule | Trade name / Code Name | Indication |
|---|---|---|
| beperminogene perplasmid (encoding HGF) | AMG001 | Parkinson’s disease Ischaemic heart disease |
| Plasmid encoding FIX | AMT-060 | haemophilia B |
| etranacogene dezaparvovec (encoding FIX) | AMT-061 | haemophilia B |
| fidanacogene elaparvovec (encoding FIX) | SPK-9001 | haemophilia B |
| eladocagene exuparvovec (encoding Aromatic-L-amino-acid decarboxylase) | | Aromatic L-amino acid decarboxylase (AADC) deficiency |
| alferminogene tadenovec (encoding hFGF4) | Generx | Myocardial ischemia |
| donaperminogene seltoplasmid (encoding hHGF) | VM202 | Diabetic neuropathy |
| Eteplirsen | Exondys 51 | Antisen oligo for treating some types of Duchenne muscular dystrophy (DMD) |
| Fomivirsen | Vitravene | Antisense antiviral drug that was used in the treatment of cytomegalovirus retinitis in immunocompromised patients |
| giroctocogene fitelparvovec (encoding FVIII) | | Haemophilia A |
| Voretigene Neparvovec (encoding hRPE65) | Luxturna | Leber’s congenital amaurosis |
| ofranergene obadenovec (encoding TNFRl-Fas) | VB-111 | ovarian cancer; thyroid cancer |
| aglatimagene besadenovec (encoding HSV-tk) | ProstAtak | Solid tumors |
| tavokinogene telseplasmid (encoding hIL-12) | TAVO | various cancers |
| Lanacogene vosiparvovec (encoding FIX) | | Hemophilia B |
| resamirigene bilparvovec (encoding MTM1) | | X-linked myotubular myopathy |
| timrepigene emparvovec (encoding REP-1) | | AAV2 gene therapy for choroideremia |
| SB-FIX | | hemophilia B |
| TAK-748 (encoding FIX) | | Hemophilia B |
| Oncoprex (TUSC2 gene) | | Lung cancer |
| givosiran (RNAi targeting ALAS1)) | Givlaari | acute hepatic porphyria (AHP) |
| golodirsen (antisense) | VYONDYS 53 | Duchenne Muscular Dystrophy (DMD) |
| inotersen (antisense) | TEGSEDI | hereditary transthyretin amyloidosis |
| mipomersen (antisense) | Kynamro | homozygous familial hypercholesterolemia |
| Nusinersen | Spinraza | Spinal muscular atrophy, |
| pegaptanib (aptamer) | Macugen | neovascular (wet) age-related macular degeneration |
| alicaforsen (antisense) | | Crohn’s disease |
| viltolarsen (antisense) | | Duchenne muscular dystrophy |
| rintatolimod (dsRNA) | Ampligen, atvogen | chronic fatigue syndrome (CFS) |
| casimersen (antisense) | SRP-4045 | Duchenne muscular dystrophy |
| fitusiran (RNAi) | | Hemophilia a or B |
| imetelstat (Oligo) | | myelofibrosis and other myeloid malignancies |
| Patisiran | Onpattro | RNAi for the treatment of polyneuropathy in people with hereditary transthyretin-mediated amyloidosis. |
| pelacarsen (antisense) | TQJ 230 | hyperlipoproteinaemia |
| teprasiran (siRNA) | | Delayed graft function (DGF) |
| tofersen (antisense) | | Amyotrophic lateral sclerosis |
| tilsotolimod (oligo) | | Melanoma |
| trabedersen (antisense) | | Pancreatic cancer. |
| vutrisiran (RNAi) | ALN-TTRSC02 | Hereditary transthyretin amyloidosis |
| avacincaptad pegol (aptamer) | Zimura | Geographic atrophy secondary to dry eye |
| Brivoligide (oligo) | | Post operative pain |
| lademirsen (antisense) | RG012 | Alport syndrome |
| olaptesed pegol | NOX-A12 | multiple myeloma |
| Prexigebersen (antisense) | | Acute myeloid leukemia |

### B. Polypeptides

In some embodiments, the LNP-MPVs disclosed herein comprise cargos, which can be protein-based, including peptides, polypeptides, and proteins. The protein-based cargo may be a naturally occurring polypeptide. Alternatively, it may be a modified version of a naturally occurring polypeptide or a non-naturally (synthetic) polypeptide. Non-limiting examples of suitable protein-based cargos include antibodies (e.g., directed against a cellular or pathogenic target), hormones, growth factors, cofactor, enzymes (e.g., metabolic enzymes, immunoregulatory enzymes, gastrointestinal enzymes, growth regulatory enzymes, coagulation cascade enzymes), cytokines, vaccine antigens, antithrombotics, antithrombolytics, toxins, or an antitoxin.

### (a) Antibodies

In some embodiments, the protein-based cargo comprises or is a therapeutic antibody, which may be directed against a cellular target. In some examples, the antibodies may target checkpoint molecules (e.g., PD-1 or PD-L1). See examples in Table 3 below. In other examples, the antibodies may target cytokines, e.g., inflammatory cytokines such as TNF-alpha or IL-6 or receptors thereof such as IL-6R. See examples in Table 3 below.

In yet other examples, the antibodies may target pathogenic antigens, for example, antibodies capable of neutralizing a pathogen such as a virus, a bacterium, a fungus, a helminth, or a parasite. Such a neutralizing antibody may be a broadly neutralizing antibody or non-broadly neutralizing antibodies. A broadly neutralizing antibody can recognize, bind to, and block many strains of a particular pathogen, such as a virus. Broadly neutralizing antibodies generally target certain conserved epitopes of the pathogen, e.g., a viral pathogen. While a virus may mutate, such conserved epitopes would still exist. In contrast, non-broadly neutralizing antibodies are specific for individual viral strains with unique epitopes. A type of neutralizing antibody may recognize and block one or more types of a pathogen from entering its target cells. Broadly neutralizing antibodies may also activate other immune cells to help destroy pathogen-infected cells. In some instances, such antibodies are isolated from patients recovered from an infection. These antibodies from recovered patients can be isolated and either be used directly as a therapeutic agent or are sequenced and subsequently produced using recombinant techniques known in the art. Alternatively, antibodies capable of binding to the pathogenic target antigens can be isolated from a suitable antibody library following routine selection processes as known in the art. Such antibodies can be made fully human (humanized) and recombinantly produced from cell lines according to methods known in the art.

In some cases, two, three or more neutralizing, e.g., broadly neutralizing, non-broadly neutralizing antibodies, or a combination thereof, can be combined in order to achieve virus control. Such antibodies may be loaded into the same LNP-MPVs, or different LNP-MPVs. They can be administered sequentially or concurrently. Thus, the LNP-MPVs disclosed herein collectively may be loaded with one or more broadly neutralizing antibodies, one or more non-broadly neutralizing antibodies, or a combination thereof. In some instances, the LNP-MPVs collectively may be loaded with a cocktail of neutralizing antibodies, e.g., broadly neutralizing antibodies, non-broadly neutralizing antibodies, or a combination thereof. For example, the cocktail may contain 2, 3, 4 or more neutralizing antibodies, e.g., broadly neutralizing antibodies, non-broadly neutralizing antibodies, or a combination thereof. In specific examples, a cocktail of non-broadly neutralizing antibodies may comprise antibodies that each neutralize different strains of a pathogen. In another example, a cocktail may comprise a combination of broadly neutralizing antibodies and non-broadly neutralizing antibodies. In yet another example, a cocktail may comprise broadly neutralizing antibodies only. Such antibodies may each be separately loaded in an LNP-MPV as described herein and administered sequentially one after the other. In other embodiments, the antibodies are administered together in a cocktail, concurrently.

In specific examples, the neutralizing antibodies disclosed herein may target a coronavirus such as SARS (e.g., SARS-CoV-2) and thus be effective in treating diseases caused by SARS infection such as COVID-19. In some cases, the neutralizing antibodies can be isolated from patients recovered from an infection, e.g., a coronavirus infection. In some examples, the antibodies can be isolated from a human patient recovered from COVID-19. Such antibodies may be sequenced and subsequently produced using recombinant techniques known in the art. In other instances, such neutralizing antibodies may be isolated from a suitable antibody library following routine selection processes as known in the art, using a suitable antigen from the virus, for example, the Spike protein of SARS-CoV-2. In some embodiments, the neutralizing antibodies are fully human (humanized) and recombinantly produced from cell lines. Non-limiting examples of neutralizing antibodies targeting SARS-CoV-2 include REGN3048 and REGN 3051 (Regeneron Pharmaceuticals).

Exemplary antibody therapeutics are provided in Table 3 below:

TABLE 3

Exemplary Antibody Therapeutics

| | | |
|---|---|---|
| Molecule | Trade name | Exemplary Indications/MOA |
| 4A8 | | SARS-CoV-2 mAb |
| 47D11 | | SARS CoV-2 mAb |
| 311mab-31BS | | SARS CoV-2 mAb |
| 311mab-31B9 | | SARS CoV-2 mAb |
| 311mab-32D4 | | SARS CoV-2 mAb |
| Abciximab | ReoPro | Adjunct to aspirin and heparin for prevention of cardiac ischaemia in patients undergoing percutaneous coronary intervention or patients about to undergo percutaneous coronary intervention with unstable angina not responding to medical therapy |
| Adalimumab | Humira, Abrilada, Hadlima, Hyrimoz, Cyltezo, Amjevita, Hulio (EU TN) | Anti-TNFalpha antibody; Rheumatoid arthritis, Crohn’s disease, ankylosing spondylitis, psoriatic arthritis; Juvenile rheumatoid arthritis, Ulcerative colitis, Plaque psoriasis, Ankylosing, spondylitis, Hidradenitis suppurativa, Spondylarthritis, Behcet’s syndrome, Uveitis, Pustular psoriasis, Unspecified, Interstitial cystitis |
| Aducanumab | | Alzheimer’s disease |
| AK-104 | | Anti-CTLA/anti-PD-1 bispecific |
| Alemtuzumab | Campath | B-cell chronic lymphocytic leukaemia in patients who have been treated with alkylating agents and who have failed fludabarine therapy; Multiple sclerosis, Chronic lymphocytic leukaemia, T cell prolymphocytic leukaemia, Graft-versus-host disease, Rheumatoid arthritis |
| Alirocumab | Praluent | high cholesterol |
| Anifrolumab | | Systemic lupus erythematosus, Scleroderma |
| anthrax immune globulin | Anthrasil | inhalational anthrax |
| anti-thymocyte globulin | Atgam | kidney transplant rejection |
| Antithymocyte globulin (rabbit) | Thymoglobulin | Acute kidney transplant rejection, aplastic anaemia |
| Apamistamab | | relapsed or refractory acute myeloid leukemia |
| Arcitumomab | CEA-scan | Colon and breast cancer detection |
| Atezolizumab | TECENTRIQ® | Anti-PD-L1 antibody, immunotherapy for cancer; locally advanced or metastatic urothelial carcinoma |
| ATOR-1015 | | Anti-CTLA-4 |
| Avelumab | BAVENCIO® | Anti-PD-L1 antibody, immunotherapy for cancer; non-small-cell lung carcinoma, Merkel-cell carcinoma |
| B38 | | SARS-CoV-2 mAb |
| Balstilimab | | Cervical cancer |
| B asiliximab | Simulect | Prophylaxis against allograft rejection in renal transplant patients receiving an immunosuppressive regimen including cyclosporine and corticosteroids |
| B atoclimab | | neuromyelitis optica spectrum disorder |
| Bavituximab | | cancer, viral infections |
| Belantamab mafodotin | | multiple myeloma |
| Belimumab | Benlysta | Systemic lupus erythematosus, Anti- neutrophil cytoplasmic antibody-associated vasculitis, Lupus nephritis, Myositis, Myasthenia gravis, Sjogren’s syndrome, Systemic scleroderma, Renal transplant rejection, Membranous glomerulonephritis, Waldenstrom’s macroglobulinaemia, Rheumatoid arthritis |
| Bemarituzumab | | gastric cancer or gastroesophageal junction adenocarcinoma |
| Benralizumab | Fasenra | asthma, eosinophilic asthma, eosinophilic oesophagitis |
| Bermekimab | Xilonix | colorectal cancer, atopic dermatitis |
| Bevacizumab | Avastin, Zirabev, Mvasi | Cancer |
| Bleselumab | | prevention of organ transplant rejection |
| Blinatumomab | Blincyto | Philadelphia chromosome-negative relapsed or refractory acute lymphoblastic leukemia |
| BMS-986218 | | Anti-CTLA-4 |
| Brentuximab vedotin | Adcetris | relapsed or refractory Hodgkin lymphoma (HL) and systemic anaplastic large cell lymphoma (ALCL) |
| Brodalumab | Siliq, Kyntheum | Psoriatic arthritis, Erythrodermic psoriasis, Pustular psoriasis, Plaque psoriasis, Asthma, Crohn’s disease, Rheumatoid arthritis, Psoriasis |
| Brolucizumab | Beovu | neovascular (wet) age-related macular degeneration |
| Budigalimab | | Anti-PD1/PD-L1 |
| Cabiralizumab | | metastatic pancreatic cancer |
| Camrelizumab | | Anti-PD1/PD-L1 |
| Canakinumab | Ilaris | Cryopyrin-associated periodic syndromes, Familial Mediterranean fever, Juvenile rheumatoid arthritis, Gouty arthritis, Peroxisomal disorders, Familial autosomal dominant periodic fever, Cardiovascular disorders, Behcet’s syndrome, Peripheral arterial occlusive disorders, Mucocutaneous lymph node syndrome, Abdominal aortic aneurysm, Pulmonary sarcoidosis, Atherosclerosis, Osteoarthritis, Diabetic retinopathy, Chronic obstructive pulmonary disease, Type 2 diabetes mellitus, Rheumatoid arthritis, Type 1 diabetes mellitus, Polymyalgia rheumatica, Asthma |
| Caplacizumab | Cablivi | thrombotic thrombocytopenic purpura and thrombosis |
| Capromab pendetide | ProstaScint | Prostate cancer detection |
| Carotuximab | | angiosarcoma |
| Catumaxomab | Removab | Malignant ascites |
| CB6 | | SARS-CoV-2 mAb |
| Cemiplimab | Libtayo® | Anti-PD-1 antibody, immunotherapy for advanced squamous cell skin cancer; myeloma, lung cancer |
| Certolizumab pegol | Cimzia | Rheumatoid arthritis, Ankylosing spondylitis, Crohn’s disease, Psoriatic arthritis, Spondylitis, Plaque psoriasis, Juvenile rheumatoid arthritis, Interstitial cystitis, Cognition disorders |
| Cetuximab | Erbitux | Colorectal cancer, head and neck cancer |
| CG-0070 | | Anti-CTLA-4 |
| Clazakizumab | | psoriatic arthritis |
| Concizumab | | bleeding |
| Cosibelimab | | Anti-PD1/PD-L1 |
| CR3033 | | SARS-CoV-2 mAb |
| Crizanlizumab | Adakveo | vaso-occlusive crisis in patients with sickle cell anemia |
| Crotalidae polyvalent immune fab | CroFab | Crotalid snakebites |
| Crovalimab | | paroxysmal nocturnal hemoglobinuria |
| CS1001 | | Anti-PD-L1 antibody |
| Cusatuzumab | | cancer |
| Daclizumab | Zenapax | Prophylaxis against acute allograft rejection in patients receiving renal transplants; Renal transplant rejection, Multiple sclerosis, Graft-versus-host disease, Asthma, Type 1 diabetes mellitus, Immune-mediated uveitis, Liver transplant rejection, Ulcerative colitis, Psoriasis, Tropical spastic paraparesis, Haematological malignancies |
| Daratumumab | Darzalex | multiple myeloma, diffuse large B cell lymphoma, follicular lymphoma, and mantle cell lymphoma |
| Denosumab | Prolia and Xgeva | osteoporosis, treatment-induced bone loss, metastases to bone, and giant cell tumor of bone |
| Dinutuximab | Unituxin | treatment for children with high-risk neuroblastoma |
| Dostarlimab | | Anti-PD-1 antibody; recurrent or advanced endometrial cancer |
| Dupilumab | Dupixent | eczema (atopic dermatitis), asthma and nasal polyps |
| Durvalumab | IMFINZI | Anti-PD-L1 antibody, immunotherapy for non-small cell lung cancer; bladder cancer |
| Eculizumab | Soliris | Paroxysmal nocturnal haemoglobinuria; Paroxysmal nocturnal haemoglobinuria, Haemolytic uraemic syndrome, Myasthenia gravis, Neuromyelitis optica, Delayed graft function, Renal transplant rejection, Guillain- Barre syndrome, Heart transplant rejection, Antiphospholipid syndrome, Rheumatoid arthritis, Autoimmune haemolytic anaemia, Age-related macular degeneration, Membranous glomerulonephritis, Glomerulonephritis, Systemic lupus erythematosus, Allergic asthma, Motor neuron disease, Lupus nephritis, Psoriasis, Dermatomyositis, Bullous pemphigoid, Adult respiratory distress syndrome, Immune thrombocytopenic purpura |
| Efalizumab | Raptiva | Adults with chronic moderate to severe plaque psoriasis who are candidates for systemic therapy or phototherapy |
| Elotuzumab | Empliciti | relapsed multiple myeloma |
| Emapalumab | Gamifant | hemophagocytic lymphohistiocytosis |
| Emicizumab | Hemlibra | haemophila A |
| Enfortumab | | locally advanced or metastatic urothelial cancer |
| Ensituximab | | cancer |
| Epratuzumab | LymphoCide | Systemic lupus erythematosus, Acute lymphoblastic leukaemia, Non-Hodgkin’s lymphoma, Cachexia |
| Eptinezumab | Vyepti | Target CGRP for the preventive treatment of migraine in adults. |
| Erenumab | Aimovig | migraine |
| Etokimab | | atopic disease |
| Etrolizumab | | ulcerative colitis and Crohn’s |
| Evinacumab | | dyslipidemia |
| Evolocumab | Repatha | hyperlipidemia |
| FAZ053 | | Anti-PD-1/PD-L1 antibody |
| Faricimab | | diabetic macular edema |
| Farletuzumab | | ovarian cancer |
| Flotetuzumab | | acute myeloid leukemia |
| Foralumab | | COVID-19 |
| Fremanezumab | Ajovy | migraines |
| Galcanezumab | Emgality | migraine |
| Ganitumab | | cancer |
| Gantenerumab | | Alzheimer’s disease |
| Garetosmab | | fibrodysplasia ossificans progressiva |
| Gemtuzumab ozogamicin | Mylotarg | Relapsed CD33+ acute myeloid leukaemia in patients who are more than 60 years old and are not candidates fo cytotoxic chemotherapy |
| Golimumab | Simponi | Anti-TNF antibody; Psoriatic arthritis, Rheumatoid arthritis, Ankylosing spondylitis, Ulcerative colitis, Juvenile rheumatoid arthritis, Hearing disorders, Type 1 diabetes mellitus, Sarcoidosis, Asthma, Uveitis, Cardiovascular disorders |
| Guselkumab | Tremfya | Plaque psoriasis, Erythrodermic psoriasis, Palmoplantar pustulosis, Rheumatoid arthritis, Psoriatic arthritis |
| H4 | | SARS-CoV-2 |
| HLX10 | | Anti-PD1/PD-L1 |
| Ianalumab | | autoimmune hepatitis |
| Ibritumomab tiuxetan | Zevalin | Relapsed or refractory low-grade, follicular, or transformed B-cell non- Hodgkin’s lymphoma (NHL), including rituximab-refractory follicular NHL |
| Idarucizumab | Praxbind | reversal of anticoagulant effects of dabigatran |
| Imciromab pentetate | Myoscint | Detects presence and location of myocardial injury in patients with suspected myocardial infarction |
| Immune globulin -CSL Behring | | Mucocutaneous lymph node syndrome, Immune thrombocytopenic purpura, Immunodeficiency disorders, Guillain-Barre syndrome, Haemolytic disease of newborn, Rabies, Hepatitis A, Varicella zoster virus infections, Chronic inflammatory demyelinating polyradiculoneuropathy, Tetanus, Hepatitis B, Encephalitis, Renal transplant rejection, Skin and soft tissue infections, Motor neuron disease, Systemic lupus erythematosus |
| Immune globulin 10% - Grifols | | Immune thrombocytopenic purpura, Immunodeficiency disorders, Chronic inflammatory demyelinating polyradiculoneuropathy, Myasthenia gravis, Multiple sclerosis; Alzheimer’s disease |
| Immunoglobulin A | | immunodeficiency |
| immunoglobulin G | | immunodeficiency |
| Immunoglobulin M | | immunodeficiency |
| Indatuximab ravtansine | | multiple myeloma |
| Infliximab | Remicade, Avsola, Ixifi, Renflexis, Inflectra | Anti-TNF antibody; Rheumatoid arthritis, Crohn’s disease, ankylosing spondylitis, psoriatic arthritis, plaque psoriasis; Psoriasis, Ulcerative colitis, Behcet’s syndrome Mucocutaneous lymph node syndrome, Hepatitis C, Pyoderma, Berylliosis |
| Infliximab | REMICADE® | anti-TNF-alpha antibody, to treat Crohn’s disease, ulcerative colitis, rheumatoid arthritis, ankylosing spondylitis, psoriasis, psoriatic arthritis, and Behçet’s disease |
| Inebilizumab | Uplizna | Target CD19; for the treatment of neuromyelitis optica spectrum disorder in adults |
| Inolimomab | | graft-versus-host disease |
| Inotuzumab ozogamicin | Besponsa | relapsed or refractory B-cell precursor acute lymphoblastic leukemia |
| Ipilimumab | Yervoy | Anti-CTLA-4 antibody; melanoma, non-small cell lung carcinoma (NSCLC), small cell lung cancer (SCLC), bladder cancer and metastatic hormone-refractory prostate cancer |
| Isatuximab | Sarclisa, | Target CD38; for the treatment of multiple myeloma |
| Ixekizumab | Taltz | Plaque psoriasis, Psoriatic arthritis, Pustular psoriasis, Erythrodermic psoriasis, Spondylarthritis, Ankylosing spondylitis, Rheumatoid arthritis |
| KN-046 | | Anti-CTLA4/anti-PD-1 bispecific |
| Labetuzumab govitecan | | colorectal cancer |
| Lanadelumab | Takhzyro | hereditary angioedema |
| Lecanemab | | Alzheimer’s disease |
| Leronlimab | | Target CCR5; HIV infection, coronavirus disease |
| Lilotomab | | non-Hodgkin lymphoma |
| Lirilumab | | acute myeloid leukemia |
| Loncastuximab tesirine | | B-cell non-Hodgkin lymphoma (NHL) and B-cell acute lymphoblastic leukemia |
| Magrolimab | | acute myeloid leukemia and myelodysplastic syndrome |
| Marstacimab | | bleeding with hemophilia |
| Mavrilimumab | | rheumatoid arthritis |
| MEDI-5752 | | Anti-CTLA-4 |
| MEDI-0680/AMP-514 | | Anti-PD1/PD-L1 |
| Mepolizumab | Nucala | Asthma, Chronic obstructive pulmonary disease, Churg-Strauss syndrome, Hypereosinophilic syndrome, Nasal polyps, Eosinophilic oesophagitis |
| MGD-019 | | Anti-CTLA-4 |
| Mirikizumab | | psoriasis |
| MK-1308 | | Anti-CTLA-4 |
| Mogamulizumab | Poteligeo | relapsed or refractory mycosis fungoides and Sézary disease, relapsed or refractory CCR4+ adult T-cell leukemia/lymphoma (ATCLL) and relapsed or refractory CCR4+ cutaneous T cell lymphoma |
| Mosunetuzumab | | Cancer |
| Moxetumomab pasudotox | Lumoxiti | relapsed or refractory hairy cell leukemia |
| Muromonab-CD3 | Orthoclone, OKT3 | Acute renal allograft rejection or steroid- resistant cardiac or hepatic allograft rejection |
| Narsoplimab | | Target MASP-2; prevent complement-mediated inflammation and endothelial damage |
| Natalizumab | Tysabri | Anti-alpha4 integrin antibody; Relapsing multiple sclerosis; Multiple sclerosis, Crohn’s disease, Stroke, Graft-versus-host disease, Rheumatoid arthritis, Multiple myeloma |
| Naxitamab | | Target GD2; high-risk neuroblastoma and refractory osteomedullary disease |
| Necitumumab | Portrazza | metastatic squamous non-small-cell lung carcinoma |
| Nimotuzumab | | glioma, squamous cell carcinomas of the head and neck |
| Nipocalimab | | Hemolytic Disease |
| Nivolumab | Opdivo | anti-PD-1 antibody; Melanoma, lung cancer, renal cell carcinoma, Hodgkin lymphoma, head and neck cancer, colon cancer, and liver cancer; |
| Nofetumomab | Verluma | Small-cell lung cancer detection and staging |
| Obexelimab | | IgG4-Related Disease and Systemic Lupus Erythematosus, rheumatoid arthritis |
| Obinutuzumab | | chronic lymphocytic leukemia |
| Ocaratuzumab | | relapsed/refractory follicular lymphoma, rheumatoid arthritis |
| Ocrelizumab | Ocrevus | Multiple sclerosis, Systemic lupus erythematosus, Rheumatoid arthritis, Lupus nephritis, Haematological malignancies, Eye disorders |
| Odronextamab | | Diffuse large B cell lymphoma; Follicular lymphoma; Non-Hodgkin’s lymphoma |
| Ofatumumab | Arzerra | Chronic lymphocytic leukaemia, Follicular lymphoma, Multiple sclerosis, Diffuse large B cell lymphoma, MALT lymphoma, Neuromyelitis optica, Pemphigus vulgaris, Rheumatoid arthritis, Waldenstrom’s macroglobulinaemia |
| Olinvacimab | | Glioblastoma, Breast cancer |
| Olokizumab | | Rheumatoid arthritis |
| Omalizumab | Xolair | Adults and adolescents (at least 12 years old) with moderate to severe persistent asthma who have a positive skin test or in vitro reactivity to a perennial aeroallergen and whose symptoms are inadequately controlled with inhaled corticosteroids |
| Ontamalimab | | Ulcerative Colitis or Crohn’s Disease |
| Ontuxizumab | | melanoma, colorectal cancer, sarcoma, and lymphoma |
| Oportuzumab monatox | Vicinium | non-muscle invasive bladder cancer |
| Oregovomab | | ovarian cancer |
| Orilanolimab | | Autoimmune haemolytic anaemia, Pemphigus, Myasthenia gravis |
| Palivizumab | Synagis | Prevention of respiratory syncytial virus infection in high-risk paediatric patients |
| Pamrevlumab | | idiopathic pulmonary fibrosis and pancreatic cancer |
| Paniturnumab | Vectibix | Metastatic colorectal cancer |
| Pembrolizumab | KEYTRUDA® | Anti-PD-1 antibody, immunotherapy to treat melanoma, lung cancer, head and neck cancer, Hodgkin lymphoma, and stomach cancer. |
| Penpulimab | | Anti-PD1/PD-L1 |
| Pepinemab | | Huntington’s disease, Non-small cell lung cancer; Osteosarcoma; Solid tumours |
| Pertuzumab | Perjeta | metastatic HER2-positive breast cancer |
| Pidilizumab | | Anti-PD-1/PD-L1 |
| Polatuzumab vedotin | Polivy | diffuse large B-cell lymphoma |
| Pozelimab | | paroxysmal nocturnal hemoglobinuria |
| Prasinezumab | | Parkinson’s disease |
| Pritumumab | | glioma |
| Ramucirumab | Cyramza | solid tumors, advanced gastric cancer or gastroesophageal junction (GEJ) adenocarcinoma |
| Ranibizumab | Lucentis | Neovascular age-related macular degeneration |
| Ravulizumab | Ultomiris | paroxysmal nocturnal hemoglobinuria and atypical hemolytic uremic syndrome |
| REGN 3051 | | MERS infection and COVID-19 |
| REGN3048 | | MERS infection and COVID-19 |
| REGN-3500 | | fully-human monoclonal antibody that inhibits interleukin-33, in trial for asthma control |
| Regneb3 | | Anti-Ebola |
| Relatlimab | | melanoma |
| Reslizumab | Cinqair (US), Cinqaero (EU) | asthma |
| rho(D) immune globulin | RhoGAM | prevent an immune response to Rh positive blood in people with an Rh negative blood type |
| Risankizumab | Skyrizi | Plaque psoriasis, Crohn’s disease, Ankylosing spondylitis Asthma, Psoriatic arthritis, Psoriasis |
| Rituximab | Rituxan, Ruxience, Truxima | Relapsed or refractory low-grade or follicular CD20+ B-cell NHL, primary low-grade or follicular CD20+ B-cell NHL in combination with CVP chemotherapy; diffuse large B-cell CD20+ NHL in combination with CHOP or other anthracyline- based chemotherapy; rheumatoid arthritis incombination with methotrexate; Non-Hodgkin’s lymphoma, Rheumatoid arthritis, Microscopic polyangiitis, Wegener’s granulomatosis, Follicular lymphoma, Chronic lymphocytic leukaemia, Nephrotic syndrome, Lymphoproliferative disorders, Diffuse large B cell lymphoma, Pemphigus vulgaris, Transplant rejection, Neuromyelitis optica, Mantle-cell lymphoma, B cell lymphoma, Multiple sclerosis, Ulcerative colitis, Sjogren’s syndrome, Ocular inflammation, Scleritis, Primary biliary cirrhosis, Lupus nephritis, Systemic lupus erythematosus, Graft-versus- host disease, Dermatomyositis, Immune thrombocytopenic purpura |
| Romilkimab | | Systemic scleroderma |
| Romosozumab | Evenity | osteoporosis |
| Rozanolixizumab | | ITP, myasthenia gravis |
| S309 | | SARS-CoV-2 mAb |
| Sacituzumab govitecan | Trodelvy | Target TROP-2; triple-negative breast cancer |
| Sarilumab | Kevzara | Rheumatoid arthritis, Juvenile rheumatoid arthritis, Uveitis, Ankylosing spondylitis |
| Sarilumab | Kevzara® | anti-IL-6 receptor antibody, for rheumatoid arthritis; Rheumatoid arthritis, Juvenile rheumatoid arthritis, Uveitis, Ankylosing spondylitis |
| SARS-CoV-2 RBD-specific IgG antibody | | Covid-19 mAbs |
| SARS-CoV-2 S1-specific IgG antibody | | Covid-19 mAbs |
| Satralizumab | Enspryng | Target IL-6R; for the treatment of neuromyelitis optica spectrum disorder |
| Satumomab pendetide | OncoScint | Colon and ovarian cancer detection |
| Secukinumab | Cosentyx | Plaque psoriasis, Psoriatic arthritis, Ankylosing spondylitis, Pustular psoriasis, Rheumatoid arthritis, Psoriasis, Atopic dermatitis, Alopecia areata, Uveitis, Asthma, Multiple sclerosis, Dry eyes, Polymyalgia rheumatica, Type 1 diabetes mellitus, Crohn’s disease |
| Semorinemab | | Alzheimer’s disease |
| Setrusumab | | Osteogenesis imperfecta |
| Siltuximab | SYLVANT® | anti-IL-6 receptor antibody, to treat people with multicentric Castleman’s disease (MCD) who do not have human immunodeficiency virus (HIV) and human herpesvirus-8 (HHV-8) infection. |
| Siltuximab | Sylvant | neoplastic diseases: metastatic renal cell cancer, prostate cancer, and Castleman’s disease |
| Sintilimab | | Anti-PD1 |
| Siplizumab | | psoriasis |
| Sirukumab | CNTO-136 | Rheumatoid arthritis, Giant cell arteritis, Lupus nephritis, Asthma, Major depressive disorder, Atherosclerosis |
| Solanezumab | | Alzheimer’s disease |
| Spartalizumab | | Anti-PD-1-antibody;Melanoma |
| Spesolimab | | active ulcerative colitis, Pustular psoriasis, Atopic dermatitis; Crohn’s disease; Palmoplantar pustulosis |
| Sutimlimab | | cold agglutinin disease |
| Tafasitamab | | Target CD19; B cell malignancies |
| Tanezumab | | Pain |
| Technetium fanolesomab | NeutroSpec | Diagnostic agent (used in patients with equivocal signs and symptoms of appendicitis) |
| Temelimab | | multiple sclerosis |
| Teplizumab | | renal allograft rejection, psoriatic arthritis |
| Teprotumumab | Epezza | Targeting IGF-1R; for treating adults with thyroid eye disease, |
| Tezepelumab | | asthma and atopic dermatitis |
| Tildrakizumab | Ilumya, Ilumetri | Plaque psoriasis, Autoimmune disorders |
| Tislelizumab | | Anti-PD1 |
| Tisotumab Vedotin | | solid tumors |
| Tocilizumab | Actemra, RoActemra | Rheumatoid arthritis, Juvenile rheumatoid arthritis, Giant lymph node hyperplasia, Giant cell arteritis, Systemic scleroderma, Vasculitis, Polymyalgia rheumatica, Polymyositis, Amyotrophic lateral sclerosis, Dermatomyositis, Chronic lymphocytic leukaemia, Ankylosing spondylitis, Multiple myeloma, Crohn’s disease, Pancreatic cancer, Systemic lupus erythematosus |
| Tocilizumab | Actemra, RoActemra | anti-IL-6 receptor antibody; an immunosuppressive drug, mainly for the treatment of rheumatoid arthritis (RA) and systemic juvenile idiopathic arthritis. |
| Tomaralimab | | Delayed graft function, Myelodysplastic syndromes |
| Toripalimab | | Anti-PD-1 antibody; unresectable or metastatic melanoma |
| Tositumomab and 131I-tositumomab | Bexxar, Bexxar I-131 | CD20+ follicular NHL, with and without transformation, in patients whose disease is refractory to rituximab and has relapsed following chemotherapy; tositumomab and then131I-tositumomab are used sequentially in the treatment regimen |
| Trastuzumab | Herceptin | Breast cancer |
| Trastuzumab deruxtecan | Enhertu | unresectable or metastatic HER2-positive breast cancer |
| Trastuzumab emtansine | Kadcyla | HER2-positive metastatic breast cancer |
| Tremelimumab | | Anti-CTLA-4 antibody; Cancer |
| Ublituximab | | multiple sclerosis, chronic lymphocytic leukemia |
| Urelumab | | cancer and solid tumors |
| Ustekinumab | Stelara | Plaque psoriasis, Psoriatic arthritis, Crohn’s disease, Spondylarthritis, Ulcerative colitis, Systemic lupus erythematosus, Atopic dermatitis, Inflammation, Palmoplantar pustulosis, Sarcoidosis, Rheumatoid arthritis, Primary biliary cirrhosis, Multiple sclerosis |
| Vedolizumab | Entyvio | ulcerative colitis and Crohn’s disease |
| Veltuzumab | | non-Hodgkin’s lymphoma |
| VHH-72 | | SARS-CoV-2 single-domain antibody |
| Vobarilizumab | | inflammatory autoimmune diseases |
| Volagidemab | | Type 1 diabetes mellitus; Type 2 diabetes mellitus |
| Vopratelimab | | non-small cell lung cancer or urothelial cancer |
| Xmab-20717 | | Anti-CTLA-4 |
| Xmab-22841 | | Anti-CTLA-4 |
| Zalifrelimab | | Cervical cancer; Non-small cell lung cancer; Soft tissue sarcoma |
| Zenocutuzumab | | Breast cancer, Colorectal cancer; Gastric cancer; Non-small cell lung cancer; Oesophageal cancer; Pancreatic cancer |
| Zolbetuximab | | gastrointestinal adenocarcinomas and pancreatic tumors |
| Zolbetuximab | IMAB362 | gastrointestinal adenocarcinomas and pancreatic tumors |
| | AMAG-423 | In trial for severe preeclampsia |

In any of the above antibody embodiments, multiple antibodies or nucleic acids encoding such may be combined and delivered sequentially or concurrently in cargo loaded milk exosome(s) described herein. In some embodiments, the therapeutic antibodies or nucleic acids encoding such are each separately loaded in an exosome as described herein and administered sequentially one after the other. In some embodiments, the antibodies or nucleic acids encoding such are administered together in a cocktail, concurrently.

### (b) Other Protein-Based Cargos

In some embodiments, the biologic agent comprises a therapeutic peptide, e.g., hormone. A non-limiting example of such biologic agents include Glucagon-like peptide 1 (GLP-1) and derivatives thereof or other GLP-1 receptor agonists, including but not limited to exenatide, liraglutide, taspoglutide, lixisenatide, semaglutide, albiglutide, dulaglutide, and langlenatide. See Table 4 below. In some examples, the protein-based cargo may be a growth factor, for example, erythropoietin. In other examples, the protein-based cargo may be a factor involved in the coagulation cascade, for example, Factor VIII, Factor IX, Factor X, Factor XI, or Factor XII. In yet other examples, the protein-based cargo can be an enzyme (e.g., metabolic enzymes, immunoregulatory enzymes, gastrointestinal enzymes, growth regulatory enzymes, coagulation cascade enzymes). Other exemplary protein-based cargos include, but are not limited to, cytokines, vaccine antigens, antithrombotics, antithrombolytics, toxins, or an antitoxin. Table 4 provides additional examples of protein-based cargos.

TABLE 4

Additional Exemplary Protein-Based Cargos

| | | |
|---|---|---|
| Molecule | Trade name | Exemplary Indication(s)/MOA |
| calaspargase pegol | ASPARLAS | acute lymphoblastic leukemia |
| (antihemophilic) Factor VIII | Bioclate, Helixate, Kogenate, Recombinate, ReFacto | Haemophilia A |
| Abatacept | Orencia | Rheumatoid arthritis (especially when refractory to TNFα inhibition); Rheumatoid arthritis, Juvenile rheumatoid arthritis, Lupus nephritis, Psoriatic arthritis, Sjogren’s syndrome, Diffuse |
| | | scleroderma, Nephrotic syndrome, Inflammation. Ulcerative colitis, Crohn’s disease, Systemic lupus erythematosus, Multiple sclerosis, Psoriasis, Graft-versus-host disease, Transplant rejection, Xenotransplant rejection |
| Abobotulinumtoxin A | Dysport | muscle spasms |
| Adenosine deaminase (pegademase bovine, PEG-ADA) | Adagen | Severe combined immunodeficiency due to adenosine deaminase deficiency |
| Aflibercept | Eylea, Zaltrap | wet macular degeneration and metastatic colorectal cancer |
| Agalsidase alfa | | Fabry disease |
| Agalsidase-P (human α-galactosidase A) | Fabrazyme | Fabry disease; prevents accumulation of lipids that could lead to renal and cardiovascular complications |
| Albiglutide | | Glucagon-like peptide 1 derivative; for type 2 diabetes |
| Albumin | | Blood |
| Albutrepenonacog alfa | Idelvion | hemophilia B |
| Aldesleukin | Proleukin | malignant melanoma, renal cell cancer) |
| Alefacept | Amevive | Adults with moderate to severe chronic plaque psoriasis who are candidates for systemic therapy or phototherapy; Psoriasis, Transplant rejection, Psoriatic arthritis |
| Alglucosidase-α | Myozyme | Pompe disease (glycogen storage disease type II) |
| alicaforsen sodium | | in trials for Crohn’s disease |
| alpha-1 proteinase inhibitor | Aralast, Prolastin, Zemaira | alpha 1-antitrypsin deficiency in people who have symptoms of emphysema. |
| Alteplase (tissue plasminogen activator: tPA) | Activase | Pulmonary embolism, myocardial infarction, acute ischaemic stroke, occlusion of central venous access devices |
| Anakinra | Antril, Kineret | Moderate to severe active rheumatoid arthritis in adults who have failed one or more disease-modifying antirheumatic drug; Rheumatoid arthritis, Cryopyrin-associated periodic syndromes, Gout, Juvenile rheumatoid arthritis, Septic shock, Ankylosing spondylitis, Osteoarthritis, Graft-versus-host disease, Pneumococcal infections |
| Andexanet alfa | Andexxa, Ondexxya | antidote for the medications rivaroxaban and apixaban |
| Anistreplase (anisoylated plasminogen streptokinase activator complex; APSAC) | Eminase | Thrombolysis in patients with unstable angina |
| antihemophilic factor | Advate, Adynovate, Eloctate, Esperoct, Helixate FS, Hemofil-M, HyateC, Jivi, Koate DVI, Obizur | hemophilia A |
| anti-inhibitor coagulant complex | Feiba, Feiba VH Immuno, Autoplex T, Feiba NF | hemophilia A and B |
| Anti-Rhesus (Rh) immunoglobulin G | Rhophylac | Routine antepartum and postpartum prevention of Rh(D) immunization in Rh(D)-negative women; Rh prophylaxis in case of obstetric complications or invasive procedures during pregnancy; suppression of Rh immunization in Rh(D)- negative individuals transfused with Rh(D)- positive red blood cells |
| antithrombin agents | | anti-coagulants |
| Antithrombin III (AT-III) | Thrombate III | Hereditary AT-III deficiency in connection with surgical or obstetrical procedures or for thromboembolism |
| apadamtase alfa | TAK 755 | Thrombotic Thrombocytopenic Purpura; recombinant ADAMTS13 (a disintegrin and metalloprotease with thrombospondin type-1 |
| Apcitide | Acutect | Imaging of acute venous thrombosis |
| aprotinin | Trasylol | prophylactic use to reduce perioperative blood loss and the need for blood transfusion |
| Argipressin | Pressyn | Manage anti-diuretic hormone deficiency. treat diabetes insipidus related to low levels of antiduretic hormone |
| Asfotase alfa | Strensiq | perinatal/infantile- and juvenile-onset hypophosphatasia |
| Asparaginase erwinia chrysanthemi | Elspar | acute lymphoblastic leukemia (ALL), acute myeloid leukemia (AML), and non-Hodgkin’s lymphoma |
| Atacicept | | Systemic lupus erythematosus, Rheumatoid arthritis, Multiple sclerosis, Lupus nephritis, Chronic lymphocytic leukaemia, Non-Hodgkin’s lymphoma, Multiple myeloma |
| Atosiban | Tractocile, Antocin, Atosiban SUN | an inhibitor of the hormones oxytocin and vasopressin. As a labour repressant (tocolytic) to halt premature labor. |
| avalglucosidase alfa | neoGAA | late-onset Pompe disease |
| Axicabtagene ciloleucel | Yescarta | large B-cell lymphoma |
| Bacitracin | Baciim | Antibiotics - disrupt Gram-positive bacteria by interfering with cell wall and peptidoglycan synthesis |
| Becaplermin (platelet-derived growth factor; PDGF) | Regranex | Debridement adjunct for diabetic ulcers |
| Bee venom -Apimeds | | Osteoarthritis, Multiple sclerosis |
| belatacept | NULOJIX | prophylaxis of organ rejection in adult patients receiving a kidney transplant |
| bempegaldesleukin | NKTR-214 | an experimental anti-cancer drug candidate; it is a PEGylated interleukin-2 (IL-2). |
| beractant | SURVANTA | Respiratory Distress Syndrome |
| bintrafusp alfa | M 7824 | in trial for various cancer, fusion protein targeting TGF-β and PD-L1 |
| Bivalirudin | Angiomax | Reduce blood-clotting risk in coronary angioplasty and heparin- induced thrombocytopaenia |
| Botulinum toxin type A | Botox | Many types of dystonia, particularly cervical; cosmetic uses |
| Botulinum toxin type B | Myoblock | Many types of dystonia, particularly cervical; cosmetic uses |
| Calcitonin (including salmon calcitonin) | | An analog of human calcitonin used in the treatment of postmenopausal osteoporosis, Paget disease of bone, and hypercalcemia. |
| Cerebrolysin | | Stroke and vascular dementia |
| Cerliponase alfa | Brineura | Batten disease |
| chorionic gonadotropin | Novarel, Pregnyl | Tumor marker, Fertility |
| Ciclosporin_ Novartis | | Psoriasis, Liver transplant rejection, Transplant rejection, Pancreas transplant rejection, Atopic dermatitis, Rheumatoid arthritis, Heart transplant rejection, Myasthenia gravis, Renal transplant rejection, Ulcerative colitis |
| Coagulation Factoe X | | Stuart-Prower factor deficiency |
| Coagulation Factor I | | fibrinogen deficiencies such as afibrinogenemia, hypofibrinogenemia, and dysfibrinogenemia |
| Coagulation Factor II | | prothrombin deficiencies such as dysprothrombinemia and hypoprothrombinemia |
| Coagulation factor V | | proaccelerin deficiencies such as Owren’s Disease, and Parahemophilia |
| Coagulation Factor VII | | proconvertin deficiencies such as Alexander’s Disease |
| Coagulation Factor XIII | | fibrin stabilizing factor deficiency |
| Coagulator Factor XI | | hemophilia C, plasma thromboplastin antecedent (PTA) deficiency and Rosenthal syndrome |
| Coagulator Factor XII | | Hageman factor deficiency |
| Collagenase | Santyl | Debridement of chronic dermal ulcers and severely burned areas |
| Collagenase clostridium histolyticum | Xiaflex, Xiapex | Dupuytren’s contracture, Peyronie’s disease |
| condoliase | SI-6603 | lumbar disc herniation |
| Conestat alfa | Cinryze, Ruconest, Berinert | Hereditary angioedema |
| Corticotropin gel -Mallinckrodt | | Membranous glomerulonephritis, Juvenile rheumatoid arthritis, Polymyositis, Infantile spasms, Rheumatoid arthritis, Adrenal cortex disorders, Nephrotic syndrome, Sarcoidosis, Systemic lupus erythematosus, Psoriatic arthritis, Ankylosing spondylitis, Multiple sclerosis, Diabetic nephropathies, Amyotrophic lateral sclerosis |
| Crotalidae polyvalent immune Fab (ovine) | Crofab | Crotalidae envenomation (Western diamondback, Eastern diamondback and Mojave rattlesnakes, and water moccasins) |
| dalcinonacog alfa | | recombinant human Factor IX, in trial for hemophilia B |
| Darbepoetin alfa LA | Aranesp | anemia |
| denileukin diftitox | Ontak | persistent or recurrent cutaneous T-cell lymphoma |
| Deninleukin diftitox | Ontak | Persistent or recurrent cutaneous T-cell lymphoma whose malignant cells express the CD25 component of the IL2 receptor |
| Desmopressin | DDAVP, Minrin | diabetes insipidus, bedwetting, hemophilia A, von Willebrand disease, and high blood urea levels |
| Desmopressin acetate | | diabetes insipidus, bedwetting, hemophilia A, von Willebrand disease, and high blood urea levels |
| Dibotermin-α (recombinant human bone morphogenic protein 2; rhBMP2) | Infuse | Spinal fusion surgery, bone injury repair |
| Digoxin immune serum Fab (ovine) | Digifab | Digoxin toxicity |
| Drotrecogin-α (activated protein C) | Xigris | Severe sepsis with a high risk of death |
| Dulaglutide | Trulicity® | Incretin therapeutic; Glucagon-like peptide 1 derivative; for type 2 diabetes |
| Ecallantide | Kalbitor | hereditary angioedema |
| efgartigimod alfa | | antibody fragment being evaluated for severe autoimmune diseases |
| efineptakin alfa | HyLeukin-7 | recombinant human interleukin-7, in trial for breast cancer; cervical intraepithelial neoplasia; glioblastoma; and human papillomavirus |
| efinopegdutide | HM 12525A, JNJ 64565111 | GLP-1/glucagon-agonist, for obesity and Diabetes Mellitus, Type 2. |
| efmitermant alfa | | fusion protein for Charcot-Marie-Tooth disease |
| elapegademase | REVCOVI | adenosine deaminase severe combined immune deficiency (ADA-SCID) in pediatric and adult patients |
| Elosulfase alfa | Vimizim | Morquio syndrome |
| Enfuvirtide | Fuzeon | Adults and children (at least 6 years old) with advanced HIV infection |
| epidermal growth factor | SLVRGEN | wound healing |
| Epoetin alfa | Epogen, Retacrit | it stimulates erythropoiesis (increasing red blood cell levels) and is used to treat anemia, commonly associated with chronic kidney failure and cancer chemotherapy |
| Epoetin zeta | Epogen, Silapo, Retacrit, Procrit | anemia caused by chronic kidney disease (CKD) or chemotherapy |
| Eptifibatide | Integrilin | reduce the risk of acute cardiac ischemic events (death and/or myocardial infarction) in patients with unstable angina or non-ST-segment-elevation (e.g., non-Q-wave) myocardial infarction (i.e., non-ST-segment elevation acute coronary syndromes) both in patients who are to receive non surgery (conservative) medical treatment and those undergoing percutaneous coronary intervention (PCI) |
| Etanercept | Enbrel, Eticovo, Erelzi | Rheumatoid arthritis, polyarticular-course juvenile rheumatoid arthritis, psoriatic arthritis, |
| | | ankylosing spondylitis, plaque psoriasis; Juvenile rheumatoid arthritis, Plaque psoriasis, Graft-versus-host disease, Discoid lupus erythematosus, Metabolic syndrome, Heart failure, Wegener’s granulomatosis, Pulmonary fibrosis, Transplant rejection, Asthma, Adult-onset Still’s disease, Myasthenia gravis, Behcet’s syndrome, Cachexia, Septic shock |
| exebacase | Lysin CF-301 | antistaphylococcal lysin in trial for Infective Endocarditis |
| Exenatide | Byetta®, Bydureon® | Incretin therapeutic; Glucagon-like peptide 1 derivative; for type 2 diabetes |
| Factor IX | Benefix | Haemophilia B |
| Factor IX Complex | Proplex-T, Alphanine SD | hemophilia B |
| Factor VIIa | NovoSeven | Haemorrhage in patients with haemophilia A or B and inhibitors to factor VIII or factor IX |
| FcFusion protein | | hemophilia B |
| fexapotide triflutate | | for benign prostatic hyperplasia (prostate enlargement, “BPH”) and for low grade localized prostate cancer |
| fibrinogen | | congenital afibrinogenemia, hypofibrinogenemia or dysfibrinogenemia |
| Filgrastim | Neupogen, Zarxio, Granix | low neutrophil count |
| Follitropin alfa | Gonal-f, Cinnal-f, Fertilex, Ovaleap, Bemfola | fertility medication for ovarian hyperstimulation and ovulation induction |
| Forigerimod | | Systemic lupus erythematosus |
| Galsulfase | Naglazyme | mucopolysaccharidosis VI |
| Galsulphase | Naglazyme | Mucopolysaccharidosis VI |
| Ganirelix acetate | Orgalutran, Antagon | fertility treatment drug for people with ovaries |
| Glatiramer acetate | Copaxone, Glatopa | Multiple sclerosis, Amyotrophic lateral sclerosis, Huntington’s disease, Neurological disorders, Glaucoma |
| Glucagon | GlucaGen | Diagnostic aid to slow gastrointestinal motility in radiographic studies; reversal of hypoglycaemia |
| Growth hormone (somatotropin) | Genotropin, Humatrope, Norditropin, NorlVitropin, Nutropin, Omnitrope, Protropin, Siazen, Serostim, Valtropin | Growth failure due to growth hormone deficiency, Prader-Willi syndrome, AIDS wasting, or cachexia with antiviral therapy |
| Growth hormone releasing hormone (GHRH) | Geref | Diagnosis of defective growth-hormone secretion |
| hemin (human) | PANHEMATIN | the symptoms of occasional attacks of porphyria related to the menstrual cycle in women |
| Hepatitis B surface antigen (HBsAg) | Engerix, Recombivax HB | Hepatitis B vaccination |
| Hepatitis C antigens | Recombinant immuno-blot assay (RIBA) | Diagnosis of hepatitis C exposure |
| Histrelin acetate (gonadotropin releasing hormone; GnRH) | Supprelin LA, Vantas | Precocious puberty |
| HIV antigens | Enzyme immunoassay, OraQuick, Uni-Gold | Diagnosis of HIV infection |
| HPV vaccine | Gardasil | Prevention of HPV infection |
| Human albumin | Albumarc, Albumin, Albumiar, AlbuRx, Albutein, Flexbumin, Buminate, Plasbumin | Decreased production of albumin (hypoproteinaemia), increased loss of albumin (nephrotic syndrome), hypovolaemia, hyperbilirubinaemia |
| Human deoxyribonuclease I, dornase-α | Pulmozyme | Cystic fibrosis; decreases respiratory tract infections in selected patients with FVC greater than 40% of predicted |
| Hyaluronidase | | adjuvant in subcutaneous fluid administration for achieving hydration |
| Hyaluronidase (bovine, ovine) (recombinant human) | Amphadase (bovine), Hydase (bovine), Vitrase (ovine), Hylenex (recombinant human) | Used as an adjuvant to increase the absorption and dispersion of injected drugs, particularly anaesthetics in ophthalmic surgery and certain imaging agents |
| Icatibant acetate | Firazyr | Hereditary angioedema (HAE) |
| idecabtagene vicleucel | bb 2121 | chimeric antigen receptor (CAR) T cell therapy, for multiple myeloma |
| Idursulfase | Elaprase | Hunter syndrome |
| Idursulphase (Iduronate-2-sulphatase) | Elaprase | Mucopolysaccharidosis II (Hunter syndrome) |
| Imiglucerase | Cerezyme | Gaucher’s disease |
| imlifidase | IdeS | enabling kidney transplantation in highly sensitised patients with chronic kidney disease (CKD) |
| inbakicept | | Fusion protein, INTERLEUKIN 15 RECEPTOR .ALPHA. CHAIN with IMMUNOGLOBULIN G1; in trial for Metastatic Non-Small Cell Lung Cancer |
| incobotulinumtoxin A | Xeomin | cervical dystonia |
| Indium- 111 -octreotide | OctreoScan | Neuroendocrine tumour and lymphoma detection |
| Insulin | Humulin, Novolin, Exubera, Novolog (aspart), Apidra (glulisine), Humalog (lispro), NPH (isophane), degludec | Diabetes mellitus, diabetic ketoacidosis, hyperkalaemia |
| insulin human zinc (combo of zinc chloride and insulin) | Humulin L, Novolin L, Iletin II Lente, Insulin Lente Pork | Diabetes mellitus |
| Interferon alfa-2a | Pegasys | hepatitis C and hepatitis B |
| Interferon alfa-2b | Intron-A | chronic hepatitis C, chronic hepatitis B, hairy cel leukemia, Behçet’s disease, chronic myelogenous leukemia, multiple myeloma, follicular lymphoma, carcinoid tumor, mastocytosis and malignant melanoma |
| Interferon beta- 1a - | Rebif Avonex | Multiple sclerosis, Hepatitis C, Human papillomavirus infections, Non-small cell lung cancer, Ulcerative colitis, Crohn’s disease, Rheumatoid arthritis |
| Interferon beta-1b_ | Extavia | Multiple sclerosis, Prostate cancer, Cardiomyopathies, HIV infections, Rhinovirus infections |
| Interferon-gamma-1b | | chronic granulomatous disease and osteopetrosis |
| IR 501 | RAVAX® | Rheumatoid arthritis vaccine |
| Kedbumin | | Blood |
| Lactase | Lactaid | Gas, bloating, cramps and diarrhoea due to inability to digest lactose |
| Langlenatide | | Glucagon-like peptide 1 derivative; in trial for diabesity (diabetic and obesity) |
| Laronidase (α-L-iduronidase) | Aldurazyme | Hurler and Hurler-Scheie forms of mucopolysaccharidosis I |
| L-Asparaginase | ELSPAR | Acute lymphocytic leukaemia, which requires exogenous asparagine for proliferation |
| Lepirudin | Refludan | Heparin-induced thrombocytopaenia |
| Leuprolide acetate | Lupron, Eligard, Lucrin | prostate cancer, breast cancer, endometriosis, uterine fibroids, and early puberty |
| lipegfilgrastim | Lonquex | prophylactic use in cancer patients receiving chemotherapy and at risk for developing chemotherapy-induced neutropenia |
| Liraglutide | Victoza®, Saxenda® | Incretin therapeutic; Glucagon-like peptide 1 receptor agonist; for diabetes |
| lisocabtagene maraleucel | | immunochemotherapy for aggressive B-cell non-Hodgkin lymphoma (NHL) |
| Lixisenatide | | Glucagon-like peptide 1 derivative; for type 2 diabetes |
| lonapegsomatropin | | sustained-release prodrug of somatropin, for growth hormone deficiency |
| luspatercept | REBLOZYL | anemia in adult patients with beta thalassemia who require regular red blood cell (RBC) transfusions |
| marzeptacog alfa | MarzAA | rFVIIa for hemophilia |
| Mecasermin | Increlex | Growth failure in children with growth hormone (GH) gene deletion or severe primary insulin-like growth factor 1 (IGF1) deficiency |
| Mecasermin rinfabate | Iplex | Growth failure in children with growth hormone (GH) gene deletion or severe primary insulin-like growth factor 1 (IGF1) deficiency |
| Menotropins | | fertility disturbances |
| Methoxy polyethylene glycol-epoetin beta | Mircera | anaemia |
| Metreleptin | Myalept, Myalepta | diabetes and various forms of dyslipidemia |
| mixed vespid venom protein | Venomil | hypovolemia, hypoalbuminemia and cardiopulmonary bypass surgery. |
| Molgramostim | Leucomax | low levels of neutrophils |
| Multikine | | Leukocyte Interleukin, for neoadjuvant therapy in patients with squamous cell carcinoma of the head and neck, or SCCHN |
| Nerinetide | | an eicosapeptide, for acute ischemic stroke |
| Nesiritide | Natrecor | Acute decompensated congestive heart failure |
| neuregulin | | in trail for cognition improvement for Alzeihmer’s disease |
| nivobotulinumtoxin A | Neuronox | adults with cervical dystonia; wrinkle reduction |
| Nomacopan | Coversin | C5 complement inhibitor, for Paroxysmal Nocturnal Haemoglobinuria (PNH) / Atypical Hemolytic Uremic Syndrome |
| NovoSeven | | recombinant coagulation Factor VIIa, in trial for Glanzmann’s thrombasthenia, and control surgical bleeding |
| Ocriplasmin | Jetrea | symptomatic vitreomacular adhesion |
| Octreotide | Sandostatin | Acromegaly, symptomatic relief of VIP-secreting adenoma and metastatic carcinoid tumours |
| Octreotide | Sandostatin | somatostatin analog, indicated for severe watery diarrhea and sudden reddening of the face and neck caused by certain types of tumors (e.g., carcinoid tumors, vasoactive intestinal peptide tumors) that are found usually in the intestines and pancreas |
| olipudase alfa | | recombinant human acid sphingomyelinase for acid sphingomyelinase deficiency |
| OnabotulinumtoxinA | Botox | Muscle spasticity, Excessive sweating, Migraine, Cosmetics |
| onasemnogene abeparvovec | Zolgensma | gene therapy medication used to treat spinal muscular atrophy. |
| OspA | LYMErix | Lyme disease vaccination |
| Oxytocin | Pitocin | Labor induction |
| Palifermin (keratinocyte growth factor; KGF) | Kepivance | Severe oral mucositis in patients undergoing chemotherapy |
| Pancreatic enzymes (lipase, amylase, protease) | Arco-Lase, Cotazym, Creon, Donnazyme, Pancrease, Viokase, Zymase | Cystic fibrosis, chronic pancreatitis, pancreatic insufficiency, post- Billroth II gastric bypass surgery, pancreatic duct obstruction, steatorrhoea, poor digestion, gas, bloating |
| Papain | Accuzyme, Panafil | Debridement of necrotic tissue or liquefication of slough in acute and chronic lesions, such as pressure ulcers, varicose and diabetic ulcers, burns, postoperative wounds, pilonidal cyst wounds, carbuncles, and other wounds |
| Parathyroid hormone | | bone disease, hypocalcaemia, and hypercalcaemia |
| Parathyroid hormone (PTH) | | To control low blood calcium due to low levels of parathyroid hormone. |
| Pegadricase | | pegylated uricase, in trial for acute gout flares |
| pegargiminase | ADI-PEG 20 | PEG-arginine deiminase, for cancer therapy |
| Peg-asparaginase | Oncaspar | Acute lymphocytic leukaemia, which requires exogenous asparagine for proliferation |
| pegaspargase | Oncaspar | acute lymphoblastic leukemia |
| Pegfilgrastim | Neulasta, Udenyca | stimulate bone marrow to produce more neutrophils to fight infection in patients undergoing chemotherapy |
| peginterferon alfa-2b | PEG Intron, Sylatron | chronic hepatitis C in patients with compensated liver disease, optionally combined with ribavirin. |
| Peginterferon beta-1a Biogen | | Multiple sclerosis |
| Pegloticase | Krystexxa, Puricase | refractory, chronic gout |
| Pegvaliase | Palynziq | phenylketonuria |
| Pegvisomant | Somavert | Acromegaly |
| pegzilarginase | AEB1102 | to reduce elevated blood arginine levels, arginase 1 deficiency |
| PF-743 | | Hematologic malignancies |
| plasminogen | Ryplazim | congenital plasminogen deficiency, and idiopathic pulmonary fibrosis |
| Pooled immunoglobulins | Octagam | Primary immunodefiencies |
| Protamine sulfate | Prosulf | reverse the effects of heparin |
| protein C | CEPROTIN | severe congenital protein C deficiency for the prevention and treatment of venous thrombosis and purpura fulminans |
| Protein C concentrate | Ceprotin | Treatment and prevention of venous thrombosis and purpura fulminans in patients with severe hereditary protein C deficiency |
| Rasburicase | Elitek | Paediatric patients with leukaemia, lymphoma, and solid tumours who are undergoing anticancer therapy that may cause tumour lysis syndrome |
| Recombinant human bone morphogenic protein 7 (rhBMP7) | Osteogenic protein 1 | Tibial fracture nonunion, lumbar spinal fusion |
| Recombinant purified protein derivative (DPPD) | DPPD | Diagnosis of tuberculosis exposure |
| Renexus | NT-501 | CNTF-secreting Cells for retinal degenerative diseases. |
| Reteplase (deletion mutein of tPA) | Retavase | Management of acute myocardial infarction, improvement of ventricular function |
| rHuIL-12 (e.g., monovalent) | HemaMax | Acute radiation syndrome |
| Rilonacept | Arcalyst | familial cold autoinflammatory syndrome, Muckle-Wells syndrome and neonatal onset multisystem inflammatory disease |
| rimabotulinumtoxin B | Myobloc | cervical dystonia (severe spasms in the neck muscles); wrinkle reduction |
| Romiplostim | Nplate | chronic idiopathic (immune) thrombocytopenic purpura |
| Salmon calcitonin | Fortical, Miacalcin | Postmenopausal osteoporosis |
| sargramostim | LEUKINE | acceleration of myeloid recovery in patients with non Hodgkin’s lymphoma (NHL), acute lymphoblastic leukemia (ALL) and Hodgkin’s disease undergoing autologous bone marrow transplantation (BMT) |
| Sebelipase alfa | Kanuma | lysosomal acid lipase deficiency |
| Secretin | ChiRhoStim (human peptide), SecreFlo (porcine peptide) | Aid in the diagnosis of pancreatic exocrine dysfunction or gastrinoma; facilitates identification of the ampulla of Vater and accessory papilla during endoscopic retrograde cholangiopancreatography |
| Semaglutide | Ozempic and Rebylsus | Incretin therapeutic; Glucagon-like peptide 1 derivative; for type 2 diabetes |
| somatrogon | Lagova | pediatric and adult growth hormone deficiency (GHD) |
| Somatropin | | HGH deficiency |
| sotatercept | | Fusion protein with activin receptor IIa, in trial of pulmonary arterial hypertension |
| Streptokinase | Streptase | Acute evolving transmural myocardial infarction pulmonary embolism, deep vein thrombosis, arterial thrombosis or embolism, occlusion of arteriovenous cannula |
| tadekinig alfa | | recombinant interleukin-18 binding protein for adult-onset Still’s disease |
| tagraxofusp | Elzonris | blastic plasmacytoid dendritic cell neoplasm (BPDCN) in adults and pediatric patients over 2 years old |
| taliglucerase alfa | Elelyso | long-term enzyme replacement therapy (ERT) for adults with a confirmed diagnosis of Type 1 Gaucher disease |
| tasonermin | Beromun | soft tissue sarcoma in combination with Melphalan |
| Taspoglutide | | Glucagon-like peptide 1 derivative; for type 2 diabetes |
| tbo-filgrastim | GRANIX | to reduce the duration of severe neutropenia in patients with non-myeloid malignancies receiving myelosuppressive anti-cancer drugs associated with a clinically significant incidence of febrile neutropenia |
| tebentafusp | | bispecific protein of a soluble T cell receptor fused to an anti-CD3 immune-effector portion, in trial of metastatic uveal melanoma |
| Teduglutide | Gattex | short bowel syndrome |
| Teicoplanin | Targocid | serious infections caused by Gram-positive bacteria |
| telitacicept | RemeGen | Systemic lupus erythematosus, Rheumatoid arthritis, Multiple sclerosi |
| Tenecteplase | TNKase® | Acute myocardial infarction |
| Teriparatide | Forteo/Forsteo, Teribone | osteoporosis |
| Terlipressin | Teripress, Glypressin | norepinephrine-resistant septic shock and hepatorenal syndrome. |
| Thyroid stimulating hormone (TSH), thyrotropin | Thyrogen | Adjunctive diagnostic for serum thyroglobulin testing in the follow-up of patients with well-differentiated thyroid cancer |
| topsalysin | PRX 302 | a pore-forming protein for localized prostate cancer |
| tralesinidase alfa | | fusion of alpha-N-acetylglucosaminidase (NAGLU) with IGF-2, in trial for mucopolysaccharidosis type IIIB |
| Trypsin | Granulex | Decubitus ulcer, varicose ulcer, debridement of eschar, dehiscent wound, sunburn |
| Urokinase | Abbokinase | Pulmonary embolism |
| Vasopressin | Vasostrict | Manage anti-diuretic hormone deficiency. treat diabetes insipidus related to low levels of antiduretic hormone |
| Velaglucerase alfa | VPRIV | Gaucher disease Type 1 |
| velmanase alfa | Lamzede | non-neurological symptoms of patients with mild-to-moderate alpha-mannosidosis. |
| Vestronidase alfa | Mepsevii | mucopolysaccharidosis type VII |
| von willebrand factor | Wilate | hemophilia A for routine prophylaxis to reduce the frequency of bleeding episodes |
| vosoritide | | a C-type Natriuretic Peptide, for achondroplasia |
| Ziv-aflibercept | Eylea and Zaltrap | wet macular degeneration and metastatic colorectal cancer |
| α-1-Proteinase inhibitor | Aralast, Prolastin,Glassia, Prolastin, Prolastin-C, Zemaira | Congenital α-1- antitrypsin deficiency |
| β-Glucocerebrosidase | Cerezyme, Ceredase (purified from pooled human placenta) | Gaucher’s disease |
| | HemaMax | a recombinant human interleukin-12, in trial for counter-acting lethal exposure of radiation |
| | Vasomera | VPAC2-selective Vasoactive Intestinal Peptide Agonist, in trial for Cardiomyopathies; Heart failure; Pulmonary arterial hypertension |
| | Sanguinate | PEGylated bovine carboxyhemoglobin, in trial for Sickle Cell Disease |

### C. Small Molecules

In some embodiments, the cargo loaded into MPVs, e.g., WPVs, disclosed herein is a small molecule, such as any of the small molecules described herein. As used herein, a “small molecule” is a low molecular weight (e.g., < 900 daltons) organic compound that may regulate a biological process. The majority of currently used therapeutic agents are small molecules, and drugs of typically function as enzyme inhibitors, receptor ligands, or allosteric modulators. A small molecule functions as an enzyme inhibitor competing with substrate binding to the catalytic cleft of an enzyme. Similarly, a small molecule may bind to a transporter preventing the substrate to be transported from binding and inhibit transport. Examples of small molecule inhibitors include metalloprotease inhibitors, heat shock protein inhibitors, proteasome inhibitors, tyrosine kinase inhibitors, and serine/threonine kinase inhibitors. Small molecules binding to receptors can function as agonists and antagonists, by competing for the same binding site (Gurevich and Gurevich, Therapeutic Potential of Small Molecules and Engineered Proteins; Handb Exp Pharmacol. 2014; 219: 1-12, and references therein). For example, the first antagonist-receptor drug to be developed was against the HER2, which is a type 1 transmembrane RTK found to be overexpressed in many cancers, and beta-agonists used in asthma are examples of agonistic small molecules. Of note, small molecules are also useful as anti-pathogenic agents, directed against parasites, such as bacteria, fungi, and viruses. Small molecule inhibitors are very effective as antimicrobials because they target enzymes performing biochemical reactions that are specific to the pathogen and have no counterpart in humans. Examples are enzymes involved in s building and maintaining bacterial cell wall or bacterial ribosomes. Viruses can be targeted by small molecules via their reverse transcriptases.

Exemplary small-molecular cargos for use in the present disclosure are provided in Table 5 below.

TABLE 5

Exemplary Small-Molecular Cargos

| | | |
|---|---|---|
| Molecule | Trade Name | Exemplary Indication(s)/MOA |
| ABT 494 | Rinvoq | Rheumatoid arthritis, Crohn’s disease, Ulcerative colitis, Atopic dermatitis |
| Acitretin | Soriatane, Neotigason | Psoriasis, Dermatitis, Cancer |
| ALKS 8700 | | Multiple sclerosis |
| Amifampridine | Firdapse, Ruzurgi | Lambert-Eaton myasthenic syndrome, Congenital myasthenic syndromes, Myasthenia gravis |
| Apremilast | Otezla | Psoriatic arthritis, Plaque psoriasis, Behcet’s syndrome, Ankylosing spondylitis, Atopic dermatitis, Ulcerative colitis, Crohn’s disease, Rheumatoid arthritis, Asthma, Cancer |
| Ardeparin sodium | Normiflo | Deep vein thrombosis |
| Baricitinib | Olumiant, Baricinix | Rheumatoid arthritis, Systemic lupus erythematosus, Diabetic nephropathies, Atopic dermatitis, Psoriasis |
| Betamethasone valerate foam - Stiefel Laboratories | | Atopic dermatitis, Psoriasis, Seborrhoeic dermatitis, Skin disorders |
| Calcipotriol -Stiefel | | Plaque psoriasis, Psoriasis |
| Calcipotriol/betamethasone dipropionate | Daivobet, Enstilar | Plaque psoriasis, Psoriasis |
| Calcitriol - Galderma | | Plaque psoriasis |
| Celecoxib | Celebrex, Onsenal, Elyxyb | Dysmenorrhoea, Acute pain, Tenosynovitis, Familial adenomatous polyposis, Back pain, Ankylosing spondylitis, Tendinitis, Dental pain, Rheumatoid arthritis, Postoperative pain, Osteoarthritis, Pain, Rheumatic disorders, Juvenile rheumatoid arthritis, Cervicobrachial syndrome, Periarthritis, Non-small cell lung cancer, Stomatitis, Gouty arthritis, Bladder cancer, Alzheimer’s disease, Prostate cancer |
| Cladribine | Leustatin, Mavenclad | Lymphoma, Leukaemia, Chronic lymphocytic leukaemia, Hairy cell leukaemia, Multiple sclerosis, Psoriasis, Transplant rejection |
| Clobetasol propionate topical - Galderma | | Atopic dermatitis, Psoriasis, Skin disorders |
| Desoximetasone topical -Taro Pharmaceuticals | Topisolone, Topicort, Emcor, Desacort | Plaque psoriasis, Atopic dermatitis |
| DFD 01 | | Plaque psoriasis |
| DFD 06 | | Plaque psoriasis |
| Dimethyl fumarate | Fumaderm; Tecfidera | Multiple sclerosis, Rheumatoid arthritis, Psoriasis |
| Enoxaparin sodium | Lovenox, Clexane, Xaparin | Deep vein thrombosis (DVT) and pulmonary embolism |
| Esomeprazole/naproxen | | Osteoarthritis, Rheumatoid arthritis, Ankylosing spondylitis |
| Fampridine sustained-release | | Multiple sclerosis, Neurological disorders, Stroke, Spinocerebellar degeneration, Spinal cord injuries, Parkinson’s disease, Cerebral palsy |
| Filgotinib | GLPG0634 | Rheumatoid arthritis, Crohn’s disease, Ulcerative colitis |
| Fingolimod | Gilenya | Multiple sclerosis, Chronic inflammatory demyelinating polyradiculoneuropathy, Amyotrophic lateral sclerosis, Renal transplant rejection, Optic neuritis, Type 1 diabetes mellitus, Rheumatoid arthritis, Graft-versus-host disease, Myocarditis |
| Fluocinonide Cream_ Valeant | | Skin disorders, Plaque psoriasis |
| Fondaparinux sodium | Arixtra | deep vein thrombosis and pulmonary embolism |
| heparin sodium | | used for anticoagulation for the following conditions: Acute coronary syndrome, e.g., NSTEMI Atrial fibrillation, Deep-vein thrombosis and pulmonary embolism, Cardiopulmonary bypass for heart surgery ECMO circuit for extracorporeal life support, Hemofiltration Indwelling central or peripheral venous catheters |
| Ibuprofen/famotidine | | Musculoskeletal pain, Osteoarthritis, Rheumatoid arthritis, NSAID-induced ulcer, Ankylosing spondylitis |
| Laquinimod | | Multiple sclerosis, Huntington’s disease, Crohn’s disease, Lupus nephritis, Systemic lupus erythematosus |
| Mahonia aquifolium extract | | Psoriasis |
| Masitinib | Masivet, Kinavet | Amyotrophic lateral sclerosis, Mastocytosis, Prostate cancer, Alzheimer’s disease, Colorectal cancer, Malignant melanoma, Pancreatic cancer, Gastrointestinal stromal tumours, Multiple myeloma, Asthma, Peripheral T-cell lymphoma, Multiple sclerosis, Crohn’s disease, Ovarian cancer, Progressive supranuclear palsy, Breast cancer, Chronic obstructive pulmonary disease, Non- small cell lung cancer, Mood disorders, Head and neck cancer, Glioblastoma, Hepatocellular carcinoma, Gastric cancer, Oesophageal cancer, Stroke, Psoriasis, Rheumatoid arthritis |
| Meloxicam | Mobic, Metacam, Anjeso | Osteoarthritis, Periarthritis, Rheumatoid arthritis Neuropathic pain, Gout, Ankylosing spondylitis, Back pain, Juvenile rheumatoid arthritis, Pretern labour |
| Methotrexate subcutaneous auto-injection - Antares Pharma | | Psoriasis, Rheumatoid arthritis, Juvenile rheumatoid arthritis |
| Mitoxantrone | Novantrone | Breast cancer, Acute nonlymphocytic leukaemia Cancer, Acute promyelocytic leukaemia, Cancer pain, Acute myeloid leukaemia, Ovarian cancer, Leukaemia, Liver cancer, Multiple sclerosis, Non-Hodgkin’s lymphoma |
| Mometasone/salicylic acid | | Psoriasis, Skin disorders |
| Ozanimod | Zeposia | Multiple sclerosis, Ulcerative colitis, Crohn’s disease |
| Pefcalcitol | | Plaque psoriasis, Palmoplantar keratoderma |
| Piclidenoson | | Psoriasis, Rheumatoid arthritis, Glaucoma, Uveitis, Osteoarthritis, Dry eyes, Colorectal cancer, Solid tumours |
| Ponesimod | ACT-128800 | Multiple sclerosis, Graft-versus-host disease, Immunological disorders, Plaque psoriasis |
| Poractant alfa (lung phospholipid extract) | Curosurf | respiratory distress syndrome in babies |
| Pramlintide | Symlin | Diabetes mellitus (in combination with insulin) |
| Prednisone | Rayos, Deltasone | Asthma, Rheumatoid arthritis, Chronic obstructive pulmonary disease, Psoriatic arthritis, Ankylosing spondylitis, Polymyalgia rheumatica, Nocturnal asthma |
| Remdesivir | Veklury | Anti-viral infection, such as Ebola infection, HCV infection and coronavirus infection (e.g., COVID-19) |
| Siponimod | Mayzent | Multiple sclerosis, Polymyositis, Dermatomyositis, Renal failure, Liver failure |
| Tazarotene topical | | Acne vulgaris, Psoriasis, Photodamage |
| Tazarotene/ulobetasol | | Plaque psoriasis |
| Teriflunomide | Aubagio | Multiple sclerosis |
| Tofacitinib | Xeljanz, Jakvinus, Tofacinix | Rheumatoid arthritis, Psoriatic arthritis, Juvenile rheumatoid arthritis, Ulcerative colitis, Plaque psoriasis, Atopic dermatitis, Ankylosing spondylitis, Crohn’s disease, Dry eyes, Renal transplant rejection, Irritable bowel syndrome, Asthma |
| Ulobetasol | 122-0551 (code name) | Plaque psoriasis |
| VAL BRO 03 | | Psoriatic arthritis |

### B. Allergen, Adjuvant, Antigen, or Immunogen

In some embodiments, the biologic agent is an allergen, adjuvant, antigen, or immunogen. In some embodiments, the allergen, antigen, or immunogen elicits a desired immune response to increase allergen tolerance or reduce the likelihood of an allergic or immune response such as anaphylaxis, bronchial inflammation, airway constriction, or asthma. In some embodiments, the allergen, antigen, or immunogen elicits a desired immune response to increase viral or pathogenic resistance or elicit an anticancer immune response. In some embodiments, the allergen or antigen elicits a desired immune response to treat an allergic or autoimmune disease. In some embodiments, an autoantigen may be used to increase immunological tolerance, thereby benefiting treatment of the corresponding autoimmune disease or decreasing an autoimmune response.

As used herein, the term “adjuvant” refers to any substance which enhances an immune response (e.g. in the vaccine, autoimmune, or cancer context) by a mechanism such as: recruiting of professional antigen-presenting cells (APCs) to the site of antigen exposure; increasing the delivery of antigens by delayed/slow release (depot generation); immunomodulation by cytokine production (selection of Th1 or Th2 response); inducing T-cell response (prolonged exposure of peptide-MHC complexes (signal 1) and stimulation of expression of T-cell-activating co-stimulators (signal 2) on an APC surface) and targeting (e.g., carbohydrate adjuvants which target lectin receptors on APCs), and the like.

In some embodiments, the allergen can be a food allergen, an animal allergen (e.g., pet such as dog, cat, or rabbit), or an environmental allergen (such as dust, pollen, or mildew). In some embodiments, the allergen is selected from abalone, perlemoen, acerola, Alaska pollock, almond, aniseed, apple, apricot, avocado, banana, barley, bell pepper, brazil nut, buckwheat, cabbage, chamomile, carp, carrot, casein, cashew, castor bean, celery, celeriac, cherry, chestnut, chickpea, garbanzo, bengal gram, cocoa, coconut, cod, cotton seed, courgetti, zucchini, crab, date, egg (e.g. hen’s egg), fig, fish, flax seed, linseed, frog, garden plum, garlic, gluten, grape, hazelnut, kiwi fruit (chinese gooseberry), legumes, lentil, lettuce, lobster, lupin or lupine, lychee, mackerel, maize (corn), mango, melon, milk (e.g.,cow), mollusks, mustard, oat, oyster, peach, peanut (or other ground nuts or monkey nuts), pear, pecan, persimmon, pistachio, pine nuts, pineapple, pomegranate, poppy seed, potato, pumpkin, rice, rye, salmon, sesame, shellfish (e.g.,crustaceans, black tiger shrimp, brown shrimp, greasyback shrimp, Indian prawn, neptune rose shrimp, white shrimp), snail, soy, soybean (soya), squid, strawberry, sulfur dioxide (sulfites), sunflower seed, tomato, tree nuts, tuna, turnip, walnut, or wheat (e.g. breadmaking wheat, pasta wheat, kamut, spelt).

In some embodiments, the allergen can be an allergenic protein, peptide, oligo- or polysaccharide, toxin, venom, nucleic acid, or other allergen, such as those listed at allergenonline.org. In other embodiments, the allergen can be an airborne fungus, mite or insect allergen, plant allergen, venom or salivary allergen, animal allergen, contact allergen, parasitic allergen, or bacterial airway allergen.

In some embodiments, the cargo loaded into the MPVs, e.g., WPVs, can be an autoimmune antigen. Exemplary autoantigens and the corresponding autoimmune disorders are provided in Table 6 below.

TABLE 6

Exemplary Autoimmune Diseases and Involved Antigens

| Molecule (Antigen) | Exemplary Indication/MOA |
|---|---|
| MOG (138) | Acute disseminated encephalomyelitis |
| Synapsin 1 (260) | Celiac disease |
| Transglutaminase | Celiac disease |
| ND | Encephalitis lethargica |
| hnRNP A1 (244) | HAM/tropical spastic paraparesis; Inhibits neuronal activity (246) |
| Aldehyde reductase (271) | Hashimoto’s encephalitis |
| Thyroglobulin (271, 272) | Hashimoto’s encephalitis |
| AMPAR (GluR1, GluR2) | Limbic encephalitis; Altered receptor location (264) |
| NMDAR (265) [NR1/NR 2B (224)] | Limbic encephalitis; Receptor internalization |
| Lgil (24) | Limbicencephalitis |
| AQP4(150, 151, 171) | Neuromyelitis optica; Receptor- mediated internalization; complement- mediated toxicity |
| Lysogan glioside dopamine D2 receptor | Poststreptoc occal movement disorders, Sydenham’s chorea, and PANDAS; Aberrant cell signaling, neurotransmitter release (216, 259) |
| Tubulin (199, 215, 216, 259) | Poststreptoc occal movement disorders, Sydenham’s chorea, and PANDAS; Aberrant cell signaling, neurotransmitter release (216, 259) |
| GluR3 (267) | Rasmussen encephalitis; Complement- mediated toxicity (270) |
| GAD (274) | Stiff-person syndrome |
| Gephryin (275) | Stiff-person syndrome |
| GABA(B) receptor (277) | Stiff-person syndrome |
| Amphiphysin (233) | Stiff-person syndrome; Synaptic inhibition (233) |
| Neuronal surface P antigen (116) | Systemic lupus erythematosus; Ca2+ influx, apoptosis (116) |
| NR2A/N R2B | Systemic lupus erythematosus; Receptor modulation, apoptosis (50, 100, 101) |

### D. Anti-Infection Cargos

Any of the LNP-MPVs disclosed herein can be loaded with one or more anti-infection cargos to form cargo-loaded LNP-MPVs.

As used herein, the term “anti-infection cargo” or “anti-infection agent” is meant to include any biomolecule or agent having anti-infection activity and can be loaded into or by an LNP-MPV, including, for example, a biologic, small molecule, therapeutic agent, and/or diagnostic agent. The anti-infection cargo (e.g., biological molecule) in the cargo-loaded LNP-MPVs described herein can be of any type. Examples include, but are not limited to, proteins, nucleic acids, lipids, carbohydrates, and small molecules. The anti-infection cargo may be a biological molecule that is not naturally-occurring in a milk vesicle, e.g., has been synthetic or modified as described herein.

In some embodiments, the anti-infection cargo is a biologic agent, for example, those described herein. In some embodiments, the biologic agent is a peptide, a polypeptide, or protein. In other embodiments, the biologic agent is a nucleic acid. In some examples, the nucleic acid may be a therapeutic agent per se, i.e., comprises a nucleic acid based biologic agent (e.g., an interfering RNA, an antisense oligonucleotide, or an aptamer) as described herein. In other examples, the nucleic acid may encode an anti-infection therapeutic agent (e.g.,, a nucleic acid or a protein-based therapeutic agent). In some embodiments, the anti-infection cargo loaded into the LNP-MPVs comprises a vaccine, for example, an anti-pathogenic vaccine (e.g., an anti-viral vaccine) as described herein.

In some embodiments, the cargo loaded into the LNP-MPVs disclosed herein comprise one or more anti-infection agents (e.g., nucleic acid-based or protein-based) targeting an infection, for example, infection caused by a virus such as a coronavirus (e.g., SARS such as SARS-CoV-2). Examples include a vaccine or a neutralizing antibody, a small molecule, a polypeptide therapeutic agent, or a nucleic acid (e.g., those designed for producing such protein-based therapeutic agents). Exemplary anti-infection agents are provided in Tables 1-12 herein.

In specific examples, the cargo loaded into LNP-MPVs comprise one or more anti-infectious agents, including, but not limited to, antiviral agents, anti-malarial, anti-inflammatory, anti-bacterial, anti-fungal, anti-protozoal, IL-6 inhibitors, Jak Inhibitors (e.g., baricitinib, fedratinib, ruxolitinib, tofacitinib, oclacitinib, peficitinib, upadacitinib, filgotinib, cerdulatatinib, gandotinib, lestaurtinib, momelotinib, pacritinib, abrocitinib, cucurbitacinI, and CHZ868), interferon, kinase inhibitor, protease inhibitor, antibodies, (such as anti-Jak or anti-IL-6 antibodies, IL-6 receptor antagonists, or anti-T cell antibodies), antibodies directed against pathogenic targets (e.g., broadly neutralizing antibodies), convalescent plasma, other polypeptides (such as decoy receptors, growth factors or cytokines (e.g., anti-inflammatory cytokines), and viral antigens).

The antiviral agents disclosed herein refer to agents capable of inhibiting viral infection by any mechanism of action. In some embodiments, an antiviral agent may suppress the activity of one or more viral proteases, leading to blockade of viral protein synthesis and/or viral replication. In other embodiments, an antiviral agent may block virus entry into the host cells, for example, via inhibition of binding of virus to cell receptor or inhibits membrane fusion. In other instances, an antiviral agent may target viral nucleic acid synthesis, for example, inhibiting RNA-dependent RNA polymerase activity. Such antiviral agent may be nucleoside analogs. In yet other instances, an antiviral agent may impair endosome trafficking within the host cells and/or limit viral assembly and release.

Exemplary antiviral agents include, but are not limited to, Abacavir, Acyclovir (Aciclovir), ACE2 inhibitor, Adefovir, Alisporivir, Amantadine, Amodiaquine, Ampligen, Amprenavir (Agenerase), Arbidol (Umifenovir), Artesunate, Atazanavir, Atripla, amiloride (EIPA), Balavir, Baloxavir marboxil (Xofluza), Berberine, Biktarvy, Brequinar, Brincidofovir, Camostat, Cepharanthine, Chloroquine, Cidofovir, Cobicistat (Prezcobix), Combivir (fixed dose drug), Cyclosporine, CYT107, Darunavir, Danoprevir, Delavirdine, Descovy, Didanosine, Diphyllin, Docosanol, Dolutegravir, Ecoliever, Edoxudine, Efavirenz, Eflomithine, Emtricitabine, Emetine, Emodin, Enfuvirtide, Entecavir, Famciclovir, Filociclovir, Fomivirsen, Fosamprenavir, Foscarnet, Fosfonet, Fusion inhibitor, Ganciclovir, Galdecivir (Galidesivir, BCX4430), Hydroxychloroquine, Ibacitabine, Idoxuridine, Imiquimod, Imunovir, Indinavir, Inosine, Integrase inhibitor, Interferon type I, Interferon type II, Interferon type III, Interferon, Ivermectin, Labyrinthopeptin A2, Labyrinthopeptin A1, Lamivudine, Letermovir, Lopinavir, Loviride, Lobucavir, Luteolin, Maraviroc, Mefloquine, Methisazone, Moroxydine, Mycophenolic acid, Nafamostat, Nelfinavir, Nevirapine, Nexavir, Niclosamide Nitazoxanide, N-MCT, Norvir, Nucleoside analogues, Oseltamivir (Tamiflu), Peginterferon alfa-2a, Penciclovir (Denavir), Peramivir (Rapivab), Pleconaril, Podophyllotoxin, Protease inhibitor (pharmacology), Posaconazole, Pyramidine, Raltegravir, Quinine, Remdesivir, Reverse transcriptase inhibitor, Ribavirin, Rimantadine, Ritonavir, Saquinavir, Sofosbuvir, Suramin, Stavudine, Silvestrol, Synergistic enhancer (antiretroviral), Telaprevir, Tenofovir alafenamide, Tenofovir disoproxil, Tenofovir, Tilorone (Amixin), Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

Exemplary anti-bacterial agents include, but are not limited to, amikacin, amoxicillin, ampicillin, arsphenamine, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clindamycin, cloxacillin, colistin, dalfopristan, dalbavancin, demeclocycline, dicloxacillin, dirithromycin, doxycycline, erythromycin, enafloxacin, ertepenem, ethambutol, flucloxacillin, fosfomycin, furazolidone, gatifloxacin, geldanamycin, gentamicin, herbimicin, imipenem, isoniazide, kanamicin, kasugamycin, levofloxacin, linezolid, lomefloxacin, loracarbef, mafenide, minocycline, moxifloxacin, meropenem, metronidazole, mezlocillin, minocycline, monensin, mupirozin, nafcillin, neomycin, novobiocin netilmicin, nitrofurantoin, norfloxacin, ofloxacin, Oritavancin, oxytetracycline, penicillin, piperacillin, platensimycin, polymixin B, prontocil, pyrazinamide, quinupristine, rifampin, roxithromycin, salinomycin, spectinomycin, streptomycin, sulfacetamide, sulfamethizole, sulfamethoxazole, teicoplanin, telithromycin, tetracycline, ticarcillin, tobramycin, trimethoprim, troleandomycin, trovafloxacin, vibativ (telavancin) and vancomycin.

Exemplary anti-fungal agents include, but are not limited to, amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole.

Table 7 provides exemplary anti-viral agents that can be loaded into vesicles described herein for oral delivery.

TABLE 7

Exemplary Anti-Viral Cargos

| | | |
|---|---|---|
| Copany | Drug | Target / Goal |
| Abivax SA | ABX464 | inhibits replication of SARS-CoV-2 ; upregulating a microRNA, miR-124, with anti-inflammatory, antiviral and tissue repair properties |
| AMRX | hydroxychloroquine | SARS-CoV-2 replication |
| Anivive Lifesciences | GC376 = small molecule protease inhibitor | inhibit 3C or 3C-like protease (3Cpro or 3CLpro, respectively) responsible for viral replication |
| Ascletis , GILD | ASC09/oseltamivir , ritonavir/oseltamivir , oseltamivir | viral proteins |
| Ascletis , ROG, Genova Biotech (trials run by The Ninth Hospital of Nanchang) | Ritonavir + Pegasys + Ganovo Ganovo® (Danoprevir) + ritonavir with or without interferon atomization Pegasys Novaferon atomization Lopinavir + ritonavir Chinese medicine + interferon atomization | Viral protease inhibitor / human type 1 interferon receptors / NS3/4A protease inhibitor / multiple others |
| ASCLF,ROG, Genova Biotech , Pharmstandard (trials run by Guangzhou 8th People’s Hospital) | Ritonavir/Pegasys/Arbidol | Viral protease inhibitor / intercalation into membrane lipids leading to the inhibition of membrane fusion between virus particles and plasma membranes |
| Atea Pharmaceuticals | AT-527 = Purine Nucleotide analogue | SARS-CoV-2 RdRp |
| Atriva Therapeutics GmbH | ATR-002 (MEK inhibitor drug) | viral replication protein(s) |
| Bayer Inc. (Mississauga, Ontario) , Population Health Research Institute (PHRI) | chloroquine + azithromycin with/without interferon beta-1b | SARS-CoV-2 |
| BCRX | Galidesivir | RNA polymerase |
| Biotron | Anti-viroporin drugs | virus protein known as viroporins |
| CANF | Piclidenoson | single stranded RNA viruses |
| COCP , Kansas State University Research Foundation (“KSURF”) | proprietary broad-spectrum antiviral compounds | SARS-CoV-2 |
| Dilafor, Karolinska Development, Liverpool University, Keele University | tafoxiparin | SARS-CoV-2 spike protein |
| ENTA | direct-acting antiviral drug candidates | SARS-CoV-2 |
| First Affiliated Hospital of Zhejiang University School of Medicine | ASC09/ritonavir, lopinavir/ritonavir, with or without umifenovir | viral proteins |
| First Hospital Affiliated to Zhejiang University’s Medical School | Baloxavir Marboxil, Favipiravir | RdRp |
| Fundação de Medicina Tropical Dr. Heitor Vieira Dourado | Chloroquine diphosphate | SARS-CoV-2 replication |
| Genomictree | antiviral treatment | SARS-CoV-2 |
| GILD | Remdesivir | RdRp |
| GILD, U.S. Army Medical Research and Development Command | Remdesivir | RdRp |
| Glenmark Pharmaceuticals | Favipiravir | SARS-CoV-2 |
| GSK, VIR | VIR-7831 and VIR-7832 = VIR′s monoclonal antibody platform technology + GSK’s functional genomics/CRISPR screening + artificial intelligence | SARS-CoV-2 + cellular host genes/proteins |
| IHU in Marseille | Plaquenil (hydroxychloroquine) | SARS-CoV-2 replication |
| ImmuneMed | HzVSFv13 (an injection of Virus Suppressing Factor (VSF)) | VSF receptors on infected cells |
| IMUX | IMU-838 = a selective oral DHODH inhibitor | Dihydroorotate dehydrogenase (DHODH) = an enzyme required for de novo pyrimidine synthesis. |
| JNJ , GILD (trials run by Shanghai Public Health Clinical Center) | Darunavir / Cobicistat | Protease inhibitor / inhibition of human CYP3A proteins |
| Kainos Medicine | antiviral drug candidate | SARS-CoV-2 enzymes (RdRp?) |
| KPTI , Precision for Medicine , PROMETRIKA | XPOVIO® (selinexor) | |
| LCTX , California Institute for Regenerative Medicine (CIRM) | VAC2 vaccine = allogeneic dendritic cell therapy | SARS-CoV-2 |
| MBRX , CNSP , WBIO , University of Texas Medical Branch at Galveston (UTMB) | WP1122 = prodrug of 2-deoxy-D-glucose, or 2-DG | inhibition of viral glycolysis and glycosylation ; WP1122 is a prodrug of 2-DG (2-deoxy-D-glucose) that, based on recently developed preclinical data, appears to overcome 2-DG’s lack of drug-like properties and is able to significantly increase tissue/organ concentration |
| MOLN | monospecific DARPin® | viral “spike” protein |
| MRK , Ridgeback Therapeutics | EIDD-2801 = orally-bioavailable form of a ribonucleoside analog | inhibits the replication of multiple RNA viruses including SARS-CoV-2 |
| MYL | hydroxychloroquine | SARS-CoV-2 replication |
| OyaGen, Inc. | OYA1 = broad-spectrum antiviral | inhibition of SARS-CoV-2 |
| People’s Hospital of Guangshan County | Azvudine | viral reverse transcriptase |
| People’s Hospital of Wuhan University , Zhang Zhan (researcher) | hydroxychloroquine | SARS-CoV-2 replication |
| Pfizer | Protease inhibitor | Inhibition of 3C-like protease of SARS-CoV-2 |
| PharmaMar | Aplidin® (plitidepsin) | EF1A protein |
| Population Health Research Institute | chloroquine + azithromycin | SARS-CoV-2 replication |
| Shanghai Public Health Clinical Center | hydroxychloroquine | SARS-CoV-2 replication |
| Sichuan Kelun Pharmaceutical Co. Ltd. | S-protein/ACE2 targeted prophylactic polypeptide (inhalant) | polypeptide targeting SARS-CoV-2 spike protein (S-protein) |
| Sirona Biochem | antiviral compounds = Neuraminidase Inhibitors, Nucleoside Analogs and Iminosugars | SARS-CoV-2 |
| TEVA | hydroxychloroquine | SARS-CoV-2 replication |
| Tongji Hospital | Abidol hydrochloride | 2019-nCoV |
| University of Lübeck in Germany , Rolf Hilgenfeld (Structural biologist) | antiviral compounds | viral proteases |
| University of Minnesota | hydroxychloroquine | SARS-CoV-2 replication |
| University of North Carolina | NHC (P-D-N4-hydroxycytidine) = ribonucleoside analogue | ribonuceloside analogue that induces lethal mutations in viral RNA but not host RNA |
| University of Oxford | hydroxychloroquine | SARS-CoV-2 replication |
| Zhejiang Hisun Pharmaceutical Co Ltd | Favipiravir / Avigan (T-705) | guanine analogue approved for influenza treatment, can effectively inhibit the RNA-dependent RNA polymerase of RNA viruses such as influenza, Ebola, yellow fever, chikungunya, norovirus and enterovirus [1], and a recent study reported its activity against 2019-nCoV (EC50 = 61.88 µM in Vero E6 cells) [2] |

Table 8 below provides exemplary anti-inflammatory agents that can be used, either alone or in combination with an anti-infection agent, in treatment of an infection. Such agents also can be loaded into LNP-MPVs for oral delivery.

TABLE 8

Exemplary Anti-Inflammatory Agents

| | | |
|---|---|---|
| Company | Drug | Target / Goal |
| Airway Therapeutics , Respiratory Diseases Branch of the National Institutes of Health (NIH), National Institute of Allergy and Infectious Diseases (NIAID) | AT-100 = recombinant human surfactant protein D (rhSP-D) | Facilitating the binding and clearance of the virus by lung immune cells ; Regulating the body’s immune cells to reduce the overwhelming inflammation that is the primary mechanism of illness in severe viral infections ; Inhibiting infectivity and replication for several types of bacteria and viruses, including the primary coronavirus infection and also the secondary bacterial and viral infections that oftencomplicate the care of patients with serious infections |
| ALDX | ADX-629 , reproxalap , library of novel reactive aldehyde species (RASP) inhibitors | reactive aldehyde species (RASP) inhibitors |
| ALXN | ULTOMIRISO (ravulizumab-cwvz) | inhibiting the C5 protein in the terminal complement cascade |
| AMPE | nebulized Ampion | upregulate the production of these healing lipid mediators’ prostaglandins in-vitro + attenuate multiple inflammatory signals (i.e. TNF α, IL6, CXCL10) |
| Azidus Brasil, Cellavita Pesquisa Científica Ltda , Hospital Vera Cruz | NestCell® (mesenchymal stem cell) | rebalance the ratio between pro-inflammatory and anti-inflammatory signals |
| BHVN | BHV3500-203 (vazegepant) = intranasal CGRP receptor antagonist | counter/reduce CGRP release as a result of viral activation of TRP channels |
| CANF | Piclidenoson | A3AR |
| Cellivery | iCP-NI (improved cell-permeable nuclear import inhibitor) | pro-inflammatory cytokines such as TNF-α, IL-6 and IFN-y |
| CERC , MYGN | CERC-002 = anti-LIGHT monoclonal antibody | Minimize a dysregulated inflammatory response and cytokine storm in patients with acute infection |
| CYDY , NIH of Mexico | PRO 140 (leronlimab) | CCR5 antagonist |
| First Affiliated Hospital of Fujian Medical University , Ning Wang, MD., PhD. (researcher) | Fingolimod | S1P receptor agonist |
| Humanigen | lenzilumab (monoclonal antibody) | neutralizes GM-CSF (human-granulocyte-macrophage colony stimulating factor) |
| IFRX | IFX-1 = monoclonal anti-C5a antibody | anti-C5a antibody |
| IFRX , Beijing Defengrui Biotechnology Co., Ltd. | BDB-1 (“target-binding molecules”) | anti-C5a monoclonal antibody |
| IMAB | TJM2 (TJ003234) | treat cytokine storm in severe and critically ill patients caused by the coronavirus disease (COVID-19) |
| INCY | Jakafi® (ruxolitinib) | supressing cytokine storm |
| Jiangxi Qingfeng Pharmaceutical Co. Ltd. | Xiyanping | N/A |
| Johns Hopkins University | prazosin = α1-AR antagonist | α1-AR antagonist |
| Komipharm | Panaphix | restrains the overproduction of immune cells and their activating compounds, cytokines |
| Korea United Pharm | inhaled steroid | N/A |
| MESO | remestemcel-L | counteract the inflammatory processes implicated in these diseases by down-regulating the production of pro-inflammatory cytokines, increasing production of anti-inflammatory cytokines, and enabling recruitment of naturally occurring anti-inflammatory cells to involved tissues |
| MNOV | MN-166 (ibudilast) | ibudilast significantly reduced the levels of inflammatory cytokines including TNF-alpha (p<0.001), IL-1beta (p<0.001), IL-6 (p<0.001), and MCP-1 (p<0.001) in a dose-dependent manner, indicating that ibudilast suppressed the inflammatory response |
| National Institute of Infectious Diseases (Tokyo, Japan) , Nippon Veterinary and Life Science University , Gunma University Graduate School of Medicine | Alvesco (ciclesonide) = inhaled glucocorticoid for asthma, allergies, etc. | viral NSP15 protein (an endonuclease) is the possible target |
| NK , Be The Match BioTherapies | BM-Allo.MSC = mesenchymal stem cells (MSC) | reduce the lung inflammation associated with ARDS |
| NVS , INCY | Jakavi® (ruxolitinib) = oral inhibitor of the JAK 1 and JAK 2 tyrosine kinases | JAK 1 and JAK 2 tyrosine kinases |
| Peking Union Medical College Hospital, Zhongda Hospital , Zhongnan Hospital , Renmin Hospital of Wuhan University | Methylprednisolone | Multiple (dampening the inflammatory cytokine cascade, inhibiting the activation of T cells, decreasing the extravasation of immune cells into the respiratory system, facilitating the apoptosis of activated immune cells, and indirectly decreasing the cytotoxic effects of nitric oxide and tumor necrosis factor alpha) |
| Pharming Group N.V. | RUCONEST® (recombinant human C1 inhibitor) | human C1 inhibitor |
| REGN , SNY | Kevzara (sarilumab) | anti-IL-6 antibody |
| ROG | Actemra (tocilizumab) | interleukin-6 inhibitor |
| ROG , Qilu Hospital of Shandong University | Bevacizumab | circulating VEGF |
| Sino Biopharmaceutical Limited, Chia-Tai Tianqing Pharmaceutical Group Co., Ltd | Diammonium Glycyrrhizinate enteric capsules combined with vitamin C tablets | anti- inflammatory + eliminate oxygen free radicals |
| SOBI | Anakinra, Emapalumab | IL-1 inhibitor, anti-interferon-gamma antibody |
| Tasly Pharmaceutical Group | T89 (Dantonic) | Multiple |
| TBPH | TD-0903 = lung-selective, nebulized Janus kinase inhibitor (JAKi) | Janus kinase inhibitor (JAKi) |
| Tiziana Life Sciences | TZLS-501 | interleukin-6 inhibitor |
| Tongji Hospital | Methylprednisolone | Multiple (dampening the inflammatory cytokine cascade, inhibiting the activation of T cells, decreasing the extravasation of immune cells into the respiratory system, facilitating the apoptosis of activated immune cells, and indirectly decreasing the cytotoxic effects of nitric oxide and tumor necrosis factor alpha) |
| Tongji Hospital, Huilan Zhang (researcher) | pirfenidone | TGF-β (transforming growth factor beta; |
| Tongji Hospital, Tongji Medical College , Huazhong University of Science and Technology | ruxolitinib (brand names: Jakafi and Jakavi) = oral selective JAK1/JAK2 inhibitor | Ruxolitinib specifically binds to and inhibits protein tyrosine kinases JAK 1 and 2, which may lead to a reduction in inflammation and an inhibition of cellular proliferation |

Table 9 below provides exemplary vaccine compositions that can be loaded into LNP-MPVs for oral delivery.

TABLE 9

Exemplary Vaccine Compositions

| | | |
|---|---|---|
| Company | Drug | Target / Goal |
| AJ Vaccines | | induce a protective immune response by delivering antigens that closely mimic the native structures of the virus » tries to mimic natural interaction of infectious pathogens with our immune system |
| AKER | novel coronavirus vaccine using Premas’ genetically engineered S. cerevisiae platform, D-Crypt® | structural proteins of the coronavirus |
| ALT , University Of Alabama At Birmingham | AdCOVID = vaccine candidate | peptide vaccine designed to provide systemic immunity following a single intranasal dose » Altimmune has completed the design and synthesis of the vaccine and is now advancing it toward animal testing and manufacturing |
| Anges | | research and development of the vaccine therapy with HGF Plasmid |
| APDN, LineaRx , Takis Biotech | PCR-produced (polymerase chain reaction vaccine) linear DNA vaccine candidates | synthetic gene that when delivered to muscles, should enable the temporary production of a designed antigen that could provoke an immune response against the virus |
| ARCT, Duke-NUS | LUNAR-COV19 | SARS-CoV-2 epitopes |
| AZN, University of Oxford , Vaccitech | AZD1222 (ChAdOx1 nCoV-19) = replication-deficient chimpanzee viral vector based on a weakened version of a common cold (adenovirus) virus that causes infections in chimpanzees and contains the genetic material of SARS-CoV-2 spike protein | stimulate immune response against SARS-CoV-2 spike protein |
| Beroni Group , Tianjin University | nanobody-based targeted treatment | coronavirus antigens |
| Betta Pharmaceuticals , Beijing Dingcheng Taiyuan Biotechnology | Dendritic cell vaccine | stimulate immune response by presenting antigens to dendritic cells |
| BNTX , PFE , Fosun Pharma, Polymun | BNT162 , BTN1626b2 | BNT162 is BioNTech’s mRNA vaccine program aimed at preventing COVID-19 infection and is the first product candidate from Project Lightspeed. Lightspeed is BioNTech’s accelerated development program encompassing the prevention and treatment of COVID-19 infection, which leverages BioNTech’s proprietary mRNA platforms for infectious diseases, its fully-owned GMP manufacturing infrastructure for mRNA vaccine production and its global clinical development capabilities, drawing on BioNTech’s broad network of global collaborators |
| Boryung Biopharma | COVID-19 vaccine | stimulate immune response |
| Boston Children’s Hospital - Precision Vaccines Program (PVP) | vaccine specially targeted toward older populations | coronavirus spike protein |
| BSGM | Vicromax(tm) (merimepodib, or MMPD) | targets RNA-dependent polymerases |
| CAPR | CAP-1002 (exosome-based vaccine) | Coronavirus antigen(s) |
| Codagenix, Serum Institute of India | | live-attenuated vaccine, which can induce ar immune response to different antigens of the virus and enables scale for mass production ; company used its deoptimisation technology and designed several nCoV vaccine candidate genomes. The next step is to grow and conduct in-vivo tests of vaccine viruses before proceeding to clinical trials |
| CureVac | Undisclosed | mRNA-based vaccine > > induce immune responses in humans with the extremely low dose of only 1 microgram |
| CVM , University of Georgia’s Center for Vaccines and Immunology | LEAPS COVID-19 immunotherapy (peptide) | stimulate immune cells (T cells and B cells) to protect against coronavirus infection |
| DVAX , Clover Biopharmaceuticals | COVID-19 S-Trimer subunit vaccine (from Clover’s Trimer-Tagⓒ technology) + CpG 1018 adjuvant (proprietary toll-like receptor 9 (TLR9) agonist) | COVID-19 S-Trimer subunit |
| DVAX, Sinovac | Dynavax’s CpG 1018 adjuvant + Sinovac’s chemically inactivated coronavirus vaccine candidate | stimulate immune response against SARS-CoV-2 |
| DVAX, Valneva SE | Dynavax’s CpG 1018 adjuvant + Valenva’s VLA2001 (inactivated, whole virus vaccine candidate) | stimulate immune response against SARS-CoV-2 |
| DYAI, The Israel Institute for Biological Research (IIBR) | rVaccine candidate based on C1 gene expression platform | stimulate immune response to SARS-CoV-2 |
| Emergex Vaccines Holding Limited (private) | gold nanoparticle vaccine | stimulate immune response |
| G+FLAS Life Sciences | immunogenic recombinant vaccine candidate | coronavirus spike protein |
| GC Pharma | subunit vaccine candidate | coronavirus epitopes |
| GemVax & KAEL | GV-1001 peptide vaccine (Tertomotide) | activate T-cell mediated immune response against coronavirus |
| Generex Biotechnology (GNBT) , China Technology Exchange , Beijing Zhonghua Investment Fund Management, Biology Institute of Shandong Academy of Sciences , Sinotek-Advocates International Industry Development (Shenzhen) | Ii-Key Peptide-based Covid-19 vaccine | coronavirus antigenic peptides |
| GNBT | | produce an Ii-Key-COVID-19 peptide vaccine that can be tested in human studies within 90 days |
| GOVX, BravoVax | GV-MVA-VLPTM vaccine | vaccine developed using a novel proprietary vaccine platform (GV-MVA-VLPTM). On this platform, MVA, a large virus capable of carrying several vaccine antigens, expresses proteins that assemble into VLP immunogens within (in vivo) the person receiving the vaccine. The production of VLPs in the person being vaccinated mimics virus production in a natural infection, stimulating both the humoral and cellular arms of the immune system to recognize, prevent, and control the target infection |
| Greffex | Non-Replicating Viral Vecto - Ad5 S(GREVAX® platform) I | stimulate immune response to SARS-CoV-2 |
| Hong Kong University of Science and Technology (HKUST) | coronavirus vaccine | explored the B-cell and T-cell spike and nucleocapsid protein epitopes to trigger an immune response to SARS-CoV-2 |
| HOTH , Voltron Therapeutics Inc., HaloVax, Vaccine and Immunotherapy | HALOVAX® = Self-Assembling Vaccine (SAV) for COVID-19 | SARS-CoV-2 |
| Center (VIC) of the Massachusetts General Hospital (MGH) | | |
| HTBX , Waisman Biomanufacturing, University of Miami | gp96-mediated vaccine | Coronavirus antigen |
| ID Pharma | SARS-CoV-2 vaccine (injectable) | stimulate immune response |
| Immune Response BioPharma (private) | IR101C (Whole killed universal CoV vaccine) | stimulate immune response |
| IMV Inc. | DPX-COVID-19 = DPX-based vaccine candidate | targeting novel epitopes from the coronavirus strain |
| INO , Ology Bioservices Inc , Richter-Helm BioLogics GmbH & Co. KG , Wistar Institute, the University of Pennsylvania, the University of Texas, Fudan University and the Laval University, Advaccine , the International Vaccine Institute , Public Health England (PHE) and Commonwealth Scientific and Industrial Research Organization (CSIRO) , VGXI, Inc | INO-4800 = DNA vaccine | optimized DNA plasmids reorganized by a computer sequencing technology and designed to produce a specific immune response in the body |
| IPATF, EVQLV | Undisclosed | Coronavirus antigen |
| IPIX | Brilacidin (defensin-mimetic drug candidate) | activate the primary innate antiviral immune response and mediate other immunomodulatory activities |
| JNJ , CTLT, Emergent BioSolutions , Beth Israel Deaconess Medical Center | Ad26 SARS-CoV-2 | COVID-19 vaccine |
| Medicago, Inc. , Laval University’s Infectious Disease Research Centre | Virus-Like Particle (VLP) vaccine | stimulate immune reaction and antibody formation |
| Medigen Biotechnology Corp., National Institutes of Health | SARS-CoV-2 vaccine | viral spike protein |
| Merck KGaA, MilliporeSigma, Baylor College of Medicine , Texas Children’s Center for Vaccine Development | CoV RBD219-N1 vaccine candidate | accelerate the development of a scalable anc affordable manufacturing process of our Covid-19 vaccine candidates and enable them to advance as quickly as possible to support vaccine production in low- and middle- income countries |
| MIGAL Galilee Research Institute | oral vaccine against COVID-19 | generate antibodies against the virus |
| Moderna | | mRNA vaccine |
| MRK , IAVI | SARS-CoV-2 vaccine using recombinant vesicular stomatitis virus (rVSV) technology | safe, effective vaccine will help prevent future outbreaks of SARS-CoV-2 |
| MRK , Themis | SARS-CoV-2 vaccine using measles vector platform | develop a vaccine candidate targeting SARS-CoV-2 for the prevention of COVID-19 |
| MRNA | mRNA-1273 | Spike protein |
| National Institutes of Health | spike protein vaccine | |
| NK, ImmunityBio | COVID-19 human adenovirus vaccine (hAd5) candidate | stimulate immune response against SARS-CoV-2 |
| NVAX, EBS | NVX-CoV2373 | enhance the immune response and stimulate high levels of neutralizing antibodies |
| OGEN , Aragen Bioscience | TerraCov2 = SARS CoV-2 vaccine candidate | provide specific immunity from the novel coronavirus (“SARS-CoV-2”), the root cause of coronavirus disease 2019 (“COVID-19”). |
| Olymvax Biopharmaceuticals , Army Medical University | genetically engineered recombinant vaccine | immunogenic epitopes ; stimulate immune response |
| ONCS , Providence Cancer Institute, a part of Providence St. Joseph Health | CORVax12 = DNA-encodable, investigational vaccine | prophylactic vaccine to prevent COVID-19 |
| OSE Immunotherapeutics | vaccine using Memopi® epitope (neo-epitope) optimization technology | SARS-CoV-2 epitopes |
| Peter Doherty Institute for Infection and Immunity | active vaccine | stimulate immune response and anti-SARS-CoV-2 anitbody production |
| Peter Doherty Institute for Infection and Immunity | passive vaccine | direct transfer of antibodies to a non-immune individual |
| ReNeuron Group plc | proprietary exosomes as a delivery vehicl for viral vaccines | stimulate immuneresponse against SARS-CoV-2 |
| Shezhen Geno-Immune Medical Institute | Lentiviral Minigene Vaccine (LV-SMENP-DC) | use viral proteins to activate dendritic cells (DCs) and T cells |
| Shenzhen Geno-Immune Medical Institute, Shenzhen Third People’s Hospital, Shenzhen Second People’s Hospital | Covid-19/aAPC (artifical antigen presenting cells) vaccine | stimulate immune response to viral antigens |
| Shizuoka University | Virus-Like Particle (VLP) vaccine | stimulate immune reaction using viral-like-proteins |
| Sichuan Clover Biopharmaceuticals, GSK | S-Trimer | Trimer-Tag technology to enable the rapid production of secreted covalently trimerized, native-like subunit >> produce an S-Trimer subunit vaccine that resembles the native trimeric viral spike via a rapid mammalian cell-culture based expression system >> evoke protective neutalizing antibody responses |
| Sichuan Clover Biopharmaceuticals Inc, GSK | protein based coronavirus vaccine candidate (COVID-19 S-Trimer) | trimeric spike-protein subunit COVID-19 |
| Sinovac Biotech | SARS-CoV units | serconversion |
| SK Bioscience | monoclonal antibody treatment | coronavirus |
| SNGX, University of Hawai’i at Ma̅noa | heat stable subunit vaccines (both monovalent and bivalent vaccine combinations) | introduce viral surface glycoprotein to stimulate an immune response |
| SNY, Biomedical Advanced Researched and Development Authority (BARDA) | Recombinant vaccine of undisclosed SARS-CoV-2 protein(s) expressed in baculovirus system | develop immune response against viral proteins |
| SNY, TBIO | mRNA vaccine for COVID-19 | SARS-CoV-2 proteins |
| SRNE | I-Cell™ COVID-19 Cellular Vaccine | SARS-CoV-2 |
| Stermina Therapeutics | Undisclosed | mRNA-based vaccine >> induce imune responses in humans |
| Symvivo Corporation | bacTRL-Spike = bifidobacteria monovalent SARS-COV-2 DNA vaccine | intiate robust mucosal and systemic humoral and cell-mediated immunity + provide neutralizing nanobodies for immediate immunity |
| Tianjin CanSino Biotechnology , Institute of Biotechnology, Academy of Military Medical Sciences | Ad5-nCoV (recombinant coronavirus vaccine) | express SARS-CoV-2 spike protein to stimulate immune response against viral antigens |
| TNXP , Southern Research | TNX-1800 | express one or more proteins or protein fragments from COVID-19 to elicit immune response |
| University of Queensland | “molecular clamp” vaccine | trigger immune system |
| Vaccitech , University of Oxford , Oxford Biomedica | VTP-500 (ChAdOx1 MERS) | full-length Spike glycoprotein |
| Vaxil BioTherapeutics | Undisclosed | signal peptide technology + VaxHit bioinformatics platform + in silico analyses |
| VBI, National Research Council of Canada (NRC) | pan-coronavirus multivalent eVLP (enveloped virus-like particle) vaccine | stimulate immune response against SARS-CoV-2, SARS-CoV, and MERS-CoV spike proteins |
| VXRT,EBS, KindredBio | five COVID-19 vaccine candidates | generate robust mucosal and systemic immune responses in humans |
| Zhejiang Science and Technology | mRNA vaccines | create immune response to coronavirus |
| Zhejiang Science and Technology | recombinant protein vaccine | stimulate antibodies to protect from coronavirus infection |
| Zy Therapeutics (private) | Z-VacciRNA (mRNA-based vaccine) | stimulate immune response using viral antigens produced from mRNA |

Table 10 below provides exemplary antibodies and immune regulators that can be used in treatment of infection. Such agents can be loaded into LNP-MPVs for oral delivery.

TABLE 10

Exemplary Antibodies and Immune Regulators

| | | |
|---|---|---|
| Company | Drug | Target |
| Adrenomed AG | Adrecizumab | Adrenomedullin (bio-ADM®) |
| AGEN | Repurposing existing drugs: saponins and QS-21 , novel allogeneic cell therapy , proprietary clinical stage checkpoint antibodies | stimulate and potentiate immune system against SARS-CoV-2 |
| AIM , Shenzhen Smoore Technology Limited | Ampligen (rintatolimod) | toll-like receptor (TLR) agonist |
| Anhui Provincial Hospital | Tocilizumab | interleukin-6 antibody |
| Aqualung Therapeutics | ALT-100 (therapeutic monoclonal antibody) | phosphoribosyltransferase (eNAMPT) and its receptor, Toll-like receptor 4 (TLR4) which are important in regulating the upstream inflammatory cascade that contributes to ARDS morbidity and mortality |
| AYTU | Healight Platform Technology (“Healight”) | |
| BCEL, BGNE , IGMS , | novel IgM and IgA antibodies | anti-SARS-CoV-2 antibodies |
| Beroni Group , Tianjin University | precision-driven nanobody treatment for COVID-19 | SARS-CoV-2 epitopes |
| BYSI | BPI-002 | T-cell co-stimulator |
| CDTX | CD377 | Multiple |
| Celltrion | monoclonal antibody (No candidate yet) | coronavirus epitopes |
| DYAI, The Israel Institute for Biological Research (IIBR) | monoclonal antibodies based on C1 gene expression platform | target coronavirus epitopes |
| EBS | polyclonal antibody therapeutics derived from plasma | anti-SARS-CoV-2 antibodies |
| EDSA , Light Chain Bioscience | monoclonal antibodies (block certain signaling proteins known as TLR4 and CXCL10) | block certain signaling proteins known as TLR4 and CXCL10 |
| EIGR | Peginterferon Lambda | SARS-CoV-2 |
| EUSA Pharma | Sylvant (siltuximab) = anti-IL6 | interleukin-6 |
| Eutilex | monoclonal anitbody treatment | coronavirus epitopes |
| Evotec SE , Ology Bioservices, Inc. | antibodies against SARS-CoV-2 | anti-SARS-CoV-2 antibodies |
| Flanders Institute for Biotechnology , Ghent University | single-domain antibody | VIB/Ghent identfied a single-domain antibody with high binding affinity to a unique, conserved conformational epitope present on the receptor-binding domain of SARS-CoV and COVID-19 |
| Formycon AG | antibody-based protein drug technology platform | anti-SARS-CoV-2 antibodies |
| GC Pharma | monoclonal antibody | coronavirus epitopes |
| Harbour BioMed, Mount Sinai Health System | monoclonal antibodies | antibodies have the potential to prevent spread of the virus by blocking infection of cells |
| INMB | dominant-negative TNF inhibitor (DN-TNF) platform | TNF in the cytokine storm of COVID-19 |
| Izana Bioscience , Ergomed plc | IZN-101 (namilumab) = monoclonal antibody therapy targeting granulocyte-macrophage colony stimulating factor (GM-CSF) | monoclonal antibody therapy targeting granulocyte-macrophage colony stimulating factor (GM-CSF) |
| KMDA | Anti-Corona (COVID-19) polyclonal Immunoglobulin | plasma-derived Anti-Corona (COVID-19) IgG product is expected to be produced from plasma derived from donors recovered from the virus, which is anticipated to include antibodies to the novel Corona virus (COVID-19). * Kamada emphasizes that the development plan and manufacturing of the product are highly dependent on the availability of hyper-immune plasma and on the regulatory path to be defined with the health authorities |
| KNSA | mavrilimumab = an investigational fully-human monoclonal antibody that targets granulocyte macrophage colony stimulating factor receptor alpha (GM-CSFRα) | targets granulocyte macrophage colony stimulating factor receptor, alpha (GM-CSFRα) |
| Korea Research Institute of Chemical Technology | neutralizing antibodies | COVID-19’s spike protein |
| LIFE | ATYR1923 (fusion protein comprised of the immunomodulatory domain of histidyl tRNA synthetase fused to the FC region of a human antibody) = a selective modulator of neuropilin-2 | selective modulator of neuropilin-2 that downregulates the innate and adaptive immune response in inflammatory disease states |
| LLY , AbCellera Biologics Inc | Antibody (No candidate yet) | anti-SARS-CoV-2 antibodies |
| LLY , Junshi Biosciences | CA1 and CB6 = human monoclonal antibodies (MAbs) | neutralization activity against SARS-CoV-2 |
| NK, ImmunityBio | N-803 (Natural Killer cells with IL-15 fusion protein) | COVID-19 antigens |
| Novacell Technology | NCP112 peptide | reduce respiratory inflammation |
| OncoImmune, Inc. | CD24Fc (biological immunomodulator) | - binds DAMPS (Danger-Associated Molecular Patterns) = trapping the inflammatory stimuli to prevent their interaction with TLR receptors - binds Siglec G/10 (Siglecs are a distinct class of pattern recognition receptors that down-regulate cellular responses = regulates host response to tissue injuries Siglec G/10-associated SHP1 inhibitory signaling |
| REGN | multi-antibody cocktail for the SARS-CoV-2 virus | SARS-CoV-2 spike protein |
| Roivant Sciences | Gimsilumab (Anti-GM-CSF Monoclonal Antibody) | targeting granulocyte-macrophage colony stimulating factor (GM-CSF) |
| Southeast University, China | Camrelizumab (AiRuiKa) + Thymosin | |
| SRNE | next-generation, gene-encoded monoclonal antibodies | coronavirus epitopes |
| SRNE , Mabpharm Limited | STI-4920 = ACE-MAB proprietary bi-specific fusion protein | targets the spike protein of SARS-CoV-2 with high affinity , arm (TR) is a truncated ACE2 protein that binds to a different epitope of the spike protein |
| SwiftScale , Centivax | neutralizing antibodies | SARS-CoV-2 epitopes |
| Synairgen | SNG001 = inhaled formulation of interferon-beta-1a | activated antiviral pathways in the lung along with improving lung function in patients with a respiratory viral infection |
| Takeda | TAK-888 | Multiple |
| VIR , BIIB | monoclonal antibodies | identified a number of monoclonal antibodies that bind to SARS-CoV-2, which were isolated from individuals who had survived a SARS (Severe Acute Respiratory Syndrome) infection. The company is conducting research to determine if its antibodies, or additional antibodies that it may be able to identify, can be effective as treatment and/or prophylaxis against SARS-CoV-2. |
| VIR , Generation Bio | non-viral gene therapy platform + genetic instructions for human monoclonal antibodies (mAb) | SARS-CoV-2 epitopes |
| VIR , National Institutes of Health | human monoclonal antibodies (mAbs) against coronaviruses | human monoclonal antibodies (mAbs) against coronaviruses, including SARS-CoV-2, the virus that causes the disease COVID-19. The joint project, which will begin this week, will augment ongoing efforts by both parties to identify antibodies that can be used to prevent or treat infection with existing and emerging viruses and help inform the development of vaccines |
| VIR , WuXi Biologics | monoclonal antibodies | antibodies have the potential to prevent spread of the virus by blocking infection of cells |
| VIR , XNCR | XmAb® engineered monoclonal antibodies using Xencor’s Xtend® Fc technology | SARS-CoV-2 epitopes |
| Xinjiang Medical University | NK Cells twice a week (0.1-2*10E7 cells/kg body weight) | Multiple |

Table 11 below provides exemplary plasma immunoglobulins. In some embodiments, these immunoglobulins or nucleic acids expressing such immunoglobulins can be loaded into LNP-MPVs for oral delivery.

TABLE 11

Exemplary Plasma Immunoglobulins

| Company | Drug | Target / Goal |
|---|---|---|
| ADMA | ASCENIV® = immunoglobulin plasma pool compositions | plasma-derived medicine that is comprised of polyclonal antibodies |
| CERS , California Department of Public Health, University of California: Irvine’s Vaccine Development Research Laboratory, Vitalant Research Institute , California National Primate Research Center, Enable Biosciences , Biomedical Advanced Research and Development Authority (BARDA) | INTERCEPT Blood System = optimized convalescent plasma therapy | SARS-CoV-2 inactivation |
| China National Biotec Group | Convalescent plasma | plasma with specific immunoglobulin taken from patients who recovered from the illness » administer plasma to infected pts to help fight the virus |
| CSL Behring , Takeda , Biotest, BPL, LFB, Octapharma | anti-SARS-CoV-2 polyclonal hyperimmune immunoglobulin | SARS-CoV-2 |
| EBS , Biomedical Advanced Research and Development Authority (BARDA) | COVID-Human Immune Globulin (COVID-HIG) = a human plasma-derived therapy COVID-Equine Immune Globulin (COVID-EIG) = a horse plasma-derived therapy | SARS-CoV-2 epitopes |
| Grifols , United States Biomedical Advanced Research Development Authority (BARDA) | plasma from convalescent COVID-19 patients + hyperimmune globulin | anti-SARS-CoV-2 hyperimmune globulin therapy |
| Mount Sinai Health System | plasmapheresis = antibody-rich plasma | SARS-CoV-2 |

Table 12 below provides exemplary nucleic acid-based anti-infection agents. In some embodiments, nucleic acid-based anti-infection agents are loaded into LNP-MPVs for oral delivery.

TABLE 12

Exemplary Nucleic Acid-Based Anti-Infection Agents

| | | |
|---|---|---|
| Company | Drug | Target / Goal |
| Mateon Therapeutics, Inc. | OT-101 = a TGF-Beta antisense drug candidate | The proposed mechanism and actions for OT-101 against COVID-19 include: 1) Inhibition of cellular binding, 2) Inhibition of viral replication and 3) Suppression of viral induced pneumonia. |
| | Fomivirsen | Antisense antiviral drug that was used in the treatment of cytomegalovirus retinitis in immunocompromised patients |

Table 13 below provides exemplary viral ligands, which can be used in blocking virus entry into host cells.. In some embodiments, viral ligands or nucleic acids expressing such ligands are loaded into LNP-MPVs for oral delivery.

TABLE 13

Exemplary Viral Ligands

| Company | Drug | Target / Goal |
|---|---|---|
| NNVC | broad-spectrum virus-binding ligands | Virus proteins that the virus uses to bind to its cognate cellular receptor, namely ACE-2 (angiotensin converting enzyme type 2) |
| SRNE | STI-4398 (COVIDTRAP) protein | S1 domain of the spike protein |
| Tristel plc , Byotrol plc | biocidal products and formulations (disinfectants that are effective against bacteria, viruses and yeasts) | Viruses, bacteria, and yeasts |
| University of Lille (France), Ruhr-University Bochum (Germany) | carbon quantum dots (CQDs) functionalized with boronic acid ligands | Coronavirus S protein |

Additional anti-infection agents are provided in Table 14 below. In some embodiments, anti-infection agents or nucleic acids expressing such agents are loaded into LNP-MPVs for oral delivery.

TABLE 14

Additional Anti-Infection Agents

| Company | Drug | Target / Goal |
|---|---|---|
| ACER | Emetine Hydrochloride | inhibition of SARS-CoV-2 |
| ADPT , MSFT | TCR-Antigen Map (using immunosequencing and machine learning to map T-cell receptor (TCR) sequences to diseases and disease-associated antigens) | aim to develop a blood test for the early and accurate detection of COVID-19 translating the natural diagnostic capability of the immune system into the clinic |
| ALDX | ADX-1612 = inhibitor of chaperone protein HSP90 | inhibitor of chaperone protein HSP90 |
| Algernon Pharmaceuticals Inc. | NP-120 (Ifenprodil) = has a new injectable and long acting oral release formulation | N-methyl-d-aspartate (NDMA) receptor glutamate receptor antagonist |
| AMRN , HLS Therapeutics Inc | Vascepa® = icosapent ethyl or “IPE” | |
| Andera Partners | Inotrem (Sepsis) , Alecra ( antinfectives against resistant bacteria), ReViral (testing for in vitro efficacy against coronavisruses ), AM Pharma ( Phase 3 in renal failure caused by sepsis is including Covid-19 positive patients), Corvidia ( antibody targeting IL6) | SARS-CoV-2 and COVID-19 symptoms |
| ANIX , OntoChem GmbH | enzymes of Covid-19 | enzymes of Covid-19 |
| AntiCancer Inc. | oral recombinant methioninase | oral recombinant methioninase targets and destroys circulating methionine in the body. |
| ANVS | ANVS401 | protect nerve cells against the ill effects of an increase of neurotoxic proteins in the brain ; help with the treatment of neurological diseases associated with COVID-19 and other infections |
| APEIRON Biologics AG | APN01 = recombinant human angiotensin-converting enzyme 2 (rhACE2) | administer decoy protein [recombinant human angiotensin-converting enzyme 2 (rhACE2)] to protect from SARS-CoV-2 infection |
| ARPO | Razuprotafib (previously AKB-9778) | inhibits vascular endothelial protein tyrosine phosphatase (VE-PTP), an important negative regulator of Tie2 |
| ATHX | MultiStemⓇ cell therapy | promote tissue repair and healing in COVID-related ARDS |
| ATOS | AT-H201 | binding to the surface of the coronavirus and inhibiting the ability of the virus to enter a cell |
| AuCuris | testing of already existing drug compounds | SARS-CoV-2 |
| Biomarck Pharmaceuticals | BIO-11006 = Inhalation Solution in ARDS patients | investigational 10-amino acid peptide that inhibits the phosphorylation of the MARCKS protein |
| BLPH | INOpulseⓇ (inhaled nitric oxide (iNO) delivery system) | Nitric oxide therapy at high concentrations targets the vascular smooth muscle cells that surround the small resistance arteries in the lungs |
| BNGO | agents that influence resistance or sensitivity to SARS-CoV2 | identify genomic variants that affect the disease + novel active substances that influence resistance or sensitivity to the SARS-CoV2 virus |
| Brii Biosciences , Columbia University | antiviral drug | SARS-CoV-2 |
| CMRX | dociparstat sodium (DSTAT) | reduce the excessive inflammation, immune cell infiltration and hypercoagulation associated with poor outcomes in patients with severe COVID-19 infection |
| CTSO | CytoSorb® blood purification technology / extracorpeal cytokine adsorber | mitigate cytokine storm |
| CWBR | CB5064 = agonists of the apelin receptor | apelin receptor = broadly expressed and abundant in lung tissue and published preclinical studies have shown that apelin signaling can reduce the severity of acute lung injury, by reducing lung fluid accumulation, hypoxemia, and cytokine secretion, which also occur in COVID-19 associated ARDS and lead to downstream injury to kidney, heart, and other organs |
| DFFN , University of Virginia Health System (UVA) , Integrated Translational Research Institute of Virginia (iTHRIV) | trans sodium crocetinate (TSC) = novel oxygen-enhancing mechanism of action | |
| ECOR | gammaCore SapphireTM | vagus nerve stimulation |
| ENZ | SK1-I | Sphingosine kinase Inhibitor |
| Esanex | SNX-5542 | orally active Hsp90 inhibitor |
| FATE , Masonic Cancer Center, University of Minnesota i | FT516 = off-the-shelf cryopreserved NK cell product | anti-viral activity of natural killer (NK) cells |
| INSM | Brensocatib | inhibitor of dipeptidyl peptidase 1 (DPP1) |
| ITMR | Continuous positive airway pressure (CPAP) | reduce progression to adult respiratory distress syndrome (ARDS) in patients with COVID-19 and mild pneumonia |
| JAGX | crofelemer (MytesiⓇ) | symptomatic relief of diarrhea and other gastrointestinal symptoms in patients with COVID-19 and for patients with COVID-19 who have diarrhea associated with certain antiviral treatments |
| University Gottingen | Camostat mesylate | serine protease TMPRSS2 |
| LGND | luminespib (AUY-922) = heat shock protein 90 (Hsp90) inhibitors | heat shock protein 90 (Hsp90) inhibitor |
| LIVN | Extracorporeal Membrane Oxygenation (ECMO) therapy | cardiopulmonary and advanced circulatory support |
| LJPC | GIAPREZA® (Angiotensin II) | GIAPREZA mimics the body’s endogenous angiotensin II peptide, which is central to the renin-angiotensin-aldosterone system, which in turn regulates blood pressure |
| Massachusetts General Hospital | Inhaled nitric oxide gas | improve oxygenation and pulmonary arterial pressure in patients suffering from COVID-19 |
| MedinCell | long-acting injectable Ivermectin | SARS-CoV-2 |
| Mithra Pharmaceuticals | Estetrol (E4) | n. Estrogen acts on a protein known as Angiotensin Converting Enzyme 2 (ACE2) and enables its expression to be reduced. |
| MNK , Massachusetts General Hospital, Novoteris LLC | INOmax® (nitric oxide) , Thiolanox® = high-dose inhaled nitric oxide therapy | Nitric oxide therapy at high concentrations targets the vascular smooth muscle cells that surround the small resistance arteries in the lungs. NO causes vasodilation |
| MNKD , Immix Biopharma, Inc | inhaled therapeutic | treat acute respiratory distress syndrome, a complication of COVID-19 |
| NeuroRx, Inc. (private) | Aviptadil | vasodilation (Alpha adrenergic receptor antagonists; Vasoactive intestinal peptide receptor agonists) |
| Oryzon Genomics | Vafidemstat (ORY-2001) | LSD1 inhibitor; reduce neuroinflammation |
| PHAS | PB1046 = once-weekly, subcutaneously-injected vasoactive intestinal peptide (VIP) receptor agonist | vasoactive intestinal peptide (VIP) receptor agonist that targets VPAC receptors in the cardiovascular, pulmonary and immune systems. VIP is a neurohormone known to have anti-inflammatory, antifibrotic, inotropic, lusitropic and vasodilatory effects and several cardiopulmonary disorders are associated with alterations in levels of VIP or its receptors, VPAC1 and VPAC2 |
| POAI | AI-generated drug or vaccine | Soluble’s computer system expects to be able to run over 12,000 computer simulations per machine to help generate new diagnostics, vaccines and therapeutics |
| PTSI , BIH Center for Regenerative Therapy (BCRT) , Berlin Center for Advanced Therapies (BeCAT) at Charité University of Medicine Berlin | PLX-PAD (intra-muscular (IM) administration of allogeneic mesenchymal-like cells) | induce the immune system’s natural regulatory T cells and M2 macrophages, and thus may prevent or reverse the dangerous overactivation of the immune system + mitigate the tissue-damaging effects |
| RDHL | RHB-107 (upamostat, WX-671) | serine protease inhibitor active against a number of human trypsins and several other related serine proteases |
| RDHL | Opaganib (ABC294640, Yeliva®) | sphingosine kinase-2 (SK2) selective inhibitor |
| Resverlogix Corp. | apabetalone | apabetalone targets human bromodomain-containing protein (BRD2) a critical interaction partner for SARS-CoV-2 ; also apabetalone inhibits expression of Angiotensin-converting enzyme 2 (ACE2), the receptor utilized by the novel coronavirus particles to gain entry into human cells |
| Secarna | next generation antisense oligonucleotide (ASO) | SARS-CoV-2 genetic information |
| Second Affiliated Hospital of Wenzhou Medical University | Bromhexine Hydrochloride | Decreases mucus viscosity by increasing lysosomal activity. This increased lysosomal activity enhances hydrolysis of acid mucopolysaccharide polymers, which significantly contribute to normal mucus viscosity |
| Second Hospital of Nanjing Medical University | 5u washed microbiota suspension | priming the immune response to viral evasion by introducing bacteria |
| SRNE | COVID-19 therapeutic product candidates | SARS-CoV-2 |
| SRNE , Celularity | CYNK-001 | anti-coronavirus allogeneic NK cell therapy |
| TFFP | TFF technology + dry powder product | dry powder product delivered directly to the lung that is capable of targeting SARS-CoV-2 and potentially similar viruses such as SARS-CoV, MERS-CoV and endemic coronaviruses |
| Tongji Hospital | Sildenafil citrate tablets | vasodilation ; increase oxygen saturation |
| Tuohua Biological Technology , Wuhan Hamilton Biotechnology Co., Ltd (trials run by ZhiYong Peng + Zhongnan Hospital + Puren Hospital Affiliated to Wuhan University of Science and Technology) | Umbilical Cord(UC)-Derived Mesenchymal Stem Cells(MSCs) | Multiple |
| University of Tokyo | Nafamostat mesylate | suppress SARS-CoV-2 S protein-initiated fusion |
| Vcanbio Cell & Gene Engineering Corp Ltd (trials run by Beijing 302 Hospital) | Mesenchymal Stem Cell | Multiple |
| VERO | GENOSYL (nitric oxide) gas | nitric oxide targets the vascular smooth muscle cells that surround the small resistance arteries in the lungs and is used in adult respiratory distress syndrome and persistent pulmonary hypertension of the neonate |
| VERU | VERU-111 = microtubule depolymerization agent | a and b tubulin subunits of microtubules |
| Vicore Pharma | C21 = a first-in-class low molecular weight angiotensin II receptor type 2 (AT2R) agonist | angiotensin II receptor type 2 (AT2R) agonist |
| VNDA | Tradipitant | neurokinin-1 receptor (NK-1R) antagonist |

Other anti-infection biological molecules for use in making the cargo-loaded LNP-MPVs described herein can be found in, e.g., WO2018102397 and references cited therein, the relevant disclosures of each of which are incorporated by reference for the purposes or subject matter referenced herein.

Table 15 below provides exemplary small molecule cargos useful in the treatment of infectious agents and which can be loaded into LNP-MPVs for oral delivery.

TABLE 15

Exemplary small molecules

| | |
|---|---|
| Drug name (Company Name) | Target / MOA |
| AB-01 (Agastiya Biotech LLC) | Angiotensin Converting Enzyme 2 (ACE Related Carboxypeptidase or Metalloprotease MPROT15 or Angiotensin Converting Enzyme Homolog or ACE2 or EC 3.4.17.23) Inhibitor |
| ALDR-491 (Aluda Pharmaceuticals Inc) | Vimentin (Epididymis Luminal Protein 113 or VIM) Inhibitor |
| AP-1189 (SynAct Pharma AB) | Melanocortin Receptor 3 (MC3R) Agonist; Melanocyte Stimulating Hormone Receptor (Melanocortin Receptor 1 or MSHR or MC1R) Agonist |
| AT-001 (Applied Therapeutics Inc) | Aldose Reductase (Aldehyde Reductase or Aldo Keto Reductase Family 1 Member B1 or AKR1B1 or EC 1.1.1.21) Inhibitor |
| ATH-201 (Atossa Therapeutics Inc) | |
| ATR-002 (Atriva Therapeutics GmbH ) | Mitogen Activated Protein Kinase Kinase (MEK or MAP2K or EC 2.7.12.2) Inhibitor |
| berdazimer sodium (Novan Inc) | Berdazimer sodium acts by releasing nitric oxide. Nitric oxide possesses a broad-spectrum of antimicrobial activity by releasing reactive nitrogen species |
| CM-4620 (CalciMedica Inc) | Calcium Release Activated Calcium Channel Protein 1 (Proteii Orai 1 or Transmembrane Protein 142A or ORAI1) Blocker |
| EIDD-2801 (Ridgeback Biotherapeutics LP) | EIDD-2801 exhibits anti-viral property by inhibiting the replication of RNA viruses |
| GBV-006 (Globavir Biosciences Inc) | GBV-006 exhibits anti-viral properties. |
| GD-31 (Guangzhou People’s Hospital Eight) | GD-31 exhibits therapeutic intervention by an undisclosed mechanism of action |
| VERU-111 (Veru Inc) | Alpha Tubulin (TUBA) Inhibitor; Beta Tubulin (TUBB) Inhibitor |
| jaktinib hydrochloride (Suzhou Zelgen Biopharmaceutical Co Ltd) | Tyrosine Protein Kinase JAK2 (Janus Kinase 2 or JAK2 or EC 2.7.10.2) Inhibitor |
| HY-008 (Helperby Therapeutics Group Ltd) | The drug candidate exhibits therapeutic intervention by an undisclosed mechanism of action. |
| ISR-50 (ISR Immune System Regulation Holding AB) | ISR-50 exhibits therapeutic intervention by an undisclosed mechanism of action. |
| Lipocurc (SignPath Pharma Inc) | Histone Deacetylase (HDAC or EC 3.5.1.98) Inhibitor |
| S-1226 (SolAeroMed Inc) | S-1226 treats the diseases like acute lung injury and asthma with the pharmacological actions exerted together by CO2 and perflubron. |
| Neumifil (Pneumagen Ltd) | Neuraminidase A (nanA or EC 3.2.1.18) Inhibitor |
| TD-0903 (Theravance Biopharma Inc) | Non Receptor Tyrosine Protein Kinase TYK2 (TYK2 or EC 2.7.10.2) Inhibitor; Tyrosine Protein Kinase JAK1 (Janus Kinase 1 or JAK1 or EC 2.7.10.2) Inhibitor; Tyrosine Protein Kinase JAK2 (Janus Kinase 2 or JAK2 or EC 2.7.10.2) Inhibitor; Tyrosine Protein Kinase JAK3 (Janus Kinase 3 or Leukocyte Janus Kinase or JAK3 or EC 2.7.10.2) Inhibitor |
| PF-00835231 (Pfizer Inc) | 3C Like Proteinase (3CL-PRO or 3CLp or EC 3.4.22.) Inhibitor |
| PRTX-007 (Primmune Therapeutics Inc) | Toll Like Receptor 7 (TLR7) Agonist |
| RHCDS-13b (German Centre for Infection Research eV) | 3C Like Proteinase (3CL-PRO or 3CLp or EC 3.4.22.) Inhibitor |
| SDC-1801 (Sareum Holdings Plc) | Non Receptor Tyrosine Protein Kinase TYK2 (TYK2 or EC 2.7.10.2) Inhibitor; Tyrosine Protein Kinase JAK1 (Janus Kinase 1 or JAK1 or EC 2.7.10.2) Inhibitor |
| SDC-1802 (Sareum Holdings Plc) | Non Receptor Tyrosine Protein Kinase TYK2 (TYK2 or EC 2.7.10.2) Inhibitor; Tyrosine Protein Kinase JAK1 (Janus Kinase 1 or JAK1 or EC 2.7.10.2) Inhibitor |
| RIG-I Activator (Spring Bank Pharmaceuticals Inc) | Probable ATP Dependent RNA Helicase DDX58 (DEAD Box Protein 58 or RIG I Like Receptor 1 or Retinoic Acid Inducible Gene 1 Protein or DDX58 or EC 3.6.4.13) Activator |
| Small Molecules to Activate STING for Coronavirus Disease 2019 (COVID-19) (Spring Bank Pharmaceuticals Inc) | Stimulator Of Interferon Genes Protein (Endoplasmic Reticulum Interferon Stimulator or Mediator Of IRF3 Activation or Transmembrane Protein 173 or STING or TMEM173) Activator |
| Small Molecules to Inhibit 3CLp for Coronavirus Disease 2019 (COVID-19) (McGill University) | 3C Like Proteinase (3CL-PRO or 3CLp or EC 3.4.22.) Inhibitor |
| Small Molecules to Inhibit 3CL-PRO for Coronavirus Disease 2019 (COVID-19) (Anixa Biosciences Inc | 3C Like Proteinase (3CL-PRO or 3CLp or EC 3.4.22.) Inhibitor |
| Small Molecules to Inhibit Angiotensin Converting Enzyme 2 for Coronavirus Disease 2019 (COVID-19) (Vanda Pharmaceuticals Inc | Angiotensin Converting Enzyme 2 (ACE Related Carboxypeptidase or Metalloprotease MPROT15 or Angiotensin Converting Enzyme Homolog or ACE2 or EC 3.4.17.23) Inhibitor |
| Small Molecules to Inhibit Cathepsin-L for Coronavirus Disease 2019 (COVID-19) (Vanda Pharmaceuticals Inc) | Cathepsin L1 (Cathepsin L or Major Excreted Protein or CTSL or EC 3.4.22.15) Inhibitor |
| Small Molecules to Inhibit Endoribonuclease for Coronavirus Disease 2019 (COVID-19) (Anixa Biosciences Inc) | Serine/Threonine Protein Kinase/Endoribonuclease IRE1 (Endoplasmic Reticulum To Nucleus Signaling 1 or Inositol Requiring Protein 1 or Ire1 Alpha or Endoribonuclease or ERN1 or EC 2.7.11.1 or EC 3.1.26.) Inhibitor |
| Small Molecules to Inhibit GSK3 for Coronavirus Disease 2019 (COVID-19) (Ohio University) | Glycogen Synthase Kinase 3 (GSK3 or EC 2.7.11.26) Inhibitor |
| Small Molecules to Inhibit NSP12 for Coronavirus Disease 2019 (COVID-19) (Arbutus Biopharma Corp) | Small molecules act as RNA-dependent RNA polymerase inhibitor (nsp12). |
| Small Molecules to Inhibit Papain-like Proteinase for Coronavirus Disease 2019 (COVID-19) (University of Alberta) | Small molecules act by inhibiting papain-like proteinase of coronavirus. |
| Small Molecules to Inhibit Protease 3C for Coronavirus Disease 2019 (COVID-19) (Pfizer Inc) | Protease 3C (P3C or EC 3.4.22.28) Inhibitor |
| Small Molecules to Inhibit RNA Directed RNA Polymerase for Coronavirus Disease 2019 (COVID-19) (Aptorum Group Ltd) | RNA Directed RNA Polymerase (EC 2.7.7.48) Inhibitor |
| Small Molecules to Inhibit Transmembrane Protease Serine 2 for Coronavirus Disease 2019 (COVID-19) (Vanda Pharmaceuticals Inc) | Small molecules act as TMPRSS2 inhibitor. |
| zanubrutinib (Brukinsa) (BeiGene Ltd) | Tyrosine Protein Kinase BTK (Bruton Tyrosine Kinase or B Cell Progenitor Kinase or Agammaglobulinemia Tyrosine Kinase or BTK or EC 2.7.10.2) Inhibitor |
| WP-1122 (Moleculin Biotech LLC) | WP-1122 inhibits glycolysis process in tumor cells. |
| niclosamide (Daewoong Co Ltd) | The drug candidate exhibits therapeutic intervention by an undisclosed mechanism of action |
| GP-1681 (CytoAgents Inc) | G Protein Coupled Receptor (Seven Transmembrane Domain Receptor or GPCR) Agonist |
| pacritinib (CTI BioPharma Corp) | Interleukin 1 Receptor Associated Kinase 1 (IRAK1 or EC 2.7.11.1) Inhibitor; Macrophage Colony Stimulating Factor 1 Receptor (CSF 1 Receptor or Proto Oncogene c Fms or CD115 or CSF1R or EC 2.7.10.1) Inhibitor; Receptor Type Tyrosine Protein Kinase FLT3 (FMS Like Tyrosine Kinase 3 or FL Cytokine Receptor or Stem Cell Tyrosine Kinase 1 or Fetal Liver Kinase 2 or CD135 or FLT3 or EC 2.7.10.1) Inhibitor; Tyrosine Protein Kinase JAK2 (Janus Kinase 2 or JAK2 or EC 2.7.10.2) Inhibitor |
| cynarine (SOM Biotech SL) | 3C Like Proteinase (3CL-PRO or 3CLp or EC 3.4.22.) Inhibitor |
| ivermectin LA (MedinCell SA) | Ivermectin acts by inhibiting IMP alpha/beta1 mediated nuclear import. |
| tradipitant (Vanda Pharmaceuticals Inc) | Substance P Receptor (Tachykinin Receptor 1 or NK 1 Receptor or NK1R or TACR1) Antagonist |
| fenretinide (Laurent Pharmaceuticals Inc) | LAU-7b (fenretinide) balances the arachidonic acid and docosahexaenoic acid (AA/DHA) levels. |
| bucillamine (Revive Therapeutics Ltd) | Bucillamine is a cysteine derivative which acts as a thiol donor. |
| azithromycin (Teva Pharmaceutical Industries Ltd) | 23S Ribosomal RNA (23S rRNA) Inhibitor |
| emetine hydrochloride (Acer Therapeutics, Inc.) | The drug candidate exhibits therapeutic intervention by an undisclosed mechanism of action. |
| galidesivir (BioCryst Pharmaceuticals Inc) | RNA Directed RNA Polymerase (EC 2.7.7.48) Inhibitor |
| verdinexor (Karyopharm Therapeutics Inc) | Exportin 1 (Chromosome Region Maintenance 1 Protein Homolog or XPO1) Inhibitor |
| ruxolitinib phosphate (Incyte Corp) | Tyrosine Protein Kinase JAK1 (Janus Kinase 1 or JAK1 or EC 2.7.10.2) Inhibitor; Tyrosine Protein Kinase JAK2 (Janus Kinase 2 or JAK2 or EC 2.7.10.2) Inhibitor |
| artemisinin (Mateon Therapeutics Inc) | |
| naltrexone hydrochloride (Lodonal) (Cytocom Inc) | Opioid Receptor (OPR) Antagonist |
| eravacycline (SOM Biotech SL) | 3C Like Proteinase (3CL-PRO or 3CLp or EC 3.4.22.) Inhibitor |
| nafamostat mesylate (Ensysce Biosciences Inc) | Nafamostat mesylate acts by inhibiting synthetic serine protease. |
| dexamethasone (AVM Biotechnology LLC) | Glucocorticoid Receptor (GR or Nuclear Receptor Subfamily 3 Group C Member 1 or NR3C1) Agonist |
| sonlicromanol (Khondrion BV) | Prostaglandin E Synthase (Microsomal Glutathione S Transferase 1 Like 1 or Microsomal Prostaglandin E Synthase 1 or p53 Induced Gene 12 Protein or PTGES or EC 5.3.99.3) Inhibitor |
| brensocatib (Insmed Inc) | Dipeptidyl Peptidase 1 (Cathepsin C or Cathepsin J or Dipeptidyl Transferase or DPPI or CTSC or EC 3.4.14.1) Inhibitor |
| selinexor (Antengene Corp) | Exportin 1 (Chromosome Region Maintenance 1 Protein Homolog or XPO1) Inhibitor |
| baloxavir marboxil (Shionogi & Co Ltd) | Baloxavir marboxil is a polymerase acidic (PA) endonuclease inhibitor |
| merimepodib (Vicromax) (BioSig Technologies Inc) | Inosine Monophosphate Dehydrogenase (Inosinic Acid Dehydrogenase or IMP Oxidoreductase or IMPDH or EC 1.1.1.205) Inhibitor |
| nitazoxanide CR (Romark Laboratories LC) | Pyruvate Synthase (Pyruvate Ferredoxin Oxidoreductase or PFOR or EC 1.2.7.1) Inhibitor |
| camostat mesylate (University of Tokyo) | Camostat mesylate acts by inhibiting SARS-CoV-2 spike protein initiated membrane fusion. |
| ibudilast (MediciNova Inc) | Cysteinyl Leukotriene Receptor 1 (Cysteinyl Leukotriene D4 Receptor or G Protein Coupled Receptor HG55 or HMTMF81 or CYSLTR1) Antagonist; Phosphodiesterase 3 (PDE3 or EC 3.1.4.17) Inhibitor; Phosphodiesterase 4 (PDE4 or EC 3.1.4.53) Inhibitor |
| bemcentinib (BerGenBio ASA) | Tyrosine Protein Kinase Receptor UFO (AXL Oncogene or AXL or EC 2.7.10.1) Inhibitor |
| XRx-101 (XORTX Therapeutics Inc) | Xanthine Dehydrogenase/Oxidase (Xanthine Dehydrogenase or Xanthine Oxidase or Xanthine Oxidoreductase or XDH or EC 1.17.1.4 or EC 1.17.3.2) Inhibitor |
| ribavirin (Virazole) (Bausch Health Companies Inc) | Inosine Monophosphate Dehydrogenase (Inosinic Acid Dehydrogenase or IMP Oxidoreductase or IMPDH or EC 1.1.1.205) Inhibitor; RNA Polymerase (EC 2.7.7.6) Inhibitor |
| clevudine (Levovir) (Bukwang Pharm Co Ltd) | DNA Polymerase (EC 2.7.7.7) Inhibitor |
| ibrutinib (AbbVie Inc) | Tyrosine Protein Kinase BTK (Bruton Tyrosine Kinase or B Cell Progenitor Kinase or Agammaglobulinemia Tyrosine Kinase or BTK or EC 2.7.10.2) Inhibitor |
| itanapraced (CereSpir Inc) | Amyloid Beta A4 Protein (ABPP or APPI or Alzheimer Disease Amyloid Protein or Amyloid Precursor Protein or Amyloid Beta Precursor Protein or Cerebral Vascular Amyloid Peptide or Prea4 or Protease Nexin II or APP) Inhibitor |
| Vidofludimus calcium (Immunic Inc) | Dihydroorotate Dehydrogenase (Quinone) Mitochondrial (Dihydroorotate Oxidase or DHODH or EC 1.3.5.2) Inhibitor |
| piclidenoson (Can-Fite BioPharma Ltd) | Adenosine Receptor A3 (ADORA3) Agonist |
| cetylpyridinium chloride (ARMS Pharmaceutical LLC) | Microbe-impermeable barrier is applied at the oropharynx, which blocks the ability of virus to reach mucosal tissue |
| reproxalap (Aldeyra Therapeutics Inc) | Reproxalap exhibits therapeutic intervention by inhibiting the formation of toxic metabolites (aldehydes) that accumulate in the back of the eye, |
| transcrocetinate sodium (Diffusion Pharmaceuticals Inc) | Trans sodium crocetinate selectively enhances the diffusion of oxygen into hypoxic tissue without hyper-oxygenating normal tissue. |
| rifaximin (ASKA Pharmaceutical Co Ltd) | DNA Directed RNA Polymerase (POLR2 or EC 2.7.7.6) Inhibitor |
| cannabidiol (Innocan Pharma Corp) | Cannabidiol plays a key step in the anti inflammatory pathway |
| cannabidiol 3 (STERO Biotechs Ltd) | Cannabidiol exhibits therapeutic intervention by an undisclosed mechanism of action |
| baricitinib (Eli Lilly and Co) | Tyrosine Protein Kinase JAK1 (Janus Kinase 1 or JAK1 or EC 2.7.10.2) Inhibitor; Tyrosine Protein Kinase JAK2 (Janus Kinase 2 or JAK2 or EC 2.7.10.2) Inhibitor |
| centhaquine (Pharmazz Inc) | Alpha 2 Adrenergic Receptor (ADRA2) Agonist; Alpha 2B Adrenergic Receptor (Alpha 2 Adrenergic Receptor Subtype C2 or Alpha 2B Adrenoreceptor or ADRA2B) Agonist |
| erdosteine (Recipharm AB) | O2 Radical Scavenger |
| hydroxychloroquine (Sanofi) | Major Histocompatibility Complex (MHC) Inhibitor; Toll Like Receptor 7 (TLR7) Antagonist; Toll Like Receptor 9 (CD289 or TLR9) Antagonist |
| remdesivir (Gilead Sciences Inc) | RNA Polymerase (EC 2.7.7.6) Inhibitor |
| tofacitinib citrate (Xeljanz) (Pfizer Inc) | Tyrosine Protein Kinase JAK3 (Janus Kinase 3 or Leukocyte Janus Kinase or JAK3 or EC 2.7.10.2) Inhibitor |
| pixatimod (Zucero Therapeutics Ltd) | Fibroblast Growth Factor 1 (Acidic Fibroblast Growth Factor or Endothelial Cell Growth Factor or Heparin Binding Growth Factor 1 or FGF1) Inhibitor; Fibroblast Growth Factor 2 (Basic Fibroblast Growth Factor or Heparin Binding Growth Factor 2 or FGF2) Inhibitor; Heparanase (Endo Glucoronidase or Heparanase 1 or HPSE or EC 3.2.1.166) Inhibitor; Toll Like Receptor 9 (CD289 or TLR9) Agonist; Vascular Endothelial Growth Factor (VEGF) Inhibitor |
| opaganib (RedHill Biopharma Ltd) | Sphingosine Kinase 2 (SK2 or SPK 2 or SPHK2 or EC 2.7.1.91) Inhibitor |
| idronoxil (Noxopharm Ltd) | DNA Topoisomerase II (EC 5.99.1.3) Inhibitor; E3 Ubiquitin Protein Ligase XIAP (Baculoviral IAP Repeat Containing Protein 4 or IAP Like Protein or Inhibitor Of Apoptosis Protein 3 or X Linked Inhibitor Of Apoptosis Protein or XIAP or EC 6.3.2.) Inhibitor; Ecto NOX Disulfide Thiol Exchanger 2 (APK1 Antigen or Cytosolic Ovarian Carcinoma Antigen 1 or Tumor Associated Hydroquinone Oxidase or ENOX2) Inhibitor |
| NOX-19 (Noxopharm Ltd) | Interleukin 6 (B Cell Stimulatory Factor 2 or BSF2 or CTL Differentiation Factor or CDF or Hybridoma Growth Factor or Interferon Beta 2 or IFNB2 or IL6) Inhibitor |
| Stannous protoporphyrin (Renibus Therapeutics Inc) | Stannous protoporphyrin (SnPP) acts by protecting the kidney from cellular damage. |
| VP-01 (Vicore Pharma AB) | Type 2 Angiotensin II Receptor (Angiotensin II Type 2 Receptor or AGTR2) Agonist |
| PP-001 (PaniJect) (Panoptes Pharma GesmbH) | Interferon Gamma Receptor 1 (CDw119 or CD119 or IFNGR1) Antagonist; Interferon Gamma Receptor 2 (Interferor Gamma Receptor Accessory Factor 1 or Interferon Gamma Transducer 1 or IFNGR2) Antagonist |
| telmisartan (Laboratorio ELEA SACIF y A) | Type 1 Angiotensin II Receptor (AT1AR or AT1BR or Angiotensin II Type 1 Receptor or AGTR1) Antagonist |
| tranexamic acid (Leading BioSciences Inc) | Tranexamic acid (LB-1148) acts as serine protease inhibitor. |
| zotatifin (eFFECTOR Therapeutics Inc) | Eukaryotic Initiation Factor 4A-I (ATP Dependent RNA Helicase eIF4A1 or DDX2A or EIF4A1 or EC 3.6.4.13) Inhibitor |
| masitinib (AB Science SA) | Fibroblast Growth Factor Receptor 3 (Tyrosine Kinase JTK4 or Hydroxyaryl Protein Kinase or CD333 or FGFR3 or EC 2.7.10.1) Antagonist; Macrophage Colony Stimulating Factor 1 Receptor (CSF 1 Receptor or Proto Oncogene c Fms or CD115 or CSF1R or EC 2.7.10.1) Antagonist; Mast/Stem Cell Growth Factor Receptor Kit (Proto Oncogene c Kit or Tyrosine Protein Kinase Kit or v Kit Hardy Zuckerman 4 Feline Sarcoma Viral Oncogene Homolog or Piebald Trait Protein or p145 c Kit or CD117 or KIT or EC 2.7.10.1) Antagonist; Platelet Derived Growth Factor Receptor Alpha (Alpha Type Platelet Derived Growth Factor Receptor or CD140 Antigen Like Family Member A or Platelet Derived Growth Factor Receptor 2 or CD140a or PDGFRA or EC 2.7.10.1) Antagonist; Platelet Derived Growth Factor Receptor Beta (Beta Type Platelet Derived Growth Factor Receptor or CD 140 Antigen Like Family Member B or Platelet Derived Growth Factor Receptor 1 or CD140b or PDGFRB or EC 2.7.10.1) Antagonist; Tyrosine Protein Kinase Fyn (Proto Oncogene Syn or Proto Oncogene c Fyn or Src Like Kinase or p59 Fyn or FYN or EC 2.7.10.2) Inhibitor; Tyrosine Protein Kinase Lyn (Lck/Yes Related Novel Protein Tyrosine Kinase or V Yes 1 Yamaguchi Sarcoma Viral Related Oncogene Homolog or p53Lyn or p56Lyn or LYN or EC 2.7.10.2) Inhibitor |
| imatinib mesylate (Exvastat Ltd) | Bcr-Abl Tyrosine Kinase (EC 2.7.10.2) Inhibitor |
| ifenprodil (Algernon Pharmaceuticals Inc) | Glutamate Receptor Ionotropic NMDA 1 (Glutamate [NMDA] Receptor Subunit Zeta 1 or N Methyl D Aspartate Receptor Subunit NR1 or GRIN1) Antagonist; Glutamate Receptor Ionotropic NMDA 2B (Glutamate [NMDA] Receptor Subunit Epsilon 2 or N Methyl D Aspartate Receptor Subtype 2B or N Methyl D Aspartate Receptor Subunit 3 or GRIN2B) Antagonist |
| azvudine (Henan Zhenshi Biotechnology Co Ltd) | Reverse Transcriptase (EC 2.7.7.49) Inhibitor |
| brilacidin (Innovation Pharmaceuticals Inc) | Bacterial Cell Membrane Disruptor |
| silmitasertib (Senhwa Biosciences Inc) | Casein Kinase 2 (CSNK2 or EC 2.7.11.1) Inhibitor |
| prexasertib (SOM Biotech SL) | 3C Like Proteinase (3CL-PRO or 3CLp or EC 3.4.22.) Inhibitor |
| UNI-911 (Union Therapeutics AS) | |
| favipiravir (Beijing Sihuan Pharmaceutical Co Ltd) | RNA Directed RNA Polymerase (EC 2.7.7.48) Inhibitor |
| 2X-121 (Oncology Venture U.S. Inc) | Poly [ADP Ribose] Polymerase 1 (ADP Ribosyltransferase Diphtheria Toxin Like 1 or NAD(+) ADP Ribosyltransferase 1 or Poly[ADP Ribose] Synthase 1 or PARP1 or EC 2.4.2.30) Inhibitor; Poly [ADP Ribose] Polymerase 2 (ADP Ribosyltransferase Diphtheria Toxin Like 2 or NAD(+) ADP Ribosyltransferase 2 or Poly[ADP Ribose] Synthase 2 or PARP2 or EC 2.4.2.30) Inhibitor; Tankyrase 1 (ADP Ribosyltransferase Diphtheria Toxin Like 5 or Poly [ADP Ribose] Polymerase 5A or TRF1 Interacting Ankyrin Related ADP Ribose Polymerase or TNKS or EC 2.4.2.30) Inhibitor; Tankyrase 2 (ADP Ribosyltransferase Diphtheria Toxin Like 6 or Poly [ADP Ribose] Polymerase 5B or TRF1 Interacting Ankyrin Related ADP Ribose Polymerase 2 or Tankyrase Like Protein or Tankyrase Related Protein or TNKS2 or EC 2.4.2.30) Inhibitor |
| duvelisib (Copiktra) (Verastem Inc) | Phosphatidylinositol 4,5 Bisphosphate 3 Kinase Catalytic Subunit Delta Isoform (Phosphatidylinositol 4,5 Bisphosphate 3 Kinase 110 kDa Catalytic Subunit Delta or PIK3CD or EC 2.7.1.153) Inhibitor; Phosphatidylinositol 4,5 Bisphosphate 3 Kinase Catalytic Subunit Gamma Isoform (Phosphatidylinositol 4,5 Bisphosphate 3 Kinase 110 kDa Catalytic Subunit Gamma or Phosphoinositide 3 Kinase Catalytic Gamma Polypeptide or Serine/Threonine Protein Kinase PIK3CG or p120 PI3K or PIK3CG or EC 2.7.11.1 or EC 2.7.1.153) Inhibitor |
| ABX-464 (Abivax SA) | ABX-464 exhibits anti-viral activity by inhibiting viral replication by preventing the Rev-mediated export of unspliced HIV-1 transcripts to the cytoplasm and by interacting with the Cap Binding Complex (CBC). |
| PAX-1 (Komipharm International Co Ltd) | Interleukin 1 Beta (IL 1 Beta or Catabolin or IL1B) Inhibitor; Interleukin 18 (Interferon Gamma Inducing Factor or Iboctadekin or Interleukin 1 Gamma or IL18) Inhibitor |
| (ASC-09 + ritonavir) (Ascletis Pharma Inc) | The drug candidate exhibits therapeutic intervention by an undisclosed mechanism of action. |
| acalabrutinib maleate (AstraZeneca Plc) | Tyrosine Protein Kinase BTK (Bruton Tyrosine Kinase or B Cell Progenitor Kinase or Agammaglobulinemia Tyrosine Kinase or BTK or EC 2.7.10.2) Inhibitor |
| FW-1022 (First Wave Bio Inc) | The drug candidate exhibits therapeutic intervention by an undisclosed mechanism of action. |
| elsulfavirine (Elpida) (Viriom Inc) | Reverse Transcriptase (EC 2.7.7.49) Inhibitor |
| PPP-003 (Panag Pharma Inc) | Cannabinoid Receptor 1 (CB1 or CANN6 or CNR1) Agonist; Cannabinoid Receptor 2 (CB2 or CX5 or CNR2) Agonist |
| dantrolene sodium (Eagle Pharmaceuticals Inc) | Ryanodine Receptor 1 (Skeletal Muscle Calcium Release Channel or Skeletal Muscle Ryanodine Receptor or Type 1 Ryanodine Receptor or RYR1) Antagonist |
| (emtricitabine + tenofovir disoproxil fumarate) (Gilead Sciences Inc) | Reverse Transcriptase (EC 2.7.7.49) Inhibitor |
| icosapent ethyl (Alfa) (GLW Pharma) | Eicosapentanoic acid (EPA) replaces arachidonic acid (AA). |
| RT-001 (Retrotope Inc) | RT-001 acts by down regulating the oxidative stress. |
| upamostat (Mesupron) (RedHill Biopharma Ltd) | Urokinase Type Plasminogen Activator (U Plasminogen Activator or PLAU or EC 3.4.21.73) Inhibitor |
| (lopinavir + ritonavir) (Kaletra) (AbbVie Inc) | HIV 1 Retropepsin (HIV Aspartyl Protease or HIV Proteinase or Retroproteinase or Gag Protease or HIV Aspartyl Protease or EC 3.4.23.16) Inhibitor; HIV 2 Retropepsin (HIV 2 Protease or EC 3.4.23.47) Inhibitor |
| BLD-2660 (Blade Therapeutics Inc) | Calpain 1 Catalytic Subunit (Calcium Activated Neutral Proteinase 1 or Calpain Mu Type or Calpain 1 Large Subunit or Cell Proliferation Inducing Gene 30 Protein or Micromolar Calpain or CAPN1 or EC 3.4.22.52) Inhibitor; Calpain 2 Catalytic Subunit (Calcium Activated Neutral Proteinase 2 or Calpain M Type or Calpain Large Polypeptide L2 or Calpain 2 Large Subunit or Millimolar Calpain or CANPL2 or CAPN2 or EC 3.4.22.53) Inhibitor; Calpain 9 (Digestive Tract Specific Calpain or New Calpain 4 or Protein CG36 or CAPN9 or EC 3.4.22.) Inhibitor |
| BHV-3500 (Biohaven Pharmaceutical Holding Company Ltd) | Calcitonin Gene Related Peptide Type 1 Receptor (Calcitonin Receptor Like Receptor or CALCRL) Antagonist |
| apremilast (Otezla) (Amgen Inc) | Phosphodiesterase 4 (PDE4 or EC 3.1.4.53) Inhibitor |
| astodrimer (VivaGel BV) (Starpharma Holdings Ltd) | VivaGel is a topical vaginal microbicide. The mechanism of action for SPL7013 for both pathogens is believed to be the prevention of attachment of the virus to human cells. |
| nitric oxide (Thiolanox) (Novoteris LLC) | Soluble Guanylate Cyclase (sGC or EC 4.6.1.2) Activator |
| ciclesonide (Alvesco) (Sunovion Pharmaceuticals Inc) | Glucocorticoid Receptor (GR or Nuclear Receptor Subfamily 3 Group C Member 1 or NR3C1) Agonist |

Table 16A below provides exemplary antibody cargos useful in the treatment of infectious agents, which can be loaded into MPV-LNPs for oral administration.

TABLE 16A

Exemplary Antibodies

| | |
|---|---|
| Drug name (Company) | Mechanism of Action (MOA) |
| Antibodies 1 for Coronavirus Disease 2019 (COVID-19) (Emergent BioSolutions Inc) | Antibodies act as a replacement therapy for primary humoral immunodeficiency and supply a broad spectrum of opsonic and neutralizing antibodies against infections. |
| SAB-185 (SAB Biotherapeutics Inc) | SAB-185 acts as a replacement therapy for primary humoral immunodeficiency and supply a broad spectrum of opsonic and neutralizing antibodies against infections. |
| Antibodies for Coronavirus Disease 2019 (COVID-19) (Kedrion SpA) | Antibodies act as a replacement therapy for primary humoral immunodeficiency and supply a broad spectrum of opsonic and neutralizing antibodies against infections. |
| Antibodies for Coronavirus Disease 2019 (COVID-19) (ImmuneCyte Inc) | The drug candidates exhibit therapeutic intervention by an undisclosed mechanism of action. |
| Antibodies for Coronavirus Disease 2019 (COVID-19) (BriaCell Therapeutics Corp) | Antibodies elicit therapeutic interventions by binding to spike proteins and neutralize them. This exhibits antiviral activity. |
| XAV-19 (Xenothera SAS) | XAV-19 neutralizes the virus by blocking its entry into the patient’s cells, and it reduces the inflammatory phenomenon. The therapeutic candidate provides an immune response similar to that of the human body to neutralize and prevent the multiplication of the virus, but avoids an immune reaction called cytokinic shock caused by the disease. |
| BT-086 (Biotest AG) | BT-086 contains antibodies against pathogens, lipopolysaccharides and the lipid A. It acts as an efficient adjunctive therapy. The drug candidate causes opsonization of causal pathogens, neutralizing of microbial pathogens and their virulence factors (endo and exo toxins) and targeting the host inflammatory response (anti-inflammatory properties). |
| BT-086 (Biotest AG) | BT-086 contains antibodies against pathogens, lipopolysaccharides and the lipid A. It acts as an efficient adjunctive therapy. The drug candidate causes opsonization of causal pathogens, neutralizing of microbial pathogens and their virulence factors (endo and exo toxins) and targeting the host inflammatory response (anti-inflammatory properties). |
| immune globulin (human) (Octapharma AG) | Human normal immunoglobulin contains mainly immunoglobulin G (IgG) with a broad spectrum of opsonising and neutralizing antibodies against infectious agents. Immunoglobulin G competitively blocks gamma Fc receptors, preventing the binding and ingestion of phagocytes and suppressing platelet depletion. Recombinant human hyaluronidase is a soluble recombinant form of human hyaluronidase that modifies the permeability of connective tissue through the hydrolysis of hyaluronan. Recombinant human hyaluronidase accelerates the break-down of hyaluronan, resulting in a temporary increase in the permeability of the interstitial matrix that facilitates more rapid dispersion and absorption and improved bioavailability of the immunoglobulins. |
| TAK-888 (Takeda Pharmaceutical Co Ltd) | TAK-888 acts as a replacement therapy for primary humoral immunodeficiency and supply a broad spectrum of opsonic and neutralizing antibodies against infections. |

Table 16B below provides exemplary monoclonal antibody cargos useful in the treatment of infectious agents. In some embodiments, the monoclonal antibody cargos are loaded into MPV-LNPs for oral delivery.

TABLE 16B

Exemplary Monoclonal Antibodies

| | |
|---|---|
| Drug name | Mechanism of Action |
| leronlimab;Vyrologix (Cytodyn Inc) | Leronlimab is a humanized monoclonal antibody directed against CCR5, a molecular portal that HIV uses to enter cells. The drug candidate binds to a distinct site on the cellular co-receptor CCR5. HIV first binds to CD4 and then binds to either the CCR5 or CXCR4 co-receptor. This enables conformational changes that permit fusion of the virus with the cell membrane. This binding facilitate entry of the viral genetic information into the cell and subsequent viral replication.The drug candidate binds to CCR5 before viral binding. This blocks HIV from binding to CCR5 and fusing with the cell membrane thereby inhibiting the viral replication process. PRO 140 also interferes with activation of the CCR5 receptor by the mediator CCL5. CCL5 is a chemokine mediator that activates CCR5 receptor. |
| avdoralimab (Innate Pharma SA) | Avdoralimab (IPH-5401) is an anti-C5aR-151 monoclonal antibody. C5aR regulates extravasation of cells into sites of inflammation. C5aR is highly expressed in autoimmune diseases. C5a activates the transcription factor, cAMP response element-binding protein (CREB). CREB activation is a part of the mechanism by which C5a delays neutrophil apoptosis. This also prolongs an inflammatory response. Blocking the C5aR would inhibit the further signaling and help in alleviating the condition.The drug candidate blocks the binding of C5a to C5aR, thereby reducing the accumulation and activation of MDSC and neutrophils in tumors. |
| LY-3127804 (Eli Lilly and Co) | LY-3127804 acts by inhibiting angiopoietin-2 (Ang2). Angiopoietin-2 plays an important role in angiogenesis during the development and growth of cancer. It exerts its effect through a member of the tyrosine kinase receptor family, tie2. The antibody binds to Ang2 with high affinity and neutralize Ang2 induced Tie2 phosphorylation. Thus drug candidate inhibits the up-regulation of ang-2 and tie-2 in the active angiogenic phase which prevents vessel differentiation. |
| NI-0101 (Light Chain Bioscience) | NI-0101 acts as toll-like receptor 4 (TLR4) anatgonist. NI-0101 binds to an epitope on TLR4 which interferes with its dimerization required for intracellular signalling and induction of numerous pro-inflammatory pathways. TLR4 activates NF-kappa B-dependent transcription of inflammatory cytokine genes in the diseased condition. The drug candidate blocks at the level of signal transduction for any ligand source and treats the condition. |
| NI-0801 (Edesa Biotech Inc) | NI-0801 is monoclonal antibody targeting the chemokine IP-10 (CXCL10). NI-0801 blocks the recruitment and activation of pathogenic cells within sites of tissue damage, interrupting the cycle of self perpetuating disease. NI-0801 inhibits the interaction of IP-10 with its cognate receptor and glycosaminoglycans, thereby neutralising IP-10 activity. IP-10 is constitutively expressed at low levels in thymic, splenic and lymph node stroma tissues, but its expression can be induced on a variety of cell types including endothelial cells, keratinocytes, fibroblasts, monocytes and neutrophils. IP-10 not only mediates leukocyte recruitment, but also drives T-cell proliferation upon antigenic stimulation. |
| RG-6149 (F. Hoffmann-La Roche Ltd) | RG-6149 (AMG-282) inhibits binding of IL-33 to the ST2 receptor. IL-33 and ST2 play important roles in allergic bronchial asthma. IL-33 contribute to the induction and maintenance of eosinophilic inflammation in the airways by acting on lung fibroblasts by binding to its ST2 receptor. ST2 is a member of the interleukin-1 receptor family and exists in a transmembrane (ST2L) and a soluble form (sST2) due to alternative splicing. Unregulated IL-33 activity leads to activation of T-helper type 2 cells, mast cells, dendritic cells, eosinophils and basophils, ultimately leading to increased expression of cytokines and chemokines. The drug candidate targets the IL-33 and inhibits its binding to the ST2 receptor thereby alleviates the condition. |
| CERC-002 (Cerecor Inc) | CERC-002 is a LIGHT ligand inhibitor. The drug candidate regulates the DcR3 levels by inhibiting the LIGHT signals via the lymphotoxin beta receptor and the herpesvirus entry mediator (HVEM) and supress the activities of T cells, NK cells, monocytes or dendric cells through several receptors which are involved in the inflammatory responses. |
| BDB-001 (Staidson (Beijing) Biopharmaceuticals Co Ltd) | BDB-001 is a monoclonal antibody that inhibits complement C5. Inhibition of C5 prevents the formation of membrane attack complex. As a result, the drug candidate prevents the inflammation mediated damage of organs. |
| COVI-SHIELD (Sorrento Therapeutics Inc) | The therapeutic candidate deliver the combination of three antibodies against coronavirus spike proteins and acts as a protective shield against SARS-CoV-2 coronavirus infection by blocking and neutralizing the activity of the virus. |
| Monoclonal Antibodies for Coronavirus Disease 2019 (COVID-19) (ImmunoPrecise Antibodies Ltd) | Monoclonal antibodies elicits therapeutic intervention by predicting mutations within the virus genome and to tackle against future variants of the virus. |
| Monoclonal Antibodies for Coronavirus Disease 2019 (COVID-19) (Imperial College London) | The therapeutic candidates specifically recognise and bind to spike protein of virus, blocks the virus entry and instruct the immune system to destroy it. |
| STI-1499 (Sorrento Therapeutics Inc) | STI-1499 acts by inhibiting spike protein. SARS-CoV-2 virus S1 spike protein binds with ACE2 receptors present on respiratory epithelial cells leads to virus entry and starts it’s life cycle. The drug candidates by inhibiting interaction of ACE2 and S1 domain of the spike protein thereby inhibits viral entry. |
| Monoclonal Antibodies for Coronavirus Disease 2019 (COVID-19) (Ossianix Inc) | Monoclonal antibodies neutralize viral particles by binding to spike protein of SARS-CoV-2. Spike protein is involved in the site of attachment of the virus with its cellular receptor ACE-2. The therapeutic antibodies block the adhesion of viral particle at that site that will neutralize its activity. |
| Monoclonal Antibodies for Coronavirus Disease 2019 (COVID-19) (European Molecular Biology Laboratory) | Monoclonal antibodies elicit therapeutic intervention by binding to a surface protein of the novel SARS-CoV-2 coronavirus, thereby prevents virus entry into the cells. |
| Monoclonal Antibodies for Coronavirus Disease 2019 (COVID-19) (Yumab GmbH) | Monoclonal antibodies exhibit therapeutic intervention by binding to a surface protein of SARS-Co-V2 that inhibit the interaction with the host cell receptor, thereby potentially blocking the virus from infection. |
| Monoclonal Antibodies for Coronavirus Disease 2019 (COVID-19) (AbClon Inc) | The drug candidates act by binding to the receptor-binding domain (RBD) of the target cell and neutralize the virus thereby inhibit the interaction with the host cell receptor and blocks the virus from infection. |
| Monoclonal Antibodies for Coronavirus Disease 2019 (COVID-19) (University of Toronto) | Monoclonal Antibodies bind to the S1 domain of the spike protein. SARS-CoV-2 virus S1 spike protein bind with ACE2 receptors present on respiratory epithelial cells leads to virus entry and starts it’s life cycle. The drug candidates by blocking S1 domain of the spike protein, viral particle can’t penetrate and replicate and spread itself. |
| Monoclonal Antibodies for Coronavirus Disease 2019 (COVID-19) (AbCellera Biologics Inc) | The drug candidate acts as a replacement therapy for primary humoral immunodeficiency and supply a broad spectrum of opsonic and neutralizing antibodies against infections. |
| Monoclonal Antibodies for Coronavirus Disease 2019 (COVID-19) (Butantan Institute) | Monoclonal antibodies exhibit therapeutic intervention by binding to a surface protein of SARS-CoV-2 that inhibit the interaction with the host cell receptor, thereby potentially blocks the virus from infection. |
| Monoclonal Antibodies to Inhibit Tetranectin for Sepsis (The Feinstein Institute for Medical Research) | Monoclonal antibodies act by inhibiting tetranectin (TN)). Sepsis is caused by high mobility group box 1 (HMGB1) protein and tetranectin. Tetranectin turns HMGB1 into a killer of the body’s immune cells. This transformation induces cell death (pyroptosis) and immunosuppression, impairing the body’s ability to eradicate microbial infections and leads to death. The therapeutic candidates by preventing TN and HMGB1 interaction reverse sepsis-induced immunosuppression and fatality. |
| pritumumab (Nascent Biotech Inc) | Pritumumab acts by targeting cells expressing vimentin. It is a human IgG1 kappa monoclonal antibody which targets ecto-domain vimentin expressed on the cell surface of a variety of adenocarcinoma. The drug candidate blocks the growth factor receptors and effectively arrest proliferation of tumor cells that lead to direct cell toxicity, known as complement dependent cytotoxicity (CDC). |
| lenzilumab (Humanigen Inc) | Lenzilumab is an engineered human IgG1 (immunoglobulin G1) antibody. It targets granulocyte-macrophage colony stimulating factor (GM-CSF). Abnormal function of this cascade and increased GM-CSF levels associated with a number of inflammatory diseases such as rheumatoid arthritis. Neutralization of GM-CSF by an antibody not only decreases inflammation but also protects the cartilage destruction from inflammatory pathways. Thus, the drug candidate checks the progression of the disease by inhibiting the granulocyte macrophage colony stimulating factor (GM-CSF). |
| otilimab (GlaxoSmithKline Plc) | Otilimab (GSK3196165, MOR103) works against GM-CSF (granulocyte macrophage-colony stimulating factor) and helps in treating inflammatory diseases. GM-CSF is part of the natural immune and inflammatory cascade but has also been identified as ar inflammatory mediator in autoimmune disorders like RA leading to an increased production of pro-inflammatory cytokines, chemokines and proteases and thereby ultimately to articular destruction. The drug candidate blocks disease-relevant processes such as GM-CSF dependent proliferation and signal transduction. |
| emapalumab; Gamifant (Swedish Orphan Biovitrum AB) | Emapalumab (NI-0501) is a monoclonal antibody that binds to soluble and receptor-bound forms of IFN?. Binding to IFN? neutralizes its activity, blocking its intracellular signaling to inhibit macrophage activation and the downstream release of proinflammatory cytokines. Emapalumab reduces the plasma concentrations of CXCL9, a chemokine induced by IFN?. |
| sirukumab; PLIVENSIA (Johnson & Johnson) | Sirukumab targets the cytokine interleukin (IL)-6, a naturally occurring protein that is believed to play a role in autoimmune conditions like RA. IL-6 is secreted by T cells and macrophages to stimulate immune response to trauma, especially burns or other tissue damage leading to inflammation. Increased levels of interleukin-6 (IL-6) contribute to the arthritis symptoms and to the full body complications of rheumatoid arthritis (RA). IL-6 is a chemical messenger in the body which contributes to the painful and persistent joint damage and chronic inflammation. Excess levels of IL-6 are produced in the joints, particularly in the thin tissue layer covering the joint. |
| clazakizumab (Bristol-Myers Squibb Co) | Clazakizumab acts as interleukin-6 (IL-6) inhibitor. IL-6 plays a key role in the inflammatory cascade leading to inflammation, swelling, pain and destruction of large and small joints associated with rheumatoid arthritis. IL-6 acts as a central early mediator in the inflammation cascade and impacts multiple signaling pathways as well as cell types. Targeting the IL-6 pathway affects tumor cells that overproduce IL-6, which goes on to stimulate inflammation-related conditions. The drug candidate inhibits the activity of IL-6 and helps in therapeutic intervention of the disease. |
| canakinumab; Illaris (Novartis AG) | Canakinumab is a fully human monoclonal anti-human interleukin-1beta (IL-1 Beta) antibody of the IgG1/k isotype. Canakinumab binds with high affinity specifically to human IL-1 beta and neutralizes its activity by blocking its interaction with IL-1 beta receptors, and thereby, prevents IL-1 1beta -induced gene activation and the production of inflammatory mediators, such as interleukin-6 or cyclooxygenase-2. |
| namilumab (Izana Bioscience Ltd) | Namilumab is a human IgG1 antibody. It neutralizes the biological activity of human and non-human primate granulocyte macrophage colony-stimulating factor (GM-CSF), a cytokine known to play a significant role in autoimmune and inflammatory diseases. MT203 binds human GM-CSF with low picomolar affinity and potently prevents GM-CSF-induced proliferation as well as production of the chemokine IL-8. |
| siltuximab (EUSA Pharma (UK) Ltd) | Siltuximab (Sylvant, CNTO-328) is an anti-IL-6 chimeric monoclonal antibody. The therapeutic candidate works by inhibiting interleukin-6 to reduce inflammation and tumor growth. IL-6 is an interleukin that acts as both a pro-inflammatory and anti-inflammatory cytokine. IL-6 can be secreted by macrophages in response to specific microbial molecules, referred to as pathogen associated molecular patterns (PAMPs). These PAMPs bind to highly important group of detection molecules of the innate immune system, called pattern recognition receptors (PRRs), including Toll-like receptors (TLRs). Circulating interleukin-6 (IL-6) concentrations correlate with disease activity in severe inflammatory conditions and in some hematological malignancies. Hence inhibition of IL-6 may find helpful in treating cancer. |
| ravulizumab; Ultomiris (Alexion Pharmaceuticals Inc) | Ravulizumab binds to terminal complement protein C5, thereby blocking C5 cleavage into pro-inflammatory components and preventing the complement-mediated destruction of red blood cells (RBCs) as seen in paroxysmal nocturnal hemoglobinuria (PNH). |
| mavrilimumab (MedImmune LLC (Inactive)) | Mavrilimumab acts as granulocyte-macrophage colony stimulating factor receptor alpha antagonist. GM-CSF is a proinflammatory cytokine thought to play a central and non-redundant role in the pathogenesis of rheumatoid arthritis (RA) through the activation, differentiation, and survival of neutrophils and macrophages. Thus, by antagonizing the effects of the granulocyte-macrophage colony stimulating factor receptor, the drug candidate is effective in the treatment of the disease. |
| olokizumab * (R Pharm ) | CDP- 6038 (olokizumab) is an IL-6 inhibitor that selectively blocks the final assembly of the IL-6 receptor signaling complex. IL-6 exerts its biological activities through two molecules: IL-6R (IL-6 receptor) and gp130. When IL-6 binds to mIL-6R (membrane-bounc form of IL-6R), homodimerization of gp130 is induced and a high-affinity functional receptor complex of IL-6, IL-6R and gp130 is formed. The soluble form of IL-6R (sIL-6R) also binds with IL-6, and the IL-6-sIL-6R complex can then form a complex with gp130. The drug candidate by blocking IL-6 signaling prevents the activation of inflammatory reaction and ameliorates the disease condition. |
| eculizumab; Soliris * ( Alexion Pharmaceuticals Inc) ) | Eculizumab is a recombinant humanised monoclonal IgG2/4k antibody that binds to the human C5 complement protein and inhibits the activation of terminal complement. It targets one of the proteins in the complement cascade. It binds to the complement protein C5 specifically and with high affinity, thereby inhibiting its cleavage to C5a and C5b and subsequent generation of the terminal complement complex C5b-9. |
| KSI-501 * ( Kodiak Sciences Inc) ) | KSI-501 acts as VEGF and IL-6 dual inhibitor. Human vascular endothelial growth factor (VEGF) promotes angiogenesis, which involves in the abnormal formation of new blood vessels in the eye, leading to the development of AMD. Up regulated levels of IL-6 contribute to subretinal inflammation. The drug candidate by blocking the activity of IL-6 inhibits the inflammation process and by preventing the interaction of VEGF to its receptors on the surface of endothelial cells, reducing endothelial cell proliferation, vascular leakage and new blood vessel formation. |
| Monoclonal Antibody Conjugate for Coronavirus Disease 2019 (COVID-19) * ( Cidara Therapeutics Inc) | Monoclonal antibody conjugate exhibits therapeutic intervention by dual mechanism where the antiviral agent neutralizes the antigen directly, while the human antibody fragment engages a patient’s immune system to accelerate elimination of the pathogen. |
| Monoclonal Antibodies to Agonize NKp46 for Coronavirus Disease 2019 (COVID-19) (Cytovia Therapeutics Inc) | Bispecific monoclonal antibodies act as NKp46 agonist. NKp46 is an activating receptor expressed on all natural killer cells and plays a major role in elimination of target cell. The drug candidates bind with one arm to NKp46 receptor on NK cells leads to activation of NK cells that will destroy the virus-infected cells while the other arm can block the entry of the virus into epithelial cells and neutralize circulating viruses. |

In some embodiments, antisense oligonucleotide cargos useful in the treatment of infectious agents are loaded into MPV-LNPs for oral delivery. Table 17 below provides non-limiting examples of such antisense oligonucleotide cargos useful in the treatment of infectious agents.

TABLE 17

Exemplary Antisense oligonucleotides

| | |
|---|---|
| Drug Name (Company) | Mechanism of Action |
| trabedersen (Oncotelic Inc) | Trabedersen (OT-101) acts as transforming growth factor beta-2 inhibitor The drug candidate binds to TGF-beta2 mRNA causing inhibition of protein translation and thereby decreasing TGF-beta2 protein levels resulting in the inhibition of tumor cell growth, migration and tumor angiogenesis. TGF-beta2 over-expressed in various malignancies. It plays an important role in promoting the growth, progression and migration of tumor cells as well as in the suppression of the body’s immune system. Trabedersen by blocking the production of TGF-beta2 enabes the body’s immune system to recover its ability to recognize the tumor and long-lasting inhibition of tumor growth and formation of metastasis. |
| Antisense RNAi Oligonucleotide (Alnylam Pharmaceuticals Inc) | Antisense RNAi oligonucleotide acts as TMPRSS2 inhibitor. The spike (S) protein of coronaviruses facilitates viral entry into target cells. The cellular serine protease TMPRSS2 primes SARS-2-S for entry. The drug candidate by inhibiting TMPRSS2 blocks the viral entry into the host cells and elicits therapeutic activity. |
| Antisense RNAi Oligonucleotide (Alnylam Pharmaceuticals Inc) | Antisense RNAi oligonucleotide acts by inhibiting ACE2. ACE2 is a viral entry receptor for SARS-CoV-2. SARS-CoV-2 virus S1 spike protein binds with ACE2 receptors present on respiratory epithelial cells leads to virus entry and starts it’s life cycle. The drug candidate by inhibiting ACE2 blocks the S1 domain of the spike protein and viral entry thereby elicits therapeutic activity. |

In some embodiments, polypeptide cargos useful in the treatment of infectious agents are loaded into MPV-LNPs for oral delivery. Table 18 below provides non-limiting examples of such polypeptide cargos useful in the treatment of infectious agents.

TABLE 18

Exemplary Polypeptides

| | | |
|---|---|---|
| Drug name (Company) | Mechanism of Action | Molecule type |
| CIGB-258 (Center for Genetic Engineering and Biotechnology) | | Peptide |
| Metablok (Arch Biopartners Inc) | Dipeptidase 1 (Dehydropeptidase I or Microsomal Dipeptidase or Renal Dipeptidase or RDP or DPEP1 or EC 3.4.13.19) Inhibitor | Peptide |
| EK-1C4 (FUDAN University) | | Peptide |
| AMY-101 (Amyndas Pharmaceuticals LLC) | Complement C3 (C3 And PZP Like Alpha 2 Macroglobulin Domain Containing Protein 1 or C3) Inhibitor | Peptide |
| ST-266 (Noveome Biotherapeutics Inc) | ST-266 is the derivative of the multipotent progenitor cells. Amnion-derived cellular cytokine solution (ACCS), a secreted product of amnion-derived multipotent progenitor cells (AMP cells) is a cocktail of cytokines existing at physiological levels. It accelerates epithelialization and saturates the wound adequately without excess and improves healing. | Protein |
| INB-03 (Inmune Bio Inc) | Tumor Necrosis Factor Receptor Superfamily Member 1A (Tumor Necrosis Factor Receptor 1 or Tumor Necrosis Factor Receptor Type I or p55 or p60 or CD120a or TNFRSF1A) Antagonist | Protein |
| APN-01 (APEIRON Biologics AG) | Angiotensin Converting Enzyme 2 (ACE Related Carboxypeptidase or Metalloprotease MPROT15 or Angiotensin Converting Enzyme Homolog or ACE2 or EC 3.4.17.23) Replacement | Recombina nt Enzyme |
| ONCase-PEG (AntiCancer Inc) | rMETase (ONCase) induces tumor apoptosis and DNA hypomethylation. Tumor cells require a higher methionine level for growth than normal cells, therefore rMETase act as a tool against the tumor cells. Recombinant L-Methioninase (rMETase) targets methionine dependent tumor cells and inhibits tumor cell growth. Methionine deprivation causes cancer cell to arrest predominantly in the G2 phase of the cell cycle and to eventually undergo apoptosis. | Recombina nt Peptide |
| interferon beta-1a (Cinnagen Co) | Interferon Alpha/Beta Receptor 1 (Cytokine Receptor Class II Member 1 or Cytokine Receptor Family 2 Member 1 or Type I Interferon Receptor 1 or IFNAR1) Agonist | Recombina nt Protein |
| CIGB-128 (Center for Genetic Engineering and Biotechnology) | | Recombina nt Protein |
| CYT-107 (RevImmune SAS) | Interleukin 7 Receptor Subunit Alpha (CDw127 or CD127 or IL7R) Agonist | Recombina nt Protein |
| Recombinant Protein VSF for Viral Infections (ImmuneMed Inc) | Recombinant protein VSF (Virus Suppressing Factor) checks the progression of infection by stimulating the innate immune response of the body against the viral infections. | Recombina nt Protein |
| Recombinant Plasma Gelsolin Replacement for Infectious Disease (BioAegis Therapeutics Inc) | Gelsolin (AGEL or Actin Depolymerizing Factor or Brevin or GSN) Replacement | Recombina nt Protein |
| Recombinant Protein for Coronavirus Disease 2019 (COVID-19) (Massachusetts Institute of Technology) | Tissue Type Plasminogen Activator (t Plasminogen Activator or Alteplase or Reteplase or Plasminogen/Activator Kringle or PLAT or EC 3.4.21.68) I | Recombina nt Protein |
| Recombinant Protein to Agonize GCSFR for Pneumonia (First Affiliated Hospital of Guangzhou Medical College) | Granulocyte Colony Stimulating Factor Receptor (CD114 or GCSFR or CSF3R) Agonist | Recombina nt Protein |
| Recombinant Surfactant Associated Protein D Replacement for SARS and RSV Infections (Trimunocor Ltd) | Pulmonary Surfactant Associated Protein D (Collectin 7 or Lung Surfactant Protein D or COLEC7 or SFTPD) Replacement | Recombina nt Protein |
| Recombinant Protein to Target Sialic Acid Receptor for Influenza A Infections (University of St. Andrews) | Recombinant protein acts by masking the sialic acid receptors present in the respiratory tract. Sialic acid receptors are used by several pathogens for entry and infection. The drug candidates by blocking sialic acid receptors and by modulating the immune system put the cells into an anti-viral state. | Recombina nt Protein |
| NT-201 (NellOne Therapeutics Inc) | Recombinant protein acts by stimulating innate regenerative pathways. NELL1 is a signaling protein that triggers pathways for tissue growth and maturation in a variety of tissues including heart muscle, skeletal muscle and blood vessels. The drug candidate triggers the production of several extracellular matrix (ECM) proteins and promotes the tissue formation thereby elicits therapeutic activity. | Recombina nt Protein |
| interferon alfa-2b (Center for Genetic Engineering and Biotechnology) | Interferon Alpha/Beta Receptor 1 (Cytokine Receptor Class II Member 1 or Cytokine Receptor Family 2 Member 1 or Type I Interferon Receptor 1 or IFNAR1) Agonist; Interferon Alpha/Beta Receptor 2 (Interferon Alpha Binding Protein or Type I Interferon Receptor 2 or IFNAR2) Agonist | Recombina nt Protein |
| anakinra (Swedish Orphan Biovitrum AB) | Interleukin 1 Receptor Type 1 (CD121 Antigen Like Family Member A or Interleukin 1 Receptor Alpha or p80 or CD121a or IL1R1) Antagonist | Recombina nt Protein |
| aldesleukin (Iltoo Pharma) | Interleukin 2 Receptor (IL2R) Agonist | Recombina nt Protein |
| conestat alfa; Ruconest (Pharming Group NV) | Complement C1s Subcomponent (C1 Esterase or Complement Component 1 Subcomponent S or C1S or EC 3.4.21.42) Inhibitor | Recombina nt Protein |
| interferon beta-1a; Rebif (Merck KGaA) | Interferon Alpha/Beta Receptor 1 (Cytokine Receptor Class II Member 1 or Cytokine Receptor Family 2 Member 1 or Type I Interferon Receptor 1 or IFNAR1) Agonist; Interferon Alpha/Beta Receptor 2 (Interferon Alpha Binding Protein or Type I Interferon Receptor 2 or IFNAR2) Agonist | Recombina nt Protein |
| peginterferon lambda-1a (Eiger BioPharmaceuticals Inc) | Interferon Lambda Receptor 1 (Cytokine Receptor Class II Member 12 or Interleukin 28 Receptor Subunit Alpha or IL28RA or IFNLR1) Agonist | Recombina nt Protein |
| ILCT-1001 (ILC Therapeutics Ltd) | Interleukin 17A (Cytotoxic T Lymphocyte Associated Antigen 8 or CTLA8 or IL17A) Inhibitor; Interleukin 17F (Cytokine ML 1 or IL17F) Inhibitor; Interleukin 22 Receptor (IL22R) Antagonist; Interleukin 23 Receptor (IL23R) Antagonist; Tumor Necrosis Factor Receptor Superfamily Member 1B (Tumor Necrosis Factor Receptor 2 or p75 or p80 TNF Alpha Receptor or CD120b or TNFRSF1B) Antagonist | Recombina nt Protein; Synthetic Peptide |
| zilucoplan (Ra Pharmaceuticals Inc) | Complement C5 (C3 And PZP Like Alpha 2 Macroglobulin Domain Containing Protein 4 or C5) Inhibitor | Synthetic Peptide |
| BIO-11006 (BioMarck Pharmaceuticals Ltd) | Myristoylated Alanine Rich C Kinase Substrate (Protein Kinase C Substrate 80 kDa Protein Light Chain or PKCSL or MARCKS) Inhibitor | Synthetic Peptide |
| APL-9 (Apellis Pharmaceuticals Inc) i | Complement C3 (C3 And PZP Like Alpha 2 Macroglobulin Domain Containing Protein 1 or C3) Inhibitor | Synthetic Peptide |
| NCP-112 (NovaCell Technology Inc) | N-Formyl Peptide Receptor 2 (FMLP Related Receptor I or Formyl Peptide Receptor Like 1 or HM63 or Lipoxin A4 Receptor or RFP or FPR2) Agonist | Synthetic Peptide |
| IK-15800 (InterK Peptide Therapeutics Ltd) | IK15800 specifically inhibits the activity of kinases known to facilitate SARS-CoV viral entry and replication within cells. | Synthetic Peptide |
| Synthetic Peptide for Coronavirus Disease 2019 (COVID-19) (Massachusetts Institute of Technology) | Synthetic peptide disrupts the binding of SARS-CoV-2-receptor binding domain with ACE2. SARS-CoV-2 initiates entry into human cells by binding to angiotensin-converting enzyme 2 (ACE2) via the receptor-binding domain (RBD) of its spike protein (S). The drug candidate by blocking the interaction of SARS-CoV-2-RBD with ACE2, inhibits viral entry thereby elicits therapeutic activity. | Synthetic Peptide |
| Synthetic Peptides for Coronavirus Disease 2019 (COVID-19) (Immupharma Plc) | Synthetic peptides act by blocking the fusion of COVID-19 and other viruses to the target cell. | Synthetic Peptide |
| plitidepsin; Aplidin (Pharma Mar SA) | Aplidin (plitidepsin) is an anti-cancer agent of marine origin exhibits a broad spectrum of anti-tumor activities. Plitidepsin inhibits elongation factor 1 alpha 2 (eEF1A2), thereby interfering with protein synthesis, and induces G1 arrest and G2 blockade, thereby inhibiting tumor cell growth. | Synthetic Peptide |
| Ampion; Ampion (Ampio Pharmaceuticals Inc) | Aryl Hydrocarbon Receptor (Class E Basic Helix Loop Helix Protein 76 or bHLHe76 or AHR) Agonist | Synthetic Peptide |
| lonodelestat (Santhera Pharmaceuticals Holding AG) | Neutrophil Elastase (Bone Marrow Serine Protease or Elastase 2 or Medullasin or PMN Elastase or Human Leukocyte Elastase or ELANE or EC 3.4.21.37) Inhibitor | Synthetic Peptide |
| solnatide (Apeptico Forschung und Entwicklung GmbH) | Epithelial Sodium Channel (ENaC or SCNN1) Activator | Synthetic Peptide |
| lucinactant; Surfaxin (Windtree Therapeutics Inc) | Lucinactant is KL4 surfactant. KL4 is precision-engineered to mimic the essential properties of human SP-B, the most important surfactant protein for lowering surface tension and promoting oxygen exchange and demonstrates significant resistance to inactivation. Low birth weight infants with severe RDS, a common, less invasive ventilatory support treatment alternative to intubation and mechanical ventilation is nasal continuous positive airway pressure (nCPAP). Endogenous pulmonary surfactant lowers surface tension at the air-liquid interface of the alveolar surfaces during respiration -and stabilizes the alveoli against collapse at resting transpulmonary pressures. A deficiency of pulmonary surfactant in premature infants results in RDS. Surfaxin compensates for the deficiency of surfactant and restores surface activity to the lungs of these infants. | Synthetic Peptide |
| FX-06 (F4 Pharma GmbH) | Cadherin 5 (7B4 Antigen or Vascular Endothelial Cadherin or CD144 or CDH5) Inhibitor | Synthetic Peptide |
| aviptadil (Relief Therapeutics Holding AG) | Vasoactive Intestinal Polypeptide Receptor 1 (Pituitary Adenylate Cyclase Activating Polypeptide Type II Receptor or VPAC1 or VIPR1) Agonist; Vasoactive Intestinal Polypeptide Receptor 2 (Helodermin Preferring VIP Receptor or Pituitary Adenylate Cyclase Activating Polypeptide Type III Receptor or VPAC2 or VIPR2) Agonist | Synthetic Peptide |
| metenkefalin (Cytocom Inc) | Opioid Receptor (OPR) Antagonist | Synthetic Peptide |
| AT-527 (Atea Pharmaceuticals Inc) | NS5B (Nonstructural Protein 5B Polymerase or EC 2.7.7.48) Inhibitor | |
| CD-24Fc (OncoImmune Inc) | CD-24Fc fusion protein targets on danger (or damage)-associated molecular patterns (DAMPs) associated pathway. The danger (or damage)-associated molecular patterns (DAMPs), a group of intracellular component released from necrotic cells, such as HMGB1 and HSP70, may be involved in the pathogenesis of RA. CD24-Siglec 10 mediate a negatively regulatory pathway that selective regulates host response to DAMP 1. Since the CD24 binds to multiple DAMPs, including HMGB1, HSP70, HSP90 and nucleolin, it is conceivable that CD24 fusion proteins can be explored for therapy of rheumatoid arthritis. | Fusion Protein |
| RPH-104 (R Pharm) | Interleukin 1 Beta (IL 1 Beta or Catabolin or IL1B) Inhibitor | Fusion Protein |
| asunercept; Apocept (Apogenix AG) | Tumor Necrosis Factor Ligand Superfamily Member 6 (Apoptosis Antigen Ligand or Fas Antigen Ligand or CD95L or CD178 or FASLG) Inhibitor | Fusion Protein |
| efineptakin alfa (NeoImmuneTech Inc) | Interleukin 7 Receptor Subunit Alpha (CDw127 or CD127 or IL7R) Agonist | Fusion Protein |
| AVA-Trap (Avalon GloboCare Corp) | Ava-Trap acts by inhibiting excessive cytokines related to coronavirus infection. Cytokine release syndrome (CRS) trigger severe lung damage and potentially lead to acute respiratory distress syndrome (ARDs). Fc-fusion cytokine receptors by binding to their respective ligand dampens excessive cytokine levels and elicits therapeutic intervention. | Fusion Protein |
| AKS-446 (Akston Biosciences Corp) | AKS-446 exhibits therapeutic intervention by an undisclosed mechanism of action. | Fusion Protein |
| CMAB-020 (Mabpharm Ltd) | CMAB-020 elicits therapeutic intervention through inhibition of viral entry by binding of one arm to the spike protein of SARS-CoV-2. The other arm (TR) is a truncated ACE2 protein that binds to a different epitope o the spike protein. The ACE-MAB fusion protein also blocks the receptor binding domain (RBD) with CD147 to mitigate lung inflammation and cytokine storm and elicits activity. | Fusion Protein |
| Fusion Protein for Coronavirus Disease 2019 (COVID-19) (GT Biopharma Inc) | Fusion protein exhibits activity by directing the NK cells towards infected cells to kill them. | Fusion Protein |
| SIF-019 (Systimmune Inc) | IgG Receptor FcRn Large Subunit p51 (IgG Fc Fragment Receptor Transporter Alpha Chain or Neonatal Fc Receptor or FCGRT) Antagonist | Fusion Protein |
| STI-4398 (Sorrento Therapeutics Inc) | STI-4398 protein binds to the S1 domain of the spike protein. SARS-CoV-2 virus S1 spike protein bind with ACE2 receptors present on respiratory epithelial cells leads to virus entry and starts it’s life cycle. The drug candidate by blocking S1 domain of the spike protein, viral particle can’t penetrate and replicate and spread itself. | Fusion Protein |
| DAS-181 (Ansun Biopharma Inc) | DAS-181 elicits anti-viral activity. It inhibits the binding of virus to sialic acid present on the host cells. Attachment to sialic acid is mediated by receptor binding proteins that are constituents of viral envelopes. The drug candidate by attaching to the epithelial cells cleaves the virus receptor, sialic acid, from cell surface glycans thereby inhibits virus binding. | Fusion Protein |
| nogapendekin alfa (ImmunityBio Inc) | Cytokine Receptor Common Subunit Gamma (Interleukin 2 Receptor Subunit Gamma or GammaC or p64 or CD132 or IL2RG) Agonist; Interleukin 2 Receptor Subunit Beta (High Affinity IL 2 Receptor Subunit Beta or p70-75 or CD122 or IL2RB) Agonist i | Fusion Protein |

Table 19 below provides non-limiting examples of anti-infectious cargos useful in the treatment of infectious agents, which can be loaded into MPV-LNPs for oral delivery.

TABLE 19

Other exemplary anti-infection cargos

| | |
|---|---|
| Drug name (Company) | Molecule Type ; Mechanism of Action |
| Gene Therapies to Inhibit RNA-Dependent RNA Polymerase for Coronavirus Disease 2019 (COVID-19) (Nanjing KAEDI Biotech Inc) | Gene Therapy; Gene therapies act as RNA-dependent RNA polymerase inhibitor (nsp12). Viral RNA-dependent RNA polymerase (nsp12) binds to ACE2 receptor and catalyzes the formation of phosphodiester bonds between ribonucleotides in a RNA template-dependent fashion which is responsible for transcription and replication of RNA virus genomes. The drug candidates by blocking the activity of RNA polymerase, inhibits the viral replication. This leads to the prevention of viral propagation which treats the disease. |
| Gene Therapy for Coronavirus Disease 2019 (COVID-19) (SmartPharm Therapeutics Inc) | Gene Therapy; Gene therapy exhibits therapeutic intervention by an undisclosed mechanism of action. |
| MV-130 (Bactek) (Inmunotek SL) | Inactivated Vaccine; MV-130 (Bactek) works by provoking the body’s immune response to these bacteria, without actually causing the diseases. When the body is exposed to foreign organisms, the immune system produces antibodies against them. Antibodies help the body to recognize and kill the foreign organisms. |
| IMT-504 (Mid-Atlantic BioTherapeutics, Inc.) | Oligonucleotide; IMT-504 stimulates the immune system to protect against various diseases. The drug candidate mimics different natural alarm signals for activation of the immune system. This oligonucleotide led to secretion of interferon gamma (IFN-gamma), tumour necrosis factor-alpha (TNF-alpha) and granulocyte/monocyte colony-stimulating factor (GM-CSF) and stimulates the immune system. It induces apoptosis in cancer cells and prevents tumour growth. IMT504 also acts by supercharging the body’s own immune response to defend against and defeat infections. |
| NI-007 (Neurimmune Holding AG) | Oligonucleotide; NI007 exhibits therapeutic intervention by an undisclosed mechanism of action. |
| rintatolimod (Ampligen) (AIM ImmunoTech Inc) | Oligonucleotide; Ampligen activates TLR3 receptors on dendritic cells which upregulates costimulatory molecules and immune enhancing cytokines, thereby generating effective immunity. Toll-like receptors such as TLR-3 serve as pattern recognition receptors in the early detection of pathogens and the establishment of early defense mechanisms (innate immunity). When the dormant alarm signals of TLRs are activated (as by exposure to a pathogen or a stimulant agent such as Ampligen), TLRs in effect cause an overreaction, driving the body to proliferate broad-spectrum defenses against many types of pathogens. Ampligen may also increase natural killer (NK) cell activity. Ampligen inhibit viral attachment to cellular receptors and/or inhibit intracellular maturation of the virus. |
| PUL-042 (Pulmotect Inc) | Oligonucleotide; Synthetic Peptide; PUL-042 is a toll-like receptor (TLR 2,6,9) agonist. TLRs are pathogen pattern recognition receptors that recognize bacterial and viral products, and provide receptor-mediated immune activation. The drug candidates mimic bacterial DNA or viral RNA and modulate immune responses through TLR agonism. Targeted stimulation boosts up immunity quickly, providing effective defense against deadly pathogens and protecting immunocompromised patients from chemotherapy treatment. |
| iota-carrageenan (Marinomed Biotech AG) | Polymer; Iota-Carrageenan inhibits the replication of virus by preventing the binding or the entry of virions into the cells and results in reduction in the viral growth. |
| KB-109 (Kaleido Biosciences Inc) | Polysaccharide; KB-109 exhibits therapeutic intervention by an undisclosed mechanism of action. |
| tafoxiparin sodium (Dilafor AB) | Polysaccharide; DF-01 (tafoxiparin) has a dual mechanism of action. The drug candidate acts by promoting the myometrial contractility of the uterus and promotes the softening of the cervix. The drug candidate belongs to the class of heparin called low anticoagulant heparin having reduced risk for bleeding complications. The drug candidate also enhances the softening of the cervix. DF-01 (tafoxiparin) has a synergistic effect with oxytocin and prostaglandin E2 which facilitate the process of parturition. |
| Polysaccharide to Inhibit Galectin for Coronavirus Disease (COVID-19) (Bioxytran Inc) | Polysaccharide; The drug candidate acts as a galectin inhibitor. Galectins fold on the spike protein that is universal to the coronavirus genus. The drug candidate by inhibiting the activity of galectin block viral entry and reduce the T-cell activity. |
| dociparstat sodium (Chimerix Inc) | Polysaccharide; CX-01 acts as neutrophil elastases inhibitor and CXCL12 inhibitor. CXCL12 chemokine binds to CXCR4 and regulates the trafficking and adhesion of normal and malignant cells. Aberrant activation promotes the metastasis of cancer cells. The therapeutic candidate binds to CXCL12 and disrupts attachment of CXCL12 to stromal cell and drives malignant cells out of protective environments and thereby alleviates the condition. CX-01 is a potential potent inhibitor of the interaction between HMGB1 and toll-like receptor 4 (TLR4). HMGB1 has been implicated in autophagy, a mechanism by which cells withstand the effects of chemotherapy, and severe traumatic brain injury, where HMGB1 release has been correlated with worsening neurologic outcomes. CX-01 binds to platelet factor 4 and neutralizes its activity. Neutrophil elastase is the strongest serine proteinase secreted from activated neutrophils and causes degranulation of eosinophils. Excess secretion of these proteins is associated with lung damage. The therapeutic candidate by inhibiting neutrophil elastases down-regulates the neutrophil level and checks the disease progression. |
| TRC-19 (VSY Biotechnology BV) | TRC-19 exhibits therapeutic intervention by an undisclosed mechanism of action. |
| COVENT-1 (Enterin Inc) | COV-ENT-1 interferes with virus entry, protein synthesis, replication and egress, essentially rendering the cell resistant to viruses. COV-ENT-1 also stimulates regenerative activity, and it could potentially promote tissue repair in lungs damaged by the SARS-CoV-2 virus. |
| CVL-218 (Convalife) | CVL-218 acts as selective PARP-½ inhibitor. PARP is a protein involved in a number of cellular processes involving mainly DNA repair and programmed cell death. PARP plays a key role in DNA repair by detecting and initiating repair if a DNA strand breaks. PARP inhibition by the CVL-218 enhance the cytotoxicity of DNA-damaging agents and reverse tumor cell chemoresistance and radioresistance. |
| P-2PAR (Pattern Pharma Inc) | P-2PAR elicits therapeutic intervention by activating monocytes or macrophages, dendritic cells (DCs) and NK cells via toll like receptor 4 (TLR4). Toll-like receptor is an innate immune receptor which control innate immune responses and further instruct development of antigen-specific acquired immunity. |
| Drug for Coronavirus Disease 2019 (COVID-19) (HDL Therapeutics Inc) | The drug candidate acts by removing the lipid layer of SARS-CoV-2 virus. Removal of the lipids leads to permit enhanced exposure of viral proteins to the immune system, leading to neutralizing antibody (nAb) production, and potentially resulting in stronger and broader cell-mediated immune responses (CMI). The cell-mediated immune response will engage T-cells to attack and destroy viruses and infected cells reducing viral load of the infected patients and elicits therapeutic intervention. |
| Drugs for Coronavirus Disease 2019 (COVID-19) (Q BioMed Inc) | The drug candidate exhibits therapeutic intervention by targeting Ang-Tie2 pathway. By modulating Ang-Tie2 pathway, the drug candidate reduces the severity of viral and bacterial infections and promotes positive host-directed therapeutic (HDT) responses |
| ENU-200 (Ennaid Therapeutics LLC) | ENU-200 blocks the S glycoprotein and main protease (Mpro) of CoV. S glycoprotein is responsible for host cell attachment and mediating host cell membrane and viral membrane fusion during infection. Mpro is a key enzyme for CoV replication and is also responsible for transforming the polypeptide into functional proteins. By blocking S glycoprotein and Mpro elicits antiviral activity against coronavirus 2 (SARS-CoV-2). |
| Drug for Coronavirus Disease 2019 (COVID-19) (St George Street Capital Ltd) | Drug candidate elicits therapeutic intervention by using the body’s own mechanism of controlling excess inflammation by activating T regulatory cells. These T regulatory cells migrate to sites of inflammation such as the lungs, effectively dampening down the excess inflammation to reduce organ damage. |

### E. Vaccine

In some embodiments, the cargo loaded into the MPVs, e.g., WPVs, comprises a vaccine, for example, an anti-pathogenic vaccine, e.g., an anti-viral vaccine. Vaccines prevent many millions of illnesses and save numerous lives every year. Millions of lives are saved every year through vaccines for diseases caused by viruses and bacteria, including Haemophilus influenzae type b (Hib), Hepatitis B, Human papillomavirus (HPV), Measles, Meningitis A, Mumps, Pneumococcal diseases, Polio, Rotaviral infections, Rubella, and Yellow fever. (WHO Global immunization coverage 2018).

Conventional protein-based vaccine approaches, such as live attenuated and inactivated pathogens and subunit vaccines, provide durable protection against a variety of dangerous diseases. Live attenuated vaccines, which use a weakened form of the pathogen that causes a disease, have been among the most powerful for the purpose of disease control and even eradication, owing to the strong antibody and cellular responses elicited by them (Potlin, Clin Vaccine Immunol. 2009 Dec; 16(12): 1709-1719). Several methods are employed, all of which involve passing virus in suitable matter can create a new version of the virus that can still be recognized by animal immune systems but cannot replicate well in a vaccinated host. One common method for creating live vaccine strains is by passing viruses in cell cultures or embryos, such as chicken embryos. For example, when a viral strain is passed in chick embryos, this results in a strain with improved replicative capability in check cells, but decreased replicative capability in the target host cells. A second method of making live vaccines is through generation of random mutations in the viral genome and subsequent selection of a non-virulent mutant incapable of causing clinical disease.

Inactivated vaccines, while safer due to the lack of replicative ability, often provide a shorter protection times than live attenuated vaccine and generally also elicit weaker immune responses. Subunit vaccines have become very attractive due to their improved safety profiles as compared to traditional vaccines based on live attenuated or whole inactivated pathogens. Subunit, recombinant, polysaccharide, and conjugate vaccines are biosynthetic vaccines containing recombinant proteins isolated from the pathogen, in which only a subset of antigens are used to stimulate the immune response. Such subunit vaccine can be produced as recombinant vaccines, i.e., in a cell culture transfected with a vector that expresses the vaccine protein. Many genes encoding surface antigens from viral, bacterial, and protozoal pathogens have been successfully cloned into bacterial, yeast, insect, or mammalian expression systems, and the expressed antigens are used for vaccine development. Conjugate vaccines, e.g., as used in children against pneumococcal bacterial infections, utilize antigenic polypeptides from the surface of bacteria, which are chemically linked to a carrier protein and are used to generate an improved immune response. The carrier protein functions as an adjuvant and promotes the immune response, while the antigenic polypeptides produce immunity against future infections.

Toxoid vaccines are made from attenuated pathogenic toxins which are capable of generating an immune response. Diphtheria and tetanus vaccines are prepared from inactivated bacterial toxins, which mount an immune response and produce antibodies that can also neutralize the actual toxins.

Nucleic acid (DNA and RNA) vaccines have characteristics that meet these challenges of constantly evolving infection, including ease of production, scalability, consistency between lots, storage, and safety. DNA vaccines consist of expression systems, e.g., nonviral or viral systems encoding antigenic proteins which are injected directly into the muscle of the recipient. For time and cost saving manufacture, the nucleic acid is synthesized and cloned into the plasmid vector, which is highly stable, such as abacterial plasmid. In some cases, DNA-vaccine constructs comprise a strong eukaryotic promoter and/or other eukaryotic enhancers of expression known in the art, e.g., one or more introns. Alternatively, the DNA-based vaccine construct may comprise a viral vector derived from a suitable virus, e.g., vaccinia, adenovirus, AAV, lentivirus, CMV, Sendai virus or others known in the art.

Vaccine cocktails, which contain the DNA vaccine and are administered in combination with plasmids encoding adjuvanting immunomodulatory proteins, such as cytokines, chemokines, or co-stimulatory molecules, have been used to increase immunogenicity. Cells transfected by molecular adjuvant plasmids secrete the adjuvant into the surrounding region, stimulating both local antigen presenting cells (APC) and cells in the draining lymph node, and resulting in steady low level, production of cytokines that promote the immune response without causing a systemic cytokine storm (Sushak et al., Advancements in DNA vaccine vectors, nonmechanical delivery methods, and molecular adjuvants to increase immunogenicity Hum Vaccin Immunother. 2017 Dec; 13(12): 2837-2848).

mRNA vaccines represent a promising alternative to conventional vaccine approaches because of their high potency, capacity for rapid development and potential for low-cost manufacture and safe administration through high yield in vitro transcription(reviewed in Pardi et al. mRNA vaccines — a new era in vaccinology Nature Reviews Drug Discovery volume 17, pages261-279(2018)).

In some embodiments, the biologic agent comprises an mRNA-based vaccine. In some embodiments, the biologic agent comprises an antiviral mRNA-based vaccine, e.g., directed against a corona virus, e.g., a SARS-CoV-2 vaccine. Non-limiting examples include BNT162 , BTN1626b2, developed by Biontech, and mRNA vaccines developed by CureVac and Moderna.

In some embodiments, the mRNA based vaccine is a conventional mRNA-based vaccine. In some embodiments, the mRNA-based vaccine encodes one or more antigen(s) of interest, e.g., a viral antigen(s). In some embodiments the mRNA-based vaccine comprises one or more of the following features: 5′ untranslated regions (UTR), 3′ UTR, polyA tail, one or more modified bases.

In some embodiments, the mRNA based vaccine is a self-amplifying RNA, encoding one or more antigen(s) of interest. In some embodiments, the mRNA based vaccine encodes an antigen and a viral replication machinery.

In some embodiments, the cargo loaded into the MPVs, e.g., WPVs, comprises an anti-viral vaccine, e.g., an anti-viral vaccine directed against a corona virus, e.g., a SARS-CoV-2 vaccine. In some embodiments, the anti-viral vaccine, e.g., directed against a corona virus, e.g., a SARS-CoV-2, comprises an antiviral protein-based vaccine, e.g., an inactivated vaccine or a live attenuated vaccine. In some embodiments, the anti-viral vaccine, e.g., directed against a corona virus, e.g., a SARS-CoV-2, comprises a subunit vaccine or a fusion protein. In some embodiments, the anti-viral vaccine, e.g., directed against a corona virus, e.g., SARS-CoV-2, is a DNA-based vaccine or an RNA-based vaccine (e.g., an mRNA vaccine) as described above and elsewhere herein. In specific examples, the cargo may be Quattro Grass (Pollinex), which can be used for alleviating pollen allergy. In other examples, the cargo may be a cancer vaccine, for example, Advesin®, or BriaVax®.

Other exemplary vaccines include Afluria (Pro) (influenza virus vaccine), Fluarix Quadrivalent (influenza virus vaccine, inactivated), Flublok Quadrivalent (influenza virus vaccine, inactivated), Fluvirin (Pro) (influenza virus vaccine, inactivated), Engerix-B (hepatitis b adult vaccine), Zostavax (Pro) (zoster vaccine live), Gardasil 9 (Pro) (human papillomavirus vaccine), Flucelvax Quadrivalent (influenza virus vaccine, inactivated), Shingrix (Pro) (zoster vaccine, inactivated), FluMist (Pro), (influenza virus vaccine, live, trivalent), Fluzone (Pro) (influenza virus vaccine, inactivated), Fluzone High-Dose (influenza virus vaccine, inactivated), Fluad (influenza virus vaccine, inactivated), Flublok (Pro) (influenza virus vaccine, inactivated), FluMist Quadrivalent, (influenza virus vaccine, live, trivalent), Stamaril (yellow fever vaccine), ACAM2000 (smallpox vaccine), Afluria Quadrivalent (influenza virus vaccine, inactivated), Agriflu (influenza virus vaccine, inactivated), Attenuvax (measles virus vaccine), Cervarix (Pro) (human papillomavirus vaccine), Dryvax (smallpox vaccine), Engerix-B Pediatric (hepatitis b pediatric vaccine), Fluarix (Pro) (influenza virus vaccine, inactivated), Flucelvax (influenza virus vaccine, inactivated), FluLaval (Pro) (influenza virus vaccine, inactivated), FluLaval Quadrivalent (influenza virus vaccine, inactivated), Fluogen (influenza virus vaccine, inactivated), Flushield (influenza virus vaccine, inactivated), Fluzone Intradermal Quadrivalent, (influenza virus vaccine, inactivated), Fluzone Quadrivalent (influenza virus vaccine, inactivated), Havrix (Pro) (hepatitis A adult vaccine), Havrix Pediatric (hepatitis a pediatric vaccine), Imovax Rabies, (rabies vaccine, human diploid cell), Ipol (Pro) (poliovirus vaccine, inactivated), Ixiaro (Pro), (japanese enceph vacc sa14-14-2, inactivated), Meruvax II (rubella virus vaccine), Mumpsvax (mumps virus vaccine), RabAvert (Pro) (rabies vaccine, purified chick embryo cell), Recombivax HB Adult (hepatitis b adult vaccine), Recombivax HB Dialysis Formulation (hepatitis b adult vaccine), Recombivax HB Pediatric / Adolescent (hepatitis b pediatric vaccine), Rotarix (Pro) (rotavirus vaccine), RotaTeq (Pro) (rotavirus vaccine), Vaqta (Pro) (hepatitis a adult vaccine), Vaqta Pediatric (hepatitis a pediatric vaccine), Varivax (Pro) (varicella virus vaccine), and YF-Vax (Pro) (yellow fever vaccine).

### F. Particles

In some embodiments, the LNP-MPV cargo, may be a particle, for example, a nucleic acid-carrying particle. The particle as disclosed herein can be any type of particles suitable for nucleic acid attachment in any suitable manner, e.g., displayed on the surface, integrated completely or partially into the particles, or encapsulated by the particle. For example, the particle may be a gold nanoparticle and one or more nucleic acid molecules can be linked on the surface of the gold nanoparticle. The attached nucleic acid attached (e.g., encapsulated) may be an RNA molecule or a DNA molecule. The nucleic acid molecule may comprise one or more nucleotide sequences coding for one or more agents of interest, for example, therapeutic nucleic acids or therapeutic proteins. See, e.g., disclosures herein. As used herein, the term “coding for” or “encoding” means that a nucleic acid comprises a nucleotide sequence that can produce an agent of interest, either directly or by transcription and optionally translation. Where applicable, the nucleic acid molecule may comprise additional components for, e.g., packaging the nucleic acid into the particle, for expressing the encoded agents of interest (e.g., promoter sequences, ribosomal entry sites, etc.) and/or for regulating such expression (e.g., enhancer, silencer, polyA tail, miRNA binding site, etc.)

In some embodiments, the nucleic acid-attaching particles can be viral particles of any suitable type. A viral particle refers to a virus like particle comprising viral capsid proteins encapsulating genetic materials (e.g., RNA or DNA). In some instances, the viral particle is an enveloped viral particle, which comprises an outer wrapping or envelope surrounding the capsid proteins. This outer wrapping or envelop may come from the budding process when newly formed virus particles are released from host cells. As such, the outer wrapping or envelope can be made, at least in part, of the cell’s plasma membrane comprising lipids and proteins existing in the cell membrane of the host cells. In other instances, the viral particle is not enveloped.

The genetic materials, e.g., an RNA molecule or a DNA molecule, may comprise viral elements necessary for packaging the viral particle and nucleotide sequences coding for an agent of interest (e.g., a nucleic acid molecule or a protein molecule or nucleic acid sequences constituting a therapeutic nucleic acid. See, e.g., disclosures herein. Preferably the viral particles disclosed herein are defective in replication. The nucleic acid molecule encapsulated in the viral particle may be of any suitable type (for example, RNA or DNA, single-strand or double strand) depending upon the type of the viral particle. The nucleic acid molecule may comprise one or more nucleotide sequences coding for one or more agents of interest, for example, therapeutic nucleic acids or therapeutic proteins. See, e.g., disclosures herein. The nucleotide sequence coding for the agents of interest may be monocistronic, i.e., each nucleic acid molecule comprises one such nucleotide sequence coding for one agent of interest. Alternatively, the nucleotide sequences coding for the agents may be polycistronic, i.e., each nucleic acid molecule comprises at least two such nucleotide sequences coding for two agents of interest. Cleavage sits (e.g., proteolytic cleavage sites) or coding sequence thereof and/or internal ribosomal entry sites may be placed between two of such nucleotide sequences so that the individual agent of interest can be released in host cells after infection by the viral particle.

In some embodiments, the viral particle is derived from an RNA virus, for example, norovirus, enterovirus, or corona virus. RNA virus is a type of virus that has RNA as its genetic material. Such a viral particle comprises an RNA molecule encapsulated by the suitable capsid proteins. In addition to the nucleotide sequences coding for the agents of interest described above, the RNA molecule may comprise one or more viral elements such as 5′ untranslated region (5′-UTR), 3′UTR, packaging site, or a combination thereof. In some instances, the RNA molecule may further comprise elements that regulate expression efficiency of the encoded agents of interest, for example, internal ribosomal entry sites, 3′ polyA tail, miRNA binding sites, etc.

In some examples, the RNA viral particle is derived from a positive single-strand RNA (ssRNA) virus, which comprises capsid proteins encapsulating a single-strand positive chain of an RNA molecule. Examples include, but are not limited to, norovirus, enterovirus, or corona virus. In other instances, the RNA viral particle is derived from a retrovirus, for example, a gamma retrovirus or a lentivirus. Such a positive RNA molecule may be a messenger RNA (mRNA) like molecule that encodes one or more proteins of interest. The RNA molecule may comprise a naturally-occurring mRNA molecule. Alternatively, it may comprise a modified mRNA molecule. In some examples, the mRNA may be modified by introduction of non-naturally occurring nucleosides and/or nucleotides. Any modified nucleosides and/or nucleotides may be used for making the modified mRNA as disclosed herein. Examples include those described in US20160256573, the relevant disclosures are incorporated by reference for the purpose and subject matter referenced herein. In other examples, the mRNA molecule may be modified to have reduced uracil content. See, e.g., US20160237134, the relevant disclosures are incorporated by reference for the purpose and subject matter referenced herein. In some instances, the coding sequences may be codon optimized, which may be performed based on the codon usage in the subject (e.g., human subject) to which the cargo is to be delivered. Alternatively, at least a portion of the RNA molecule may comprise precursors of an RNA molecule of interest (e.g., a therapeutic RNA), for example, a miRNA, a shRNA, or a lncRNA. The RNA molecule may produce such therapeutic RNAs or precursors thereof directly, or via transcription.

In some examples, the RNA viral particle can be derived from a negative strand ssRNA virus, which comprises capsid proteins encapsulating a single-strand negative chain of an RNA molecule. Examples include, but are not limited to, bunya virus and mononega virus. In some instances, such an RNA viral particle may comprise a viral RNA-dependent RNA polymerase, which may convert the negative RNA chain into the positive strand. The positive RNA strand can then produce any of the agents of interest as disclosed herein. In some instances, the negative RNA strand may comprise viral elements and/or regulatory elements (e.g., those described herein) such that it can produce a positive RNA strand comprising coding sequences for the agents of interest, 5′UTR, 3′UTR, and/or polyA tail, etc., to produce the agents of interest, e.g., therapeutic nucleic acid agents, or therapeutic protein agents. In some specific examples, the positive strand converted from the RNA molecule in the viral particle can express proteins in host cells. In other specific examples, the RNA positive strand may produce therapeutic RNAs (e.g., a miRNA, a shRNA, or a lncRNA) or precursors thereof directly, or via transcription.

In some examples, the RNA viral particle can be derived from a double-strand RNA (dsRNA) virus, for example, reovirus (e.g., rotavirus). Upon infection, the genomic dsRNA can be transcribed into mRNAs that serve for both translation and replication purposes. In some instances, such an RNA viral particle may comprise a viral RNA-dependent RNA polymerase, which may produce mRNAs from the dsRNA molecule in the viral particles upon infection. The mRNA can then produce any of the agents of interest as disclosed herein. In some instances, the dsRNA molecule may comprise viral elements and/or regulatory elements (e.g., those described herein) such that it can produce mRNAs comprising coding sequences for the agents of interest, 5′UTR, 3′UTR, and/or poly A tail, etc., to produce the agents of interest, e.g., therapeutic nucleic acid agents, or therapeutic protein agents. In some specific examples, the mRNAs converted from the dsRNA molecule in the viral particle can express proteins in host cells. In other specific examples, the mRNAs may produce therapeutic RNAs (e.g., a miRNA, a shRNA, or a lncRNA) or precursors thereof.

In some embodiments, the viral particle is derived from a DNA virus. A DNA virus is a type of virus that contains DNA as its genetic material and replicates the genetic material using DNA-dependent DNA polymerase. Such a viral particle may comprise suitable capsid proteins encapsulating a DNA molecule, which may comprise one or more nucleotide sequences encoding agents of interest. Such coding sequences may be in operable linkage to a suitable promoter, which drives expression of the encoded agents of interest, e.g., therapeutic nucleic acids such as miRNA, shRNA, or lncRNA or precursors thereof, or therapeutic proteins. The nucleotide sequence coding for the agents of interest may be monocistronic, i.e., each nucleic acid molecule comprises one such nucleotide sequence coding for one agent of interest. Alternatively, the nucleotide sequences coding for the agents may be polycistronic, i.e., each nucleic acid molecule comprises at least two such nucleotide sequences coding for two agents of interest. Cleavage sits (e.g., proteolytic cleavage sites) or coding sequence thereof and/or internal ribosomal entry sites may be placed between two of such nucleotide sequences so that the individual agent of interest can be released in host cells after infection by the viral particle.

In some examples, the viral particle is derived from a single strand DNA (ssDNA) virus, which is a type of virus using a single strand DNA as its genetic materials. Examples include virus of the parvoviridae family. Such a viral particle may comprise suitable capsid proteins encapsulating a single strand DNA molecule. The single DNA molecule may comprise one or more nucleotide sequences coding for one or more agents of interest (e.g., therapeutic nucleic acids or therapeutic proteins), which may be in operable linkage to a suitable promoter. The coding sequences may contain one or more introns. Alternatively, the coding sequences may contain no intron sequences. In addition, the single strand DNA molecule may comprise 5′ UTR, 3′ UTR, transcription regulatory elements such as enhancers, silencers, nucleotide sequence coding for a poly A tail, miRNA binding site, etc.

In specific examples, the viral particle is an adeno-associated viral (AAV) particle. AAVs are a family of small, non-enveloped, replication-defective, ssDNA virus. AAVs can infect both dividing and resting human cells and cause mild immune responses, making it a suitable vesicle for delivering transgenes in gene therapy. The single strand DNA in an AAV particle may comprise a 5′ invert terminal repeat (5′ ITR), a 3′ ITR (e.g., a wild-type ITR or a modified version such as an internal ITR lacking a terminal resolution site), one or more nucleotide sequences encoding one or agents of interest (e.g., therapeutic nucleic acids or therapeutic proteins), which may be in operable linkage to a suitable promoter, and optionally one or more transcriptional regulatory elements, such as enhancers, poly A segment, miRNA binding site, etc.

In some instances, the nucleic acid in an AAV viral particle may be a self-complementary viral vector engineered from a naturally-occurring AAV genome. A self-complementary vector contains an intra-molecule double-stranded DNA template. Upon infection, the two complementary halves of the self-complementary vector can associate to form one self-annealing, partially double stranded DNA (dsDNA) unit that is ready for immediate replication and transcription, thereby leading to fast expression of the encoded agents of interest in most of the infected cells.

In some instances, the nucleic acid in an AAV viral particle may comprise a modified 5′ ITR and/or 3′ ITR relative to a wild-type counterpart so as to expand transgene packaging capacity. In other instances, the nucleic acid in an AAV viral particle may comprise a naturally-occurring 5′ ITR and/or 3′ ITR of AAV virus.

Any of the AAV viral particles may be of a suitable serotype. Capsid proteins from different serotypes would exhibit differential binding to specific cell surface receptors. Thus, use of a specific serotype of an AAV viral particle could achieve infection of a specific type of cells.

Table 34 below provides a list of optimal serotypes of AAV virus for infecting specific tissues.

TABLE 34

AAV Serotypes and Corresponding Tissues for Infection

| Tissue | Optimal Serotype |
|---|---|
| CNS | AAV1, AAV2, AAV4, AAV5, |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal Pigment | AAV1, AAV2, AAV4, AAV5, |
| Skeletal Muscle | AAV1, AAV6, AAV7, AAV8, |

In some examples, the AAV particle disclosed herein is a serotype capable of infecting enterocytes (also known as intestinal absorptive cells). For example, the AAV particle may infect specifically enterocytes of the villus in the small intestine, e.g., in the duodenum. Alternatively, the AAV particle may infect specifically enterocytes of the crypt in the small intestine. “Infect specifically” means that the AAV particle can infect the target cell or tissue in a much greater level compared to other types of cells or tissue (e.g., at least 1 fold greater, at least 2 fold greater, at least 5 folder greater, or at least 10 fold greater). One or more AAV serotypes optimal for infecting a specific type of cells or tissues may be determined via routine practice of the screening methods disclosed herein.

The AAV particles used in the present disclosure may be of a naturally-occurring serotype. Alternatively, it may be an engineered serotype (e.g., having an engineered capsid protein content, for example, a mixture of capsid proteins from different serotype AAV virus). In specific examples, the AAV particles used in the present disclosures can be of AAV1, AAV2, AAV2.5, AAV2.5T, or AAV8. AAV2.5 is a chimera of the VP1 region of AAV2 and the VP2 and VP3 regions of AAV5. AAV2.5T additionally bears a single A581T amino acid substitution (AAV5 VP1 numbering).

In some examples, the viral particle disclosed herein is derived from a double-strand DNA (dsDNA) virus, which are the type of virus using double-strand DNA as their genetic materials. Examples include, but are not limited to, adenovirus, polyoma virus (e.g., SV40), and herpes virus. In some instances, a dsDNA may replicate through a single-stranded RNA intermediate, for example, hepatitis B virus. A viral particle derived from a dsDNA virus may comprise capsid proteins encapsulating a double strand DNA molecule. Like other nucleic acids disclosed herein, the dsDNA molecule may comprise one or more nucleotide sequences coding for one or more agents of interest (e.g., therapeutic nucleic acids or therapeutic proteins), which may be in operable linkage to a suitable promoter. The coding sequences may contain one or more introns. Alternatively, the coding sequences may contain no intron sequences. In addition, the single strand DNA molecule may comprise 5′ UTR, 3′ UTR, transcription regulatory elements such as enhancers, silencers, nucleotide sequence coding for a poly A tail, miRNA binding site, etc.

In any of the nucleic acid encapsulated in a viral particle that carries a promoter for driving expression of the agent of interest, the promoter may be tissue-specific. Tissue-specific promoters for controlling gene expression in specific types of tissues and/or cells are known in the art and can be used in the present disclosure. In some examples, the tissue-specific promoter is for driving gene expression only in enterocytes or other intestinal cells. Examples include, but are not limited to, intestinal alkaline phosphatase promoter, an epithelial-specific ETS-1 promoter, or a Kruppel-like factor 4 (KLF4) promoter.

### G. Exemplary Therapeutic Cargos

In some embodiments, the cargo loaded into the LNP-MPVs, disclosed here comprise one or more therapeutic agents (e.g., nucleic acid-based or protein-based) targeting an infection, for example, infection caused by a virus such as a coronavirus (e.g., SARS such as SARS-CoV-2). Examples include a vaccine or a neutralizing antibody, a small molecule, a polypeptide therapeutic agent, or a nucleic acid (e.g., those designed for producing such protein-based therapeutic agents).

In some embodiments, the cargo loaded into the LNP-MPVs disclosed here comprise one or more therapeutic agents (e.g., nucleic acid-based or protein-based) targeting a metabolic disease. Examples include a therapeutic antibody, a small molecule, a polypeptide anti-pathogenic agent, or a nucleic acid (e.g., those designed for producing such protein-based therapeutic agents). Exemplary agents for treating a metabolic disease are provided in Tables 1-6 herein.

In some embodiments, the cargo loaded into the LNP-MPVs disclosed here comprise one or more therapeutic agents (e.g., nucleic acid-based or protein-based) targeting a cancer. Examples include a therapeutic antibody, a chemotherapeutic agent, a polypeptide anti-cancer agent, or a nucleic acid (e.g., those designed for producing such protein-based therapeutic agents). Exemplary anti-cancer agents are provided in Tables 1-6 herein.

In some embodiments, the cargo loaded into the LNP-MPVs disclosed here comprise one or more therapeutic agents (e.g., nucleic acid-based or protein-based) targeting an immune disorder. Examples include a therapeutic antibody, a small molecule immunomodulator, a polypeptide (e.g., an autoantigen), or a nucleic acid (e.g., those designed for producing such protein-based therapeutic agents). Exemplary anti-immune disorder agents are provided in Tables 1-6 herein.

In some embodiments, the cargo loaded into the LNP-MPVs disclosed here comprise one or more anti-infection agents (e.g., nucleic acid-based or protein-based) targeting an infection as described herein. Examples of types of anti-infection cargors include a therapeutic antibody, a small molecule immunomodulator, a polypeptide (e.g., an autoantigen), a nucleic acid (e.g., those designed for producing such protein-based therapeutic agents) or a small molecule. Exemplary anti-infection agents are provided in Tables 7-19 herein.

In some embodiments, the LNP-MPV cargo loaded comprises one or more checkpoint blockade inhibitors, for example, an anti-CTLA4 antibody, or an anti-PD1/PD-L1 antibody. Exemplary anti-CTLA-4 antibodies include Yervoy (ipilimumab), tremelimumab, AK-104 (PD-1 bispecific), KN-046 (PD-1 bispecific), BMS-986218, CG-0070, MK-1308, zalifrelimab, ATOR-1015, MEDI-5752, MGD-019, XmAb-20717, and XmAb-22841. Exemplary anti-PD-1/PD-L1 antibodies include Pembrolizumab, Nivolumab, Atezolizumab, Avelumab, Durvalumab, Sintilimab, Toripalimab, Tislelizumab, Camrelizumab, Cemiplimab, HLX10, Balstilimab, Dostarlimab, Budigalimab, Penpulimab, MEDI0680/AMP-514, Pidilizumab, Cosibelimab, CS1001, and FAZ053. See also Table 3 for additional examples.

Other biological molecules for use in making the cargo-loaded LNP-MPVs described herein can be found in, e.g., WO2018102397 and references cited therein, the relevant disclosures of each of which are incorporated by reference for the purposes or subject matter referenced herein.

### III. Methods for Producing LNP-MPVs

In some aspects the present disclosure provides novel vesicles, comprising one or more components originating from an MPV and one or more components from an LNP, and having the cargo encapsulated therein, referred to as “fused vesicles”, fused LNP-MPVs″, “LNP-MPVs” or “duosomes.” One non-limiting example of such an LNP-MPV is a liposome-WPV, which comprises one or more components from a liposome and one or more components from a WPV, and having a cargo encapsulated therein. In some embodiments, the present disclosure provides a method of producing such vesicles. In some aspects, the disclosure provides method for loading any of the MPVs, e.g., WPVs, disclosed herein with any of the cargos also disclosed herein. In some embodiments, methods disclosed herein comprise contacting a lipid nanoparticle (LNP) carrying a cargo with a composition comprising MPVs, e.g., WPVs, under suitable conditions that allow for fusion of the LNP with the MPV, e.g., WPV, thereby producing a vesicle of the disclosure, i.e., comprising one or more components originating from the MPV and one or more components from the LNP, and having the cargo encapsulated therein. In some embodiments, methods disclosed herein comprise contacting a liposome carrying a cargo with a composition comprising WPVs, under suitable conditions that allow for fusion of the liposome with the WPV, thereby producing a vesicle comprising one or more components originating from the liposome and one or more components from the WPV, and having the cargo encapsulated therein. In some embodiments, the method further comprises collecting the LNP-MPV, e.g., liposome WPV. Alternatively or in addition, the method further comprises modifying the LNP-MPV, e.g., liposome-WPV, for example, by attaching a targeting moiety for delivering cargos to specific cells, e.g., cells of the intestinal lining of the gut. An LNP-MPV which is further modified by attaching a a targeting moiety, are referred to herein as “surface programmed LNP-MPV.” A surface programmed liposome-WPV is one example of a surface programmed LNP-MPV. Surface programmed LNP-MPVs, e.g., surface programmed liposome-WPVs, can be used for cargo delivery via oral administration. In some embodiments, glycan residues are removed from the surface of the surface programmed LNP-MPVs or the surface programmed liposome-WPVs. Surface programmed LNP-MPV are one type of vesicle that can be produced using Orasome Technology.

In any of the method embodiments described herein, the MPVs, e.g., WPVs, or compositions of MPVs, e.g., WPVs, used in the methods can comprise a relative abundance of casein less than about 40%, about 35%, about 30%, about 25%, about 20%, about 15%, about 10%, about 5% or less. In some of these embodiments, the MPVs, e.g., WPVs, or compositions of MPVs, e.g., WPVs, are substantially free of casein. In some of these embodiments, the MPVs, e.g., WPVs, or compositions of MPVs, e.g., WPVs, comprise lactoglobulin at a relative abundance of no greater than 25% (e.g., less than about 25%, about 20%, about 15%, about 10%, about 5% or less). In some embodiments, the MPVs, e.g., WPVs, or the composition comprising such may be substantially free of lactoglobulins. In some embodiments, the MPVs, e.g., WPVs, are not modified from their naturally occurring state. In some embodiments, the MPVs, e.g., WPVs, are modified from their natural state. In some embodiments, the MPVs, e.g., WPVs, are modified by altering the quantity, concentration, or amount of a biomolecule naturally present, e.g., the addition or complete or partial removal of a biomolecule naturally present (e.g., carbohydrate, such as a glycan and/or glycan residue; fatty acid, lipid). In some embodiments, the MPV, e.g., WPV, is modified by the addition of a biomolecule not naturally present (e.g., carbohydrate, such as a glycan; fatty acid; lipid; or protein, e.g., a glycoprotein).

In some embodiments, the size of the MPVs, e.g., WPVs, is about 20-1,000 nm. In some embodiments, the size of the MPVs, e.g., WPVs, is about 100-160 nm. In some of these above embodiments, the MPVs, e.g., WPVs, comprise a lipid membrane to which one or more proteins described herein are associated. In some embodiments, the MPVs, e.g., WPVs, comprise one or more proteins selected from BTN1A1, CD81 and XOR. In some embodiments, one or more proteins associated with the lipid membrane of the MPVs, e.g., WPVs, are glycosylated. In some embodiments, the MPVs, e.g., WPVs, demonstrate stability under freeze-thaw cycles and/or temperature treatment. In some embodiments, the MPVs, e.g., WPVs, demonstrate colloidal stability when loaded with the biological molecule. In some embodiments, the MPVs, e.g., WPVs, demonstrate stability under acidic pH, e.g., pH of ≤ 4.5 or pH of ≤2.5. In some embodiments, the MPVs, e.g., WPVs, demonstrate stability upon sonication. In some embodiments, the MPVs, e.g., WPVs, demonstrate resistance to enzyme digestion, e.g., resistance to one or more digestive enzymes described herein and/or resistance to nuclease treatment. In any of these embodiments, the beneficial properties of the MPV, e.g., WPV, can be conferred to the LNP-MPV produced by the methods described herein, and accordingly make the LNP-MPV suitable to be used for oral delivery of a cargo, e.g., a cargo encapsulated in the LNP-MPV. In some embodiments, the LNP-MPVs are formulated to form a suitable composition for use in oral delivery of the cargo encapsulated therein to a subject, for example, a human patient. In some embodiments, the cargo can be a peptide, a protein, a nucleic acid, a polysaccharide, or a small molecule. See descriptions in the instant disclosure.

### Lipid Nanoparticles

As used herein, the term “lipid nanoparticle” or “LNP” refers to a particle comprising one or more lipids. In some embodiments, the lipid nanoparticle comprises a monolayer lipid membrane. Examples of such LNPs include micelle and reverse micelles. In other embodiments, the LNP comprises one or more bilayer lipid membranes. In some embodiments, the LNP disclosed herein is a liposome (also known as unilamellar liposome). Liposome refers to a spherical chamber or vesicle, which contains a single bilayer of an amphiphilic lipid or a mixture of such lipids surrounding an aqueous core. In other embodiments, the LNP is a multilamellar vesicle, which contains multiple lamellar phase lipid bilayers. Still in other embodiments, the LNP is solid lipid nanoparticle, which comprises a solid lipid core matrix that can solubilize lipophilic molecules. In some instances, a solid lipid nanoparticle can also be used to solubilize molecules such as nucleic acid, which may be encapsulated based on charges. In a solid lipid nanoparticle, the lipid core can be stabilized by surfactants (emulsifiers) and cargos can be distributed into lipid core.

In particular embodiments, a nanoparticle includes a lipid. Lipid nanoparticles include, but are not limited to, liposomes and micelles. Any of a number of lipids may be present, including cationic lipids, ionizable lipids, anionic lipids, neutral lipids, amphipathic lipids, conjugated lipids (e.g., PEGylated lipids), and/or structural lipids. Such lipids can be used alone or in combination.

### (i) Ionizable Cationic Lipids and Non-Ionizable Cationic Lipids

In some embodiments, the lipid nanoparticle comprises a cationic lipid. Such cationic lipids can be ionizable or non-ionizable. As used herein, the term “cationic lipid” refers to any lipid that can be positively charged.

As used herein, the term “ionizable lipid” has its ordinary meaning in the art and may refer to a lipid comprising one or more ionizable moieties. An ionizable moiety has its ordinary meaning in the art and refers a moiety that can act as proton-donor or proton acceptor. Accordingly, an ionizable lipid may comprise one or more ionizable moieties, which are charged under certain conditions. In some embodiments, an ionizable lipid may be positively charged under certain conditions (i.e., an ionizable cationic lipid). In other embodiments, an ionizable lipid may be negatively charged under certain conditions. Under other conditions, the ionizable cationic lipid may have a neutral charge under certain conditions. For example, an ionizable cationic lipid may have a positive charge at a certain pH and have a neutral charge at another pH. In some examples, an ionizable cationic lipid may have a positive charge at a pH below physiological pH and a neutral charge at physiological pH and above. The pH at which an ionizable cationic lipid is positively charged or neutral depends on its pKa value. Of note, charge dependent on pH or other conditions, is subject to an equilibrium, i.e., in a composition of lipids, such as comprised in an LNP particle, the charge status of specific moieties may vary. Reference herein to “positive”, “negative” or “neutral” charge means the overall charge status of the moieties in the composition under that particular condition. Also, under some conditions, e.g., under certain pH conditions, a moiety may be referred to as “partially deprotonated” or “partially protonated” or “partially charged”, meaning that a certain percentage of the overall moieties in the composition are charged.

As used herein, the term “non-ionizable lipid” refers to a lipid which comprises one or more charged moieties, which can be positively or negatively charged moieties. The charge of non-ionizable lipid remains constant across certain conditions, e.g., a wide pH range. For example, a non-ionizable lipid can have a permanent charge across a broad pH range, e.g., pH 1 to pH 14including at physiological pH and above. Physiological pH has its ordinary meaning and is approximately pH 7.4. In some embodiments, the non-ionizable lipid is pH insensitive and has a permanent positive charge, i.e., a non-ionizable cationic lipid.

As used herein, a “charged moiety” is a chemical moiety that carries a formal electronic charge, e.g., monovalent (+1, or -1), divalent (+2, or -2), trivalent (+3, or -3), etc. The charged moiety may be anionic (i.e., negatively charged) or cationic (i.e., positively charged). In some embodiments, the lipid nanoparticles comprise ionizable or non-ionizable lipids with a positive charge. Examples of positively-charged moieties include amine groups (e.g., primary, secondary, tertiary, and or quarternary amines), ammonium groups, pyridinium group, guanidine groups, and imidizolium groups. In a particular embodiment, the charged moieties comprise amine groups. In some embodiments, the lipid nanoparticles comprise ionizable or non-ionizable lipids with a charged charge.

In some embodiments, the lipid is an amino lipid. In certain embodiments, an ionizable lipid or non-ionizable lipid molecule may comprise an amine group, and can be referred to as an “ionizable amino lipid” or “non-ionizable amino lipids”, respectively. In some embodiments, the lipid nanoparticles comprise an ionizable lipid, i.e., an ionizable cationic lipid, comprising one or more amine groups. In some embodiments, the lipid nanoparticle comprises a non-ionizable lipid, i.e., a non-ionizable cationic lipid, comprising one or more amine groups. In some embodiments, the non-ionizable amino lipid is pH insensitive and has a permanent positive charge. In some embodiments, the lipid nanoparticle does not comprise an ionizable lipid. In some embodiments, the lipid nanoparticle does not comprise an ionizable cationic lipid. Examples of negatively- charged groups or precursors thereof, include carboxylate groups, sulfonate groups, sulfate groups, phosphonate groups, phosphate groups, hydroxyl groups, and the like.

The charge of the charged moiety may vary, for example for ionizable lipids, in some cases, with the environmental conditions, for example, changes in pH may alter the charge of the moiety, and/or cause the moiety to become charged or uncharged. In general, the charge density of the molecule may be selected as desired. In other cases, for example for non-ionizable lipids, the charge of moiety may remain constant across these conditions.

In some embodiments, the lipid nanoparticles comprise an ionizable lipid, e.g.,, an ionizable cationic lipid, comprising one or more amine groups. In some embodiments, the lipid nanoparticle comprises a non-ionizable lipid, e.g.,, a non-ionizable cationic lipid, comprising one or more amine groups. In some embodiments, the non-ionizable amino lipid is pH insensitive and has a permanent positive charge.

In some embodiments, the lipid nanoparticles comprise an ionizable lipid, e.g., an ionizable cationic lipid, for example, DODMA. In some examples, the ionizable lipid is an ionizable amino lipid. The ionizable amino lipid may have at least one protonatable group. In some embodiments, the lipid nanoparticle comprises a non-ionizable lipid, e.g., a non-ionizable cationic lipid, for example, DOTAP. In some embodiments, the lipid nanoparticle does not comprise an ionizable lipid, e.g., does not comprise an ionizable cationic lipid.

### Ionizable Lipids

In one embodiment, the ionizable amino lipid may have a positively charged hydrophilic head (amino head group, including an alkylamino or dialkylamino group) and a hydrophobic tail (e.g., one or two fatty acid or fatty alkyl chains) that are connected via a linker structure. In addition to these, an ionizable lipid may also be a lipid including a cyclic amine group. In some embodiments, the ionizable amino lipid is positively charged at a pH at or below physiological pH (e.g., below pH 7.4), and neutral at a second pH, for example at or above physiological pH (pH 7.4 or greater).

In one embodiment, the ionizable lipid may be selected from, but not limited to, an ionizable lipid described in International Publication Nos. WO2013086354 and WO2013116126, the relevant disclosures of which are incorporated by reference for the subject matter and purpose referenced herein. Such ionizable lipids may be used in for making lipid nanoparticles comprising nucleic acid-based agents such as siRNAs. In yet another embodiment, the ionizable lipid may be selected from, but not limited to, formula CLI-CLXXXXII disclosed in U.S. Pat. No. 7,404,969, the relevant disclosures of which are incorporated by reference for the subject matter and purpose referenced herein. Such lipids may be used for making lipid nanoparticles comprising nucleic acid therapeutics such as antisense oligonucleotides, siRNAs, or mRNAs.

In some embodiments, the lipid nanoparticle may include one or more (e.g., 1, 2, 3, 4, 5, 6, 7, or 8) ionizable lipids, e.g., cationic ionizable lipids.

Such cationic ionizable lipids include, but are not limited to, 3-(didodecylamino)-N 1 ,N 1 ,4-tridodecyl-1-piperazineethanamine (KL 10) , N 1 -[2-(didodecylamino)ethyl] -N 1 ,N4,N4-tridodecyl- 1 ,4-piperazinediethanamine (KL22), 14,25-ditridecyl- 15 , 18 ,21 ,24-tetraaza-octatriacontane (KL25), 1,2-dilinoleyloxy-N,N-dimethylaminopropane (DLin-DMA), 2.2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2.2-dilinoleyl-4-(2-dimethylaminoethyl)-[1,3]-dioxolane (DLin-KC2-DMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8- [(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)); 3-b-(N- (N′,N′-dimethylaminoethane)-carbamoyl)cholesterol (DC-Chol); 1,2-dioleoyl-3-dimethylammonium propane (“DODAP”), N,N-dimethyl-2,3-dioleyloxy)propylamine (DODMA); 1,2-distearyloxy-N,N-dimethyl-3-aminopropane (DSDMA); N-(4-carboxybenzyl)-N,N-dimethyl-2,3-bis(oleoyloxy)propan-1-aminium (DOBAQ); YSK05; 4-(((2,3-bis(oleoyloxy)propyl)(methyl)amino)methyl)benzoic acid (DOBAT); 3-((2,3-bis(oleoyloxy)propyl)(methyl)amino)propanoic acid (DOPAT); and Alny-100.

In some embodiments, KL10, KL22, and KL25 described, for example, in U.S. Pat. No. 8,691,750, can be used.

In some embodiments, the ionizable cationic lipid is has a neutral charge at neutral or physiological pH. In one non-limiting example, the lipid is DODMA. DODMA is an ionizable cationic lipid, which has a pKa=7 with a tertiary amine head group.

In some embodiments, the ionizable cationic lipid is has a positive charge at neutral or physiological pH. In some embodiments, the ionizable cationic lipid is DC-Chol. DC-Chol is an ionizable lipid having a tertiary amine group and a pKa = 7.8, i.e., DC-cholesterol has a positive charge at neutral or physiological pHs (pH 7 or pH 7.4). Other examples of ionizable cationic lipids which are positively charged at neutral or physiological pH include and DODMA. In one embodiment, the lipid nanoparticles may comprise an ionizable cationic lipid, which may be is DODMA. DODMA is a cationic lipid, which is a pH-sensitive lipid with a cationic charge at physiologic pH.

In some embodiments, the lipid nanoparticles comprises a combination of ionizable cationic lipids described above.

### Non-Ionizable Lipids

In some embodiments, the lipids for use in making the lipid vesicles disclosed herein can be non-ionizable cationic lipids. Such lipids are positively charged at a wide range of pH (e.g., pH of 1-12). In some embodiments, the non-ionizable lipid is an amino lipid, i.e., a “non-ionizable cationic lipid” or “non-ionizable amino lipid.” In one embodiment, the non-ionizable cationic lipid is pH-insensitive with a permanent positive charge.

In one embodiment, the non-ionizable amino lipid may have a positively charged hydrophilic head (amino head group) and a hydrophobic tail (e.g., one or two fatty acid or fatty alkyl chains) that are connected via a linker structure. In some embodiments, non-ionizable amino lipid comprises a tetraalkyl or trialkyl amino group connected through a linker (such as alkyl) to the lipid tails. In addition to these, a non-ionizable lipid may also be a lipid including a cyclic amine group.

In one embodiment, the non-ionizable lipid may be selected from, but not limited to, N-(2,3-dioleoyloxy)propyl)-N,N,N-trimethylammonium chloride (DOTAP); 1,2-Dioleyloxy-3-trimethylaminopropane chloride salt (DOTAP.Q); N-(1,2-dimyristyloxyprop-3-yl)-N,N-dimethyl-N-hydroxyethyl ammonium bromide (DMRIE), dioctadecylamidoglycyl carboxyspermine (DOGS); DODAC; N-(2,3-dioleyloxy)propyl-N,N- N-triethylammonium chloride (DOTMA); N-(1-(2,3-dioleyloxy)propyl)-N-2-(sperminecarboxamido)ethyl)-N,N-dimethyl-ammonium trifluoracetate (DOSPA); N-(2-carboxypropyl)-N,N-dimethyl-2,3-bis(oleoyloxy)propan-1-aminium (DOMPAQ); N-(carboxymethyl)-N,N-dimethyl-2,3-bis(oleoyloxy)propan-1-aminium (DOAAQ); O,O′-ditetradecanoyl-N-(alpha-trimethyl ammonio acetyl) diethanolamine chloride (DC-6-14); (1,2-dioleyloxypropyl)-3 dimethylhydroxyethyl ammoniumbromide) (DORIE), DODMA-An, N,N-distearyl-N,N-dimethylammonium chlorideHEPES, N-(2-hydroxyethyl)piperazine-N′-2-ethanesulfonic acid (DSDAC), and N,N-distearyl-N,N-dimethylammonium bromide (DDAB). In some embodiments, the lipid nanoparticle comprises a combination of non-ionizable cationic lipids described above.

Additionally, a number of commercial preparations of cationic can be used, such as, e.g., LIPOFECTIN® (including DOTMA and DOPE, available from GIBCO/BRL), and LIPOFECT AMINEⓇ (including DOSPA and DOPE, available from GIBCO/BRL).

In one specific embodiment, the lipid nanoparticle comprises a non-ionizable cationic lipid, which may be DOTAP. DOTAP is a cationic lipid which is not ionizable; it is a pH-insensitive lipid with a permanent cationic charge.

In some embodiments, the lipid nanoparticle comprises a combination of one or more non-ionizable cationic lipids and one or more ionizable cationic lipids described above.

### (ii) Anionic Lipids

In some embodiments, the lipid nanoparticle comprises an anionic lipid. Anionic lipids suitable for use in lipid nanoparticles of the disclosure include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, phosphatidylserine, and other anionic modifying groups joined to neutral lipids.

### (iii) Neutral Lipids

In some embodiments, the lipid nanoparticle comprises a neutral lipid. Neutral lipids (including both uncharged and zwitterionic lipids) suitable for use in lipid nanoparticles of the disclosure include, but are not limited to, diacylphosphatidylcholine (or 1,2-Distearoyl-sn-glycero-3-phosphocholine (DSPC)), diacylphosphatidylethanolamine, ceramide, cephalin, sterols (e.g., cholesterol) and cerebrosides. Other non-limiting examples of neutral lipids include dioleoylphosphatidylcholine (DOPC), dipalmitoylphosphatidylcholine (DPPC), Dipalmitoylphosphatidylcholine (DOPG), 1,2-Dipalmitoyl-sn-glycero-3-phosphoglycerol (DPPG), 1,2-Dioleoyl-sn-glycerol-3-phosphoethanolamine (DOPE), 1-Palmitoyl-2-oleoyl-sn-glycero-3-phosphocholine (POPC), Palmitoyl-2-oleoyl-sn-glycero-3-phosphoethanolamine (POPE) and 1,2-Dipalmitoyl-sn-glycero-3-phosphoethanolamine (DPPE), 1,2-Dimyristoyl-sn-glycero-3-phosphoethanolamine (DMPE), 1,2-Distearoyl-sn-glycero-3-phosphoethanolamine (DSPE), 16-O-monomethyl PE, 16-O-dimethyl PE, 18-1-trans PE, 1-stearoyl-2-oleoyl-phosphatidyethanolamine (SOPE), and 1 ,2-dielaidoyl-sn-glycero-3-phophoethanolamine (transDOPE), dipalmitoylphosphatidylcholine (DMPC), milk sphingomyelin, and 1,2-dilauroyl-sn-glycero-3-phosphocholine (DLPC).

Lipids having a variety of acyl chain groups of varying chain length and degree of saturation are available or may be isolated or synthesized by well-known techniques. Additionally, lipids having mixtures of saturated and unsaturated fatty acid chains and cyclic regions can be used. In some embodiments, the neutral lipids used in the disclosure are DOPE, DSPC, DPPC, POPC, DOPC, or any related phosphatidylcholine. In specific examples, the lipid nanoparticle disclosed herein comprises cholesterol.

### (iv) Amphipathic Lipids

In some embodiments, the lipid nanoparticle comprises one or more amphiphatic lipid, i.e., a lipid having a polar part and a non-polar part. Exemplary amphipathic lipids suitable for use in nanoparticles of the disclosure include, but are not limited to, sphingolipids, phospholipids, fatty acids, and amino lipids.

Particular amphipathic lipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition to pass through the membrane permitting.

Non-natural amphipathic lipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

In some examples, the lipid nanoparticle may comprise one or more amphiphatic lipids, which may be phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids..

Other phosphorus-lacking compounds, such as sphingolipids, glycosphingolipid families, diacylglycerols, and b-acyloxyacids, may also be used. Additionally, such amphipathic lipids can be readily mixed with other lipids, such as triglycerides and sterols.

### (v) PEGylated Lipids

In some embodiments, the lipid nanoparticle comprises PEGylated lipid. The lipid component of a lipid nanoparticle composition may include one or more molecules comprising polyethylene glycol, such as PEG or PEG-modified lipids. Such species may be alternately referred to as PEGylated lipids. A PEGylated lipid (also known as a PEG lipid or a PEG-modified lipid) is a lipid modified with polyethylene glycol. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEGylated lipid may be PEG-c-DOMG, PEG-DMG, PEG-DLPE, PEG-DMPE, PEG-DPPC, PEG-DSG, or a PEG-DSPE lipid.

In some embodiments the PEG-modified lipids are a modified form of PEG DMG. PEG-DMG has the following structure:

[symbol]

In one embodiment, PEG lipids useful in the present invention are PEGylated lipids described in International Publication No. WO2012099755, the relevant disclosures of which are incorporated by reference for the subject matter and purpose referenced herein. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (-OH) groups on the lipid. In certain embodiments, the PEG-OH lipid includes one or more hydroxyl groups on the PEG chain. In certain embodiments, a PEG-OH or hydroxy-PEGylated lipid comprises an -OH group at the terminus of the PEG chain. In some examples, the PEG lipids may be modified to comprise a methoxy group (methoxy PEG or mPEG), which is a functional group consisting of a methyl moiety bound to oxygen.

Each possibility represents a separate embodiment of the present invention. In some embodiments, the length of the PEG chain comprises about 250, about about 500, about 1000, about 2000, about 3000, about 5000, about 10000 ethylene oxide units.

### (vi) Structural Lipids

The lipid nanoparticle disclosed herein can comprise one or more structural lipids. As used herein, the term “structural lipid” refers to sterols and also to lipids containing sterol moieties. Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol.

### (vii) Targeting Moieties

In some embodiments, the nanoparticle comprises a targeting moiety. In certain embodiments, it is desirable to target a nanoparticle, e.g., a lipid nanoparticle, of the disclosure using a targeting moiety that is specific to a cell type and/or tissue type. In some embodiments, a nanoparticle may be targeted to a particular cell, tissue, and/or organ using a targeting moiety. In particular embodiments, a nanoparticle comprises a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Fipid Res. 42(5):439-62, 2003 and Abra et al., J. Fiposome Res. 12: 1-3, 2002.

In some embodiments, a lipid nanoparticle (e.g., a liposome) may include a surface coating of hydrophilic polymer chains, such as polyethylene glycol (PEG) chains (see, e.g., Allen et al., Biochi mica et Biophysica Acta 1237: 99-108, 1995; DeFrees et al., Journal of the American Chemistry Society 118: 6101-6104, 1996; Blume et al., Biochimica et Biophysica Acta 1149: 180-184,1993; Klibanov et al., Journal of Fiposome Research 2: 321-334, 1992; U.S. Pat. No. 5,013,556; Zalipsky, Bioconjugate Chemistry 4: 296-299, 1993; Zalipsky, FEBS Fetters 353: 71-74, 1994; Zalipsky, in Stealth Fiposomes Chapter 9 (Fasic and Martin, Eds) CRC Press, Boca Raton Fla., 1995). In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Fiposome Research 2: 321-334, 1992; Kirpotin et al., FEBS Fetters 388: 115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265: 16337-16342, 1990 and Leonetti et al., Proc. Natl. Acad. Sci. (USA), 87:2448-2451, 1990). Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle of the disclosure includes a targeting moiety that targets the lipid nanoparticle to a cell including, but not limited to, hepatocytes, colon cells, epithelial cells (e.g., a mucosal epithelial cells, such as mucosal enterocytes), hematopoietic cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the lipid nanoparticle comprises a targeting moiety directed to a cell type present in the intestinal mucosa, e.g., in the small intestine. In some embodiments, the lipid nanoparticle comprises a targeting moiety directed to an epithelial cell of the intestine, e.g., a mucosal enterocyte.

In some embodiments, the targeting moiety comprises one or more lectins selected from Con A, RCA, WGA, DSL, Jacalin, or any combination thereof.

### (viii) Polymers

In some embodiments, the nanoparticle comprises a pH-responsive polymer. pH-sensitive polymers are polymers that respond to changes in pH by changing their structures. In some non-limiting embodiments of the disclosure the polymers can be made of homopolymers of alkyl acrylic acids, such as butyl acrylic acid (BAA) or propyl acrylic acid (PAA), or can be copolymers of ethyl acrylic acid (EAA). Polymers of alkyl amine or alkyl alcohol derivatives of maleic-anhydride copolymers with methyl vinyl ether or styrene may also be used.

In general, the pH-responsive polymer is composed of monomeric residues with particular properties. Anionic monomeric residues comprise a species charged or charge-able to an anion, including a protonatable anionic species. Anionic monomeric residues can be anionic at an approxi-mately neutral pH of 7.2-7.4. Cationic monomeric residues comprise a species charged or chargeable to a cation, including a deprotonatable cationic species. Cationic monomeric residues can be cationic at an approximately neutral pH of 7.2-7.4.

In some embodiments, the nanoparticle comprises polymers, which are not pH-responsive. Non-limiting examples of such positively charged polymers include, but are not limited to, positive polymers are PEI, poly-lysine, and dendrimers, such as PAMAM.

In some embodiments, the polymers can be made as copolymers with other monomers. The addition of other monomers can enhance the potency of the polymers, or add chemical groups with useful functionalities to facilitate association with other molecular entities, including the targeting moiety and/or other adjuvant materials such as poly(ethylene glycol). These copolymers may include, but are not limited to, copolymers with monomers containing groups that can be cross-linked to a targeting moiety.

Hydrophobic monomeric residues comprise a hydrophobic species. Hydrophilic monomeric residues comprise a hydrophilic species.

### (ix) Other Components

The nanoparticles disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Pat. Application Publication No. 2005/0222064. Carbohydrates can include simple sugars (e.g., glucose) and polysaccharides (e.g., glycogen and derivatives and analogs thereof).

In some embodiments, the nanoparticle comprises a helper lipid. As used herein, “helper lipid” refers to stabilizing lipids. Helper lipids may be neutral (e.g., have no charged moieties or zwitterionic). In some embodiments, the lipid nanoparticle disclosed herein may comprise one or more of the following helper lipids: 1,2-distearoyl-sn-glycero-3-phosphoethanolamine-N-[ amino(polyethylene-glycol)-2000] ( amine- PEG-DSPE), 1,2-dioleoyl-sn-glycero-3-phosphoetha- nolamine-N-(7-nitro-2-1,3-benzoxadiazol-4-yl)] (NBD- PE), 1,2-distearoyl-sn-glycero-3-phosphoethanolamine-N-[ maleimide (polyethylene glycol)-2000] (mal-PEG-DSPE), Distearoyl-phosphatidylcholine (DSPC), 1,2-dioleoyl-3-di-methylammonium-propane (DODAP), N-palmitoyl-sphin-gosine-1-succinyl[ methoxy(polyethylene glycol)2000] (PEG-Cer). In some examples, the lipid nanoparticles disclosed herein may comprise one or more helper lipids, such as DOPC, DSPC, DOPE, or a combination thereof, at a concentration of about 10-20 mol%.

Other lipids known in the art for preparing lipid nanoparticles such as liposomes can also be used in the present disclosure. Examples include those disclosed in US20110256175A1, US8642076B2, US20120225434A1, US20150190515A1, US10195291B2, US20150165039A1, US20150306039A1, US10369226B2, US20130338210A1, US20190374646A1, US20140308304A1, US9463247B2, US8034376B2, US20130202652A1, US20180169268A1, US20180170866A1, US20150239926A1, US9834510B2, US20180000953A1, US20180085474A1, US20120251618A1, US20150166462A1, US20150086613A1, US20160151409A1, US20140288160A1, US9629804B2, US20150366997A1, US20170246319A1, US20170196809A1, US10125092B2, US20180290965A1, US20190358170A1, US10124065B2, US20180296677A1, US20190136231A1, US20170079916A1, US20150140070A1, US20160067346A1, US10086013B2, US20190240349A1, US9840479B2, US9556110B2, US9895443B2, US10086013B2, US9439968B2, US9556110B2, US20170349543A1, US20160220681A1, US20170354672A1, US20120253032A1, US20120149894A1, US20130274523A1, US20130053572A1, US20100048888A1, and US20140162934A1. The relevant disclosures of each of these patents and patent application publications are incorporated by reference for the purpose and subject matter referenced herein.

### (x) Exemplary Lipid Nanoparticles

Any of the lipid nanoparticles (e.g., liposomes) disclosed herein may have a suitable size for carrying a cargo of interest. In some embodiments, the lipid nanoparticle may have a size ranging from about 20-150 nm. For example, the lipid nanoparticles may have a size of about 20-120 nm, about 20-100 nm, about 20-80 nm, about 40-150 nm, about 40-100 nm, about 40-80 nm, about 60-150 nm, about 60-120 nm, about 60-100 nm, about 80-150 nm, about 80-120 nm, or about 100-150 nm.

In some embodiments, the lipid nanoparticle disclosed herein is a cationic lipid nanoparticle. Such a lipid nanoparticle may comprise one or more ionizable cationic lipids one or more non-ionizable cationic lipids, or a combination thereof. Any of the ionizable and non-ionizable cationic lipids provided herein can be used for making the lipid nanoparticles.

Exemplary ionizable cationic lipids and non-ionizable cationic lipids are described above herein and include, but are not limited to, DOSPA, DOGS, DOTMA, DOTAP, DC-Chol, DMRIE, 98N12-5, C12-200, DLin-KC2-DMA (KC2), DLin-MC3-DMA (MC3),. In some embodiments, the cationic lipid nanoparticle comprises one or more of such ionizable cationic lipids and/or non-ionizable cationic lipids. In one embodiment, a cationic lipid nanoparticle (e.g., a cationic liposome) comprises DOTAP or DOTMA. Such a cationic lipid nanoparticle may optionally further comprise DSPC, DSPE-mPEG, DOPC, or a combination thereof.

In some embodiments, the lipid nanoparticle disclosed herein is a neutral lipid nanoparticle. For example, a neutral lipid nanoparticle may comprise one or more neutral lipids, which can be hydrophobic molecules lacking charged groups. Exemplary neutral lipids include, but are not limited to, DPPC, DOPC, DOPE, cholesterol, and SM. In one embodiment, a neutral lipid nanoparticle (e.g., a neutral liposome) comprises DSPC, cholesterol, and DSPE-mPEG.

In some embodiments, the lipid nanoparticle disclosed herein comprises similar lipid content (i.e., variation no more than 30%) as the MPV, e.g., WPV, (also referred to as WEVs) to be fused with. Lipid contents of naturally occurring MPVs, e.g., WPVs, are disclosed above. In some embodiments, the lipid content in the nanoparticle is at least 80% identical to the lipid content of the MPV, to be fused with. In further embodiments, the lipid content in the nanoparticle is at least 90% identical to the lipid content of the MPV to be fused with.

In other embodiments, the lipid nanoparticles disclosed herein comprises naturally-occurring lipid components but its lipid content (e.g., type of lipids and mole percentage thereof) does not mimic that of the MPV, e.g., WPV, to be fused with. Alternatively, in some embodiments, the lipid nanoparticles comprise non-naturally occurring lipids (synthetic) and/or lipidoids. In some examples, the lipid nanoparticles comprise a combination of naturally-occurring lipids and synthetic lipids.

Mole percent or mole percentage refers to the percentage of the total munber of molecules (total moles) of one component in the total number of molecules of a whole mixture. For example, a mole percentage of 5% of Lipid A of the total lipid molar concentration (i.e., 5 mol% of Lipid A) refers to the percentage of the total molecule number of Lipid A in the total molecule number of all lipid molecules in a composition.

In some embodiments, a lipid nanoparticle as disclosed herein may comprise a mole percentage of a non-ionizable cationic lipid of about 5% to about 50% of the total lipid molar concentration (i.e., about 5 mol% to about 50 mol%). In some embodiments, a lipid nanoparticle comprises a mole percentage of a non-ionizable cationic lipid of less than 30% of the total lipid molar concentration, e.g., about 5% to about 25%, about 5% to about 29%, about 5% to about 10%, about 10% to about 20% or about 20% to about 25% or about 25% to about 29% of the total lipid molar concentration. In some embodiments, a lipid nanoparticle comprises a mole percentage of a non-ionizable cationic lipid of about 30% to about 40% or about 40% to about 50% of the total lipid molar concentration.

For example, a lipid nanoparticle disclosed herein comprises a mole percentage of DOTAP of about 5% to about 50% of the total lipid molar concentration (e.g., about 10 mol% to about 50 mol%). In some examples, the mole percentage of DOTAP in the the total lipid molar concentration of the lipid nanoparticle may be less than 30%, e.g., about 5% to about 25%, about 5% to about 29%, about 5% to about 10%, about 10% to about 20% or about 20% to about 25%. In some embodiments, a lipid nanoparticle comprises a concentration of DOTAP of about 30% to about 40% or about 40% to about 50% of the total lipid molar concentration.

In some embodiments, a lipid nanoparticle disclosed herein comprises a mole percentage of an ionizable cationic lipid of about 5% to about 50% of the total lipid molar concentration. For example, the mole percentage of the ionizable cationic lipid in the the total lipid molar concentration of the lipid nanoparticle may range from about 30% to about 50%, e.g., about 35% to about 50%, about 40% to about 50%, or about 45% to about 50%.

For example, a lipid nanoparticle disclosed herein may comprise a mole percentage of DODMA ranging from about 5% to about 50% of the total lipid molar concentration. In some examples, a lipid nanoparticle (e.g., a liposome) comprises a mole percentage of DODMA of about 30% to about 50% of the total lipid molar concentration, e.g., about 35% to about 50%, about 40% to about 50%, or about 45% to about 50%. Lipid nanoparticles comprising DODMA can be used for carrying nucleic acid-based cargos, such as antisense oligonucleotides, siRNAs, or mRNAs.

In other examples, a lipid nanoparticle (e.g., a liposome) as disclosed herein may comprise about 50 mol % to about 70 mol % of DOPC. In some embodiments, the lipid nanoparticle comprises about 10 mol % to about 50 mol % of cholesterol. In some embodiments, the lipid nanoparticle comprises about 5 mol % to about 50 mol % of DOTAP and/or DODMA.

In some embodiments, any of the lipid nanoparticles disclosed herein (e.g., liposomes) may comprise about 5 mol % to about 30 mol % of DOPE, DSPC, DOPC, or a combination thereof. In some embodiments, the lipid nanoparticle comprises about 0.5-10 mol % of DPPC-PEG and/or DSPE-PEG. In some examples, the PEG moieties are PEG2000. In other embodiments, the lipid nanoparticle comprises a combination of any of the above lipids at the defined concentrations.

In specific embodiments, a lipid nanoparticle (e.g., a liposome) as disclosed herein comprises about 50 mol % to about 70 mol % of DOPC, about 10 mol % to about 30 mol % by weight of cholesterol, about 5 mol % to about 15 mol % of DOTAP, from about 5 mol % to about 15 mol % of DOPE, and about 0.5 mol % to about 5.0 mol % of DPPE-PEG2000 (e.g., about 0.5 mol % to about 3.0 mol %).

In other examples, the lipid nanoparticles disclosed herein may comprise one or more cationic lipids (e.g., ionizable or non-ionizable) at a concentration of about 10 mol% to about 50 mol%, and optionally cholesterol at a concentration of about 25-40 mol%, lipid-mPEG2000 (e.g., lipid being DSPE, DMPE, and/or DMPG) at a concentration of about 0.5-3 mol%.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ± 20 %, preferably up to ± 10 %, more preferably up to ± 5 %, and more preferably still up to ± 1 % of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In some embodiments, the lipid mix of the particle comprises 40:17.5:40:2.5 molar ratio of DlinDMA:DSPC:Chol:PEG-Cer.). In some embodiments, the lipid mix of the particle comprises 40:17.5:40:2.5 molar ratio of DODAP:DSPC:Chol:PEG-Cer. In some embodiments, DLinDMA liposomes (DSPC/ Chol/PEG) are used. In some embodiments, DLinDMA was substituted by the ionizable lipid DODAP. In some embodiments, the nanoparticle comprises DlinDMA:Chol:DSPC:PEG-S-DMG:NBD-PC 40:40:17.5:2:0.5.

Any of the the lipid nanoparticles described herein may be lipidoid-based. The synthesis of lipidoids has been extensively described and formulations containing these compounds are particularly suited for delivery of polynucleotides (see Mahon et al., Bioconjug Chem. 2010 21: 1448-1454; Schroeder et al., J Intern Med. 2010 267:9-21; Akinc et al., Nat. Biotechnol. 2008 26:561-569; Love et al., Proc Natl Acad Sci USA. 2010 107: 1864-1869; Siegwart et al., Proc Natl Acad Sci USA. 2011 108: 12996-3001)

Exemplary lipidoids include, but are not limited to, DLin-DMA, DLin-K-DMA, DLin-KC2-DMA, 98N12-5, C12-200 (including variants and derivatives), DLin-MC3-DMA and analogs thereof.

Any of the lipid nanoparticles described herein, optionally loaded with a cargo, can be used to contact a MPV, e.g., WPV, described herein allowing for fusion of the lipid nanoparticle with the MPV, thereby producing an LNP-MPV, e.g., a liposome-WPV, having the cargo encapsulated therein.

### Preparation of Cargo-Carrying Lipid Nanoparticles

A variety of methods are available for preparing lipid nanoparticles such as liposomes. See, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984), Liposomes: from Physics to Applications (1993) and Lipid Delivery Systems for Nucleic-Acid-Based-Drugs: From Production to Clinical Applications). Suitable methods include, for example, sonication, extrusion, high pressure/homogenization, microfluidization, detergent dialysis, calcium-induced fusion of small liposome vehicles and ether fusion methods, all of which are well known in the art. Any of such methods may be performed in the presence of a suitable cargo such that the resultant lipid nanoparticles such as liposomes would carry the suitable cargo.

One technique for liposome preparation and cargo loading into the liposome is the Thin Film Hydration (TFH), where lipids are dissolved in an organic solvent and subsequently evaporated (e.g., through the use of a rotary evaporator) resulting in a thin lipid layer formation. After hydration of the layer using an aqueous buffer containing the cargo, multilamellar vesicles are formed, which are reduced in size to produce unilamellar vesicles (larger or small, LUV and SUV) by extrusion through membranes or by the sonication of the starting multilamellar vesicles.

Liposomes can be also prepared through a double emulsion method where lipids are disolved in a water/organic solvent mixture. The organic solution, comprising water droplets, is mixed with an excess of aqueous medium, resuling in water-in-oil-in-water (W/O/W) double emulsion formation.

Using reverse phase evaporation, large unilamellaer liposome vesicles can be loaded with cargo. In this technique a two-phase system is formed by phospholipids dissolution in organic solvents and aqueous solution. The resulting suspension is then sonicated until it is a clear one-phase dispersion. The liposome formation is performed when the organic solvent is evaporated under reduced pressure.

Microfluidics (e.g., continuous-flow microfluidic and droplet-based microfluidic methods) can be used to improve control over lipid hydration. Dual Asymmetric Centrifugation (DAC) is another method for producing cargoloaded liposomes. In addition, the usual centrifugation the sample is subjected to an additional rotation around its own vertical axis, resulting in efficient homogenization. Alternatively, unilamellar cargo loaded liposomes can be generatefd using ethanol injection (EI) This method utilizes the rapid injection of an ethanolic solution, in which lipids are dissolved, into an aqueous medium containing nucleic acids to be encapsulated, through the use of a needle, resuling in sponanteous formation of carglo loaded liposome vesicles. Additional methods include (1) detergent dialysis, where lipid and cargo are solubilized in detergent of appropriate ionic strength, and where cargo loaded vesicles are formed once detergent is removed by dialysis and (2) spontaneous Vesicle Formation by Ethanol Dilution, where dropwise ethanol dilution allows the spontaneous formation of liposomes loaded with cargo by the controlled addition of lipid dissolved in ethanol to a rapidly mixing aqueous buffer containing the cargo.

In some embodiments, a cargo-carrying lipid nanoparticle as disclosed herein is prepared as follows. One or more suitable lipids are placed in an alcohol solvent (e.g., in ethanol) to form an alcohol solution. A suitable cargo is dissolved in an aqueous solution. The lipid-containing alcohol solution can be mixed with the cargo-containing aqueous solution under suitable conditions under which lipid nanoparticles form with the cargo embedded in the lipid nanoparticles. In some embodiments, each of the lipid-containing alcohol solution and the cargo-containing aqueous solution flow through tubes via pumps and the two solutions interact with each other at Y or T junctions of the tubes, wherein cargo-carrying lipid nanoparticles form. In some embodiments, the tubes have a diameter of about 0.2-2 mm. In some embodiments, production of cargo-carrying lipid nanoparticles are performed using a microfluidic device. Microfluidics involves manipulating and controlling fluids, usually in the range of microliters (10⁻⁶) to picoliters (10⁻¹²), in networks of channels with dimensions from tens to hundreds of micrometers. Fluid handling can be manipulated by components such as microfluidic pumps or microfluidic valves. Microfluidic pumps can supply fluids in a continuous way or can be used for dosing. Microfluidic valves can inject precise volumes of sample or buffer. In some instances, the microfluidic device used herein may comprise one or more channels (e.g., of glass and/or polymer materials) having a diameter of about less than 2 mm (e.g., 0.02-2 mm).

In some embodiments, a cargo-carrying lipid nanoparticle as disclosed herein may be prepared as follows. One or more suitable lipids can be dissolved in a suitable solvent (e.g., an organic solvent such as chloroform) to form a solution. The solvent can then be evaporated from the solution using methods known in the art, for example, under a stream of air, and the container containing the solution may be rotated to form a thin lipid film on the wall of the container. If needed, the lipid film may be dried under vacuum for a suitable period for remove any trace amount of the solvent. The lipid film is then rehydrated in a solution containing a suitable cargo. The rehydrated lipid film is then subject to vortexing, sonication, extrusion, freeze-thaw cycles, or a combination thereof, to allow for formation of lipid nanoparticles carrying the cargo.

Any suitable cargos such as those disclosed herein can be used for making the cargo-carrying LNPs. Examples include, but are not limited to, nucleic acid-based cargos, protein-based cargos, small molecule-based cargos, allergen, adjuvant, antigen, or immunogen, vaccine, or particles such as viral particles. Nucleic acid-based cargo may be single or double-stranded DNA, iRNA shRNA, siRNA, mRNA, non-coding RNA (ncRNA including lncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof, DNA-based cargos such as an expression system (e.g., a viral vector or a non-viral vector), closed-end DNA (ceDNA). Protein-based cargos include antibodies, hormone, GLP-1 peptide, growth factor, a factor involved in the coagulation cascade, enzyme (e.g., metabolic enzymes, immunoregulatory enzymes, gastrointestinal enzymes, growth regulatory enzymes, coagulation cascade enzymes), cytokine, chemokine, vaccine antigens, antithrombotics, antithrombolytics, toxins, or antitoxin. Small molecule-based cargos can be small molecule enzyme inhibitors, receptor ligands, or allosteric modulators. Examples include metalloprotease inhibitors, heat shock protein inhibitors, proteasome inhibitors, tyrosine kinase inhibitors, and serine/threonine kinase inhibitors. Specific examples for suitable cargos are provided in Tables 1-19. LNPs loaded with any of such cargos are also within the scope of the present disclosure.

The lipid nanoparticles prepared following any of the methods known in the art or disclosed herein can be analyzed to determine concentration and/or particle size distribution (e.g., by NTA). Alternatively or in addition, the lipid nanoparticles can be fractionated and particles having suitable sizes may be collected for use in the fusion method disclosed herein.

Any of the processes for producing cargo-carrying lipid nanoparticles as disclosed herein is within the scope of the present disclosure, e.g., as part of the methods for producing cargo-loaded MPVs, e.g., WPVs, via fusion as disclosed herein.

### Preparation of LNP-MPVs

Any of the MPVs, e.g., WPVs, and any of the cargo-carrying lipid nanoparticles disclosed herein can be mixed under conditions allowing for fusion of the MPVs, e.g., WPVs, and the lipid nanoparticles to produce LNP-MPVs, in which the cargo is encapsulated. This approach is particularly suitable for making luminal loading of a cargo into MPVs.

As used herein, the term “cargo-loaded vesicle” is meant to be inclusive of the loading of one or more cargos, e.g., therapeutic agents and diagnostic agents, into a vesicle (e.g., a MPV, e.g., WPV, disclosed herein). As used herein, the term “loaded” or “loading” as used in reference to a “cargo-loaded vesicle,” refers to a vesicle having one or more cargos (which can be biological molecules such as therapeutic agents or diagnostic agents) that are either (1) encapsulated inside the vesicle; (2) associated with or partially embedded within the lipid membrane of the vesicle (i.e. partly protruding inside the interior of the vesicle); (3) associated with or bound to the outer portion of the lipid membrane and associated components (i.e., partly protruding or fully outside the vesicle); or (4) entirely disposed within the lipid membrane of the vesicle (i.e., entirely contained within the lipid membrane).

The term “cargo-loading” refers to the process of loading, adding, or including exogenous cargo or therapeutic to the MPV, e.g., WPV, such that any one or more of the above (1)-(4) resultant cargoloaded or therapeutic-loaded vesicles is accomplished, e.g., an LNP-MPV. Thus, in some embodiments, the cargo is encapsulated inside the vesicle. In some embodiments, the cargo is associated with or partially embedded within the lipid membrane of the vesicle (i.e., partly protruding inside the interior of the vesicle). In some embodiments, the cargo is associated with or bound to the outer portion of the lipid membrane (i.e., partly protruding outside the vesicle). In some embodiments, the cargo is entirely disposed within the lipid membrane of the vesicle (i.e., entirely contained within the lipid membrane).

In some embodiments, one or more cargos, e.g., therapeutic agents or diagnostic agents, are present on the interior or internal surface of the LNP-MPV. In some embodiments, the one or more cargos present on the interior or internal surface of the LNP-MPV, are associated with the LNP-MPV, e.g., via chemical interaction, electromagnetic interaction, hydrophobic interaction, electrostatic interaction, van der Waals interaction, linkage, bond (hydrogen bond, ionic bond, covalent bond, etc.). In some embodiments, the one or more cargos present on the interior or internal surface of the LNP-MPV, are not associated with the LNP-MPV, e.g., the cargo is unattached to the vesicle. In some embodiments, the LNP-MPV has a cavity and/or forms a sac. In some embodiments, the LNP-MPV can encapsulate one or more cargos.

In some embodiments, the LNP-MPVs, are modified to display a lectin, which is capable of binding to a glycan, e.g., a glycoprotein or glycolipid present on a nanoparticle that comprise the glycan. Accordingly, in some embodiments, the LNP-MPVs, display lectins on their surface. In some embodiments, the LNP-MPV s, display one or more lectins selected from Con A, RCA, WGA, DSL, Jacalin, or any combination thereof. Alternatively, the LNP-MPV s, may be modified to display a binding moiety capable of binding to another binding moiety that is conjugated to the surface of the lipid nanoparticle. Such binding moiety pairs may be any ligand-receptor pairs such as biotin-streptavidin.

The fusion-based method disclosed herein allows for luminal loading of a suitable cargo into an LNP-MPV. To perform this method, any of the lipid nanoparticles (e.g., liposomes) that carry a suitable cargo as disclosed herein may be brought in contact with any of the MPV, e.g., WPV, as also disclosed herein under conditions allowing for fusion of the two particles to produce a fused vesicle (a.k.a., a duosome or LNP-MPV). Optionally, the fused vesicle, in which the cargo is encapsulated, can be collected, for example, by negative selection or by positive selection.

In any of the cargo loading embodiments described herein, the MPVs, e.g., WPVs, or compositions of MPVs, e.g., WPVs, used in the loading methods can comprise a relative abundance of casein less than about 40%, about 35%, about 30%, about 25%, about 20%, about 15%, about 10%, about 5% or less, e.g., about 4%, about 3%, about 2%, about 1%, or substantially free of any casein. In some embodiments, the MPVs, e.g., WPVs, or compositions of MPVs, e.g., WPVs, are substantially free of casein. In some embodiments, the MPVs, e.g., WPVs, or compositions of MPVs, e.g., WPVs, comprise lactoglobulin at a relative abundance of no greater than 25% (e.g., less than about 25%, about 20%, about 15%, about 10%, about 5% or less). In some embodiments, the MPVs, e.g., WPVs, or the composition comprising such may be substantially free of lactoglobulins. In some embodiments, the size of the MPVs, e.g., WPVs, is about 20-1,000 nm. In some embodiments, the MPVs, e.g., WPVs, are not modified from their naturally occurring state. In some embodiments, the MPVs, e.g., WPVs, are modified from their natural state. In some embodiments, the MPVs, e.g., WPVs, are modified by altering the quantity, concentration, or amount of a biomolecule naturally present, e.g., the addition or complete or partial removal of a biomolecule naturally present (e.g., carbohydrate, such as a glycan and/or glycan residue; fatty acid, lipid). In some embodiments, the MPV, e.g., WPV, is modified by the addition of a biomolecule not naturally present (e.g., carbohydrate, such as a glycan; fatty acid; lipid; or protein, e.g., glycoprotein). In some embodiments, the size of the MPVs, e.g., WPVs, is about 100-160 nm. In some embodiments, the MPVs, e.g., WPVs, comprise a lipid membrane to which one or more proteins described herein are associated. In some embodiments, the MPVs, e.g., WPVs, comprise one or more proteins selected from BTN1A1, CD81 and XOR. In some embodiments, one or more proteins associated with the lipid membrane of the MPVs, e.g., WPVs, are glycosylated. In some embodiments, the MPVs, e.g., WPVs, demonstrate stability under freeze-thaw cycles and/or temperature treatment. In some embodiments, the MPVs, e.g., WPVs, demonstrate colloidal stability when loaded with the biological molecule. In some embodiments, the MPVs, e.g., WPVs, demonstrate stability under acidic pH, e.g., pH of ≤ 4.5 or pH of ≤2.5. In some embodiments, the MPVs, e.g., WPVs, demonstrate stability upon sonication. In some embodiments, the MPVs, e.g., WPVs, demonstrate resistance to enzyme digestion, e.g., resistance to one or more digestive enzymes described herein and/or resistance to nuclease treatment. In any of these embodiments, the beneficial properties of the MPV, e.g., WPV, can be conferred to the LNP-MPV produced by the methods described herein, and accordingly make the LNP-MPV suitable to be used for oral delivery of a cargo, e.g., a cargo encapsulated in the LNP-MPV. In some embodiments, the LNP-MPVs are formulated to form a suitable composition for use in oral delivery of the cargo encapsulated therein to a subject, for example, a human patient. In some embodiments, the cargo can be a peptide, a protein, a nucleic acid, a polysaccharide, or a small molecule.

### (i) Fusion Methods

Fusion of the cargo-carrying lipid nanoparticle and MPVs, e.g., WPVs, can be performed following methods known in the art or those disclosed herein, e.g., incubation under suitable conditions for a suitable period, extrusion, sonication, and/or PEG-facilitated fusion.

In some embodiments, fusion of the cargo-carrying lipid nanoparticle and MPVs, e.g., WPVs, can be performed by incubating the two types of particles under a suitable temperature for a suitable period. It is reported herein that heating could facilitate fusion of the particles. In some embodiments, the two types of particles are incubated for at least one hour (e.g., for 1 hour, 2 hours, 3 hours, 4 hours, 5 hours, 6 hours, 8 hours, 10 hours, 12 hours or longer) at a temperature of about 4° C. to about 50° C. In some embodiments, the incubation temperature is about 10° C. to about 40° C. In some embodiments, the incubation temperature is about 15° C. to about 35° C. In some embodiments, the incubation temperature is about 20° C. to about 40° C. In some embodiments, the incubation temperature is about 25° C. to about 40° C. In some embodiments, the incubation temperature is about 35° C. to about 45° C. In some embodiments, the incubation temperature is about 40° C. to about 50° C. In some embodiments, the two types of particles are incubated for at least one hour and the incubation temperature is at least 35° C. and no more than 50° C. In one embodiment the two types of particles are incubated for at least one hour and the incubation temperature is at least 35° C. and no more than 40° C.

In some examples, the fusion step may be performed at room temperature (e.g., 25° C.) to 37° C. for up to 2 hours. When the fusion step involves lipid nanoparticles comprising helper lipids such as DSPC, the fusion step may be performed at up to 50° C. for 2 hours.

In any of the methods disclosed herein, the fusion step may be performed in a solution comprising polyethylene glycol (PEG) having a suitable molecular weight (e.g., about 2 kD to about 50 kD) and a suitable concentration (e.g., about 2% to about 50%) to improve fusion efficiency. In some embodiments, the PEG solution comprises PEG molecules having a molecular weight ranging from about 5% to about 40%, for example, about 10% to about 35%, about 15% to about 35%, about 20% to about 40%, or about 20% to about 35%. In specific embodiments, the PEG concentration is about 25%. In other embodiments, the PEG concentration is about 30%. In yet other embodiments, the PEG concentration is about 35%. Alternatively or in addition, in some embodiments, the suitable molecular weight of the PEG ranges from about 5 kD to about 20 kD, e.g., about 5 kD to about 18 kD, about 5 kD to about 15 kD, or about 5 kD to about 12 kD. In some embodiments, the PEG concentration is about 6 kD, about 8 kD, about 10 kD, or about 12 kD.

In some embodiments, the fusion reaction is performed in a solution comprising PEG having a molecular weight of about 6 kD to about 12 kD and a PEG concentration for about 10% to about 35%. In some embodiments, the fusion step is performed for at least 1 hour (e.g., 2 hours or 3 hours) at a temperature of about 25° C. to about 50° C. (e.g., about 35° C. to about 45° C.). In specific embodiments, the fusion reaction is performed in a solution comprising PEG having a molecular weight of about 8 kD to about 12 kD (e.g., about 8 kD) and a PEG concentration for about 20% to about 30% (e.g., about 30%) by weight.

In other examples, the fusion step may be performed in a buffer solution, for example, a citrate buffer solution (e.g., 10 mM citrate, pH 5-6.5). Buffer solutions such as PBS, sodium phosphate, potassium phosphate, citrate buffer, may be used for fusion at pH > 7.

Alternatively or in addition, the fusion is carried out at a particular pH or within a particular pH range. In some embodiments, the fusion is carried out below neutral pH or below physiological pH. In some embodiments, the fusion is carried out at neutral pH or at physiological pH. In some embodiments, the fusion is carried out above neutral pH or at physiological pH. In some embodiments, the fusion is carried out at within a wide range of pH (e.g., pH of 1-12). In some embodiments, the fusion is carried out at acidic or neutral or physiological pH (e.g., pH of 1-7.5). In some embodiments, the fusion is carried out at a pH below pH 7, e.g., at about pH 6.5 to about pH 4.5, or at about pH 1 to about pH 4.5. In some embodiments, the fusion is carried out at a physiological pH or neutral pH or at a pH above neutral pH, e.g., at about pH pH 7 to about pH 7.4, at about pH 7 to about pH 8, at about pH 8 to about pH 9, or at about pH 9 to about pH 12.

In some embodiments, the lipid nanoparticles such as liposomes comprise one or more ionizable cationic lipids (e.g., DODMA), the fusion step may be carried out at a pH below 7, for example, at a pH between 5-6.5. Such lipid nanoparticles may carry a nucleic acid-based cargo, such as antisense oligonucleotides, siRNAs, or mRNAs. In other embodiments, the lipid nanoparticles such as liposomes comprise one or more non-ionizable cationic lipids (e.g., DOTAP), the fusion step may be carried out at any pH conditions. In some embodiments, the LNP or liposome comprises PEGylated lipids. In some embodiments, the LNP or liposome does not comprise PEGylated lipids. In some embodiments, the helper lipid is selected from DOPC or DSPE.

In some embodiments, fusion of the cargo-carrying lipid nanoparticle and the MPV, e.g., WPV, is achieved by extrusion. For example, a suspension comprising the cargo-carrying lipid nanoparticle and the MPV, e.g., WPV, can be prepared via routine methodology and subject to extrusion for one or multiple times through a suitable filter under pressure. The ratio between the cargo-carrying lipid nanoparticle and the MPV, e.g., WPV, in the suspension may range from 10:1 to 1:10, for example, 5:1 to 1:5. For example, in some embodiments, the ratio between the cargo-carrying lipid nanoparticle and the MPV, e.g., WPV, in the suspension is 5:1, 4:1, 3:1, 2:1, 1:1, 1:2, 1:3, 1:4, or 1:5. In some embodiments, the LNP to WPV ratio is 10:1 or greater. In some embodiments, the LNP to WPV ratio is 20:1, 30:1, 40:1, 50:1, 60:1, 70:1, 80:1, 90:1, 100:1 or any increment therein. In some embodiments the LNP to WPV ratio is 100:1 or greater. In one embodiment, the ratio is 1:1. In some embodiments, the filter comprises a polycarbonate membrane. Alternatively or in addition, in some embodiments, the membrane of the filter has a pore size of about 50 nm to about 200 nm (e.g., about 50 nM to about 150 nm, about 50 to about 100 nm, about 100 to about 200 nm, or about 150 nm to about 200 nm). In some embodiments, the filter comprises more than one membrane, each having a different pore size. For example, in some embodiments, the filter comprises three membranes having pore sizes of 50 nm, 100 nm, and 200 nm. During extrusion, the suspension goes through the three membranes sequentially to form the LNP-MPVs. In some embodiments, the extrusion step is repeated, for example, for 2-10 times (e.g., 2-8 times, 2-6 times, or 2-5 times).

In some examples, the lipid nanoparticles used in the fusion method have a size of below 50 nm. The ratio between such lipid nanomarticles and MPVs, e.g., WPVs, may range from 1:1 to 10:1. In other examples, the lipid nanoparticles have a size of above 50 nm and the ratio between the lipid nanoparticles and MPVs, e.g., WPVs, may rnage from 1:2 to 5:1.

In some embodiments, the fusion step disclosed herein is performed using a device containing multiple tubes forming a Y junction or a T junction. In some embodiments, the cargo-carrying lipid nanoparticles and the MPVs, e.g., WPVs, flow through tubes via pumps and the two types of particles interact with each other at Y or T junctions of the tubes, wherein LNP-MPVs encapsulating the cargo form. In some embodiments, the tubes have a diameter of about 0.2-2 mm. In some embodiments, the fusion step utilizes a microfluidic device as disclosed herein. In some embodiments, the microfluidic device used herein comprises one or more channels (e.g., of glass and/or polymer materials) having a diameter less than 2 mm, for example, about 0.02-2 mm. In some examples, the one or more channels may have a diameter of about 0.05-2 mm. In some examples, the one or more channels may have a diameter of about 0.1-2 mm. In some examples, the one or more channels may have a diameter of about 0.2-2 mm. In some examples, the one or more channels may have a diameter of about 0.5-2 mm. In some examples, the one or more channels may have a diameter of about 0.8-2 mm.

In any of the fusion methods disclosed herein (e.g., extrusion-mediated or PEG-mediated fusion), lipid nanoparticles and MPVs, e.g., WPVs, capable of binding to each other may be selected to enhance fusion efficiency. In some examples, the lipid nanoparticles may be modified to carry a surface targeting moiety that is capable of binding to the MPV, e.g., WPV, so as to enhance fusion efficiency. For example, the lipid nanoparticles may be modified to display a lectin, which is capable of binding to glycoproteins on naturally-occurring MPVs. Accordingly, in some embodiments, the lipid nanoparticles display lectins on their surface. Exemplary lectins for use in this targeted fusion include Con A, RCA, WGA, DSL, Jacalin, or any combination thereof. Accordingly, in some embodiments, the lipid nanoparticles display one or more lectins selected from Con A, RCA, WGA, DSL, Jacalin, or any combination thereof. Alternatively, the lipid nanoparticles may be modified to display a binding moiety capable of binding to another binding moiety that is conjugated to the surface of the MPVs. Such binding moiety pairs may be any ligand-receptor pairs such as biotin-streptavidin. Alternatively, lipid nanoparticles and MPVs, e.g., WPVs, having lipid contents with opposite electrostatic charges may be used. For example, fusion may be carried out between cargo-carrying lipid nanoparticles comprising positively charged lipids and MPVs, e.g., WPVs, comprising negatively charged lipids. In some examples, the positively charged lipids are ionizable cationic lipids. In other examples, the positively charged cationic lipids are non-ionizable cationic lipids. When an ionizable cationic lipid is used, a suitable pH range may be selected, under which the ionizable cationic moiety of the lipid predominantly has a positive charge status.

In some embodiments, the glycan residues and/or glycoproteins, as well as glycolipids, provide a charge on the MPV, e.g., WPV, that is opposite to the electric charge of the lipid nanoparticle. For example, fusion may be carried out between cargo-carrying lipid nanoparticles comprising positively charged lipids and MPVs, comprising negatively charged lipids and/or glycan residues which may be in a glycoprotein or glycolipid.

In some embodiments, the LNP-MPVs encapsulating the cargo have substantially similar physical and/or chemical features as the MPV, e.g., WPV, used in the fusion such that the resultant LNP-MPV would retain the advantageous features as MPVs, e.g., WPVs, for oral delivery of the cargo to a subject. This goal may be achieved by using lipid nanoparticles having similar lipid contents and/or protein contents as the MPVs, e.g., WPVs, for fusion. Accordingly, in some embodiments, lipid nanoparticles and MPVs, e.g., WPVs, employed for fusion have similar lipid contents and/or protein contents. Alternatively, one may use lipid nanoparticles that are much smaller than the MPVs, e.g., WPVs, such that the lipid and/or protein contents of the MPVs, e.g., WPVs, would not have significant change after being fused with the lipid nanoparticle.

### (ii) Enrichment of LNP-MPVs Encapsulating the Cargo

After the fusion step, the resultant fused vesicles, which carry the cargo, may be enriched by conventional methods or approached disclosed herein, e.g., ion-exchange chromatography, affinity chromatography, tangential flow filtration (TFF), or a combination thereof. For example, the LNP-MPVs may be selectively collected by negative selection (e.g., excluding lipid nanoparticles) or positive selection (e.g., collecting specifically the LNP-MPVs). In some examples, the LNP-MPVs may be enriched by fractionation based on particle size, for example, SEC. In other examples, the LNP-MPVs may be enriched via an affinity binding approach, using a target molecule that specifically binds LNP-MPVs. Such target molecule may be a lectin, for example, Con A, RCA, WGA, DSL, Jacalin, and any combination thereof. In yet other examples, the LNP-MPVs may be enriched using one or more columns (e.g., an ion-exchange column and/or an affinity column) that selectively bind unfused lipid nanoparticles and/or MPV, e.g., WPVs. Alternatively, the LNP-MPVs may be enriched using one or more columns (e.g., an ion-exchange column and/or an affinity column) that selectively bind the LNP-MPVs.

In some embodiments, the LNP-MPVs derived from fusion of MPVs, e.g., WPVs, and cargo-loaded lipid nanoparticles may be further modified to produce surface programmed LNP-MPVs, which are the final product for use in oral delivery of the cargo loaded therein to a subject in need thereof.

### IV. LNP-MPVs

Any of the LNP-MPVs produced, isolated, enriched, purified by any of the methods disclosed herein, and/or surface modified, are also within the scope of the present disclosure. In some embodiments, the LNP-MPVs are a fusion product resulting from any of the fusion-based methods disclosed herein. Such fused vesicles, i.e., LNP-MPVs a.k.a., duosomes, may be modified to attach a surface targeting moiety capable of binding to specific gut cells such as small intestinal cells, to produce surface programmed LNP-MPVs, such as surface programmed liposome-WPVs. Such surface programmed LNP-MPVs can be prepared in a composition for oral administration. Alternatively, LNP-MPVs may be used directly for oral administration.

In some embodiments, MPVs, e.g., WPVs, described herein and used in the methods described herein confer certain biological components to the LNP-MPV. Accordlingly, the fused vesicles, i.e. LNP-MPVs, e.g., liposome-WPVs, generated according to the methods described herein comprise certain biological components characteristic of the MPVs, e.g., WPVs, used in the methods. Such biological components are described herein and include but are not limited to lipid, protein, glycoprotein, glycolipid, lipoprotein, phospholipid, phosphoprotein, peptide, glycan, fatty acid, sterol, steroid, and combinations thereof. In some embodiments, MPVs, e.g., WPVs, described herein and used in the methods described herein bestow certain properties, which are characteristic of the MPV, to the fused vesicle, i.e., the LNP-MPV, including but not limited to stability to chemical and mechanical stress. These properties are not characteristic of the original LNP used in the fusion method, i.e., the LNP into which the cargo was originally loaded. Such properties include stability at low pH and resistance to digestive enzymes. These and other properties make the LNP-MPV, e.g., a liposome-WPV, a suitable vehicle for oral administration and/or delivery of a cargo, such as the cargos described herein. In some embodiments, the LNP-MPVs or compositions of LNP-MPVs provided herein are used for oral delivery of a cargo, e.g., a cargo encapsulated in the LNP-MPV. In some embodiments, the LNP-MPVs, e.g., liposome-WPVs, are formulated to form a suitable composition for use in oral delivery of the cargo encapsulated therein to a subject, for example, a human patient.

In some embodiments, the relative abundance of casein in the composition comprising the LNP-MPVs, e.g., liposome-WPVs, is less than about 40% as compared with the total protein in the composition comprising the LNP-MPVs. In some embodiments, the relative abundance of casein in the composition comprising the LNP-MPVs, e.g., liposome-WPVs,is less than about 30% as compared with the total protein in the composition comprising the LNP-MPVs. In some embodiments, the relative abundance of casein in the composition comprising the LNP-MPVs, e.g., liposome-WPVs, is less than about 20% as compared with the total protein in the composition comprising the LNP-MPVs. In some embodiments, the relative abundance of casein in the composition comprising the LNP-MPVs, e.g., liposome-WPVs, is less than about 10% as compared with the total protein in the composition comprising the LNP-MPVs. In some embodiments, the relative abundance of casein in the composition comprising the LNP-MPVs, e.g., liposome-WPVs, is less than about 5% as compared with the total protein in the composition comprising the LNP-MPVs. In some embodiments, the relative abundance of casein in the composition comprising the LNP-MPVs, e.g., liposome-WPVs, is less than about 4% as compared with the total protein in the composition comprising the LNP-MPVs. In some embodiments, the relative abundance of casein in the composition comprising the LNP-MPVs, e.g., liposome-WPVs, is less than about 3% as compared with the total protein in the composition comprising the LNP-MPVs. In some embodiments, the relative abundance of casein in the composition comprising the LNP-MPVs, e.g., liposome-WPVs, is less than about 2% as compared with the total protein in the composition comprising the LNP-MPVs. In some embodiments, the relative abundance of casein in the composition comprising the LNP-MPVs, e.g., liposome-WPVs, is less than about 1% as compared with the total protein in the composition comprising the LNP-MPVs. In some embodiments, the relative abundance of lactoglobulin in the composition comprising the LNP-MPVs, e.g., liposome-WPVs, is less than about 25% (e.g., less than about 20%, less than about 15%, less than about 10%, less than about 5%, less than about 4%, less than about 3%, less than about 2%, less than about 1%) as compared with the total protein in the composition comprising the LNP-MPVs. In some embodiments, the relative abundance of casein in the composition comprising the LNP-MPVs, e.g., liposome-WPVs, is less than about 40% (e.g., less than about 30%, less than about 20%, less than about 10%, less than about 5%,less than about 4%, less than about 3%, less than about 2%, less than about 1% as compared with the total protein in the composition comprising the LNP-MPVsand/or the relative abundance of lactoglobulin in the composition comprising the LNP-MPVs, e.g., liposome-WPVs,is less than about 25% (e.g., less than about 20%, less than about 15%, less than about 10%, less than about 5%, less than about 4%, less than about 3%, less than about 2%, less than about 1%) as compared with the total protein in the composition comprising the LNP-MPVs..

The radius of an LNP-MPV, e.g., liposome-WPV, produced according to the methods described herein can be calculated according to the following formula, if it is assumed that one fused vesicle particle is fusing with one LNP: R(fused)³=R (fused vesicle)³ +R(LNP)³. In some embodiments, the LNP-MPV, e.g., liposome-WPV, produced by any of the methods described herein is a result of one MPV, e.g., WPV, particle fusing with one LNP. In some embodiments, the LNP-MPV, e.g., liposome-WPV, produced by any of the methods described herein is a result of one MPV, e.g., WPV, particle fusing with more than one LNP. In some embodiments, the LNP-MPV, e.g., liposome-WPV, produced by any of the methods described herein is a result of one MPV, e.g., WPV, particle fusing with 2, 3 or 4 LNPs. In some embodiments, the LNP-MPV, e.g., liposome-WPV, produced by any of the methods described herein is a result of more than one MPV, e.g., WPV, particle, e.g., 2, 3 or 4 MPVs, e.g., WPVs, fusing with one LNP. In some embodiments, the LNP-MPV, e.g., liposome-WPV, produced by any of the methods described herein is a result of more than one MPV, e.g., WPV, particle fusing with more than one LNP.

In some embodiments, the LNP-MPV, e.g., liposome-WPV, are derived from MPVs, e.g., WPVs, that are modified from their natural state. In some embodiments, the MPV, e.g., WPV, from which the LNP-MPV, e.g., liposome-WPV, is derived is modified to alter one or more lipids, proteins, glycoproteins, glycolipids, lipoproteins, phospholipids, phosphoproteins, peptides, glycans, fatty acids, and/or sterols present in the natural MPV, e.g., WPV. In some embodiments, the MPVs, e.g., WPVs, are modified by altering the quantity, concentration, or amount of a biomolecule naturally present, e.g., the addition or complete or partial removal of a biomolecule naturally present (e.g., carbohydrate, such as a glycan and/or glycan residue; fatty acid, lipid). In some embodiments, the MPV, e.g., WPV, from which the LNP-MPV, e.g., liposome-WPV, is derived is modified by the addition of a biomolecule not naturally present (e.g., carbohydrate, such as a glycan; fatty acid; lipid; or protein, e.g., glycoprotein). Accordlingly, in some embodiments, the LNP-MPVs, e.g., liposome-WPVs, comprise an altered quantity, concentration, or amount of a biomolecule (e.g., lipids, proteins, glycoproteins, glycolipids, lipoproteins, phospholipids, phosphoproteins, peptides, glycans, fatty acids, and/or sterols) naturally present relative to an LNP-MPV derived from an unmodified, naturally occurring MPV, e.g., WPV. In some embodiments, the LNP-MPV, e.g., liposome-WPV, comprises additional biomolecules (e.g., additional lipids, proteins, glycoproteins, glycolipids, lipoproteins, phospholipids, phosphoproteins, peptides, glycans, fatty acids, and/or sterols) relative to an LNP-MPV derived from an unmodified, naturally occurring MPV, e.g., WPV.

In some embodiments, the LNP-MPV, e.g., liposome-WPV, comprises one or more additional proteins relative to an LNP-MPV, e.g., liposome-WPV, derived from an unmodified, naturally occurring MPV, e.g., WPV.

In some embodiments, the MPV, e.g., WPV, and/or the resultant fused MPV, e.g., WPV, comprises a targeting moiety for tissue specific localization and/or delivery. Exemplary targeting moieties include, but are not limited to, a compound comprising at least one N-acetylgalactosamine (GalNAc) moiety (e.g., a compound comprising two or three GalNAc moieties), folate, an antibody (e.g., a Fab fragment), a nucleic acid aptamer, a RGD peptide, or a lectin. Accordlingly, in some embodiments, the LNP-MPV is a surface loaded or surface programmed LNP-MPV. In some embodiments, the LNP-WPV is a surface loaded or surface programmed liposome-WPV. In some embodiments, a cargo is a targeting moiety. In some embodiments, the surface of the MPV, e.g., WPV, and/or LNP-MPV, e.g., liposome-WPV, is programmed or functionalized with ligands or targeting moieties to improve intestinal uptake for improved oral delivery. In some embodiments, the targeting moiety promotes LNP-MPVs, e.g., liposome-WPVs, binding to the intestinal lining within the intestine. In some embodiments, the targeting moiety promotes localization of the MPV, e.g., WPV, or LNP-MPV, e.g., liposome-WPV, to a specific section of the intestine. In some embodiments, the targeting moiety promotes vesicle binding and localization within the intestine. In some embodiments, the surface of the vesicle is programmed to target and/or bind to specific intestinal mucosal cell types, including, but not limited to, enterocytes, M cells or immune cells. In some embodiments, the targeting moiety targets a specific area in the intestine or gut, e.g., for targeted oral delivery or administration of an LNP-MPV, e.g., liposome-WPV, (which comprises a cargo), e.g., the small or the large intestine. In some embodiments, the targeting moiety targets the duodenum. In some embodiments, the targeting moiety targets the jejunum. In some embodiments, the targeting moiety targets the stomach. In some embodiments, the targeting moiety targets the colon. In some embodiments, the ligand or targeting moiety comprises one or more lectin(s), alone or in combination with one or more other targeting moieties, e.g., antibodies. Non-limiting examples of suitable lectins are listed elsewhere herein and for example described in Diesner et al., Therapeutic Delivery (2012) 3(2). In some embodiments, the same one or more lectin(s) are used both as a targeting moiety displayed on a MPV, e.g., WPV, and/or LNP-MPV, e.g., liposome-WPV, and for targeted fusion of a MPV, e.g., WPV, with a nanoparticle as described herein. In some embodiments, different lectin(s) are used as a targeting moiety displayed on a MPV, e.g., WPV, and/or LNP-MPV, e.g., liposome-WPV, and for targeted fusion of a MPV, e.g., WPV, with a nanoparticle as described herein.

In some embodiments, the LNP-MPV, e.g., liposome-WPV, are modified to display a lectin, which is capable of binding to glycoproteins, e.g., a glycoprotein present on a nanoparticle. Accordingly, in some embodiments, the LNP-MPVs, e.g., liposome-WPVs, display lectins on their surface. In some embodiments, the LNP-MPVs, e.g., liposome-WPVs, display one or more lectins selected from Con A, RCA, WGA, DSL, Jacalin, or any combination thereof. In some embodiments, the MPVs, e.g., WPVs, used in the methods described herein comprise one or more lectins, which are then conferred to the LNP-MPV, e.g., liposome-WPV, produced by the methods described herein. Accordlingly, in some embodiments, a method of producing an LNP-MPV, e.g., liposome-WPV, comprising one or more lectins comprises contacting a MPV, e.g., WPV, comprising one or more lectins or a composition comprising such MPVs, e.g., WPVs, with a lipid nanoparticle or a composition comprising such lipid nanoparticles, e.g., a nanoparticles comprising a cargo, as described herein and optionally collecting the resulting LNP-MPVs. In some embodiments, the one or more lectins naturally occur on the MPV, e.g., WPV. In some embodiments, the one or more lectins do not naturally occur on the MPV, e.g., WPV, In some embodiments, the lipid nanoparticles used in the methods described herein for fusion comprise one or more lectins, which are then conferred to the LNP-MPV, e.g., liposome-WPV, produced by the methods described herein. Accordlingly, in one embodiment, a method of a method of producing an LNP-MPV, e.g., liposome-WPV, comprising one or more lectins comprises contacting a nanoparticle comprising one or more lectins or a composition comprising such nanoparticles, e.g., a nanoparticles comprising a cargo, as described herein, with a MPV, e.g., WPV, or a composition comprising such MPV and optionally collecting the resulting LNP-MPV, e.g., liposome-WPV. In some embodiments, a method of producing an LNP-MPV, e.g., liposome-WPV, comprising one or more lectins comprises contacting the LNP-MPVs, e.g., liposome-WPVs, directly with a lectin, thereby producing the desired vesicle comprising a lectin.

In some embodiments, the LNP-MPV, e.g., liposome-WPV, size, or LNP-MPVaverage size is greater than the size of the MPV, e.g., WPV, or average size of the MPV, used in the fusion method. In some embodiments, the LNP-MPV, size, or average size is not significantly greater or essentially equivalent to the size or average size of the MPV, e.g., WPV, used in the fusion method. In some embodiments, the LNP-MPV is about 20 nm - 1000 nm in diameter or size. In some embodiments, the LNP-MPV, e.g., liposome-WPV, is about 20 nm to about 200 nm in size. In some embodiments, the LNP-MPV, e.g., liposome-WPV, is about 20 nm to about 190 nm or about 25 nm to about 190 nm in size. In some embodiments, the LNP-MPV, e.g., liposome-WPV, is about 30 nm to about 180 nm in size. In some embodiments, the LNP-MPV, e.g., liposome-WPV, is about 35 nm to about 170 nm in size. In some embodiments, LNP-MPV, e.g., liposome-WPV, is about 40 nm to about 160 nm in size. In some embodiments, the LNP-MPV, e.g., liposome-WPV, is about 50 nm to about 150 nm, about 60 nm to about 140 nm, about 70 nm to about 130 nm, about 80 nm to about 120 nm, or about 90 nm to about 110 nm in size. In some embodiments, the LNP-MPV, e.g., liposome-WPV, is about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in size or diameter. In some embodiments, an average size of an LNP-MPV, e.g., liposome-WPV, in an LNP-MPV composition or plurality of LNP-MPVs produced according to the methods described herein is about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in average size. In some embodiments, an average size of an LNP-MPV, e.g., liposome-WPV, in an LNP-MPV composition or plurality of LNP-MPV is about 20 nm to about 200 nm, about 20 nm to about 190 nm, about 25 nm to about 190 nm, about 30 nm to about 180 nm, about 35 nm to about 170 nm, about 40 nm to about 160 nm, about 50 nm to about 150, about 60 to about 140 nm, about 70 to about 130, about 80 to about 120, or about 90 to about 110 nm in average size.

In some embodiments, the LNP-MPV, e.g., liposome-WPV, is about 20 nm to about 100 nm in size. In some embodiments, the LNP-MPV, e.g., liposome-WPV, is about 25 nm to about 95 nm in size. In some embodiments, the LNP-MPV, e.g., liposome-WPV, is about 20 nm to about 90 nm in size. In some embodiments, the LNP-MPV, e.g., liposome-WPV, is about 20 nm to about 85 nm in size. In some embodiments, the LNP-MPV, e.g., liposome-WPV, is about 20 nm to about 80 nm in size. In some embodiments, the LNP-MPV, e.g., liposome-WPV, is about 20 nm to about 75 nm in size. In some embodiments, the LNP-MPV, e.g., liposome-WPV, is about 20 nm to about 70 nm in size. In some embodiments, the LNP-MPV, e.g., liposome-WPV, is about 25 nm to about 80 nm in size. In some embodiments, the LNP-MPV, e.g., liposome-WPV, is about 30 nm to about 70 nm in size. In some embodiments, the LNP-MPV, e.g., liposome-WPV, is about 30 nm to about 60 nm in size. In some embodiments, the LNP-MPV, e.g., liposome-WPV, is about 40 nm to about 70 nm in size. In some embodiments, the LNP-MPV, e.g., liposome-WPV, is about 40 nm to about 60 nm in size. In some embodiments, an average vesicle size in a vesicle composition or plurality of vesicles isolated or purified from milk is about 20 nm to about 100 nm, about 20 nm to about 95 nm, about 20 nm to about 90 nm, about 20 nm to about 85 nm, about 20 nm to about 80 nm, about 20 to about 75 nm, about 25 nm to about 85 nm, about 25 nm to about 80, about 25 to about 75 nm, about 30 to about 80 nm, about 30 to about 85 nm, about 30 to about 75 nm, about 40 to about 80, about 40 to about 85 nm, about 40 to about 75 nm, about 45 to about 80 nm, about 45 to about 85, about 45 to about 75 nm, about 50 to about 75 nm, about 50 to about 80 nm, about 50 to about 85 nm, about 55 to about 75 nm, about 55 to about 80 nm, about 55 to about 85 nm, about 60 to about 75 nm, about 60 to about 80 nm, about 60 to about 85 nm, about 25 to about 70 nm, about 30 to about 70 nm, about 40 to about 70 nm, about 50 to about 70 nm, about 30 to about 60 nm, about 30 to about 50 nm in average size.

In some embodiments, the LNP-MPV, e.g., liposome-WPV, is about 80 nm to about 200 nm in size. In some embodiments, the LNP-MPV, e.g., liposome-WPV, is about 85 nm to about 195 nm in size. In some embodiments, the LNP-MPV, e.g., liposome-WPV, is about 90 nm to about 190 nm in size. In some embodiments, the LNP-MPV, e.g., liposome-WPV, is about 95 nm to about 185 nm in size. In some embodiments, the LNP-MPV, e.g., liposome-WPV, is about 100 nm to about 180 nm in size. In some embodiments, the LNP-MPV, e.g., liposome-WPV, is about 105 nm to about 175 nm in size. In some embodiments, the LNP-MPV, e.g., liposome-WPV, is about 110 nm to about 170 nm in size. In some embodiments, the LNP-MPV, e.g., liposome-WPV, is about 115 nm to about 165 nm in size. In some embodiments, the LNP-MPV, e.g., liposome-WPV, is about 120 nm to about 160 nm in size. In some embodiments, the LNP-MPV, e.g., liposome-WPV, is about 125 nm to about 155 nm in size. In some embodiments, the LNP-MPV, e.g., liposome-WPV, is about 130 nm to about 150 nm in size. In some embodiments, the LNP-MPV, e.g., liposome-WPV, is about 135 nm to about 145 nm in size. In some embodiments, the LNP-MPV, e.g., liposome-WPV, is about 110 nm to about 150 nm in size. In some embodiments, an average vesicle size in a vesicle composition or plurality of vesicles isolated or purified from milk is about 80 nm to about 200 nm, about 80 nm to about 190 nm, about 80 nm to about 180 nm, about 80 nm to about 170 nm, about 80 nm to about 160 nm, about 80 to about 150 nm, about 80 nm to about 140 nm, about 80 nm to about 130, about 80 to about 120 nm, about 80 to about 110 nm, about 80 to about 100 nm, about 30 to about 75 nm, about 40 to about 80, about 40 to about 85 nm, about 40 to about 75 nm, about 45 to about 80 nm, about 45 to about 85, about 45 to about 75 nm, about 50 to about 75 nm, about 50 to about 80 nm, about 50 to about 85 nm, about 55 to about 75 nm, about 55 to about 80 nm, about 55 to about 85 nm, about 60 to about 75 nm, about 60 to about 80 nm, about 60 to about 85 nm, about 25 to about 70, about 30 to about 70, about 40 to about 70 nm, about 50 to about 70 nm, about 30 to about 60 nm, about 30 to about 50 nm in average size.

In some embodiments, the LNP-MPV, e.g., liposome-WPV, is greater than 200 nm in size. In some embodiments, the LNP-MPV, e.g., liposome-WPV, is about 200 to about 1000 nm in size. In some embodiments, the LNP-MPV, e.g., liposome-WPV, is about 200 to about 400 nm in size, e.g., about 200 nm to about 250 nm, about 250 nm to about 300 nm, about 300 to about 350 nm, about 350 nm to about 400 nm in size. In some embodiments, the LNP-MPV, e.g., liposome-WPV, is about 400 to about 600 nm in size, e.g., about 400 nm to about 450 nm, about 450 nm to about 500 nm, about 500 to about 550 nm, about 550 nm to about 600 nm in size. In some embodiments, the LNP-MPV, e.g., liposome-WPV, is about 600 to about 800 nm in size, e.g., about 600 nm to about 650 nm, about 650 nm to about 700 nm, about 700 to about 750 nm, about 750 nm to about 800 nm in size. In some embodiments, the LNP-MPV, e.g., liposome-WPV, is about 800 to about 1000 nm in size, e.g., about 800 nm to about 850 nm, about 850 nm to about 900 nm, about 900 to about 950 nm, about 950 nm to about 1000 nm in size. In some embodiments, an average vesicle size in an LNP-MPV , e.g., liposome-WPV composition or plurality of LNP-MPVs isolated or purified from milk is about 200 nm to about 1000 nm, about 200 nm to about 900 nm, about 200 nm to about 800 nm, about 200 nm to about 700 nm, about 200 nm to about 600 nm, about 200 to about 500 nm, about 200 nm to about 400 nm, about 200 nm to about 300, about 300 to about 1000 nm, about 300 to about 900 nm, about 300 to about 800 nm, about 300 to about 700 nm, about 300 to about 600, about 300 to about 500 nm, about 300 to about 400 nm, about 400 to about 1000 nm, about 400 to about 900, about 400 to about 800 nm, about 400 to about 700 nm, about 400 to about 600 nm, about 400 to about 500 nm, about 500 to about 1000 nm, about 500 to about 900 nm, about 500 to about 800 nm, about 500 about 700 nm, about 500 to about 600 nm, about 600 to about 1000 nm, about 600 to about 900 nm, about 600 to about 800 nm, about 600 to about 700 nm, about 700 to about 1000 nm, about 700 to about 900 nm, about 700 to about 800 nm, about 800 to about 1000 nm, about 800 to about 900 nm, about 900 to about 1000 nm in average size.

In any of the above embodiments relating to LNP-MPV, e.g., liposome-WPV, size, the LNP-MPVs , e.g., liposome-WPVs, or compositions of LNP-MPVscomprise a relative abundance of casein less than about 40%, about 35%, about 30%, about 25%, about 20%, about 15%, about 10%, about 5% or less. In some of these above embodiments, the LNP-MPVs, e.g., liposome-WPVs, or compositions of LNP-MPVs produced by the fusion methods described herein are substantially free of casein. In some of these above embodiments, the LNP-MPVs, e.g., liposome-WPVs, or compositions of LNP-MPVscomprise lactoglobulin at a relative abundance of no greater than 25% (e.g., less than about 25%, about 20%, about 15%, about 10%, about 5% or less). In some embodiments, the LNP-MPVs, e.g., liposome-WPVs, or compositions of LNP-MPVsmay be substantially free of lactoglobulins. In some of these above embodiments, the LNP-MPVs, e.g., liposome-WPVs, comprise a lipid membrane to which one or more proteins described herein are associated. In some embodiments, the LNP-MPVs, e.g., liposome-WPVs, are derived from MPVs, e.g., WPVs, that are not modified from their naturally occurring state. In some embodiments, the the LNP-MPVs, e.g., liposome-WPVs, are derived from MPVs, e.g., WPVs, that are modified from their natural state. In some embodiments, the MPVs, e.g., WPVs, are modified by altering the quantity, concentration, or amount of a biomolecule naturally present, e.g., the addition or complete or partial removal of a biomolecule naturally present (e.g., carbohydrate, such as a glycan and/or glycan residue; fatty acid, lipid). In some embodiments, the MPV, e.g., WPV, is modified by the addition of a biomolecule not naturally present (e.g., carbohydrate, such as a glycan; fatty acid; lipid; or protein, e.g., glycoprotein). Accordlingly, in some embodiments, the the LNP-MPVs , e.g., liposome-WPVs, comprise an altered quantity, concentration, or amount of a biomolecule naturally present relative to an LNP-MPV derived from an unmodified, naturally occurring MPV, e.g., WPV. In some embodiments, the LNP-MPV, e.g., liposome-WPV, comprises additional biomolecules relative to an LNP-MPV derived from an unmodified, naturally occurring MPV, e.g., WPV. In some embodiments, the LNP-MPVs, e.g., liposome-WPVs comprise one or more proteins selected from BTN1A1, CD81 and XOR. In some embodiments, one or more proteins associated with the lipid membrane of the LNP-MPVs, e.g., liposome-WPVs are glycosylated. In some embodiments, the LNP-MPVs, e.g., liposome-WPVs demonstrate stability under freeze-thaw cycles and/or temperature treatment. In some embodiments, the LNP-MPVs, e.g., liposome-WPVs demonstrate colloidal stability when loaded with the biological molecule. In some embodiments, the LNP-MPVs, e.g., liposome-WPVs demonstrate stability under acidic pH, e.g., pH of ≤ 4.5 or pH of ≤2.5. In some embodiments, the LNP-MPVs, e.g., liposome-WPVs demonstrate stability upon sonication. In some embodiments, the LNP-MPVs, e.g., liposome-WPVs demonstrate resistance to enzyme digestion, e.g., resistance to one or more digestive enzymes described herein and/or resistance to nuclease treatment. In any of these embodiments, the LNP-MPVs, e.g., liposome-WPVs can be used for oral delivery of a cargo, e.g., a cargo encapsulated in the LNP-MPVs. In some embodiments, the LNP-MPVs, e.g., liposome-WPVs are formulated to form a suitable composition for use in oral delivery of the cargo encapsulated therein to a subject, for example, a human patient. In some embodiments, the cargo can be a peptide, a protein, a nucleic acid, a polysaccharide, or a small molecule.

In some embodiments, the LNP-MPV, e.g., liposome-WPV, comprises one or more polypeptides comprised in the MPV, e.g., WPV, used in the fusion method. In some embodiments, the LNP-MPV, e.g., liposome-WPV comprises lower levels of the one or more polypeptides comprised in the MPV, e.g., WPV, used in the fusion method. In some embodiments, the LNP-MPV, e.g., liposome-WPV comprises essentially the same or similar levels, e.g., not significantly lower levels of the one or more polypeptides comprised in the MPV, e.g., WPV, used in the fusion method. In some embodiments, the LNP-MPV, e.g., liposome-WPV comprises one or more polypeptides selected from the following polypeptides: butyrophilin subfamily 1, butyrophilin subfamily 1 member A1, butyrophilin subfamily 1 member A1 isoform X2, butyrophilin subfamily 1 member A1 isoform X3, serum albumin, fatty-acid binding protein, fatty acid binding protein (heart), lactadherin, lactadherin isoform X1, beta-lactoglobin, beta-lactoglobin precursor, lactotransferrin precursor, alpha-S1-casein isoform X4, alpha-S2-casein precursor, casein, kappa-casein precursor, alfa-lactalbumin precursor, platelet glycoprotein 4, xanthine dehydrogenase oxidase, ATP-binding cassette sub-family G, perilipin, perilipin-2 isoform X1, RAB1A (member RAS oncogene family), peptidyl-prolyl cis-trans isomerase A, ras-related protein RAB-18, EpCam, CD81, TSG101, HSP70, polymeric immunoglobulin receptor, lactoferrin, CD63, Tsg101, Alix, CD81, and lactoperoxidase isoform X1. In some embodiments, the LNP-MPV, e.g., liposome-WPV comprises butyrophilin. In some embodiments, the LNP-MPV, e.g., liposome-WPV comprises butyrophilin subfamily 1. In some embodiments, the LNP-MPV, e.g., liposome-WPV comprises butyrophilin subfamily 1 member A1(BTN1A1). In some embodiments, the LNP-MPV, e.g., liposome-WPV comprises lactadherin. In some embodiments, the LNP-MPV, e.g., liposome-WPV comprises one or more of the following polypeptides: CD81, CD63, Tsg101, CD9, Alix, EpCAM, and XOR. In some embodiments, the LNP-MPV, e.g., liposome-WPV comprises CD81. In some embodiments, the LNP-MPV, e.g., liposome-WPV comprises XOR. In some embodiments, the LNP-MPV, e.g., liposome-WPV comprises BTN1A1 and CD81. In some embodiments, the LNP-MPV, e.g., liposome-WPV comprises BTN1A1 and XOR. In some embodiments, the LNP-MPV, e.g., liposome-WPV comprises XOR and CD81. In some embodiments, the LNP-MPV, e.g., liposome-WPV comprises BTN1A1, CD81, and XOR. In some embodiments, the LNP-MPV, e.g., liposome-WPV may comprise a fragment of any of the proteins disclosed herein, for example, the transmembrane fragment. In particular examples, the LNP-MPV, e.g., liposome-WPV may comprise BTN1A1, BTN1A2, or a combination thereof. In some embodiments, one or more of these polypeptides may enhance the stability, loading of cargo, transport, uptake into cells or tissues, and/or bioavailability of the LNP-MPV, e.g., liposome-WPV.

Any of the protein moieties in the LNP-MPV, e.g., liposome-WPV may be glycosylated, i.e., linked to one or more glycans, e.g., such as those described elsewhere herein, at one or more glycosylation sites, e.g., in a manner described elsewhere herein. In some embodiments, the LNP-MPV, e.g., liposome-WPV comprises one or more glycoproteins or glycopolypeptides having a glycan selected from: galactose, mannose, O-glycans, N-acetyl- glucosamines, and/or N-glycan chains or any combination thereof. In some embodiments, the LNP-MPV, e.g., liposome-WPV comprises one or more glycoproteins or glycopolypeptides having a glycan selected from: D- or L- glucose, erythrose, fucose, galactose, mannose, lyxose, gulose, xylose, arabinose, ribose, 2′-deoxyribose, glucosamine, lactosamine, polylactosamine, glucuronic acid, sialic acid, sialyl-Lewis X (SLex), N-acetyl-glucosamine, N- acetyl-galactosamine, neuraminic acid, N-glycolylneuraminic acid (Neu5Gc), N- acetylneuraminic acid (Neu5Ac), an N-glycan chain, an O-glycan chain, a Core 1, Core 2, Core 3, or Core 4 structure, or a phosphate- or acetate-modified analog thereof or a combination thereof. In some embodiments, the LNP-MPV, e.g., liposome-WPV comprises a glycoprotein having one or more of the following glycans: terminal b-galactose, terminal a-galactose, N-acetyl-D-galactosamine, N-acetyl-D-galactosamine, and N-acetyl-D-glucosamine. In some embodiments, any of the glycans described herein may exist in free form in the LNP-MPV, e.g., liposome-WPV.

In any of the above embodiments relating LNP-MPVor compositions of LNP-MPVs, the LNP-MPVs may comprise a relative abundance of casein less than about 40%, about 35%, about 30%, about 25%, about 20%, about 15%, about 10%, about 5% or less. In some of these above embodiments, the LNP-MPVs, e.g., liposome-WPVs or compositions of LNP-MPVs produced by the methods described herein are substantially free of casein. In some of these above embodiments, the LNP-MPVs, e.g., liposome-WPVsor compositions of LNP-MPVs comprise lactoglobulin at a relative abundance of no greater than 25% (e.g., less than about 25%, about 20%, about 15%, about 10%, about 5% or less). In some embodiments, the LNP-MPVs, e.g., liposome-WPVs or compositions of LNP-MPVs may be substantially free of lactoglobulins. In some of these embodiments, the size of the LNP-MPVis about 20-1,000 nm. In some embodiments, the size of the LNP-MPV is about 100-160 nm. In some embodiments, the LNP-MPVs, e.g., liposome-WPVsdemonstrate stability under freeze-thaw cycles and/or temperature treatment. In some embodiments, the LNP-MPVs, e.g., liposome-WPVs demonstrate colloidal stability when loaded with the biological molecule. In some embodiments, the LNP-MPVs, e.g., liposome-WPVs demonstrate stability under acidic pH, e.g., pH of ≤ 4.5 or pH of ≤2.5. In some embodiments, the LNP-MPVs, e.g., liposome-WPVs demonstrate stability upon sonication. In some embodiments, the LNP-MPVs, e.g., liposome-WPVs demonstrate resistance to enzyme digestion, e.g., resistance to one or more digestive enzymes described herein and/or resistance to nuclease treatment. In any of these embodiments, the LNP-MPVs, e.g., liposome-WPV scan be used for oral delivery of a cargo, e.g., a cargo encapsulated in the LNP-MPVs. In some embodiments, the LNP-MPVs, e.g., liposome-WPV sare formulated to form a suitable composition for use in oral delivery of the cargo encapsulated therein to a subject, for example, a human patient. In some embodiments, the cargo can be a peptide, a protein, a nucleic acid, a polysaccharide, or a small molecule.

In some embodiments, the LNP-MPVs, e.g., liposome-WPVs or compositions of LNP-MPVs contain proteins having a molecule weight of about 25-30 kDa, e.g., caseins, at a relative abundance of no greater than 40% (e.g., less than about 40%, about 35%, about 30%, about 25%, about 20%, about 15%, about 10%, about 5% or less). In some embodiments, the LNP-MPVs, or compositions of LNP-MPVs comprise a lower amount of proteins per vesicle having a molecule weight of about 25-30 kDa, e.g., caseins, than the MPV, e.g., WPV, or MPV, e.g., WPV, composition used in the fusion method. In some embodiments, the LNP-MPVs or compositions of LNP-MPVs comprise a similar amount or proteins per vesicle, e.g., not significantly lower amount of proteins having a molecular weight of about 25-30 kDa, e.g., caseins, than the MPVor MPVcomposition used in the fusion method. In some embodiments, the MPVs, e.g., WPVs, used in methods resulting in the LNP-MPVs or compositions of LNP-MPVs are substantially free of casein, e.g., casein is not detected by a conventional method or only a trace amount can be detected by the conventional method. Accordingly, in some examples, the LNP-MPVsor compositions of LNP-MPVs may be substantially free of casein, e.g., are not detected by a conventional method or only a trace amount can be detected by the conventional method. Alternatively or in addition, the LNP-MPVs or compositions of LNP-MPVs contain proteins having a molecular weight of about 10-20 kDa, e.g., lactoglobulins, at a relative abundance of no greater than 25% (e.g., less than about 25%, about 20%, about 15%, about 10%, about 5% or less). In some examples, the LNP-MPVs or compositions of LNP-MPVs may be substantially free of proteins having a molecular weight of about 10-20 kDa, e.g., lactoglobulins.

In any of the above embodiments relating to casein and/or lactoglobulin abundance, the size of the LNP-MPVs, e.g., liposome-WPVs is about 20-1,000 nm. In some embodiments, the size of the LNP-MPVs, e.g., liposome-WPVs is about 100-160 nm. In some embodiments, the LNP-MPVs, e.g., liposome-WPVsare derived from MPVs, e.g., WPVs, that are not modified from their naturally occurring state. In some embodiments, the LNP-MPVs, e.g., liposome-WPVsare derived from MPVs, e.g., WPVs, that are modified from their natural state. In some embodiments, the MPVs, e.g., WPVs, are modified by altering the quantity, concentration, or amount of a biomolecule naturally present, e.g., the addition or complete or partial removal of a biomolecule naturally present (e.g., carbohydrate, such as a glycan and/or glycan residue; fatty acid, lipid). In some embodiments, the MPV, e.g., WPV, is modified by the addition of a biomolecule not naturally present (e.g., carbohydrate, such as a glycan; fatty acid; lipid; or protein, e.g., glycoprotein). Accordlingly, in some embodiments, the LNP-MPVs, e.g., liposome-WPVscomprise an altered quantity, concentration, or amount of a biomolecule naturally present relative to an LNP-MPV, e.g., liposome-WPV derived from an unmodified, naturally occurring MPV, e.g., WPV. In some embodiments, the LNP-MPV, e.g., liposome-WPVcomprises additional biomolecules relative to an LNP-MPV derived from an unmodified, naturally occurring MPV, e.g., WPV. In some of these above embodiments, the LNP-MPVs, e.g., liposome-WPVscomprise a lipid membrane to which one or more proteins described herein are associated. In some embodiments, the LNP-MPVs, e.g., liposome-WPVs, comprise one or more proteins selected from BTN1A1, CD81 and XOR. In some embodiments, one or more proteins associated with the lipid membrane of the LNP-MPVs, e.g., liposome-WPVsare glycosylated. In some embodiments, the LNP-MPVs, e.g., liposome-WPVs demonstrate stability under freeze-thaw cycles and/or temperature treatment. In some embodiments, the LNP-MPVs, e.g., liposome-WPVs demonstrate colloidal stability when loaded with the biological molecule. In some embodiments, the LNP-MPVs, e.g., liposome-WPVs demonstrate stability under acidic pH, e.g., pH of ≤ 4.5 or pH of ≤2.5. In some embodiments, the LNP-MPVs, e.g., liposome-WPVs demonstrate stability upon sonication. In some embodiments, the LNP-MPVs, e.g., liposome-WPVs demonstrate resistance to enzyme digestion, e.g., resistance to one or more digestive enzymes described herein and/or resistance to nuclease treatment. In any of these embodiments, the LNP-MPVs, e.g., liposome-WPVscan be used for oral delivery of a cargo, e.g., a cargo encapsulated in the LNP-MPVs. In some embodiments, the LNP-MPVs, e.g., liposome-WPVs are formulated to form a suitable composition for use in oral delivery of the cargo encapsulated therein to a subject, for example, a human patient. In some embodiments, the cargo can be a peptide, a protein, a nucleic acid, a polysaccharide, or a small molecule.

In some embodiments, the LNP-MPVs, e.g., liposome-WPVs described herein and/or produced by the methods described herein are stable under, for example, harsh conditions, e.g., low or high pH, sonication, enzyme digestion, freeze-thaw cycles, temperature treatment, etc. In some embodiments, a substantial portion of the LNP-MPVs, e.g., liposome-WPVs (e.g., at least 60%, at least 70%, at least 80%, at least 90%, or above) have no substantial structural changes when they are placed under an acidic condition (e.g., pH ≤ 6.5) for a period of time. In some embodiments, the LNP-MPVs, e.g., liposome-WPVs are resistant to enzymatic digestion such that a substantial portion of the LNP-MPVs, e.g., liposome-WPVs (e.g., at least 60%, at least 70%, at least 80%, at least 90%, or above) have no substantial structural changes in the presence of enzymes such as digestive enzymes. In some embodiments, the LNP-MPVs, e.g., liposome-WPVs that are stable after multiple rounds of freeze-thaw cycles (e.g., up to 6 cycles) have a substantial portion (e.g., at least 60%, at least 70%, at least 80%, at least 90%, or above) that has no substantial structural changes and/or functionality changes after the multiple freeze-thaw cycles. Acccordlingly, in some embodiments, the LNP-MPVs, e.g., liposome-WPVs are able to deliver their cargo while withstanding stressed conditions or conditions under which the therapeutic agent would become deactivated, metabolized, or decomposed, e.g., saliva, digestive enzymes, acidic conditions in the stomach, peristaltic motions, and/or exposure to the various digestive enzymes, for example, proteases, peptidases, lipases, amylases, and nucleases that break down ingested components in the gastrointestinal tract. Accordlingly, in some embodiments, the LNP-MPVs, e.g., liposome-WPVs produced by the methods described herein are used for oral administration or deliver of a cargo, e.g., a cargo encapsulated in the LNP-MPVs, e.g., liposome-WPVs.

In some embodiments, the LNP-MPV, e.g., liposome-WPV, is stable in the gut or gastrointestinal tract of a mammalian species. In some embodiments, the LNP-MPV, e.g., liposome-WPV, is stable in the esophagus of a mammalian species. In some embodiments, the LNP-MPV, e.g., liposome-WPV, is stable in the stomach of a mammalian species. In some embodiments, the LNP-MPV, e.g., liposome-WPV, is stable in the small intestine of a mammalian species. In some embodiments, the LNP-MPV, e.g., liposome-WPV, is stable in the large intestine of a mammalian species. In some embodiments, the LNP-MPV, e.g., liposome-WPV, is stable at a pH range of about pH 1.5 to about pH 7.5. In some embodiments, the LNP-MPV, e.g., liposome-WPV, is stable at a pH range of about pH 2.5 to about pH 7.5. In some embodiments, the LNP-MPV, e.g., liposome-WPV, is stable at a pH range of about pH 4.0 to about pH 7.5. In some embodiments, the LNP-MPV, e.g., liposome-WPV, is stable at a pH range of about pH 4.5 to about pH 7.0. In some embodiments, the LNP-MPV, e.g., liposome-WPV, is stable at a pH range of about pH 1.5 to about pH 3.5. In some embodiments, the LNP-MPV, e.g., liposome-WPV, is stable at a pH range of about pH 2.5 to about pH 3.5. In some embodiments, the LNP-MPV, e.g., liposome-WPV, is stable at a pH range of about pH 2.5 to about pH 6.0. In some embodiments, the LNP-MPV, e.g., liposome-WPV, is stable at a pH range of about pH 4.5 to about pH 6.0. In some embodiments, the LNP-MPV, e.g., liposome-WPV, is stable at a pH range of about pH 6.0 to about pH 7.5. In some embodiments, the LNP-MPV, e.g., liposome-WPV, is stable at a pH range of 1.5 - 7.5. In some embodiments, the LNP-MPV, e.g., liposome-WPV, is stable at a pH range of 2.5 - 7.5. In some embodiments, the LNP-MPV, e.g., liposome-WPV, is stable at a pH range of 4.0 - 7.5. In some embodiments, the LNP-MPV, e.g., liposome-WPV, is stable at a pH range of 4.5 - 7.0. In some embodiments, the LNP-MPV, e.g., liposome-WPV, is stable at a pH range of 1.5 - 3.5. In some embodiments, the LNP-MPV, e.g., liposome-WPV, is stable at a pH range of 2.5 - 3.5. In some embodiments, the LNP-MPV, e.g., liposome-WPV, is stable at a pH range of 2.5 - pH 6.0. In some embodiments, the LNP-MPV, e.g., liposome-WPV, is stable at a pH range of 4.5 - 6.0. In some embodiments, the LNP-MPV, e.g., liposome-WPV, is stable at a pH range of 6.0 - 7.5. In some embodiments, the LNP-MPV, e.g., liposome-WPV, is stable at about pH 1.5, pH 2.0, pH 2.5, pH 3.0, pH 3.5, pH 4.0, pH 4.5, pH 5.0, pH 5.5, pH 6.0, pH 6.5, pH 7.0, or pH 7.5, and increments between about pH of 1.5 and about pH 7.5.

In some embodiments, the LNP-MPV, e.g., liposome-WPV, is stable in the presence of digestive enzymes, such as, for example, proteases, peptidases, nucleases, pepsin, pepsinogen, lipase, trypsin, chymotrypsin, amylase, bile and pancreatin (digestive enzymes in pancreas). In some embodiments, the LNP-MPV, e.g., liposome-WPV, is stable in the presence of pepsin or pancreatin. In particular embodiments, the LNP-MPVs, e.g., liposome-WPVs, disclosed herein can protect cargo loaded therein (e.g., oligonucleotides) from enzyme digestion (e.g., nuclease digestion).

In some embodiments, the LNP-MPVs, e.g., liposome-WPVs, disclosed herein are stable after multiple rounds of freeze-thaw cycles. For example, the LNP-MPVs, e.g., liposome-WPVs, are stable after at least two freeze-thaw cycles, e.g., at least 3 cycles, at least 4 cycles, at least 5 cycles, or at least 6 cycles. In some instances, the LNP-MPVs, e.g., liposome-WPVs, are stable up to 10 freeze-thaw cycles, e.g., up to 9 cycles, upto to 8 cycles, up to 7 cycles, or up to 6 cycles.

In some embodiments, the LNP-MPVs, e.g., liposome-WPVs, disclosed herein are stable after temperature treatment, e.g., incubated at a low temperature (e.g., at 4° C.) for a period (e.g., 1-3 days) or at a high temperature for period, e.g., at 60-80° C. for 30 minutes to 2 hours or at 100-120° C. for 5-20 minutes.

In some embodiments, the LNP-MPVs, e.g., liposome-WPVs, disclosed herein have colloidal stability. In some embodiments, the LNP-MPVs, e.g., liposome-WPVs, are stable under physical processes, for example, sonication, centrifugation, and filtration.

In any of the above embodiments relating to LNP-MPV stability, the LNP-MPVs, e.g., liposome-WPVs or compositions of LNP-MPVs comprise a relative abundance of casein less than about 40%, about 35%, about 30%, about 25%, about 20%, about 15%, about 10%, about 5% or less. In some of these above embodiments, the LNP-MPVs, e.g., liposome-WPVs or compositions of LNP-MPVs produced by the fusion methods described herein are substantially free of casein. In some of these above embodiments, the LNP-MPVs, e.g., liposome-WPVs or compositions of LNP-MPVs lactoglobulin at a relative abundance of no greater than 25% (e.g., less than about 25%, about 20%, about 15%, about 10%, about 5% or less). In some embodiments, the LNP-MPVs, e.g., liposome-WPVs or compositions of LNP-MPVs comprising such may be substantially free of lactoglobulins. In some of these embodiments, the size of the LNP-MPVs, e.g., liposome-WPVs, is about 20-1,000 nm. In some embodiments, the size of the LNP-MPVs, e.g., liposome-WPVs, is about 100-160 nm. In some embodiments, the LNP-MPVs, e.g., liposome-WPVs, are derived from MPVs, e.g., WPVs, that are not modified from their naturally occurring state. In some embodiments, the LNP-MPVs, e.g., liposome-WPVs, are derived from MPVs, e.g., WPVs, that are modified from their natural state. In some embodiments, the MPVs, e.g., WPVs, are modified by altering the quantity, concentration, or amount of a biomolecule naturally present, e.g., the addition or complete or partial removal of a biomolecule naturally present (e.g., carbohydrate, such as a glycan and/or glycan residue; fatty acid, lipid). In some embodiments, the MPV, e.g., WPV, is modified by the addition of a biomolecule not naturally present (e.g., carbohydrate, such as a glycan; fatty acid; lipid; or protein, e.g., glycoprotein). Accordlingly, in some embodiments, the LNP-MPVs, e.g., liposome-WPVs, comprise an altered quantity, concentration, or amount of a biomolecule naturally present relative to an LNP-MPV derived from an unmodified, naturally occurring MPV, e.g., WPV. In some embodiments, the LNP-MPV comprises additional biomolecules relative to an LNP-MPV derived from an unmodified, naturally occurring MPV, e.g., WPV. In some of these above embodiments, the LNP-MPVs, e.g., liposome-WPVs, comprise a lipid membrane to which one or more proteins described herein are associated. In some embodiments, the LNP-MPVs, e.g., liposome-WPVs, comprise one or more proteins selected from BTN1A1, CD81 and XOR. In some embodiments, one or more proteins associated with the lipid membrane of the LNP-MPVs, e.g., liposome-WPVs, are glycosylated. In any of these embodiments, the LNP-MPVs, e.g., liposome-WPVs, can be used for oral delivery of a cargo, e.g., a cargo encapsulated in the LNP-MPV. In some embodiments, the LNP-MPVs, e.g., liposome-WPVs, are formulated to form a suitable composition for use in oral delivery of the cargo encapsulated therein to a subject, for example, a human patient. In some embodiments, the cargo can be a peptide, a protein, a nucleic acid, a polysaccharide, or a small molecule.

It is contemplated that LNP-MPVs transfer the components, modifications, and properties to the corresponding surface loaded LNP-MPVs. In a non-limiting example, a corresponding surface loaded liposome-WPVs.

In specific examples, the present disclosure provides LNP-MPVs loaded with therapeutic agents such as DNA, RNA, iRNA, mRNA, siRNA, antisense oligonucleotides, analogs of nucleic acids, antibodies, hormones, and other peptides and proteins. Such LNP-MPVs may be loaded with diagnostics or imaging agents.

In some embodiments, the LNP-MPVs disclosed herein may be approximately round or spherical in shape. In some embodiments, the LNP-MPVs is approximately ovoid, cylindrical, tubular, cube, cuboid, ellipsoid, or polyhedron in shape.

In some embodiments, the LNP-MPVs described herein are able to transport one or more agents, e.g., therapeutic agent, through a mammalian gut such that the agent has systemic and/or tissue bioavailability. In some embodiments, the LNP-MPVs described herein is able to deliver one or more agents, e.g., therapeutic agent, to one or more mammalian tissue(s).

### V. Oral Delivery of Cargos

Any of the LNP-MPVs, e.g., liposome-WPVs or surface programmed LNP-MPVs or LNP-WPVs disclosed herein, loaded with a suitable cargo, may be formulated to form a composition for oral administration. Such a composition may further comprise one or more pharmaceutically acceptable carriers. “Acceptable” means that the carrier must be compatible with the active ingredient of the composition (and preferably, capable of stabilizing the active ingredient) and not deleterious to the subject to be treated. Pharmaceutically acceptable carriers (excipients), including buffers, are well known in the art. See, e.g., Remington: The Science and Practice of Pharmacy 20^{th} Ed. (2000), Lippincott Williams and Wilkins, Ed. K.E. Hoover. Suitable carriers include microcrystalline cellulose, mannitol, glucose, defatted milk powder, polyvinylpyrrolidone, and starch, or a combination thereof.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

To deliver a suitable cargo (e.g., a therapeutic agent) to a subject, an effective amount of any of the compositions disclosed herein, comprising LNP-MPVs loaded with the cargo, may be administered orally to a subject (e.g., a human patient) in need of the treatment. In some embodiments, the composition given to the subject comprises an amount of the LNP-MPVs sufficient to deliver a therapeutically effective amount of the cargo loaded therein to achieve the intended therapeutic effects. Such amounts may depend on the particular condition being treated, the severity of the condition, the individual patient parameters including age, physical condition, size, gender and weight, the duration of the treatment, the nature of concurrent therapy (if any), which would be within the knowledge and expertise of a health practitioner. These factors are well known to those of ordinary skill in the art and can be addressed with no more than routine experimentation.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B.D. Hames & S.J. Higgins eds.(1985»; Transcription and Translation (B.D. Hames & S.J. Higgins, eds. (1984»; Animal Cell Culture (R.I. Freshney, ed. (1986»; Immobilized Cells and Enzymes (1RL Press, (1986»; and B. Perbal, A practical Guide To Molecular Cloning (1984); F.M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Fusion Between Liposomes and Exosomes Facilitated by Temperature

Liposomes comprised of DOPC (60 mol%), Cholesterol (20 mol %), DOTAP (10 mol %), DOPE (10 mol %), with or without DPPE-PEG2000 (1.5 mol%), and with 20 uM dye DiI were prepared via extrusion. All the components were dissolved in chloroform in a 2 dram glass vial. The chloroform was evaporated under a stream of air while the vial was manually rotated in order to form a thin film on the walls of the vial. The lipid film was dried under vacuum for 1 h to remove trace amounts of chloroform. The lipid film was hydrated with PBS, pH=7.4. The suspension was vortexed for 5 min followed by extrusion. The extrusion was done using Avanti Polar Lipids extruder with 100 nm Polycarbonate Membranes. The mixture was passed 11 times through the extruder.

Milk exosome vesicles (MEVs) isolated from milk using ultracentrifugation and casein depletion were incubated with 20 uM DiR dye in ethanol. The particle concentration was 1×10¹³ particles/ ml. The sample was incubated at room temperature for 1.5 h. No further purification was performed.

Alternatively, MEVs isolated from milk using ultracentrifugation and depletion were incubated with cholesterol-siRNA-DY6771. The particle concentration was 1×10¹³ particles/ ml and the ratio of ON/EV was 250/1. The sample was incubated at room temperature for 1.5 h. No further purification was performed.

The DiI labeled liposomes were mixed 1/1 with DiR labeled MEVs. The samples were incubated at 37° C. for 1 h. Fusion of the liposome and MEVs was evaluated by Forster Resonance Energy Transger (FRET). Briefly, FRET between DiI and DiR was measured at 0 and 1 h in a 96-well black, clear bottom well plate using Tecan plate reader. The fluorescence spectra for all samples was measured upon excitation at 525 nm, cut off at 535 nm and recorded between 550 nm and 850 nm. Separately, DiR direct excitation was measured upon excitation at 690 nm, cut off at 695 nm and recorded between 710 nm and 850 nm. Separately, DY677 direct excitation was measured upon excitation at 640 nm, cut off at 665 nm and recorded between 665 nm and 850 nm.

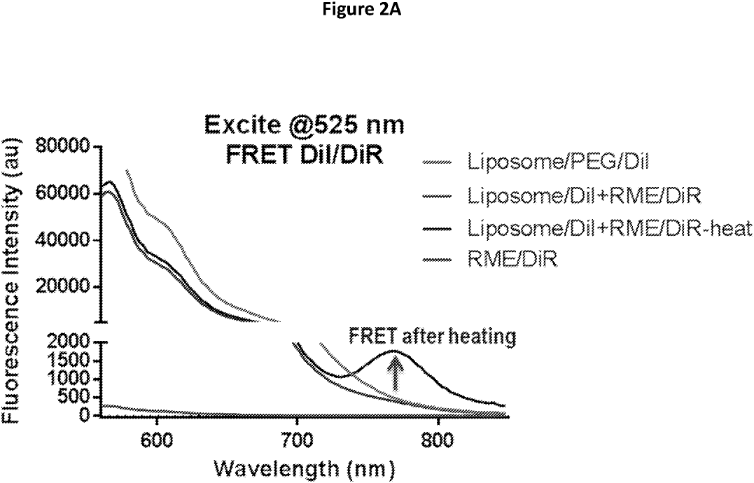

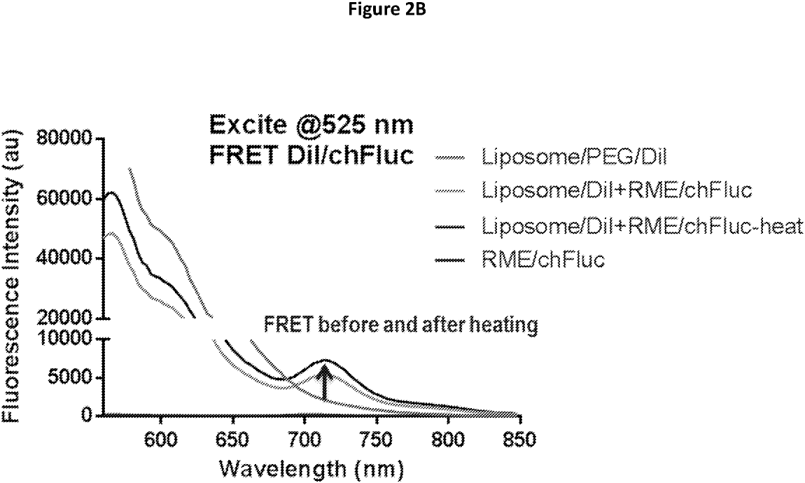

The results show that the fusion between liposomes and milk exosome vesicles is slow. Incubation time and heat facilitate the fusion. FIG. 2A. When MEVs are attached to siRNA conjugated with DY677, mixing the MEV and liposome and heating did not show major difference. This may be due to the fact that electrostatic interaction favors interaction between liposomes and siRNA. FIG. 2B.

### Example 2: Fusion Between Liposomes and Exosomes Facilitated by Polyethylene Glycol (PEG)

This experiment harnesses the fusion capability of PEG where liposomes and milk exosome vesicles were mixed in a 1:1 ratio of particle count in the presence of different concentration of PEG (0-30%) of varying molecular weight (6, 8 10 and 12 kD). Loss in the number of total particles was followed as a parameter to monitor the extent of fusion.

Liposomes were prepared by extrusion process using DOPC:DOPE:Cho in 35:35:30 mole ratio 1.5% NBD-DPPE and RHO-DPPE. Liposomes and MEVs were enumerated by nanoparticle tracking analysis (NTA) to obtain their average particle size and concentration. Liposome and MEVs were mixed in 1:1 ratio with 1E+11 particles/mL each and suitable volume of 60% stock of PEG in water was added to obtain the final desired concentration. The mixture was incubated at 40° C. for 2 h at constant vortexing to enable uniform mixing. After 2 h, the samples were immediately diluted to negate any further effect of PEG. The particle size distribution and concentration were measured using NTA.

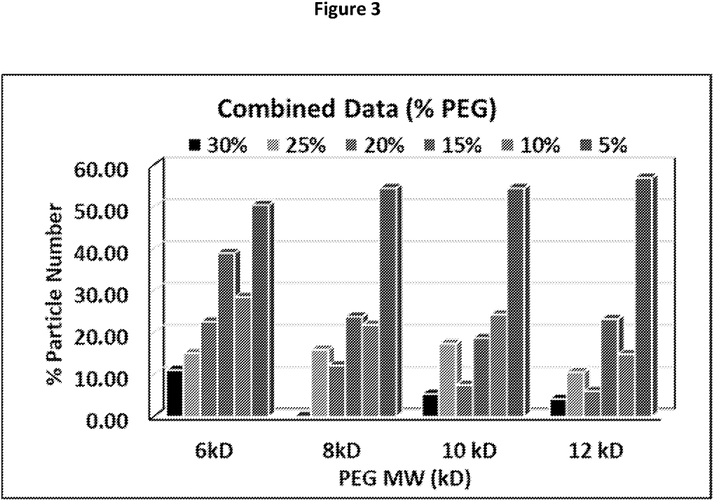

Total particle concentration from all the reaction mixtures was calculated as total percent of the control experiment without PEG and the percent particle values plotted as a function of PEG concentration as well as PEG molecular weight. Critical analysis of the data reveals that at higher concentration of PEG (20-30%), there is a significant reduction in the particle number in all the samples. 8 kD PEG at 30% concentration shows the most dramatic reduction in the particle number, confirming maximum fusion events. FIG. 3. Particle numbers can be used as an indicator of fusion events (reduced particle numbers indicate greater levels of fusion) show that with a decrease of the concentration of PEG, an increase in total number of particles was observed. As a function of PEG MW, fusion events were observed at concentrations above 20%, with strongest fusion events occuring at 30%.

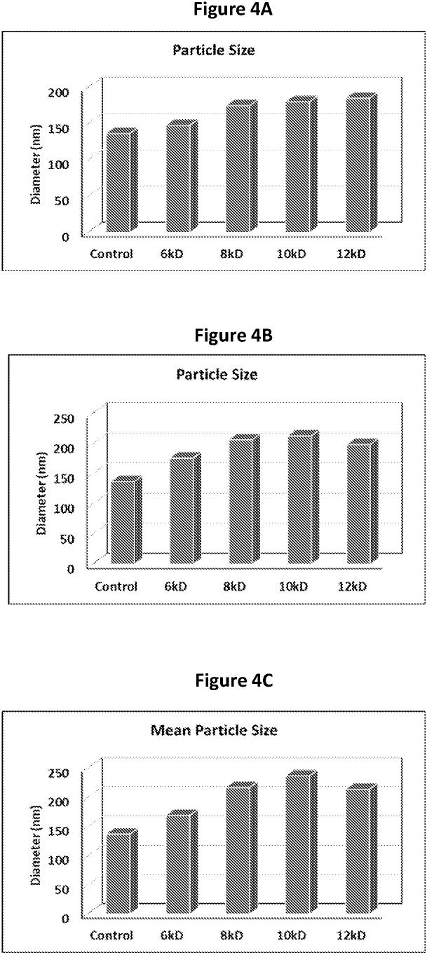

The mean particle size of the various reaction mixtures was also monitored and a distinct increase in the mean particle size was observed, which is consistent with the expected fusion dependent size increase. FIGS. 4A-4C (PEG 10%, 20%, and 30%, respectively).

### Example 3: Fusion Between Liposomes and Exosomes Facilitated by Extrusion

This experiment was designed to facilitate fusion of MEVs with cargo loaded liposome by mechanical force during the process of extrusion. The fusion events were followed by monitoring the transfer of cargo from the liposome to the exosome. The cargo in this experiment was 5(6)-carboxyfluorescein (5-CF), loaded into the liposomes at a self-quenching concentration of 50 mM. When liposome-exosome fusion occurs, it is expected that this event will lead to the dilution of the dye and result in an increase in fluorescence.

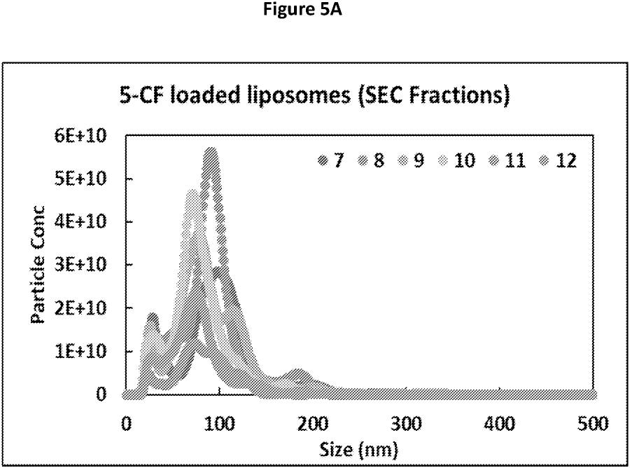

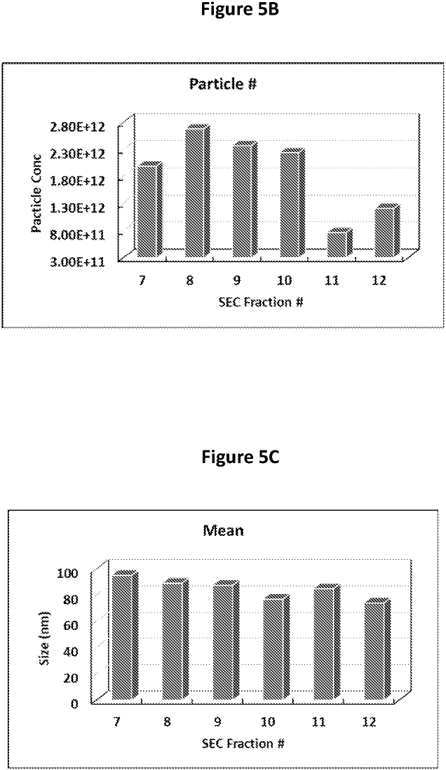

Liposome loaded with 50 mM 5(6)-carboxyfluorescein (5-CF) were prepared by extrusion process using DOPC:DOPE:Cho in 37.5:37.5:25 mole ratio. The liposomes were purified by size exclusion chromatography to remove all unencapsulated free dye. FIGS. 5A-5C. The purified liposome fractions and exosomes were enumerated by NTA to obtain their average particle size and concentration. Liposome and MEVs were mixed in a 1:1 ratio with 1E+11 particles/mL each and extruded using syringe filter assembly with 200, 100 and finally 50 nm polycarbonate membrane filters. After extrusion, the samples were measured for particle size distribution and concentration using NTA. The reaction mixture was also incubated with 25 µg WGA lectin to preferentially bind to the exosomes to crosslink them and facilitate centrifugation based purification.

Two independent extrusion trials were performed with two different fractions of purified 5-CF and the transfer of dye from liposome to exosome was measured by monitoring the fluorescence in the purified exosomes. The 5′-CF loaded liposomes were consequently extruded through 200 nm and 100 nm filters and mixed with milk exosomes (isolated by ATFF/SEC method) one to one ratio at the concentration 1-5E11 particles/ml.

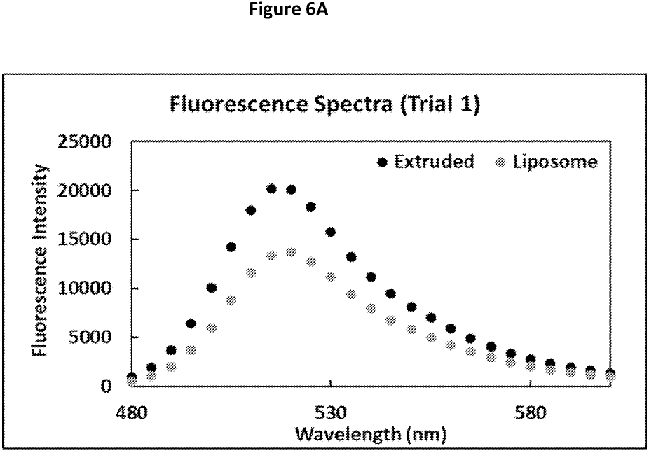

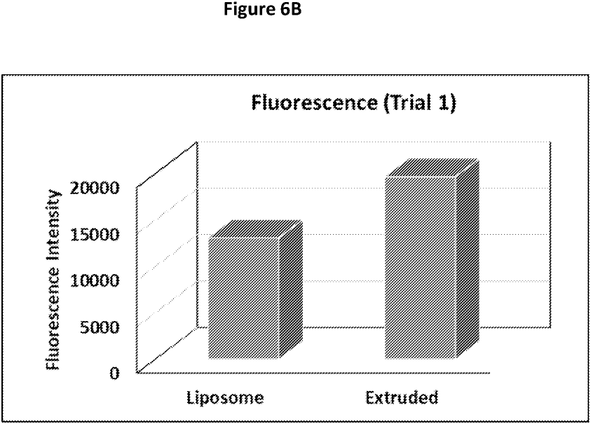

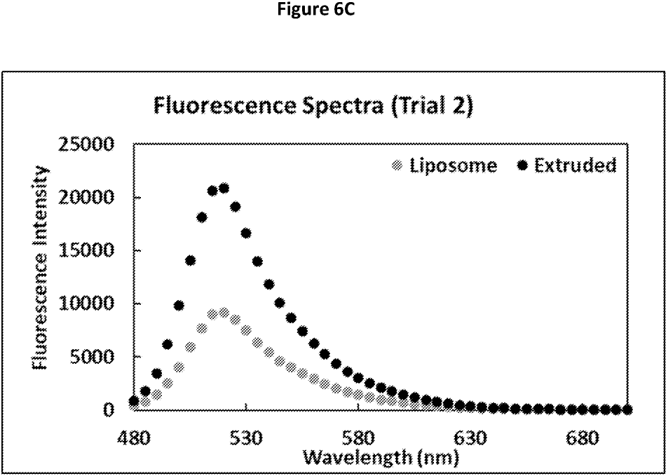

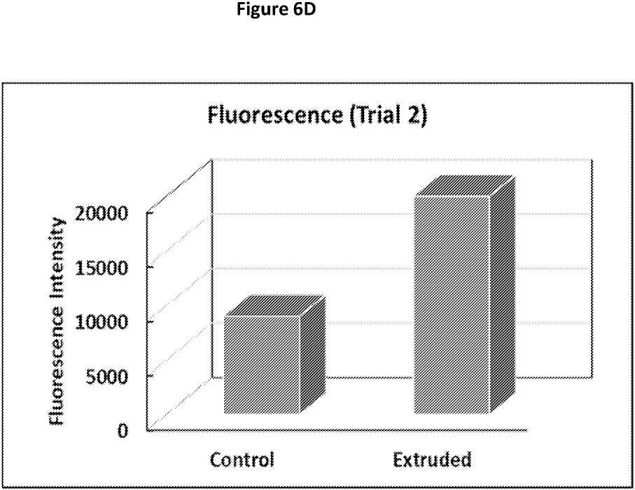

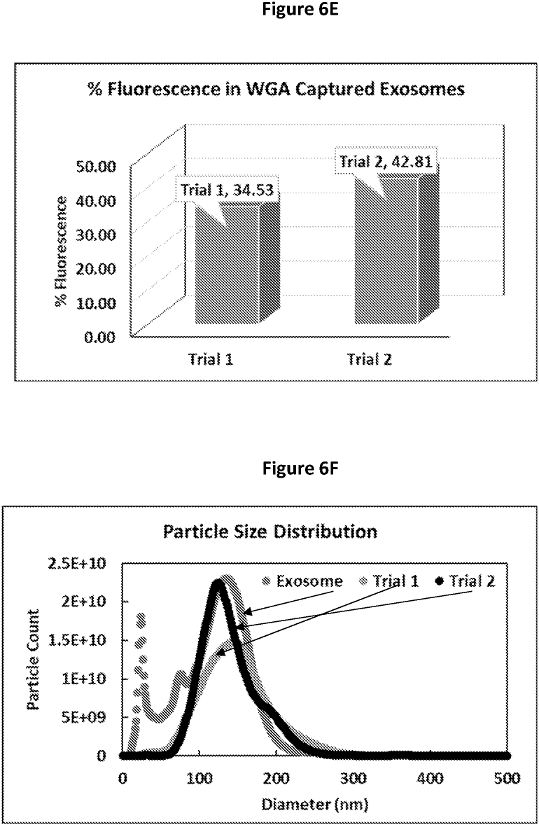

Exosomes from both the trials showed a positive fluorescence signal from 5-CF, confirming that the dye was transferred to the exosomes by virtue of liposome-exosome fusion. FIGS. 6A-6E. Loss of FITC self-quenching indicates liposome/MEV fusion.

The mean particle size distribution of the various reaction mixtures was also monitored. No distinct change was observed, as expected given the fused particles were extruded. FIG. 6F.

### Example 4: PEG Mediated Fusion of Cationic Liposomes With Exosomes Cargo Transfer

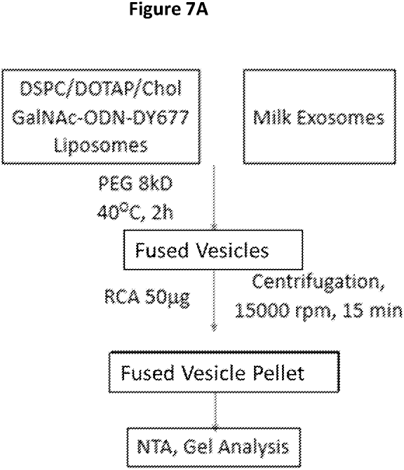

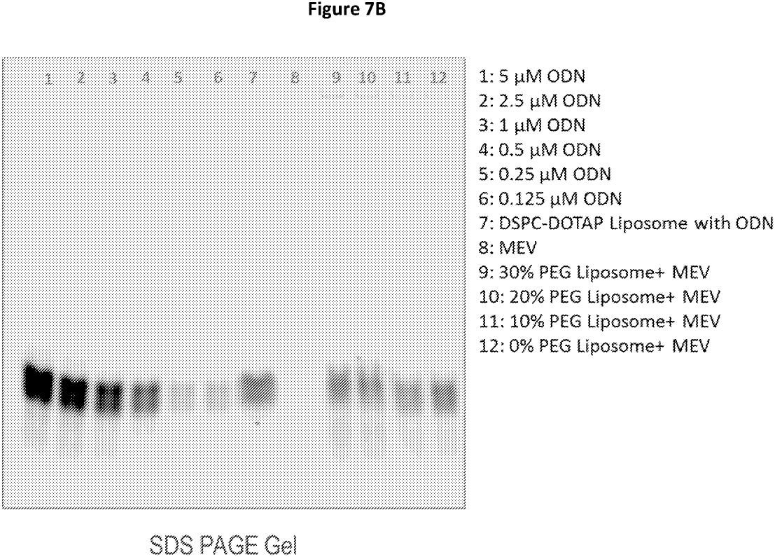

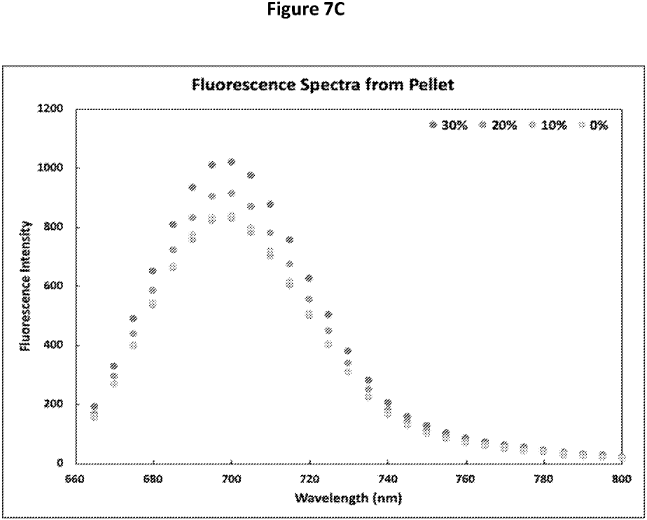

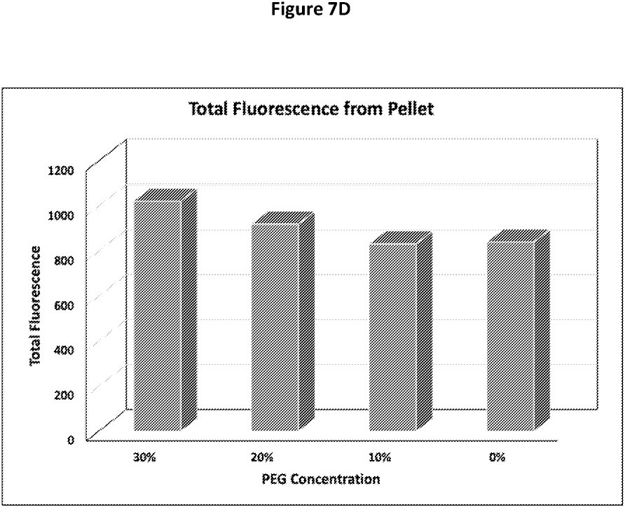

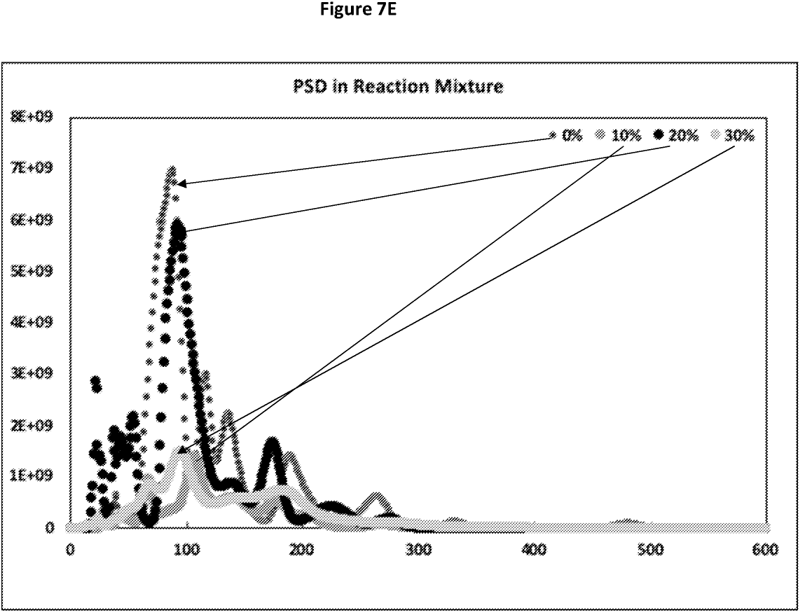

The cationic liposomes were used as a model liposomal system for efficient encapsulation of nucleic acid by charge-based interaction in order to study the transfer of payload from liposome to exosome by PEG-mediated fusion. GalNAc-ON-DY677 oligonucleotide was used as a model payload (which is modified by an exemplary targeting moiety GalNAx) to monitor the material transfer by gel electrophoresis as well as fluorescence measurement. A schematic illustration showing an exemplary process of cationic liposome-exosome fusion in the presence of PEG is provided in FIG. 7A.

Cationic liposomes were prepared by using thin film hydration followed by an extrusion method as disclosed herein. DSPC:DOTAP:Cho:DSPE-mPEG was used in 40:35:24:1 % mole ratio. Lipid film was prepared by chloroform evaporation following which it was hydrated overnight in 100 µL of 50 µM ON. Finally, the volume was made to 1 mL using PBS buffer and extruded through 200, 100 and 50 µm pore size polycarbonate filters. The liposome and exosome were measured for their particle size distribution and concentration using NTA. 1E+12 liposome and milk exosome were mixed and suitable volume of 60% stock of 8 kD PEG was added to achieve a final concentration of 0, 10, 20 and 30%. The mixture was incubated at 40° C. for 2 h at constant vortexing to enable uniform mixing. After 2 h, the samples were immediately diluted to negate any further effect of PEG. The particle size distribution and concentration were measured using NTA. The fused vesicles were purified by crosslinking using RCA lectin (50 µg) followed by simple centrifugation at 15000 rpm for 10 min. The pellet was washed in PBS and finally lysed using 4% Proteinase K and 1% SDS incubated for 25 min at 65° C. The lysed samples were tested for ON transfer by fusion against standard ON on a 20% PAGE gel. Presence of an ON band in the purified fused vesicles samples confirms that the material could be transferred by our fusion approach. FIG. 7B. A strong signal for the presence of ON in fused vesicles captured by lectin was observed in the PAGE assay. Fluorescence measurement from lysed purified fused vesicles also show the presence of fluorescently tagged ON. FIGS. 7C and 7D. Material analysis was confirmed by transfer of fluorescent OD to fused vesicles. As expected, maximum fluorescence was seen from fusion sample in the presence of 30% PEG.

Particle size and concentration analysis clearly indicates that fusion efficiency of PEG is concentration dependent, consistent with the prior observation. FIG. 7E.

### Example 5: PEG Mediated Fusion of Neutral Liposomes With Exosomes Cargo Transfer

The oligonucleotide (ON,) was used as a model payload for encapsulation into the neutral liposomes by thin film hydration method of encapsulation in order to study the transfer of payload from liposome to exosome by PEG-mediated fusion.

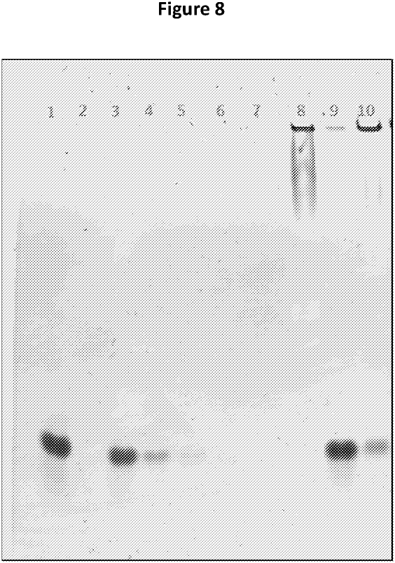

Neutral liposomes were prepared by using thin film hydration followed by extrusion method. DSPC:Cho:DSPE-mPEG was used in 70:20:1 % mole ratio. Lipid film was prepared by chloroform evaporation following which, it was hydrated overnight in 40 µL of 100 µM ON. Finally, the volume was adjusted to 1 mL using PBS buffer and extruded through 200, 100 and 50 µm pore size polycarbonate filters. The liposome and exosome were measured for their particle size distribution and concentration using NTA. 1E+12 liposomes and exosomes were mixed and suitable volume of 60% stock of 8 kD PEG was added to achieve a final concentration of 0, 10, 20 and 30%. The mixture was incubated at 40° C. for 2h at constant vortexing to enable uniform mixing. After 2h, the samples were immediately diluted to negate any further effect of PEG. The particle size distribution and concentration was measured using NTA. The fused vesicles were purified by crosslinking using RCA lectin (50 µg) followed by simple centrifugation at 15000 rpm for 10 min. The pellet was washed in PBS and finally lysed using 4% Proteinase K and 1% SDS incubated for 25 min at 65° C. The lysed samples were tested for ON transfer by fusion against standard ON on a 20% PAGE gel. Presence of an ON band in the purified fused vesicle samples confirms that the material could be transferred by the fusion approach disclosed herein. FIG. 8.

Particle size and concentration analysis indicated that fusion efficiency of PEG is concentration dependent, consistent with the prior observation. 30% PEG showed the maximum ON transfer from the liposome to the exosome.

### Example 6. Fused Vesicle Protects Encapsulated Oligonucleotide (ON) Cargo in the Presence of Detergent

An oligonucleotide (ON) cargo was used as a model payload for encapsulation into the cationic lipid nanoparticles disclosed herein (see Examples above) using a microfluidic system. The cargo-carrying lipid nanoparticles (LNP) were fused with vesicles purified from milk to form fused vesicles.

Both LNP and LipoMEV carrying the ON cargo were exposed to S1 nuclease. Briefly, a S1 nuclease (Aspergillus oryzae) degradation assay was conducted in acetate buffer pH=4.6 (60 mM NaOAc, 1 mM ZnSO4). Each oligonucleotide (ON) sample in buffer, in LNP (lipid nanoparticles), or in LipoEVs was split into 3 aliquots. To a first aliquot, S1 nuclease was added to a final nuclease concentration of 10 U/ul in presence of 1% Triton X-100. A second aliquot was supplemented with S1 nuclease at a final nuclease concentration of 10 U/ul without the detergent. The third aliquot was supplemented with the same amount of buffer (60 mM NaOAc, 1 mM ZnSO4, 1% Triton-X100, pH=4.6) as a blank control. All samples were incubated for 45 min at 37° C. All reactions were quenched with 30 mM EDTA. All samples were incubated for 10 min at room temperature; analyzed on 20% TBE PAGE and run at 200 V using XCell SureLock® Mini-Cell. The gel was stained with SYBR Gold (10,000x in TBE buffer) for 10 min on a shaker at 4° C. The gel was imaged using a boxed UV light to visualize the dye.

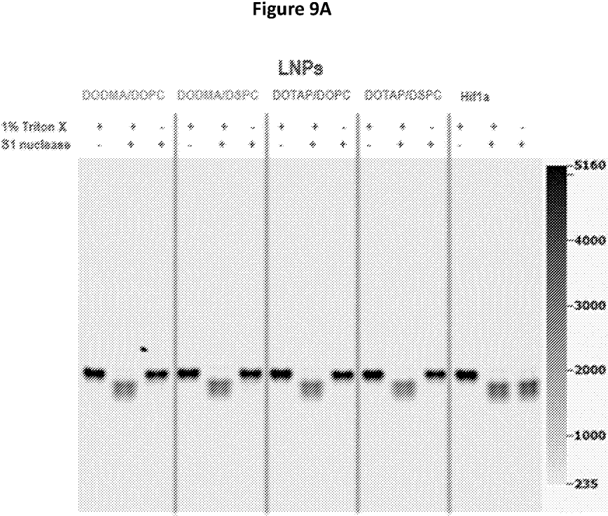

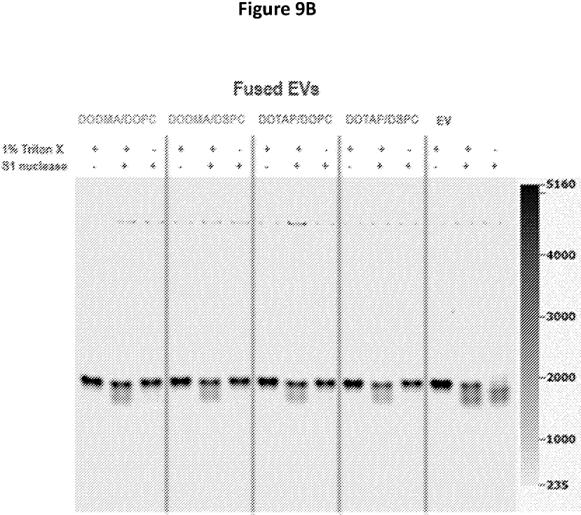

As shown in FIG. 9A, LNP efficiently protects ON from S1 degradation. Triton-X-100 is a standard reagent widely used to disrupt liposomes and lipid nanoparticles and release the payload, thus Triton-X100 treated LNP do not protect ON from degradation. Contrary, milk extracellular vesicles fused with LNP, are stable under these coditions and provide significant protection to the ON. FIG. 9B. See also Tables 20 and 21 below.

TABLE 20

LNP Protection from S1 Nuclease Degradation

| % left after degradation | +S1/+TritonX | +S1/-TritonX |
|---|---|---|
| DODMA/DOPC | 4.1 | 79.6 |
| DODMA/DSPC | 4.6 | 95.3 |
| DOTAP/DOPC | 4.6 | 81.4 |
| DOTAP/DSPC | 3.4 | 73.9 |
| ASO | 4.3 | 4.5 |

TABLE 21

LNP/EV Protection from S1 Nuclease Degradation

| % left after degradation | +S1/+TritonX | +S1/-TritonX |
|---|---|---|
| EV+DODMA/DOPC | 59.6 | 80.5 |
| EV+DODMA/DSPC | 53.2 | 77.9 |
| EV+DOTAP/DOPC | 63.6 | 72.4 |
| EV+DOTAP/DSPC | 53.4 | 74.2 |
| EV | 36.2 | 6.9 |

### Example 7: Lyophilization of Milk Exosome Vesicles (MEV) and Milk Exsosome Vesicles Fused with Lipid Nanoparticles (LipoMEV) Lyophylization

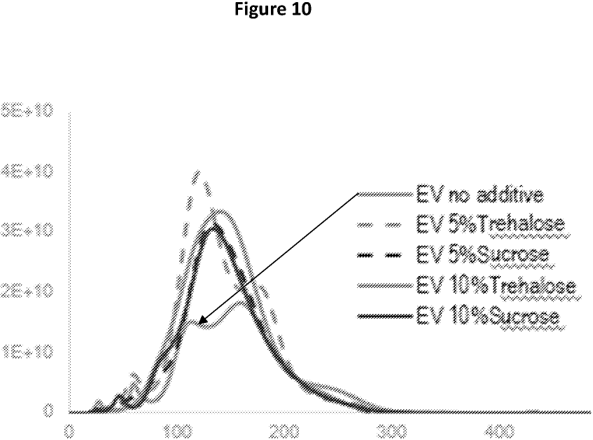

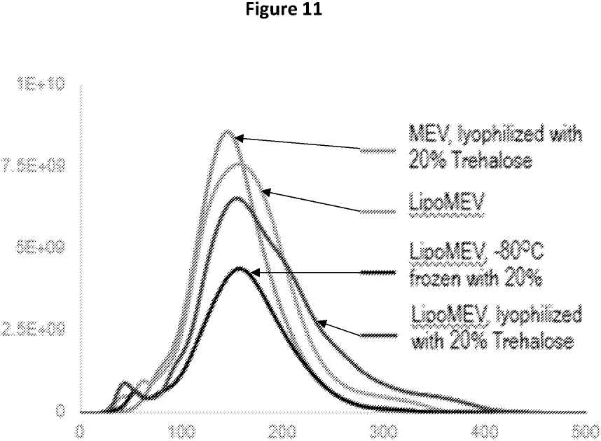

An oligonucleotide (ON) was used as a model payload for encapsulation into the cationic liposomes using a microfluidic system. The ON-carrying lipid nanoparticles were fused with milk exosomes to form fused vesicles. The fused vesicles and MEVs were lyophilized with or without cryoprotectant and resuspended in water equivalent to initial volume. Nanoparticle tracking analysis confirmed efficient resuspension of both MEV (FIG. 10) and fused vesciles (“LipoMEV”) (FIG. 11) without significant change in size distribution.

### Example 8: Lipid Nanoparticles With Either Cationic or Ionizable Lipids Are Fused with Milk Exosome Vesicles

An oligonucleotide (ON) was used as a model payload in this example for encapsulation into cationic liposomes using a microfluidic system. The lipid nanoparticles were fused with milk exosomes (MEVs). Tables 22-24 show particle sizes before and after fusion.

TABLE 22

Size Analysis of Lipid Nanoparticles Carrying Oligonucleotide

| | Mean size (nm) | Mode size (nm) | SD | Concentration (particle/ml) |
|---|---|---|---|---|
| DODMA/DOPC LNP | 55.9±1.4 | 53.3±0.2 | 25.5±1.1 | 3.92E+11±3.61E+10 |
| DODMA/DSPC LNP | 89±0.4 | 89.5±2.8 | 29.8±1.5 | 2.90E+11±1.18E+10 |
| DOTAP/DOPC LNP | 63.5±0.4 | 58.1±1 | 26.2±0.9 | 4.04E+11±2.15E+10 |
| DOTAP/DSPC LNP | 70.5±0.4 | 62.4±2 | 33.1±1.6 | 4.72E+11±2.68E+10 |

TABLE 23

Particle Sizes of Lipid Nanoparticles and Milk Exosomes Before and After Fusion and Cargo-Loading

| Sample | Mean size (nm) | Mode size (nm) | SD | Concentration (particle/ml) |
|---|---|---|---|---|
| MEV | 141.4±1.5 | 126.6±2.3 | 36.9±0.5 | |
| DOTAP LNP | 61.5±0.3 | 56±1.4 | 22.2±0.8 | 4.13E+11±1.35E+10 |
| LNP/MEV ratio 3/1 | 146.4±1 | 133.6±4.1 | 39.1±1.1 | 1.51E+12±4.26E+10 |
| LNP/MEV ratio 4/1 | 149.5±1.4 | 131.3±1.5 | 41.5±1.4 | 2.12E+12±2.68E+10 |

TABLE 24

Size Analysis of Milk Exosomes Fused with Lipid Nanoparticles

| | Expected fusion size (nm) | Mean size (nm) | Mode size (nm) | SD | Concentration, (particle/ml) |
|---|---|---|---|---|---|
| MEV before fusion | | 145.3±0.8 | 125.8±6.3 | 38.2±0.7 | 1.54E+12±4.84E+10 |
| MEV+DODMA/DOPC | 148.0 | 152.8±0.6 | 139.7±6.7 | 41.9±0.8 | 1.31E+12±4.55E+10 |
| MEV+DODMA/DSPC | 155.7 | 154±0.8 | 141±11.4 | 43.7±1 | 1.23E+12±2.57E+10 |
| MEV+DOTAP/DOPC | 149.2 | 148.7±2.1 | 117.2±9.4 | 47.6±1.4 | 1.58E+12±3.69E+10 |
| MEV+DOTAP/DSPC | 150.6 | 159.7±1.4 | 158.9±8.8 | 50.5±1 | 1.48E+12±2.43E+10 |

1- Expected lipoMEV size is calculated as Dₑₓₚ=(D_{MEV}³+D_{LNP}³)^{⅓}

Size analysis results indicated in Table 24 above show particle size changes after fusion, which is indicative of efficient LNP:MEV fusion.

### Example 9: Effect of MEV:LNP Ratios on Fusion Efficiency

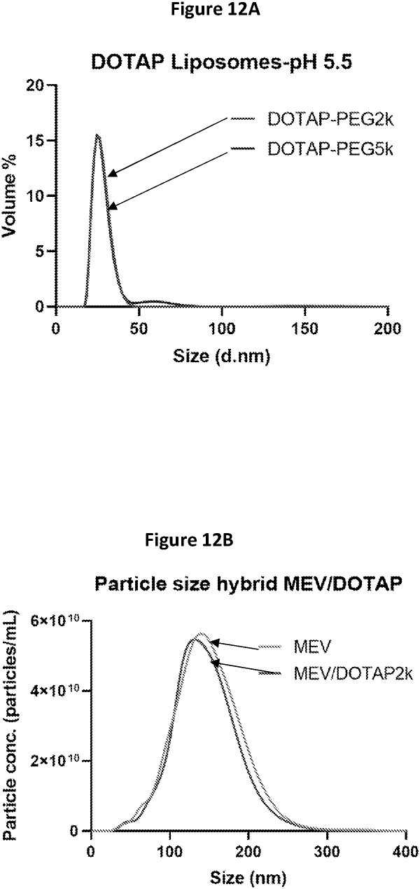

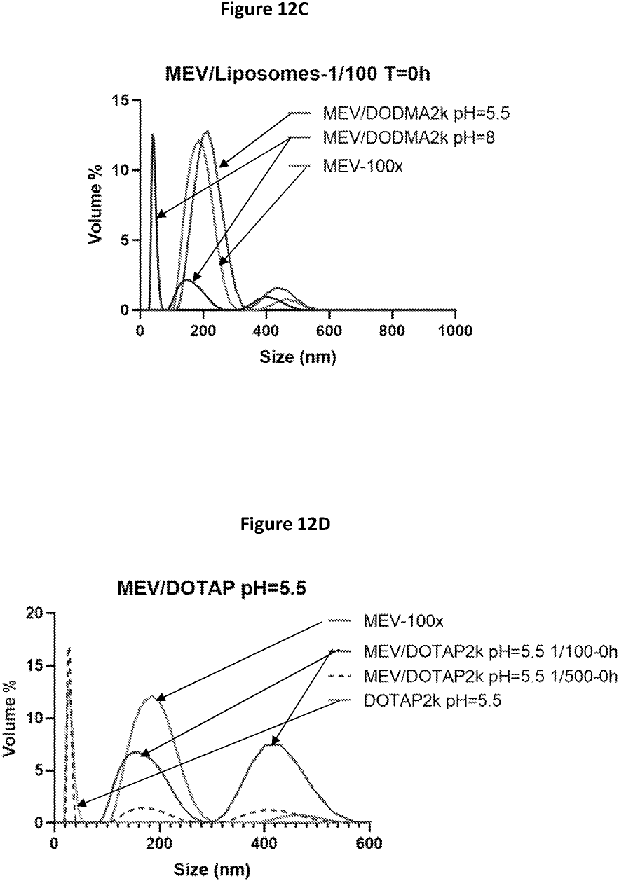

Cationic liposomes comprising DOTAP and DSPE-mPEG2k or DOTAP and DSPE-mPEG5k were prepared by using thin film hydration followed by an extrusion method as disclosed herein. The liposome and exosome were measured for their particle size distribution and concentration using NTA. Liposomes and MEVs were mixed together at ratios of 1:1, 10:1, 100:1 and 500:1. The mixture was incubated at 40° C. for 2 h at constant vortexing to enable uniform mixing. Results are shown in FIGS. 12A-12D. DOTAP liposomes are approximately 30 nm in size. No significant difference in size was observed between MEVs and fused vesicles with a 10:1 ratio. At 10:1, no peak is detected at 30 nm, indicating that fusion is complete. Even at the higher ratio of 100:1 significant fusion occurred. At 500:1 less fusion occurred than at 100:1.

### Example 10. Effect of pH on Fusion

Fusion of vesicles was evaluated using ultracentrifugation (UC). Upon high speed UC with 100 mM NaCl, only MEV-containing particles are pelleted and unfused liposomes remain in the supernatant. Liposomes are labeled with fluorescence, and fluorescence of supernatant post UC is measured to determine the level of fusion.

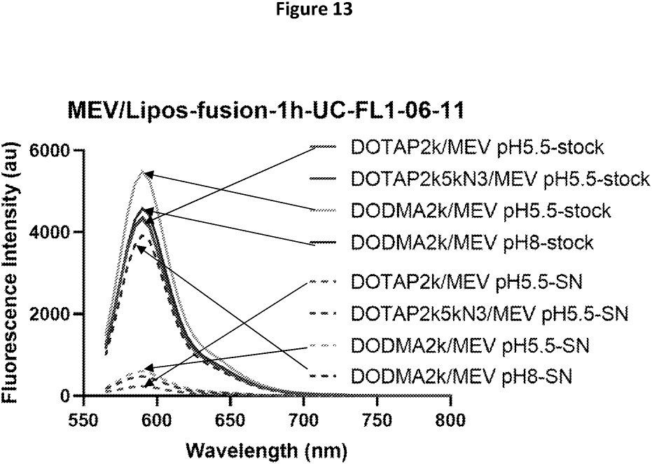

Liposomes (DOTAP (or DODMA) / Cholesterol / DOPC / RhDPPE / DSPE-PEG2k (50:27.7:20:0.3:2 mol%) were incubated for 15 min with MEVs at a ratio Liposome: MEV of 10:1. Next, the samples were centrifuged at 10,000 g for 15 minutes or 100,000 g for 1 hour. Results are shown in FIG. 13 and Table 25.

TABLE 25

Percent fusion as assessed by UC method

| Sample | % Fused 15 min (10k x g) | % Fused Ih (UC) |
|---|---|---|
| DOTAP2k/MEV pH5.5-stock | 83.1 | 94.2 |
| DOTAP5kN3/MEV pH5.5-stock | 41.1 | 88.7 |
| DODMA2k/MEV pH5.5-stock | 32.2 | 88.9 |
| DODMA2k/MEV pH8-stock | 1.5 | 14.1 |

Results show fusion at pH 5.5. But little fusion at pH 8 in samples measured.

### Example 11. Fusion of Lipid Nanoparticles Loaded With ASO or siRNA With Milk Exosome Vesicles

Oligonucleotide (ON) or siRNA was used as a model payload in this example for encapsulation into cationic liposomes using a microfluidic system. The lipid nanoparticles were fused with milk exosomes (MEVs). Following similar procedures as disclosed herein, ON and siRNA as a was first encapsulated into lipid nanoparticles (LNPs) comprising DOTAP or DODMA, a helper lipid (DOPC or DSPC), and optionally cholesterol and DSPE-mPEG2000 (Lipid composition: DOTAP (or DODMA)/Cholesterol/DOPC (or DSPC)/DSPE-mPEG2000 50/38.5/10/1.5 mol%) The ON or siRNA loaded LNPs were then fused with MEVs.

Table 26 below summarizes general statistics on size and entrapment efficiency of ASO and siRNA LNP formulation.

TABLE 26

ASO and LNP Formulations

| pH=5.5 | Total flow rate ml/min | Buffer/Lipid flow rate rati | Mean size | Mode size | SD | Encapsulation efficiency | |
|---|---|---|---|---|---|---|---|
| ASO at 0.13 mg/ml; Lipid conc. is 1.5 mM; cationic Lipids/ASO 5/1 | | | | | | | |
| DOTAP/DOPC | 1 | 1/1 | 58.4 | 51 | 16.8 | 98.9 | 1.4 |
| DOTAP/DOPC | 1 | 3/1 | 60.8 | 64.2 | 22.5 | 97.2 | 1.7 |
| DOTAP/DSPC | 1 | 1/1 | 70.5 | 62.4 | 33.1 | | |
| DODMA/DOP | 1 | 1/1 | 56.0 | 53 | 13.7 | 91.5 | 3.6 |
| DODMA/DOP | 1 | 3/1 | 64.4 | 57 | 17 | 92.8 | 4.6 |
| DODMA/DSP | 1 | 1/1 | 89.0 | 89.5 | 29.8 | | |

* Lipid composition: DOTAP (or DODMA)/Cholesterol/DOPC (or DSPC)/DSPE-mPEG2000 50/38.5/10/1.5 mol%

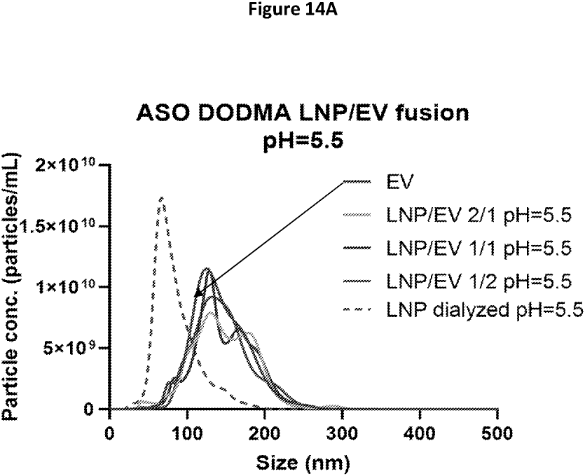

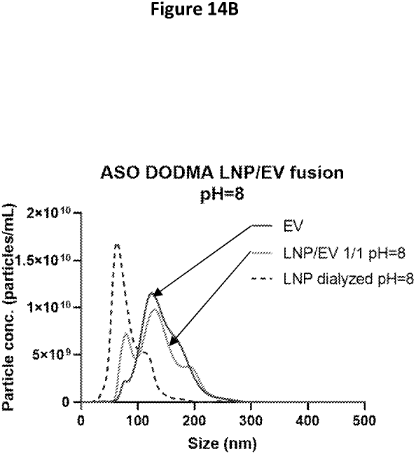

Fusion of the ON-LNP and siRNA-LNP and EV were carried at various LNP/EV ratios (2:1, 1:1, and 1:2). Table 27 and FIGS. 14A and 14B shows results of fusion of MEVs with ON-loaded LNPs.

TABLE 27

Fusion of ON-LNP with EV at Different Ratios

| SN | Mean size | | Mode size | | SD | | EV concentration (in SN) | |
|---|---|---|---|---|---|---|---|---|
| EV | 142.2 | 0.8 | 124.8 | 3.7 | 36.9 | 1 | 9.12E+11 | 3.72E+10 |
| LNP/EV 2/1 pH=5.5 | 146.8 | 3.2 | 137.6 | 16.3 | 40.6 | 2.2 | 7.54E+11 | 1.19E+10 |
| LNP/EV 1/1 pH=5.5 | 147.3 | 1.3 | 142.9 | 8.9 | 37.4 | 0.9 | 7.69E+11 | 7.33E+09 |
| LNP/EV ½ pH=5.5 | 145.8 | 1.7 | 134.8 | 4.8 | 36.9 | 0.7 | 7.82E+11 | 3.28E+10 |
| LNP/EV 1/1 pH=8 | 135.5 | 2.4 | 104.7 | 17 | 43.2 | 1.5 | 8.95E+11 | 4.31E+10 |
| LNP pH=5.5 after dialysis | 85.6 | 0.4 | 68.3 | 2.6 | 30 | 0.8 | 7.93E+11 | 1.16E+10 |

Results show that the size of the fused MEV/LNP at pH=5.5 increases relative to the native MEVs. The size of fused MEV/LNP at pH=8 is lower and a smaller size shoulder, indicating unfused LNPs.

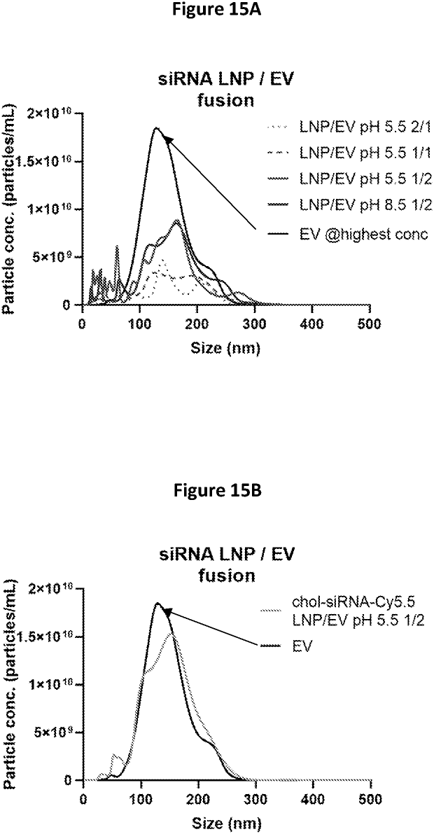

Table 28 and FIGS. 15A and 15B show results of fusion of MEVs with siRNA -loaded LNPs. Results show that higher LNP/EV ratios led to larger and less uniform particle sizes.

TABLE 28

Fusion of LNP with EV at Different Ratios

| | Mean size | | Mode size | | SD | | EV concentration | |
|---|---|---|---|---|---|---|---|---|
| EV | 146.8 | 0.7 | 140.8 | 4.9 | 39.1 | 0.6 | 1.61E+12 | 3.55E+10 |
| siRNA-LNP/EV 2/1 -pH 5.5 | 154.6 | 1 | 115.8 | 28 | 56.4 | 2.8 | 3.41E+11 | 1.50E+10 |
| siRNA-LNP/EV 1/1 -pH 5.5 | 159.7 | 4.3 | 133 | 39.3 | 60.7 | 1 | 4.77E+11 | 2.28E+10 |
| siRNA-LNP/EV ½-pH 5.5 | 144.8 | 3.7 | 145.2 | 7.7 | 61.1 | 3.4 | 9.15E+11 | 6.29E+10 |
| siRNA-LNP/EV ½ -pH 8.5 | 155.7 | 2 | 141.4 | 10.2 | 55.3 | 1.3 | 9.57E+11 | 3.76E+10 |
| chol-siRNA-Cy5.5 LNP/EV ½ - pH 5.5 | 150.4 | 1.1 | 154.4 | 4.9 | 45.7 | 1 | 1.64E+12 | 1.64E+10 |

### Example 12. Role of Helper Lipids and pH

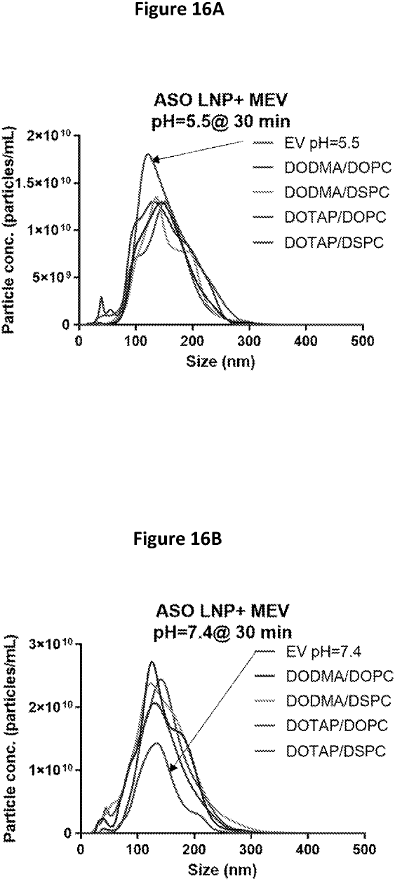

The effect of helper lipids DSPC and DOPC on fusion of liposomes with MEVs was assessed. Liposomes were prepared according to methods described herein and incubated with MEVs at 40 C for 30 minutes at pH 5.5 or pH 7.4. At pH 5.5., EV Particle concentration did not change but size increased. At pH 7.4, EV Particle concentration doubled and size did not change significantly. Results are shown in Table 29 and Table 30 and in FIGS. 16A and 16B.

TABLE 29

Particle size and EV concentration with fusion at pH 5.5

| pH=5.5 | Expected fusion size | Mean size | | Mode size | | SD | | EV concentration | |
|---|---|---|---|---|---|---|---|---|---|
| EV | | 145.3 | 0.8 | 125.8 | 6.3 | 38.2 | 0.7 | 1.54E+12 | 4.84E+10 |
| EV+DODMA/DOPC | 148 | 152.8 | 0.6 | 139.7 | 6.7 | 41.9 | 0.8 | 1.31E+12 | 4.55E+10 |
| EV+DODMA/DSPC | 155.7 | 154 | 0.8 | 141 | 11.4 | 43.7 | 1 | 1.23E+12 | 2.57E+10 |
| EV+DOTAP/DOPC | 149.2 | 148.7 | 2.1 | 117.2 | 9.4 | 47.6 | 1.4 | 1.58E+12 | 3.69E+10 |
| EV+DOTAP/DSPC | 150.6 | 159.7 | 1.4 | 158.9 | 8.8 | 50.5 | 1 | 1.48E+12 | 2.43E+10 |

TABLE 30

Particle size and EV concentration with fusion at pH 7.4

| pH=7.4 | Expected fusion size | Mean size | | Mode size | | SD | | EV concentration | |
|---|---|---|---|---|---|---|---|---|---|
| EV | | 139.9 | 0.9 | 129.6 | 4.4 | 36.3 | 1.7 | 1.16E+12 | 4.72E+10 |
| EV+DODMA/DOPC | 144.1 | 142.4 | 0.3 | 125.7 | 2.8 | 43.9 | 1.7 | 2.49E+12 | 8.55E+10 |
| EV+DODMA/DSPC | 150.4 | 146.6 | 1.1 | 128.4 | 5.2 | 52.3 | 1.8 | 2.76E+12 | 9.09E+10 |
| EV+DOTAP/DOPC | 146.2 | 146.7 | 1.4 | 129.1 | 6.1 | 41.1 | 0.7 | 1.92E+12 | 2.63E+10 |
| EV+DOTAP/DSPC | 146.2 | 142.8 | 0.6 | 143.5 | 3.9 | 47.5 | 0.6 | 2.51E+12 | 3.33E+10 |

### Example 13. Lectin Pulldown Assay for Assessment of siRNA Loading Effciency

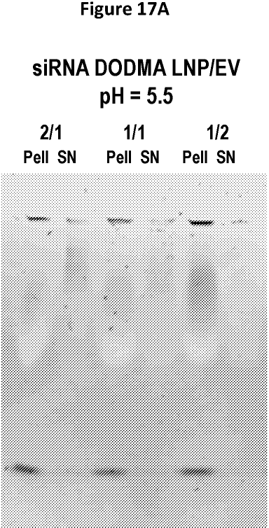

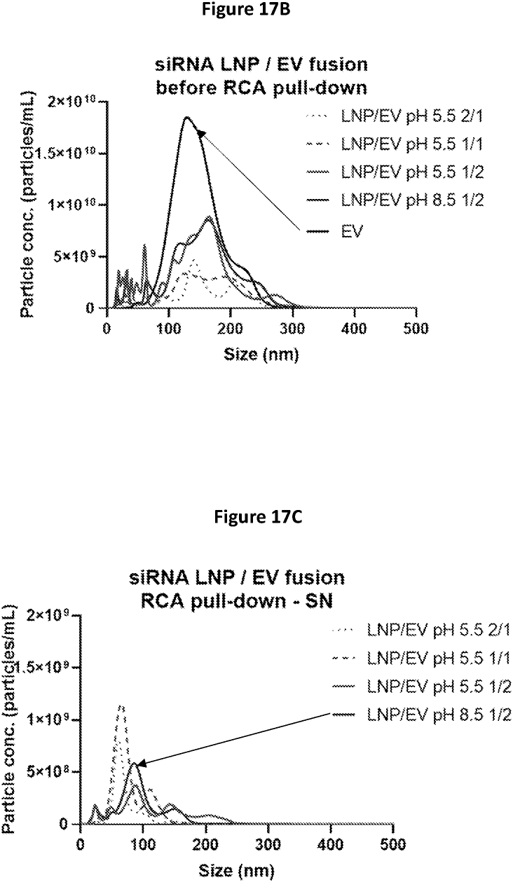

After fusion, the particles (such as those obtained as described in Example 11) were mixed with RCA, which binds EVs and the fusion product, and presence of ONs or siRNAs in the supernatant (SN) and pellets was analyzed as shown in FIG. 17A.

Particle sizes before RCA pull-down (FIG. 17B) and in SN (FIG. 17C) were also analyzed. See also Table 31 below. The results show significant transfer of siRNA from LNP to EVs even at higher LNP/EV ratios. Some LNPs were detected in SN after RCA pull-down when fusion was done at higher LNP/EV ratios.

TABLE 31

Particle Sizes After RCA Pull-Down

| SN | Mean size | | Mode size | | SD | | EV concentration (in SN) | | Fold reductio in concentration in SN |
|---|---|---|---|---|---|---|---|---|---|
| siRNA-LNP/EV 2/1 pH 5.5 | 72.8 | 0.9 | 60.8 | 1.4 | 25.2 | 2.2 | 2.09E+10 | 2.47E+09 | 16.32 |

| | | | | | | | | | |
|---|---|---|---|---|---|---|---|---|---|
| siRNA-LNP/EV 1/1 pH 5.5 | 76.6 | 0.4 | 66.1 | 2.2 | 26.2 | 0.9 | 4.38E+10 | 3.09E+09 | 10.89 |
| siRNA-LNP/EV ½ pH 5.5 | 114.9 | 2.6 | 83.1 | 3.4 | 57.6 | 2.9 | 2.93E+10 | 2.75E+09 | 31.23 |
| siRNA-LNP/EV ½ pH 8.5 | 94.7 | 1.5 | 82.7 | 3.2 | 39.2 | 4.2 | 3.25E+1C | 3.84E+09 | 29.45 |
| chol-siRNA-Cy5.5 LNP/EV ½ pH 5.5 | 124.6 | 1.2 | 121.7 | 12 | 51.9 | 0.5 | 4.81E+1C | 8.56E+08 | 34.10 |

* DODMA/Cholesterol/DSPC/DSPE-PEG2k at 50/38.5/10/1.5 mol %

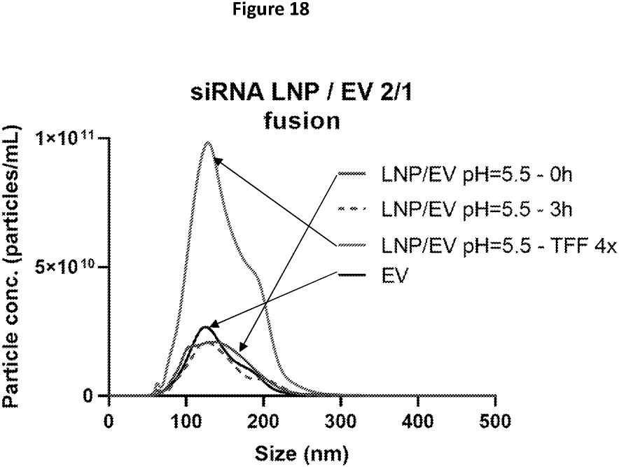

The fused EVs were concentrated using tangential flow filtration (TFF) and the results are shown in FIG. 18 and Table 32. Little or no particles were found in the waste from TFF. Samples were concentrated by around 4 folds using TFF by volume. NTA analysis shows about 4X increase in particle concentration after TFF.

TABLE 32

Concentration of Fused EVs Using TFF

| Sample | Mean size | | Mode size | | SD | | EV concentration | |
|---|---|---|---|---|---|---|---|---|
| WTFF/SEC | 139.9 | 1 | 125.7 | 1 | 36.5 | 1.1 | 2.03E+12 | 2.95E+10 |
| siRNA-LNP/EV - pH 5.5 - 0h | 144.8 | 1.4 | 127.9 | 9.8 | 41.9 | 0.8 | 2.17E+12 | 5.75E+10 |
| siRNA-LNP/EV - pH 5.5 - 3h | 143.6 | 1.8 | 124.2 | 6.2 | 38 | 1.3 | 1.72E+12 | 9.97E+09 |
| siRNA-LNP/EV - pH 5.5 - TFF-4x concentrated | 145.9 | 2.8 | 127 | 4.8 | 37.3 | 1.6 | 8.40E+12 | 8.55E+10 |

### Example 13. Lectin Pulldown Assay for Assessment of ASO Loading Effciency

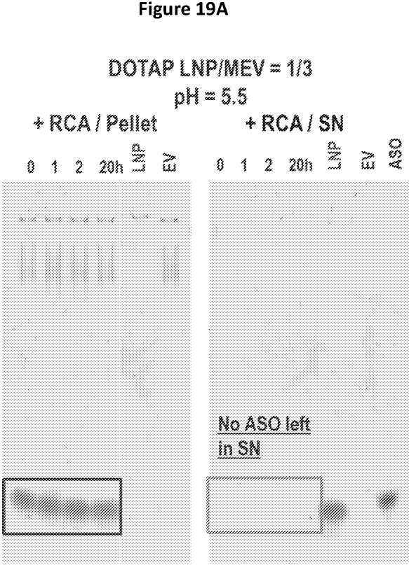

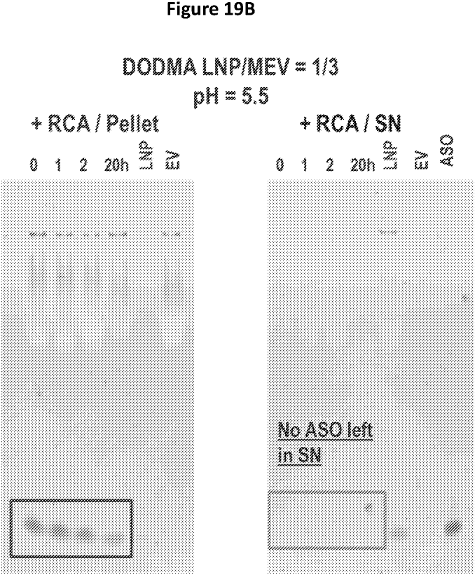

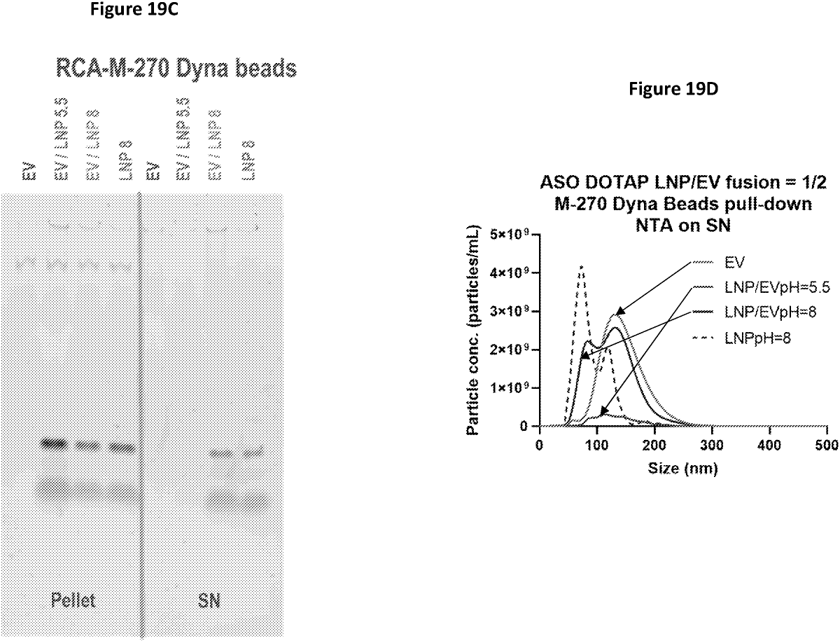

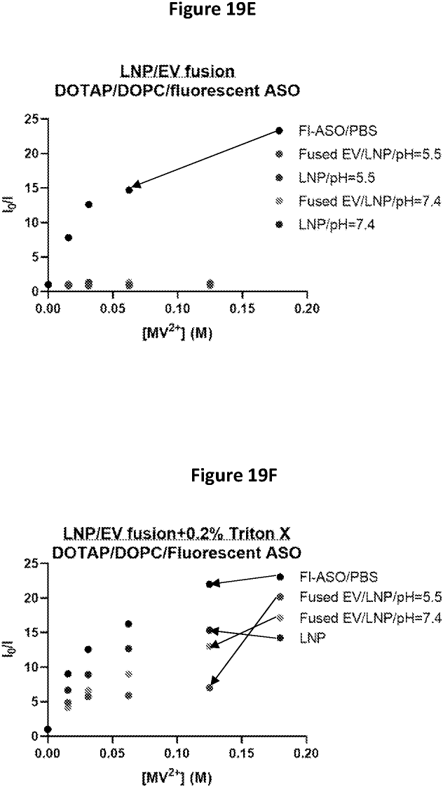

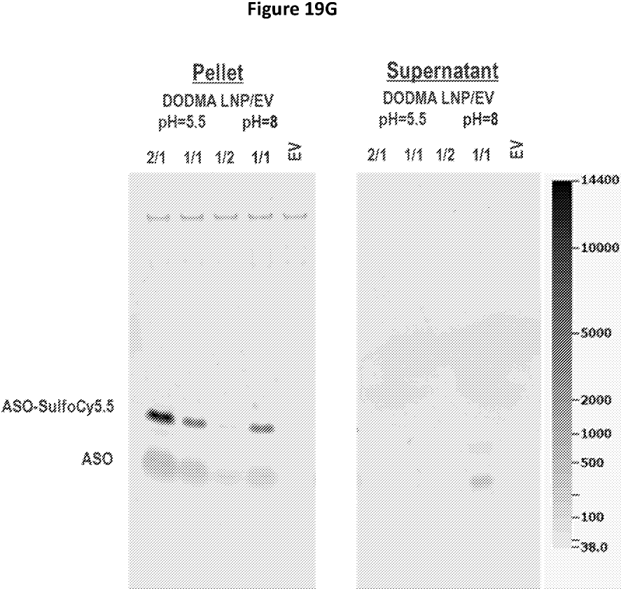

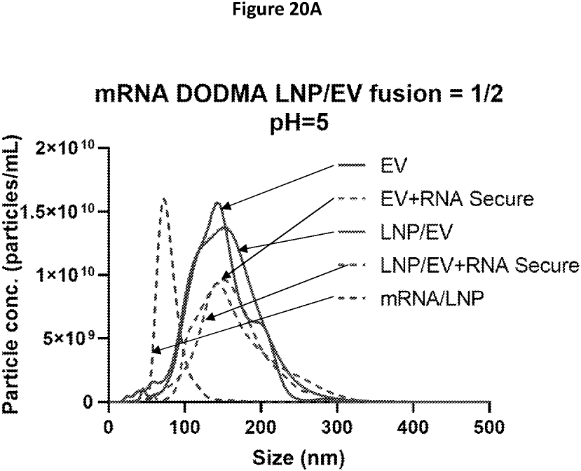

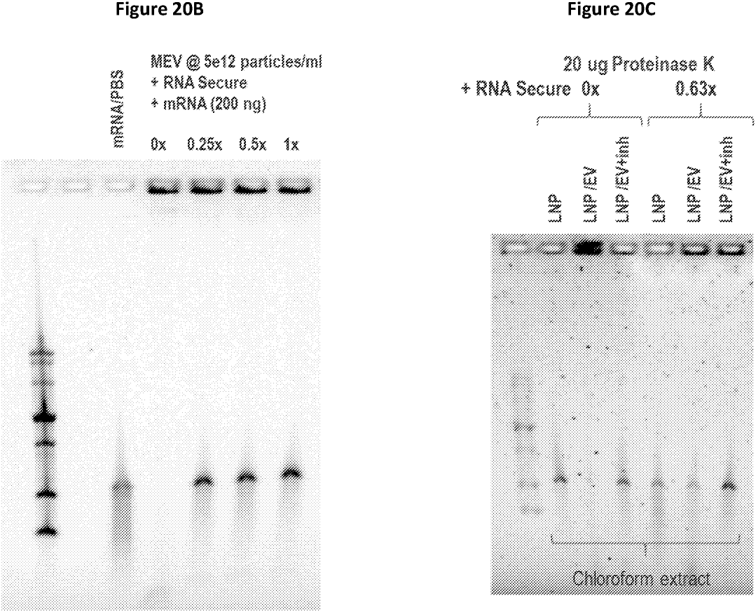

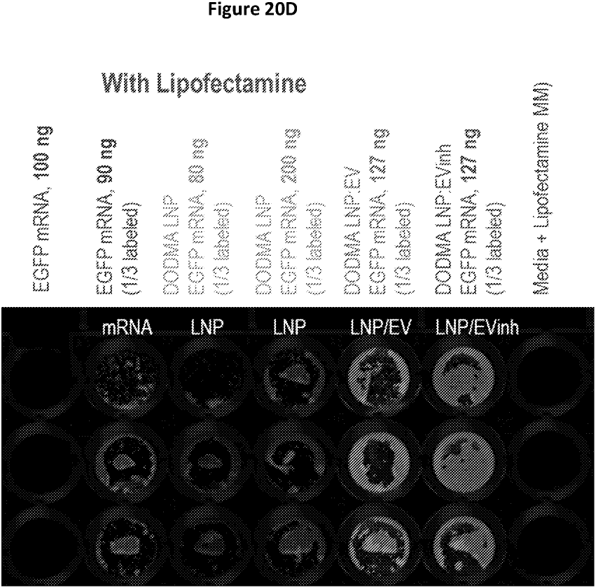

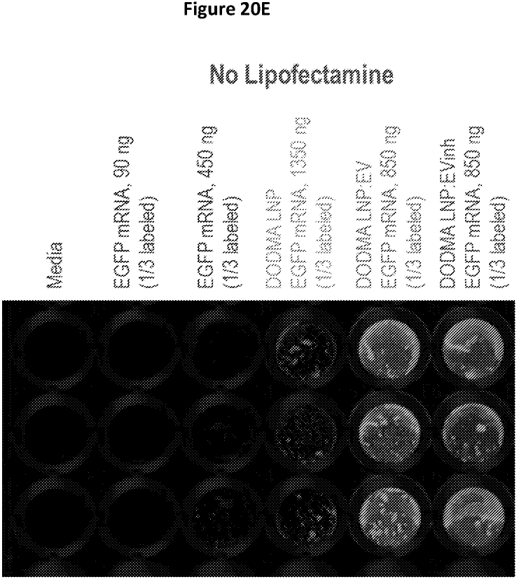

Following similar procedures as disclosed herein, ASO (also referred to herein as ON) as a model cargo was first encapsulated into lipid nanoparticles (LNPs) comprising DOTAP or DODMA, a helper lipid (DOPC or DSPC), and optionally cholesterol and DSPE-mPEG2000 (e.g., DODMA or DOTAP/Cholesterol/DOPC/DSPE-PEG2k at 50/38.5/10/1.5 mol %). After fusion, the particles were subject to RCA precipitation and presence of the ASO in the supernatant and pellet was analyzed by electrophoresis. The results are showin in FIGS. 19A and 19B. Fully transferred ASO from LNP to MEVs upon fusion (~ 1,400 ASOs per EV) as evaluated by RCA precipitation. ASO in LNPs alone were found in the supernatant and not in the pellet (no glycocalyx). MEVs were pulled in the pellet. Similar results were observed in RCA-Dyna beads pull-down assay as shown in FIGS. 19C and 19D.

Results from an MV²⁺ quenching assay also confirmed encapsulation of ASO into EVs via fusion. See FIGS. 19E and 19F. Fluorescence is quenched outside by MV²⁺ but not inside since MV²⁺ does not cross the membrane.

Lectin pull-down assay was performed at various ASO concentrations and pH and the results are shown in FIG. 19G and Table 33.

TABLE 33

Results from Lectin Pull-Down Assay

| pH | LNP/EV | LNP Volume (ul) | LNP Particle cone | EV Volume (ul) stock (2.2 e13 | EV Particle conc after mixing |
|---|---|---|---|---|---|
| 5.5 | 2/1 | 300 | 1.7 e12 | 9 | 1e12 |
| 5.5 | 1/1 | 300 | 1.0 e12 | 9 | 1e12 |
| 5.5 | ½ | 300 | 0.5 e12 | 9 | 1e12 |
| 8 | 1/1 | 300 | 1.0 e12 | 9 | 1e12 |

### Example 10: Preparation of AAV-Loaded Milk Extracellular Vesicles (MEV)

AAV-loaded MEVs are prepared through a two-step process: (1) liposome loading of AAV particles, and (2) fusion of AAV-loaded liposomes with milk vesicles.

Liposomes comprising of DOPC (60 mol%), Cholesterol (20 mol%), DOTAP (10 mol%), DOPE (10 mol%) re prepared via extrusion. All the components are dissolved in choroform in a 2 and ram glass vial. The chloroform is evaporated under a stream of air while the vial is manually rotated in order to form a thin film on the walls of the vial. The lipid film is dried under vacuum to remove trace amounts of chloroform. The lipid film is hydrated with PBS, pH=7.4, then adding AAV particles. The mixed suspension is vortexed followed by extrusion. The extrusion is done using Avanti Polar Lipids extruder with 100 nm Polycarbonate Membranes.

Fresh raw milk was defatted using centrifugation 7-20 kg for 20-40 minutes. Casein was coagulated in raw milk (or defatted milk) using vegetable rennet. Coagulated casein was removed following the standard procedure. The resultant EVs were washed and concentrated using tangential flow filtration. The permeate was further purified using size exclusion chromatography and the resultant EV composition was collected. The AAV-carrying liposomes is then fused with MEV suspension through incubation.

### Example 11: AAV Encapsulation Using Aqueous Suspension of Cationic Lipids

An aqueous suspension comprising DOTAP was used as a cationic lipid to bind to negatively charged AAVs for producing lipid vesicles loaded with AAV particles. In addition to DOTAP, the aqueous suspension further comprises DSPC as a helper lipid and cholesterol for providing rigidity to the lipid coat, as well as mPEG-DSPE to provide colloidal stability to the lipid coated AAVs. The lipid compositions are provided in Table 35 below:

TABLE 35

Lipid Compositions

| Lipid Mix | MW | Mol Ratio | Total Lipid (µmol) | µmole | Weight (µg) | Stock (µg/mL) | Vol (µL) |
|---|---|---|---|---|---|---|---|
| DSPC | 789.63 | 10 | 2 | 0.2 | 157.93 | 10000 | 15.8 |
| DOTAP | 697.58 | 50 | | 1 | 697.58 | 10000 | 69.8 |
| Cholesterol | 386.86 | 39 | | 0.78 | 301.7508 | 10000 | 30.18 |
| DSPE-PEG | 2805.5 | 1 | | 0.02 | 56.11 | 10000 | 5.61 |

The concentration of the lipid-AAV particles thus formed was measured by NTA. The lipid-AAV particles were mixed with milke exosome vesicles (MEVs) at a 1:1 particle concentration, vortexed, and then incubated at 40° C. for 2 hours with mixing to facilitate fusion. The lipid-AAVMEV fusion was performed using a 5-channel linear flow chip and the fusion conditions are provided in Table 36 below.

TABLE 36

Formulation Conditions

| | Flow Rate Ratio (FRR) | | Total Flow Rate (TFR) |
|---|---|---|---|
| Formulation | AAV | Aq Lipid | µL/min |
| 4 µmoles lipid | 2 | 1 | 150 |
| 2 µmoles lipid | 2 | 1 | 150 |
| | 1 | 1 | 100 |
| 1 µmoles lipid | 2 | 1 | 150 |
| | 1 | 1 | 100 |

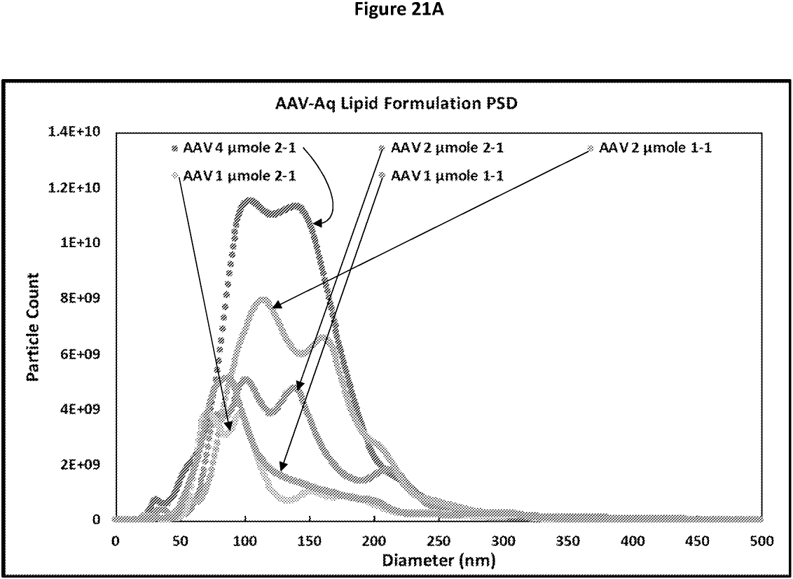

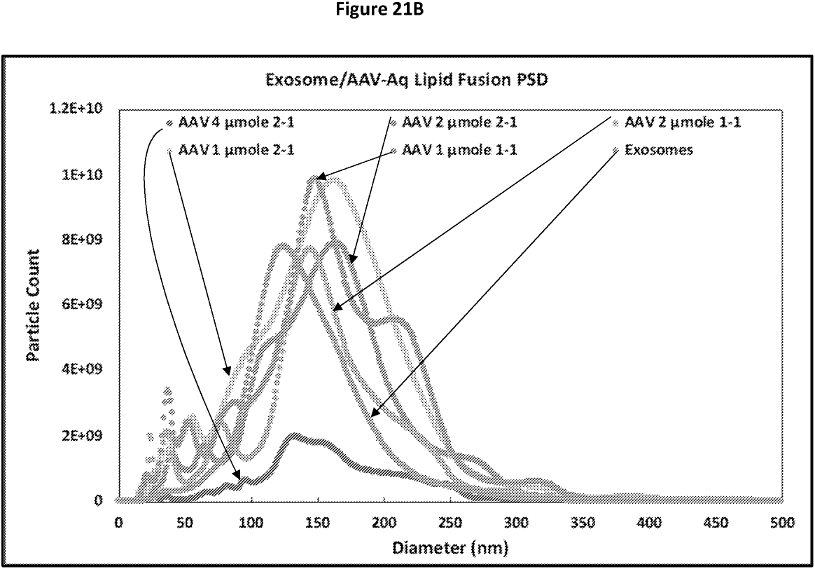

The fused sample was stored overnight at 4° C. Particle concentration and size were measured by NTA. Particle size distribution of AAV mixed with lipid suspension has bimodal distribution not typical for liposomes, indicating that some of the liposomes effectively encapsulated AAV. Compare FIG. 21A with FIG. 21B. AAV infectivity and transduction capability were confirmed in an in vitro HEK cell system.

### Example 12: PEG-Mediated Fusion Between Liposome and MEV as Assessed by FRET Assay

### Liposome Formulations

Four different composition of liposomes were prepared by lipid film rehydration and extrusion method: (1) 67% POPC, 30% DOPE, 1.5% NBD-PS, 1.5% Rho-PE; (2) 62% POPC, 30% DOPE, 1.5% NBD-PS, 1.5% Rho-PE, 5% PEG 2000-PE; (3) 50% DOTAP, 47% DOPE, 1.5% NBD-PS, 1.5% Rho-PE; (4) 50% DOTAP, 42% DOPE, 1.5% NBD-PS, 1.5% Rho-PE, 5% PEG 2000-PE. The final lipid concentration was 1mM for all liposome formulations. The lipid mixture was dissolved in chloroform and a dry lipid film was prepared by evaporation with a rotatory evaporator under reduced pressure at 60 C. The lipid film was rehydrated with 1x PBS and vortexed vigorously at room temperature for 1 hour. The formulation was extruded seven times through polycarbonate membrane (0.1 um) by Lipex.

### FRET-Based Liposome-WEV Assay

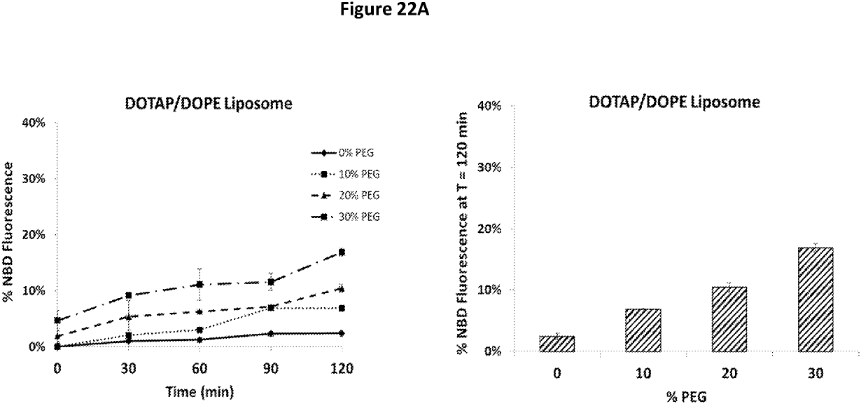

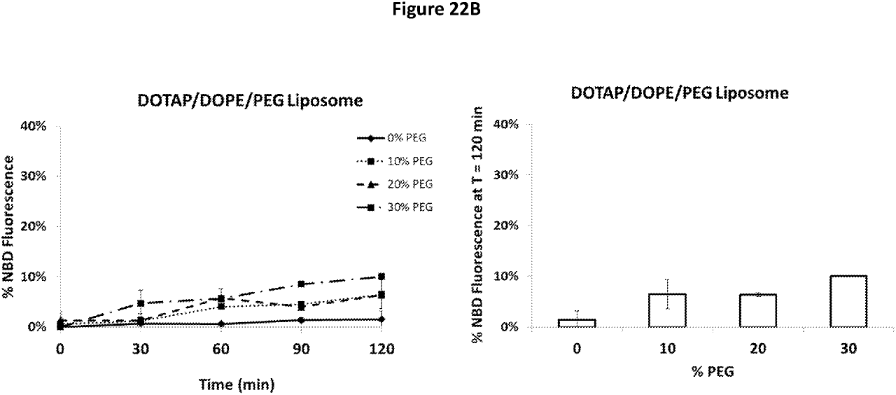

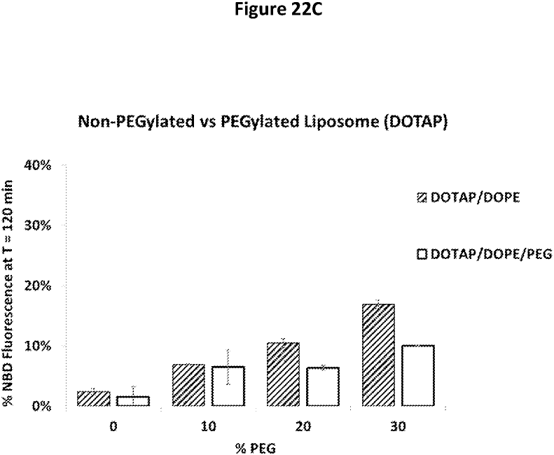

Each liposome and WEV were incubated in 8-ml clear vial maintained 40 C with continuous stirring. Liposome and WEV were mixed at 1:1 particle ratio. PEG 8000 was added at a final concentration of 0, 10, 20, and 30 % (w/v). The fusion was monitored by FRET assay by measuring NBD fluorescence with SpectraMax (excitation at 460 nm, emission at 535 nm, cutoff at 530 nm) at t=0, 30, 60, 90, and 120 minutes after starting incubation. NBD fluorescence increase % was calculated by following equation: NBD fluorescence increase (%) = [NBD – Min(NBD)]/[Max(NBD) – Min(NBD)] (Min(NBD) = NBD fluorescence at t=0; Max(NBD) = NBD fluorescence measured after solubilizing all liposome using 2.5% (w/v) DDM). Results are showin in FIGS. 22A-22C.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A vesicle, comprising:
(i) one or more component(s) of a lipid nanoparticle (LNP); and
(ii) one or more component(s) of a milk purified vesicle (MPV); wherein the vesicle is loaded with a cargo.

2. The vesicle of claim 1, wherein the MPV is a whey purified vesicle (WPV).

3. The vesicle of claim 1, wherein the LNP is a liposome, a multilamellar vesicle, or a solid lipid nanoparticle.

4. The vesicle of claim 1, wherein the LNP comprises one or more cationic lipids.

5. The vesicle of claim 4, wherein the one or more cationic lipids are non-ionizable cationic lipids.

6. The vesicle of claim 6, wherein the one or more non-ionizable cationic lipids are selected from the group consisting of DOTAP, DODAC, DOTMA, DDAB, DOSPA, DMRIE, DORIE, DOMPAQ, DOAAQ, DC-6-14, DOGS, and DODMA-AN.

7. The vesicle of claim 4, wherein the one or more cationic lipids are ionizable cationic lipids.

8. The vesicle of claim 7, wherein the one or more ionizable cationic lipids are selected from the group consisting of KL10, KL22, DLin-DMA, DLin-K-DMA, DLin-MC3-DMA, DLin-KC2-DMA, DODAP, DODMA, and DSDMA.

9. The vesicle of claim 1, wherein the LNP comprises one or more phospholipids.

10. The vesicle of claim 9, wherein the one or more phospholipids are selected from the group consisting of:
1,2-Dioleoyl-sn-glycero-3-phosphocholine (DOPC),
1,2-distearoyl-sn-glycero-3-phosphocholine (DSPC),
1,2-Dioleoyl-sn-glycero-3-phosphoserine (DOPS),
PEG-1,2-Distearoyl-sn-glycero-3-phosphoethanolamine (PEG-DSPE),
1,2-Dioleoyl-sn-glycero-3-phosphoethanolamine (DOPE),
1,2-Dipalmitoyl-sn-glycero-3-phosphoethanolamine-PEG,
1,2-Bis(diphenylphosphino)ethane (DPPE)-PEG
GL67A-DOPE-DMPE-PEG, and
any combination thereof.

11. The vesicle of claim 1, wherein the LNP comprises cholesterol, or DC-cholesterol.

12. The vesicle of claim 1, wherein the LNP comprises:
(a) about 50 mol % to about 70 mol % of DOPC,
(b) about 10 mol % to about 50 mol % of cholesterol,
(c) about 5 mol % to about 50 mol % of DOTAP and/or DODMA,
(d) about 5 mol % to about 30 mol % of DOPE, DSPC, and/or DOPC,
(e) about 0.5-10 mol % of DPPC-PEG and/or DSPE-PEG; or
(f) a combination thereof.

13. The vesicle of claim 1, wherein the LNP comprises about 50 mol % to about 70 mol % of DOPC, about 10 mol % to about 30 mol % of cholesterol, about 5 mol % to about 15 mol % of DOTAP, about 5 mol % to about 15 mol % of DOPE, and about 0.5 mol % to about 3.0 mol % of DPPE-PEG2000.

14. The vesicle of claim 1, wherein the LNP comprises about 10-50 mol% of a cationic lipid, about 20-40 mol% cholesterol, and about 0.5-3.0 mol% lipid-mPEG2000.

15. The vesicle of claim 14, wherein the cationic lipid is DOTAP or DODMA.

16. The vesicle of claim 13, wherein the lipid in the lipid-mPEG2000 is DSPE, DMPE, DMPG, or a combination thereof.

17. The vesicle of claim 1, wherein the LNP further comprises a dye-conjugated helper lipid at about 0.2-1 mol%.

18. The vesicle of claim 17, wherein the helper lipid is DPPE.

19. The vesicle of claim 1, wherein the lipid content in the LNP is substantially similar to the lipid content in the MPV.

20. The vesicle of claim 1, wherein the vesicle further comprises one or more binding moieties on the surface of the vesicle.

21. The vesicle of claim 20, wherein the binding moiety is a lectin.

22. The vesicle of claim 21, wherein the lectin is selected from the group consisting of Con A, RCA, WGA, DSL, Jacalin, and any combination thereof.

23. The vesicle of claim 21, wherein the lectin is covalently attached to the vesicle surface.

24. The vesicle of claim 21, wherein the lectin is attached to the vesicle surface through a biotin-streptavidin linkage.

25. The vesicle of claim 1, wherein the size of the MPV is about 20-1,000 nm, optionally wherein the size of the MPV is about 80-200 nm, or about 100-160 nm.

26. The vesicle of claim 1, wherein the MPV comprises a lipid membrane, to which one or more proteins are associated.

27. The vesicle of claim 26, wherein the one or more proteins associated with the lipid membrane of the MPV comprises Butyrophilin Subfamily 1 Member A1 (BTN1A1) or a transmembrane fragment thereof, Butyrophilin Subfamily 1 Member A2 (BTN1A2) or a transmembrane fragment thereof, fatty acid binding protein, lactadherin, platelet glycoprotein 4, xanthine dehydrogenase, ATP-binding cassette subfamily G, perilipin, RAB1A, peptidyl-prolyl cis-transisomerase A, Ras-related protein Rab-18, EpCAM, CD63, CD81, TSG101, HSP70, lactoferrin or a transmembrane fragment thereof, ALG-2-interacting protein X, alpha-lactalbumin, serum albumin, polymeric immunoglobulin, lactoperoxidase, or a combination thereof.

28. The vesicle of claim 27, wherein the MPV comprises BTN1A1 CD81, and/or XOR.

29. The vesicle of claim 27, wherein the one or more proteins associated with the lipid membrane of the MPVs comprise glycans attached to glycoproteins and/or glycolipids.

30. The vesicle of claim 1, wherein the MPV is obtained from cow milk, goat milk, camel milk, buffalo milk, yak milk, or human milk.

31. The vesicle of claim 1, wherein the MPV is selected from the group consisting of lactosome, milk fat globule (MFG), exosome, extracellular vesicles, whey-particle, whey-derived particle, aggregates thereof, and combinations thereof.

32. The vesicle of claim 1, wherein the MPVs comprise one or more of the following features:
(i) stability under freeze-thaw cycles and/or temperature treatment;
(ii) colloidal stability when the MPVs are loaded with the biological molecule;
(iii) a loading capacity of at least 5000 cholesterol modified oligonucleotides per MPV;
(iv) stability under acidic pH;
(v) stability upon sonication;
(vi) resistance to enzyme digestion; and
(vii) resistance to nuclease treatment upon loading of the MPVs with oligonucleotides.

33. The vesicle of claim 32, wherein the acidic pH of (iv) is ≤ 4.5, optionally wherein the acidic pH of (iv) is ≤ 2.5.

34. The vesicle of claim 33, wherein the enzyme digestion of (vi) comprises digestion by one or more digestive enzymes.

35. The vesicle of claim 1, wherein the cargo is a peptide, a protein, a nucleic acid, a polysaccharide, or a small molecule.

36. The vesicle of claim 1, which is stable at pH ≤ 4.5, or pH ≤2.5.

37. The vesicle of claim 1, which is resistant to digestive enzymes.

38. The vesicle of claim 1, which is suitable for oral administration.

39. The vesicle of claim 1, comprising BTN1A1, CD81, XOR, or a combination thereof.

40. A composition comprising a vesicle of claim 1, wherein the composition is formulated into a pharmaceutical composition, which further comprises a pharmaceutically acceptable carrier.

41. The composition of claim 40, wherein the composition is formulated for oral administration.

42. A method of preparing cargo-loaded vesicle comprising LNP and MPV (LNP-MPV), the method comprising:
(i) contacting a LNP comprising a cargo with a MPV, thereby causing fusion of the LNP and the MPV to produce LNP-MPV loaded with the cargo; and
(ii) collecting the LNP-MPV loaded with the cargo.

43. The method of claim 42, further comprising (iii) attaching a targeting moiety to the LNP-MPV loaded with the cargo.

44. The method of claim 42, wherein the LNP is a liposome, a multilamellar vesicle, or a solid lipid nanoparticle.

45. The method of claim 42, wherein step (i) is performed for at least one hour at a temperature of about 4° C. to about 50° C., optionally wherein step (i) is performed for at least one hour at a temperature of about 35° C. to about 45° C.

46. The method of claim 42, wherein step (i) is performed in a solution comprising about 5 to about 40% (w/v) polyethylene glycol (PEG).

47. The method of claim 46, wherein the solution comprises about 10% to about 35% (w/v) PEG, optionally wherein the solution comprises about 20% to about 30% (w/v) PEG.

48. The method of claim 46, wherein the PEG in the solution has an average molecular weight of about 6 kD to about 12 kD, optionally wherein the PEG in the solution has an average molecular weight of about 8 kD to about 10 kD.

49. The method of claim 42, wherein the LNP comprises polyethylene glycol (PEG).

50. The method of claim 42, wherein the LNP comprises one or more of cationic lipids.

51. The method of claim 50, wherein the one or more cationic lipids are ionizable cationic lipids.

52. The method of claim 51, wherein the one or more ionizable cationic lipids are selected from the group consisting of KL10, KL22, DLin-DMA, DLin-K-DMA, DLin-MC3-DMA, DLin-KC2-DMA, DODAP, DODMA, and DSDMA.

53. The method of claim 42, wherein the one or more cationic lipids are non-ionizable cationic lipids.

54. The vesicle of claim 53, wherein the one or more non-ionizable cationic lipids are selected from the group consisting of DOTAP, DODAC, DOTMA, DDAB, DOSPA, DMRIE, DORIE, DOMPAQ, DOAAQ, DC-6-14, DOGS, and DODMA-AN.

55. The method of claim 49, wherein the lipid nanoparcle comprises one or more phospholipids.

56. The method of claim 55, wherein the one or more phospholipids are selected from the group consisting of:
1,2-Dioleoyl-sn-glycero-3-phosphocholine (DOPC),
1,2-distearoyl-sn-glycero-3-phosphocholine (DSPC),
1,2-Dioleoyl-sn-glycero-3-phosphoserine (DOPS),
PEG-1,2-Distearoyl-sn-glycero-3-phosphoethanolamine (PEG-DSPE),
1,2-Dioleoyl-sn-glycero-3-phosphoethanolamine (DOPE),
1,2-Dipalmitoyl-sn-glycero-3-phosphoethanolamine-PEG,
1,2-Bis(diphenylphosphino)ethane (DPPE)-PEG
GL67A-DOPE-DMPE-PEG, and
any combination thereof.

57. The method of claim 42, wherein the LNP comprises cholesterol, or DC-cholesterol.

58. The method of claim 42, wherein the LNP comprises:
(a) about 50 mol % to about 70 mol % of DOPC,
(b) about 10 mol % to about 50 mol % of cholesterol,
(c) about 5 mol % to about 50 mol % of DOTAP and/or DODMA,
(d) about 5 mol % to about 30 mol % of DOPE, DSPC, and/or DOPC,
(e) about 0.5-10 mol % of DPPC-PEG and/or DSPE-PEG; or
(f) a combination thereof.

59. The method of claim 42, wherein the LNP comprises about 50 mol % to about 70 mol % of DOPC, about 10 mol % to about 30 mol % of cholesterol, about 5 mol % to about 15 mol % of DOTAP, from about 5 mol % to about 15 mol % of DOPE, and about 0.5 mol % to about 3.0 mol % of DPPE-PEG2000.

60. The method of claim 42, wherein the LNP comprises about 10-50 mol% of a cationic lipid, about 20-40 mol% cholesterol, and about 0.5-3.0 mol% lipid-mPEG2000.

61. The method of claim 60, wherein the cationic lipid is DOTAP or DODMA.

62. The method of claim 60, wherein the lipid in the lipid-mPEG2000 is DSPE, DMPE, DMPG, or a combination thereof.

63. The method of claim 42, wherein the LNP further comprises a dye-conjugated helper lipid at about 0.2-1 mol%.

64. The method of claim 63, wherein the helper lipid is DPPE.

65. The method of claim 42, wherein the lipid content in the LNP is substantially similar to the lipid content in the MPVs.

66. The method of claim 42, wherein the MPVs comprise a negative (-) electrostatic charge and the lipid particle comprises a positive (+) electrostatic charge.

67. The method of claim 42, wherein the LNP is a neutral LNP.

68. The method of claim 67, wherein the neutral LNP comprises one or more of neutral lipids selected from the group consisting of DPPC, DOPC, DOPE, and SM.

69. The method of claim 42, wherein the LNP comprising the cargo is produced by a process comprising: mixing an alcohol solution comprising one or more lipids and an aqueous solution comprising the cargo to form the cargo-loaded LNP.

70. The method of claim 69, wherein in the mixing step, the alcohol solution comprising one or more lipids contacts the aqueous solution comprising the cargo at a T junction or a Y junction in one or more tubes, which are connected to one or more pumps, optionally wherein the one or more tubes have a diameter of about 0.2-2 mm.

71. The method of claim 70, wherein the mixing step is performed using a microfluidic device, wherein optionally the microfluidic device comprises one or more channels having a diameter of about 0.02-2 mm, and/or optionally the microfluidic device comprises glass and/or polymer materials.

72. The method of claim 42, wherein the LNP comprising the cargo is produced by a process comprising: rehydrating a lipid film with a solution comprising the cargo followed by vortexing, sonication, extrusion, or a combination thereof.

73. The method of claim 42, wherein step (i) comprises extruding a suspension comprising the LNP and the MPVs through a filter under pressure.

74. The method of claim 73, wherein the filter is a polycarbonate membrane filter having a pore size of about 50 nm to about 200 nm.

75. The method of claim 42, wherein step (i) comprises sonication.

76. The method of claim 42, wherein step (i) is performed using a microfluidic device, wherein optionally the microfluidic device comprises one or more channels having a diameter of about 0.02-2 mm, and/or optionally the microfluidic device comprises glass and/or polymer materials.

77. The method of claim 42, wherein in step (ii), the LNP-MPVs are collected by positive selection.

78. The method of claim 42, wherein in step (ii), the LNP-MPVs are collected by negative selection.

79. The method of claim 49 where step (ii) is performed using a lectin to collect the LNP-MPVs.

80. The method of claim 79, wherein the lectin is selected from the group consisting of Con A, RCA, WGA, DSL, Jacalin, and any combination thereof.

81. The method of claim 42, wherein step (ii) comprises ion-exchange chromatagraphy and/or affinity chromatography.

82. The method of claim 42, wherein the method further comprises (iii) modifying the cargo-loaded LNP-MPV collected in step (ii) to attach a target moiety that binds gut cells, optionally small intestinal cells.

83. The method of claim 42, wherein the MPV comprises a lipid membrane to which one or more proteins are associated, and wherein the MPV comprises a relative abundance of casein less than about 40%, and/or a relative abundance of lactoglobulin less than about 25%.

84. The method of claim 83, wherein the relative abundance of casein in the composition is less than about 20%, optionally wherein the relative abundance of casein in the composition is less than about 5%.

85. The method of claim 84, wherein the MPV is substantially free of casein.

86. The method of claim 42, wherein the MPV comprises a relative abundance of lactoglobulin less than about 15%, optionally wherein the relative abundance of lactoglobulin in the composition is less than about 10%.

87. The method of claim 42, wherein the size of the MPV is about 20-1,000 nm.

88. The method of claim 87, wherein the size of the MPV is about 80-200 nm, optionally about 100-160 nm.

89. The method of claim 42, wherein the MPV comprises a lipid membrane to which one or more proteins are associated, optionally wherein the one or more proteins comprise Butyrophilin Subfamily 1 Member A1 (BTN1A1) or a transmembrane fragment thereof, Butyrophilin Subfamily 1 Member A2 (BTN1A2) or a transmembrane fragment thereof, fatty acid binding protein, lactadherin, platelet glycoprotein 4, xanthine dehydrogenase, ATP-binding cassette subfamily G, perilipin, RAB1A, peptidyl-prolyl cis-transisomerase A, Ras-related protein Rab-18, EpCAM, CD63, CD81, TSG101, HSP70, lactoferrin or a transmembrane fragment thereof, ALG-2-interacting protein X, alpha-lactalbumin, serum albumin, polymeric immunoglobulin, lactoperoxidase, or a combination thereof.

90. The method of claim 89, wherein the MPV comprises BTN1A1 CD81, and/or XOR.

91. The method of claim 89, wherein the one or more proteins associated with the lipid membrane of the MPV comprise glycans attached to glycoproteins and/or glycolipids.

92. The method of claim 42, wherein the MPV is obtained from cow milk, goat milk, camel milk, buffalo milk, yak milk, or human milk.

93. The method of claim 42, wherein the MPVs are selected from the group consisting of lactosome, milk fat globule (MFG), exosome, extracellular vesicles, whey-particle, whey-derived particle, aggregates thereof, and combinations thereof.

94. The method of claim 42, wherein the cargo is a peptide, a protein, a nucleic acid, a polysaccharide, or a small molecule.

95. The method of claim 94, wherein the cargo comprises a targeting moiety.

96. The method of claim 95, wherein the targeting moiety is a compound comprising at least one N-acetylgalactosamine (GalNAc) moiety, folate, an antibody, which optionally is a Fab fragment, a nucleic acid aptamer, a RGD peptide, or a lectin.

97. The method of claim 42, wherein the MPV comprises one or more of the following features:
(i) stability under freeze-thaw cycles and/or temperature treatment;
(ii) colloidal stability when the MPVs are loaded with the biological molecule;
(iii) a loading capacity of at least 5000 cholesterol modified oligonucleotides per MPV;
(iv) stability under acidic pH;
(v) stability upon sonication;
(vi) resistance to enzyme digestion; and
(vii) resistance to nuclease treatment upon loading of the MPVs with oligonucleotides.

98. The method of claim 97, wherein the acidic pH of (iv) is ≤ 4.5, optionally wherein the acidic pH of (iv) is ≤ 2.5.

99. The method of claim 98, wherein the enzyme digestion of (vi) comprises digestion by one or more digestive enzymes.

100. A method of loading MPVs with a cargo, the method comprising:
(i) contacting a LNP comprising a cargo with a composition comprising MPVs, wherein the MPVs are modified as compared to their natural counterpart MPVs, thereby causing fusion of the LNP and the modified MPVs to produce LNP-MPVs loaded with the cargo; and
(ii) collecting the LNP-MPVs loaded with the cargo.

101. The method of claim 100, wherein the LNP-MPVs are stable at pH ≤4.5, or pH ≤ 2.5.

102. The method of claim 100, wherein the LNP-MPVs are resistant to digestive enzymes.

103. The method of claim 100, wherein the LNP-MPVs are suitable for oral administration.

104. The method of claim 100, wherein the LNP-MPVs comprise BTN1A1, CD81, XOR, or a combination thereof.

105. The method of claim 100, wherein the LNP is set forth in any one of claims 52-68.

106. A vesicle, which is produced by a method of claim 42.

107. A pharmaceutical composition comprising the vesicle of claim 106 and a pharmaceutically acceptable carrier.

108. The pharmaceutical composition of claim 107, which is formulated for oral administration.
